(19) United States
(12) Patent Application Publication
(10) Pub. No.: US 2003/0148298 A1
(43) Pub. Date: Aug. 7, 2003

(54) Methods for diagnosing and treating systemic lupus erythematosus disease and compositions thereof

(76) Inventors: Margot O''Toole, Newton, MA (US); Holly Legault, Woburn, MA (US)

Correspondence Address: MINTZ, LEVIN, COHN, FERRIS, GLOVSKY and POPEO, P.C., One Financial Center, Boston, MA, 02111, US

(21) Appl. No.: 10/115,717

(22) Filed: Apr. 3, 2002

Related U.S. Application Data
(60) Provisional application No. 60/281,515, filed on Apr. 3, 2001.
(60) Provisional application No. 60/281,086, filed on Apr. 3, 2001.

(51) Int. Cl. C12Q 1/68; G01N 33/567

(52) U.S. Cl. 435/6; 435/7.21

(57) ABSTRACT

The present invention is directed to novel methods for diagnosis and prognosis of Systemic lupus erythematosus by identifying differentially expressed genes. Moreover, the present invention is also directed to methods that can be used to screen test compounds and therapies for the ability to inhibit systemic lupus erythematosus. Additionally, methods and molecule targets (genes and their products) for therapeutic intervention in systemic lupus erythematosus are described.

### RELATED APPLICATIONS

This application claims priority to U.S. Ser. No. 60/281,515, filed Apr. 3, 2001. The contents of this application are incorporated herein by reference in their entirety.

### FELD OF THE INVENTION

The present invention is directed to novel methods for diagnosis and prognosis of Systemic Lupus Erythematosus by identifying differentially expressed genes. The present invention is further directed to methods and molecular targets (genes and their products) for therapeutic intervention in systemic lupus erythematosus. In particular, the present invention is directed to a method of modulating the expression levels of genes associated with systemic lupus erythematosus by administration of rapamycin or antibodies to B7 molecules.

### BACKGROUND OF THE INVENTION

Systemic Lupus Erythematosus (SLE) is a chronic automimmmune disorder in which patients suffer a number immunological abnormalities that is not specific to any one organ. SLE is manifested in various forms, including facial lesions, nephritis, endocarditis, hemolytic anemia and leukopenia. Specifically, SLE has been linked to disruption of complex T-cell mediated pathways, thus presenting a challenge to researchers attempting to elucidate the mechanism of the disease.

Many immunological phenomena are connected to SLE. In SLE patients, antibodies form against certain endogenous antigens, such as the basement membrane of the skin, against lymphocytes, erythrocytes and nuclear antigens. Antibodies may be directed against double-stranded DNA (ds-DNA) to form complexes, which are then deposited together on small blood vessels, resulting in vasculitis. These deposits are especially dangerous when they occur on the renal glomeruli, and may lead to glomerulonephritis and kidney failure. The incidence of clinically detectable involvement of the kidneys ranges from 50 to 80%.

T cells react against endogenous antigens in SLE patients. In order for T-lymphocytes to respond, antigen-presenting cells (APCs) must provide two signals to trigger resting T cells. (Jenkins, M. and Schwarts, R. J. Exp. Med. 165: 302-319 (1987); Mueller D. L. et al, J. Immunol. 144: 3701-3709 (1990)). The first signal, which confers specificity to the immune response, is transduced via the T cell receptor (TCR) for antigenic peptide presented in the context of the major histocompatibility complex (MHC). The second signal, termed costimulation, induces T cells to proliferate and become functional (Lenschow et al., Annu. Rev. Immunol. 14:233 (1996)). Unlike the first signal pathway, costimulation is neither antigen-specific nor MHC restricted, and is thought to be provided by one or more distinct cell surface molecules expressed by APCs. (Jenkins. M. K., et al., J. Immunol., 140:3324-3330 (1988); Linsley, P. S., et al. J. Exp. Med. 173: 721-730 (1991); Gimmi, C. D. et al., Proc.Natl.Acad.Sci. 88:6575-6579 (1991); Young, J. W. et al J. Clin. Invest. 90:229-237(1992); Koulova et al. J. Exp. Med. 173: 759-762 (1991); Reiser, H. et al, Proc. Natl. Acad. Sci 89:271-275 (1992); van Seventer, G. A. et al., J. Immunol. 144: 4579-4586 (1990); LaSalle, J. M. et al., J. Immunol. 147: 774-80 (1991); Dustin, M. I. et al, J. Exp. Med. 169: 503( 1989); Armitage, R. J. et al. Nature 357: 80-82 (1992); Liu, Y. et al. J. Exp. Med. 715: 437-445 (1992). It is widely believed that genes involved in regulating T cell response play a critical role in patients suffering from SLE.

The CD80 (B7-1) and CD86 (B7) proteins, expressed on APCs, are critical molecules in the costimulatory pathway as shown in two mouse models of autoimmune kidney disease, a model believed to be analogous to human SLE. Sypek et al. (Freeman et al. J. Exp.Med. 174: 625(1991); Freeman et al., J.Immunol. 143:2714(1989); Azuma et al. Nature 366:76 (1993); Freeman et al. Science 262: 909 (1993)). B7 appears to play a predominant role during primary immune responses, while B7-1, which is upregulated later in the course of an immune response, may be important in prolonging primary T cell responses or costimulating secondary T cell responses (Bluestone, Immunity 2:555 (1995)). One receptor to which B7-1 and B7 bind, CD28, is constitutively expressed on resting T cells and increases in expression after activation. After signaling through the T cell receptor, ligation of CD28 and transduction of a costimulatory signal induces T cells to proliferate and secrete IL-2. (Linsley, P. S., et al. J.Exp.Med. 173:721-730 (1991); Gimmi, C. D. et al. Proc.Natl.Acad.Sci. 88:6575-6579 (1991); June, C. J. et al. Immunol. Today 11:211-6 (1990); Harding, F. A., et al. Nature 356: 607-609 (1992)). A second receptor, termed CTLA4 (CD152) is homologous to CD28 but is not expressed on resting T cells and appears following T cell activation (Brunet, J. F. et al., Nature 328, 267-270 (1987)). CTLA4 appears to be critical in negative regulation of T cell responses. (Waterhouse et al, Science 270-985 (1995)). Blockade of CTLA4 has been found to remove inhibitory signals, while aggregation of CTLA4 has been found to provide inhibitory signals that downregulate T cell responses (Allison and Krummel, Science 270: 932 (1995)). The B7 molecules have a higher affinity for CTLA4 than for CD28 (Linsley, P. S. et al, J.Exp.Med. 174: 561-569 (1991)), and B7-1 and B7 have been found to bind to distinct regions of the CTLA4 molecules and have different kinetics of binding to CTLA4 (Linsley et al. immunity, 1:793 (1994)). If T-cells are only stimulated through the T cell receptor, without receiving an additional costimulatory signal, they become nonresponsive, anergic, or die, resulting in downmodulation of the immune response.

In addition, a new molecule related to CD28 and CTLA4, ICOS, has been identified and seems important in IL-10 production. (Hutloff et al., Nature 397:263 (1999); WO 98/38216; Tamatani, T. et al, Int. Immunol. 12:51-55). The ICOS ligand, GL50 has also been identified (also called by the names ICOSL, B7h, LICOS, and B7RP-1) which is a new B7 family member (Ling, V et al, J. Immunol. 164:1653-7 (2000); Swallow, M. M. et al Immunity 11:423-432 (1999); Aicher, A. et al, J. Immunol. 164:4689-96 (2000); Mages, H. W. et al, Eur. J. Immunol. 30:1040-7 (2000); Brodie, D. et al, Curr. Biol. 10:333-6 (2000); Yoshinaga, S. K. et al., Nature 402:827-32 (1999)). Moreover, an additional B7 family member, B7-H1, also known as PD-LI, interacts with the immunoinhibitory receptor PD-1 (Freeman, G. J. et al., J. Exp. Med. 192:1027-34).

The importance of the B7:CD28/CTLA4 costimulatory pathway has been demonstrated in vitro and in several in vivo model systems. Blockade of this costimulatory pathway results in the development of antigen specific tolerance in murine and human systems. (Harding, F. A. et al Nature 356: 607-609 (1992); Lenschow, D. J. et al, Science 257: 789-792 (1992); Turka, L. A. et al. Proc.Natl.Acad.Sci 89:1102-11105 (1992); Gimmi, C. D. et al. Proc.Natl.Acad.Sci. 90:6586-6590 (1993); Boussiotis, V. et al. J.Exp.Med. 178: 1753-1763 (1993)). Conversely, expression of B7 by B7 negative murine tumor cells induces T-cell mediated specific immunity accompanied by tumor rejection and long lasting protection to tumor challenge. (Chen, L. et al Cell 71: 1093-1102 (1992); Towsend, S. E. and Allison, J. P. Science 259: 368-370 (1993); Baskar, S. et al. Proc.Natl.Acad.Sci. 90: 5687-5690 (1993)). Therefore manipulation of the costimulatory pathway offers great potential to stimulate or suppress immune responses in humans.

Systemic lupus erythematosus (SLE) involves the complex interaction of many genes in cell-mediated immune responses. The nature and variability of SLE as expressed in different patients has proven to be a challenge in characterizing the disease and in developing a prognosis for each patient. The present invention therefore addresses these issues by using differentially expressed genes to provide methods for diagnosis, prognosis and for assaying therapeutic intervention.

### SUMMARY OF THE INVENTION

In one embodiment, the invention provides a method of diagnosing a subject with systemic lupus erythematosus by comparing the level of expression of a marker in a sample from a subject, where the marker is selected from the group of markers set forth in Tables 1 and 3-8, to the normal level of expression of the marker in a control sample, where a substantial difference between the level of expression of the marker in the sample from the subject and the normal level is an indication that the subject is afflicted with systemic lupus erythematosus. In a preferred embodiment, the marker corresponds to a transcribed polynucleotide or a portion thereof. Preferably, the marker corresponds to a transcribed polynucleotide or a portion thereof, and the sample is collected from kidney tissue. In another preferred embodiment, the control sample is from non-involved tissue from the subject. Alternatively, the control sample is from the tissue of a nondiseased subject. In a further preferred embodiment, the level of expression of the marker in the sample differs from the normal level of expression of the marker in a subject not afflicted by a factor of at least two, and in an even more preferred embodiment, the expression levels differ by a factor of at least five.

In another preferred embodiment, the level of expression of the marker in the sample is assessed by detecting the presence in the sample of a protein corresponding to the marker. In a particularly preferred embodiment, the presence of the protein is detected using a reagent which specifically binds with the protein. In an even more preferred embodiment, the reagent comprises an antibody or fragments thereof In another preferred embodiment, the method comprises a marker selected from markers listed in Table 3-4, Table 7 or Table 8. In another preferred embodiment, the level of expression of the marker in the sample is assessed by detecting the presence in the sample of a transcribed polynucleotide or portion thereof, where the transcribed polynucleotide includes the marker. In a particularly preferred embodiment, the transcribed polynucleotide is an mRNA or a cDNA.

In yet another preferred embodiment, the level of expression of the marker in the sample is assessed by detecting the presence in the sample of a transcribed polynucleotide or a portion thereof which hybridizes with a labeled probe under stringent conditions, wherein the transcribed polynucleotide comprises the marker.

In another preferred embodiment for diagnosing a subject with systemic lupus erythematosus, the level of expression in the sample of each of a panel of markers independently selected from the markers listed in Tables 1 and 3-8 is compared with the normal level of expression of the same panel of markers in a control sample, where the level of expression of more than one of the markers is substantially different, relative to the corresponding normal levels of expression of the markers, indicating that the subject is afflicted with systemic lupus erythematosus. In a particularly preferred embodiment, the plurality includes at least five of the markers set forth in Tables 1 and 3-8.

In another embodiment, the invention provides a method of monitoring the progression of systemic lupus erythematosus in a subject, including detecting in a subject sample at a first point in time the expression of marker, where the marker is selected from the group including the markers listed in Tables 1 and 3-8, repeating this detection step at a subsequent point in time with the same marker, and detecting a substantial difference between the levels of expression, thus indicating that the subject has progressed to a different stage of systemic lupus erythematosus. In a preferred embodiment, at least 5 markers are selected from the group of markers Tables 1 and 3-8 and combinations thereof. In another preferred embodiment, the marker corresponds to a transcribed polynucleotide or portion thereof, where the polynucleotide includes the marker. In a particularly preferred embodiment, the cells are collected from kidney tissue.

In another embodiment, the invention provides a method of assessing the efficacy of a test compound for inhibiting systemic lupus erythematosus in a subject, including comparing expression of a marker in a first sample obtained from the subject which is exposed to or maintained in the presence of the test compound, where the marker is selected from the group including the markers listed in Tables 1 and 3-8, to expression of the marker in a second sample obtained from the subject, where the second sample is not exposed to the test compound, where a substantially different level of expression of the marker in the first sample relative to that in the second sample is an indication that the test compound is efficacious for inhibiting systemic lupus erythematosus in the subject. In a preferred embodiment, the first and second samples are portions of a single sample obtained from the subject. In a particularly preferred embodiment, the substantially different level of expression is a lower level of expression in the first sample.

In another embodiment, the invention provides a method of assessing the efficacy of a therapy for inhibiting systemic lupus erythematosus in a subject, the method including comparing expression of a marker in the first sample obtained from the subject prior to providing at least a portion of the therapy to the subject, where the marker is selected from the group including the markers listed in Tables 1 and 3-8, to expression of the marker in a second sample obtained from the subject following provision of the portion of the therapy, where a substantially different level of expression of the marker in the second sample relative to the first sample, is an indication that the therapy is efficacious for inhibiting systemic lupus erythematosus in the subject. In a preferred embodiment, the substantially different level of expression is a substantially lower level of expression in the second sample. In a particularly preferred embodiment, the method further comprises a step of comparing expression of the marker in a control sample, where a substantially similar level of expression in the second sample, relative to the control sample, is an additional indication that the test compound is efficacious for inhibiting systemic lupus erythematosus.

In another embodiment, the invention provides a method of screening test compounds for inhibitors of systemic lupus erythematosus in a subject, the method including obtaining a sample including cells from a subject, separately maintaining aliquots of the sample in the presence of a plurality of test compounds, comparing expression of a marker in each of the aliquots, where the marker is selected from the group including the markers listed in Tables 1 and 3-8, and selecting one of the test compounds which induces a substantially different level of expression of the marker in the aliquot containing that test compound, relative to other test compounds. In a particularly preferred embodiment, the substantially different level of expression is a substantially lower level of expression. In an alternative preferred embodiment, the substantially different level of expression is a substantially enhanced level of expression.

In another embodiment, the invention provides a kit for diagnosing a subject with systemic lupus erythematosus, including reagents for assessing expression of a marker selected from the group including the markers listed in Tables 1 and 3-8.

In another embodiment, the invention provides a kit for diagnosing systemic lupus erythematosus in a subject, the kit including a nucleic acid probe where the probe specifically binds with a transcribed polynucleotide corresponding to a marker selected from the group including the markers listed in Tables 1 and 3-8.

In another embodiment, the invention provides a kit for assessing the suitability of each of a plurality of compounds for inhibiting systemic lupus erythematosus, the kit including a plurality of compounds and a reagent for assessing expression of a marker selected from the group including the markers listed in Tables 1 and 3-8.

In another embodiment, the invention provides a kit for diagnosing a subject with systemic lupus erythematosus, the kit including an antibody which specifically binds with a protein corresponding to a marker selected from the group including the markers listed in Tables 1 and 3-8.

In another embodiment, the invention provides a method of modulating the level of expression of a marker selected from the markers listed in Tables 1 and 3-8, the method comprising providing to diseased cells of the subject an antisense oligonucleotide complementary to a polynucleotide corresponding to the marker.

In yet another embodiment, the invention provides a method of modulating the level of expression of a marker selected from the markers listed in Tables 1 and 3-8, the method comprising providing to diseased cells of a subject a protein. In a particularly preferred embodiment, the invention further provides a vector which comprises a polynucleotide encoding the protein.

In another embodiment, the invention provides a method of modulating a level of expression of a marker selected from the markers listed in Tables 1 and 3-8, where the method comprises providing to diseased cells of a subject an antibody. In a preferred embodiment, the method further comprises a therapeutic moiety conjugated to the antibody. In another preferred embodiment the method comprises providing to the diseased cells an additional antibody.

In another preferred embodiment, the invention provides a method of localizing a therapeutic moiety to diseased tissue of a subject comprising exposing the tissue to an antibody which is specific to a protein encoded by a marker listed in Tables 1 and 3-8. Alternatively, the method may be practices by exposing the tissue to a plurality of antibodies which are each specific to a protein encoded by a marker listed in Tables 1 and 3-8.

In another preferred embodiment, the present invention provides a method of screening for a test compound capable of modulating the activity of a protein encoded from a marker listed in Tables 1 and 3-8, said method comprising combining said protein and test compound, and determining the effect of said test compound on the therapeutic efficacy of said protein.

In yet another preferred embodiment, the present invention provides a method of screening for a bioactive agent capable of interfering with the binding of a protein or a fragment thereof and an antibody which binds to said protein or fragment thereof, where the method combines a protein or fragment thereof, a bioactive agent and an antibody which binds to the protein or fragment thereof, wherein the method further includes determining the binding of the protein or fragment thereof and the antibody.

In another preferred embodiment, the present invention provides an antibody which specifically binds to a protein encoded from a marker listed in Tables 1 and 3-8. In particularly preferred embodiment, the antibody is monoclonal and humanized.

In yet another preferred embodiment, the present invention provides a peptide encoded from markers listed in Tables 1 and 3-8. Furthermore, the present invention is also directed to a composition comprising the peptide.

In an alternative embodiment, the present invention provides a composition capable of modulating an immune response in a subject, where the composition comprises a protein encoded from a marker listed in Tables 1 and 3-8 and a pharmaceutically acceptable carrier.

In yet another embodiment, the present invention provides a biochip comprising a panel of markers selected from the group of markers listed in Tables 1 and 3-8. Furthermore, in a particularly preferred embodiment, the markers for a biochip may be selected for subjects suspected of having systemic lupus erythematosus with different manifestations of the disease, in particular nephritis or facial lesions.

Other features and advantages of the invention will be apparent from the following detailed description and claims.

### BRIEF DESCRIPTION OF THE DRAWINGS

FIG. 1 is a graphical representation of the expression levels of genes listed in Table 5, as found in asymptomatic mice at 12 weeks, diseased mice at 36 weeks, and in rapamycin-treated, diseased mice at 36 weeks (see Example 2 below).

FIG. 2 is a graphical representation of the expression levels of the indicated genes as normalized by antibodies to B7 molecules at 50 weeks, as compared to untreated mice at 12 weeks and 24 weeks.

FIG. 3 is a graphical representation of the expression levels of the indicated genes, as normalized by rapamycin or antiB7 and compared to untreated mice at 12 weeks and 36 weeks.

### DETAILED DESCRIPTION OF THE INVENTION

The present invention provides methods for diagnosis and prognosis evaluation for systemic lupus erythematosus (SLE) in subjects, as well as methods and molecular targets for therapeutic intervention.

In one aspect of the invention, the expression levels of genes are determined in a particular patient sample for which either diagnosis or prognosis information is desired. The level of expression of a number of genes simultaneously provides an expression profile, which is essentially a “fingerprint” of the activity of a gene or plurality of genes that is unique to the state of the cell. Comparison of relative levels of expression have been found to be indicative of the presence of systemic lupus erythematosus, and as such permits for diagnostic and prognostic analysis. Moreover, by comparing relative expression profiles of systemic lupus erythematosus tissue in subjects suffering different manifestations (i.e. nephritis, facial lesions, endocarditis, hemolytic anemia or leukopenia), information regarding which genes are important (including both up- and down-regulation of genes) in each of these states is obtained. The identification of gene markers that are differentially expressed in diseased versus non-diseased tissue, as well as differential expression resulting in different prognostic outcomes, allows the use of this invention in a number of ways. For example, the evaluation of a particular treatment regime may be evaluated: will a particular drug act to improve the long-term prognosis in a particular patient? The discovery of these differential expression patterns for individual genes allows for screening of drug candidates with an eye to mimicking or altering a particular expression pattern; for example, screening can be done for drugs that will alter the SLE differential expression pattern or convert a poor prognosis pattern to a better prognosis pattern. This may be done by making biochips comprising sets of the significant SLE genes, which can then be used in these screens. These methods can also be done on the protein basis; that is protein expression levels of the SLE-associated proteins can be evaluated for diagnostic and prognostic purposes or to screen test compounds. In addition, the markers can be administered for gene therapy purposes, including the administration of antisense nucleic acids, or proteins (including antibodies and other modulators thereof) administered as therapeutic drugs.

Moreover, while murine markers are provided in the present invention for disease and drug evaluation, it is well-appreciated in the art that expression levels from human subjects may also be measured. Furthermore, markers from other organisms may be useful as animal models for study of SLE and for drug evaluation. Markers from other organisms may be obtained using the techniques outlined below.

The present invention is based, at least in part, on the identification of a number of genetic markers, set forth in Tables 1 and 3-8, which are differentially expressed between diseased samples (SLE-associated) and non-diseased samples. Autoimmune kidney disease (“AKD”) is a well-accepted murine model for SLE, and genes which are significant in AKD will likely play a role in human SLE. Consequently, a panel of 11,000 known murine genes was screened for expression in diseased versus non-diseased tissue from twelve different mice afflicted with the disease (see Example 1). The full list of novel genes that were differentially regulated between onset and peak are set forth in Table 1. This differential expression was observed either as an increase in expression in a subset of markers(Table 3), or a decrease in expression in a further subset of markers (Table 4). In addition, to narrow the subset of diseased-related, immune-mediated genes, diseased cells were subjected to treatment by rapamycin or to antibodies which bind to B7 molecules (“anti-B7”), to yield a further subset of genes (Tables 5-6 for rapamycin, and Table 7 for anti-B7).

Included among the genes used to screen diseased versus non-diseased tissue in the murine panel were several genes known in the art to be implicated in SLE, as listed in Table 2. These genes served as an internal control. Each of these genes were found to be substantially increased in expression in the diseased cells as opposed to non-diseased cells, thus validating the method as a means for identifying significant genes involved in the disease pathology. Correspondingly, the genes which are known in the art to be linked to SLE (Table 2) may also serve as validation in expression studies for SLE. Moreover, the differentially regulated genes of the invention, as listed in Table 1 and in particular, in Tables 3-8, have not been previously associated with AKD or systemic lupus erythematosus.

Accordingly, the present invention pertains to the use of the genes set forth in Tables 1 and 3-8, the corresponding mRNA transcripts, and the encoded polypeptides as markers for the presence or risk of development of SLE. These markers are further useful to correlate the extent and/or severity of disease. In particular, the present invention is directed to the genes set forth in Table 3-4, Table 7 and Table 8.

Panels of the markers can be conveniently arrayed on solid supports, i.e. biochips for use in kits. Markers can also be useful for assessing the efficacy of a treatment or therapy of SLE.

In one aspect, the invention provides markers whose level of expression, which signifies their quantity or activity, is correlated with the presence of SLE. The markers of the invention may be nucleic acid molecules (e.g., DNA, cDNA or MRNA) or peptide(s). Preferably the invention is performed by detecting the presence of a transcribed polynucleotide or a portion thereof, wherein the transcribed polynucleotide comprises the marker. Alternatively, detection may be performed by detecting the presence of a protein which corresponds to the marker. The markers of the invention are either increased or decreased in quantity or activity in SLE tissue as compared to non-diseased tissue. For example, the gene designated ‘ACTC1’ is increased in expression level in diseased murine kidney cells, relative to control cells, while the gene designated ‘LPL’ is decreased in expression level in the diseased murine kidney cells, relative to control cells. Both the presence of increased or decreased mRNA for these genes (and for other genes set forth in Tables 1 and 3-8), and also increased or decreased levels of the protein products of these genes (and other genes set forth in Tables 1 and 3-8) serve as markers for either AKD or SLE. Preferably, increased or decreased levels of the markers of the invention are increases and decreases of a magnitude that are statistically substantial as compared to appropriate control samples (i.e., non-involved tissue or from non-diseased subjects.) In particularly preferred embodiments, the marker is increased or decreased relative to control samples by at least 2-, 3-, 4-, 5-, 6-, 7-, 8-, 9-, or 10-fold or more. Similarly one skilled in the art will be cognizant of the fact that a preferred detection methodology is one in which the resulting detection values are above the minimum detection limit of the methodology.

Detection and measurement of the relative amount of a nucleic acid or peptide marker of the invention may be by any method known in the art (see, i.e., Sambrook, J., Fritsh, E. F., and Maniatis, T. Molecular Cloning: A Laboratory Manual. 2^{nd}, ed., Cold Spring Harbor Laboratory, Cold Spring Harbor Laboratory Press, Cold Spring Harbor, N.Y. (1989), and Current Protocols in Molecular Biology, eds. Ausubel et al, John Wiley & Sons (1992)). Typical methodologies for detection of a transcribed polynucleotide include RNA extraction from a cell or tissue sample, followed by hybridization of a labeled probe (i.e., a complementary nucleic acid molecule) specific for the target RNA to the extracted RNA and detection of the probe (i.e. Northern blotting). Typical methodologies for peptide detection include protein extraction from a cell or tissue sample, followed by hybridization of a labeled probe (i.e., an antibody) specific for the target protein to the protein sample, and detection of the probe. The label group can be a radioisotope, a fluorescent compound, an enzyme, or an enzyme co-factor. Detection of specific peptide(s) and nucleic acid molecules may also be assessed by gel electrophoresis, column chromatography, direct sequencing, or quantitative PCR (in the case of nucleic acid molecules) among many other techniques well known to those skilled in the art.

In certain embodiments, the genes themselves (i.e., the DNA or cDNA) may serve as markers for SLE. For example, the absence of nucleic acids corresponding to a gene (i.e. a gene from Table 8) such as by deletion of all or part of the gene, may be correlated with disease. Similarly an increase of nucleic acid corresponding to a gene (i.e. a gene from Tables 1 and 3-8), such as by duplication of the gene, may also be correlated with disease.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labeled probe (i.e. a complementary DNA molecules), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

The invention also encompasses nucleic acid and peptide molecules which are structurally different from the molecules described above (i.e. which have a slight altered nucleic acid or amino acid sequence), but which have the same properties as the molecules above (e.g., encoded amino acid sequences, or which are changed only in nonessential amino acid residues). Such molecules include allelic variants, and are described in greater detail in subsection I.

In another aspect, the invention provides markers whose quantity or activity is correlated with different manifestations or severity of SLE: facial lesions, nephritis, endocarditis, hemolytic anemia and leukopenia. These markers are either increased or decreased in quantity or activity in SLE tissue in a fashion that is either positively or negatively correlated with the degree of severity of the SLE. A method of monitoring progression of SLE in subjects may be devised by detecting a substantial difference between the levels of expression in a diseased subject at different points in time. The subsequent level of expression may further be compared to different expression profiles of various SLE manifestations to confirm whether the subject has a matching profile. In yet another aspect, the invention provides markers whose quantity or activity is correlated with a risk in a subject for developing SLE. These markers are either increased or decreased in activity or quantity in direct correlation to the likelihood of the development of SLE in a subject.

Each marker may be considered individually, although it is within the scope of the invention to provide combinations of two or more markers for use in the methods and compositions of the invention to increase the confidence of the analysis. In another aspect, the invention provides panels of the markers of the invention. In a preferred embodiment, these panels of markers are selected such that the markers within any one panel share certain features. For example, the markers of a first panel may each exhibit a decrease in quantity or activity in SLE tissue as compared to samples from non-involved samples from the same subject or tissue from a non-diseased subject. Similarly, different panels of markers may be composed of markers from different tissues (i.e., skin or kidney tissue, or may represent different components of an SLE manifestation or severity (i.e., facial lesions, nephritis, endocarditis, hemolytic anemia and leukopenia). Panels of the markers of the invention may be made by independently selecting markers from any of Tables 1 and 3-8, and may further be provided on biochips, as discussed below.

It will be appreciated by one skilled in the art that the panels of markers of the invention may conveniently be provided on solid supports, as a biochip. For example, polynucleotides may be coupled to an array (e.g., a biochip using GeneChip® for hybridization analysis), to a resin (e.g., a resin which can be packed into a column for column chromatography), or a matrix (e.g. a nitrocellulose matrix for northern blot analysis). The immobilization of molecules complementary to the marker(s), either covalently or noncovalently, permits a discrete analysis of the presence or activity of each marker in a sample. In an array, for example, polynucleotides complementary to each member of a panel of markers may individually be attached to different, known locations on the array. The array may be hybridized with, for example, polynucleotides extracted from a kidney sample from a subject. The hybridization of polynucleotides from the sample with the array at any location on the array can be detected, and thus the presence or quantity of the marker in the sample can be ascertained. In a preferred embodiment, an array based on a biochip is employed. Similarly, Western analyses may be performed on immobilized antibodies specific for different polypeptide markers hybridized to a protein sample from a subject.

It will also be apparent to one skilled in the art that the entire marker protein or nucleic acid molecule need not be conjugated to the biochip support; a portion of the marker or sufficient length for detection purposes (i.e., for hybridization), for example a portion of the marker which is 7, 10, 15, 20, 25, 30, 35, 40, 45, 50, 55, 60, 65, 70, 75, 100 or more nucleotides or amino acids in length may be sufficient for detection purposes.

The nucleic acid and peptide markers of the invention may be isolated from any tissue or cell of a subject. In a preferred embodiment, the tissue is kidney tissue. However, it will be apparent to one skilled in the art that other tissue samples, including bodily fluids such as blood, may also serve as sources from which the markers of the invention may be assessed. The tissue samples containing one or more of the markers themselves may be useful in the methods of the invention, and one skilled in the art will be cognizant of the methods by which such samples may be conveniently obtained, stored and/or preserved.

Several markers were known prior to the invention to be associated with SLE and are provided in Table 2. These markers are not to be considered as markers of the invention. However, these markers may be conveniently be used in combination with the markers of the invention (Tables 1 and 3-8) in the methods, panels and kits of the invention.

In another aspect, the invention provides methods of making an isolated hybridoma which produces an antibody useful for diagnosing′a patient with SLE. In this method, a protein corresponding to a marker of the invention is isolated (e.g., by purification from a cell in which it is expressed or by transcription and translation of a nucleic acid encoding the protein in vivo or in vitro using known methods). A vertebrate, preferably a mammal such as a mouse, rabbit or sheep, is immunized using the isolated protein or protein fragment. The vertebrate may optionally (and preferably) be immunized at least one additional time with the isolated protein or protein fragment, so that the vertebrate exhibits a robust immune response to the protein or protein fragment. Splenocytes are isolated from the immunized vertebrate and fused with an immortalized cell line to form hybridomas, using any of a variety of methods well known in the art. Hybridomas formed in this manner are then screened using standard methods to identify one or more hybridomas which produce an antibody which specifically binds with the protein or protein fragment. The invention also includes hybridomas made by this method and antibodies made using such hybridomas.

The invention provides methods of diagnosing SLE, or determining the risk of developing SLE. These methods involve isolating a sample from a subject (e.g., a sample containing skin cells or kidney tissue), detecting the presence, quantity and/or activity of one or more markers of the invention in the sample relative to a second sample from a non-diseased subject, or from a non-involved tissue in the same subject. The levels of markers in the two samples are compared, and a substantial increase or decrease in one or more markers in the test sample indicates the presence or risk of presence of SLE in the subject.

The invention also provides methods of assessing the efficacy of a test compound or therapy for inhibiting SLE in a subject. These methods involve isolating samples from a subject suffering from SLE who is undergoing treatment or therapy, and detecting the presence, quantity, and/or activity of one or more markers of the invention in the first sample relative to a second sample. Where a test compound is administered, the first and second samples are preferably sub-portions of a single sample taken from the patient, wherein the first portion is exposed to the test compound and the second portion is not. In one aspect of this embodiment, the substantially different level of expression is a substantially lower level of expression in the first sample, relative to the second. Most preferably, the level of expression in the first sample approximates (i.e., less than a two fold difference from a control) the level of expression in a third control sample, taken from either a non-diseased subject or non-involved tissue.

Where the efficacy of a therapy is being assessed, the first sample obtained from the subject is preferably obtained prior to provision of at least a portion of the therapy, whereas the second sample is obtained following provision of the portion of the therapy. The levels of markers in the samples are compared, preferably against a third control sample as well, and correlated with the presence, risk of presence, or severity of SLE. Most preferably, the level of markers in the second sample approximates the level of expression of a third control sample. By assessing whether expression of SLE has been lessened or alleviated in the sample, the ability of the treatment or therapy to treat SLE is determined.

The invention also provides a method of screening test compounds for inhibitors of SLE, and to the pharmaceutical compositions comprising the test compounds. The method of screening comprises obtaining samples of diseased or involved cells, maintaining separate aliquots of the samples with a plurality of test compounds, and comparing expression of a marker in each of the aliquots to determine whether any of the test compounds provides a substantially different level of expression from a control. In addition, methods of screening may be devised by combining a test compound with a protein and thereby determining the effect of the test compound on the protein. Alternatively, the invention is further directed to a method of screening for bioactive agents capable of interfering with the binding of a protein encoded by the markers of Tables 1 and 3-8, and an antibody, by combining the bioactive agent, protein, and antibody together and determining whether binding of the antibody and protein occurs.

Moreover, the invention is directed to pharmaceutical compositions comprising the test compound, or bioactive agent, which may further include a marker protein and/or nucleic acid of the invention (e.g., for those markers in Tables 1 and 3-8 which are decreased or increased in quantity or activity in SLE versus non-diseased tissue), and can be formulated as described herein. Alternatively, these compositions may include an antibody which specifically binds to a marker protein of the invention and/or an antisense nucleic acid molecule which is complementary to a marker nucleic acid of the invention (e.g., for those markers which are increased in quantity in SLE tissue) and can be formulated as described herein.

The invention further provides methods of modulating a level of expression of a marker of the invention, comprising administration to the diseased cells of the subject a variety of compositions which correspond to the markers of Tables 1 and 3-8, including proteins or antisense oligonucleotides. The protein may be provided to the diseased cells by further providing a vector comprising a polynucleotide encoding the protein to the cells. Alternatively, the expression levels of the markers of the invention may be modulated by providing an antibody, a plurality of antibodies or an antibody conjugated to a therapeutic moiety. Treatment with the antibody may further be localized to the diseased tissue. In another aspect, the invention provides methods for localizing a therapeutic moiety to diseased tissue comprising exposing the tissue to an antibody which is specific to a protein encoded from the markers of the invention. This method may therefore provide a means to inhibit or enhance expression of a specific gene corresponding to a marker listed in Tables 1 and 3-8. Where the gene is up-regulated as a result of SLE pathology, it is likely that inhibition of SLE progression would involve inhibiting expression of the up-regulated gene. As a corollary to this method, where the gene is down-regulated, inhibition of SLE progression would therefore likely require enhancing expression of the down-regulated gene.

In another aspect, the invention includes antibodies that are specific to proteins corresponding to markers of the invention. Preferably the antibodies are monoclonal, and most preferably, the antibodies are humanized, as per the description of antibodies described below.

In still another aspect of the invention, the invention includes peptides or proteins which are encoded from the markers of the invention, and to compositions thereof.

The invention also provides kits for diagnosing a subject with SLE, the kit comprising reagents for assessing expression of the markers of the invention. Preferably, the reagents may be an antibody or fragment thereof, wherein the antibody or fragment thereof specifically binds with a protein corresponding to a marker from Tables 1 and 3-8. Optionally, the kits may comprise a nucleic acid probe wherein the probe specifically binds with a transcribed polynucleotide corresponding to a marker selected from the group consisting of the markers listed in Tables 1 and 3-8.

The invention further provides kits for assessing the suitability of each of a plurality of compounds for inhibiting progression of SLE in a subject. Such kits include a plurality of compounds to be tested, and a reagent (i.e. antibody specific to corresponding proteins of the invention) for assessing expression of a marker listed in Tables 1 and 3-8.

Modifications to the above-described compositions and methods of the invention, according to standard techniques, will be readily apparent to one skilled in the art and are meant to be encompassed by the invention.

To facilitate an understanding of the present invention, a number of terms and phrases are defined below:

As used herein, the term “modulation” includes, in its various grammatical forms (e.g., “modulated”, “modulation”, “modulating”, etc.), up-regulation, induction, stimulation, potentiation, and/or relief of inhibition, as well as inhibition and/or down-regulation.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine(C); guanine (G); thymine (T); and uracil (U) for guanine when the polynucleotide is RNA. This, the term “polynucleotide sequence” is the alphabetical representation of a polynucleotide molecule. This alphabetical representation can be inputted into databases in a computer having a central processing unit and used for bioinformatics applications such as functional genomics and homology searching.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of sill in the art, some of which are described herein.

A “gene product” includes an amino acid sequence(e.g., peptide or polypeptide) generated when a gene is transcribed and translated.

As used herein, a “polynucleotide corresponds to” another (a first) polynucleotide if it is related to the first polynucleotide by any of the following relationships:

1) The second polynucleotide comprises the first polynucleotide and the second polynucleotide encodes a gene product.

2) The second polynucleotide is 5′ or 3′ to the first polynucleotide in cDNA, RNA, genomic DNA, or fragments of any of these polynucleotides. For example, a second polynucleotide may be a fragment of a gene that includes the first and second polynucleotides. The first and second polynucleotides are related in that they are components of the gene coding for a gene product, such as a protein or antibody. However, it is not necessary that the second polynucleotide comprises or overlaps with the first polynucleotide to be encompassed within the definition of “corresponding to” as used herein. For example, the first polynucleotide may be a fragment of a 3′ untranslated region of the second polynucleotide. The first and second polynucleotide may be fragments of a gene coding for a gene product. The second polynucleotide may be an exon of the gene while the first polynucleotide may be an intron of the gene.

3) The second polynucleotide is the complement of the first polynucleotide.

As used herein, the term, “transcribed” or “transcription” refers to the process by which genetic code information is transferred from one kind of nucleic acid to another, and refers in particular to the process by which a base sequence of mRNA is synthesized on a template of cDNA.

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set or primers” consisting of “upstream” and a “downstream” primer, and a catalyst of polymerization, such as a DNA polymerase, and typically a thermally-stable polymerase enzyme. Methods for PCR are well known in the art, and are taught, for example, in MacPherson et al., IRL Press at Oxford University Press (1991)). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria (e.g., lambda phage). The library can then be probed for the specific cDNA (and thus mRNA) of interest.

A “gene delivery vehicle” includes a molecule that is capable of inserting one or more polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, viruses and viral vectors, such as baculovirus, adenovirus, and retrovirus, bacteriophage, cosmid, plasmid, fungal vector and other recombination vehicles typically used in the art which have been described for replication and/or expression in a variety of eukaryotic and prokaryotic hosts. The gene delivery vehicles may be used for replication of the inserted polynucleotide, gene therapy as well as for simply polypeptide and protein expression.

A “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above function.

A “host cell” is intended to include any individual cell or cell culture which can be or has been a recipient for vectors or for the incorporation of exogenous nucleic acid molecules, polynucleotides and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, e.g., murine, rat, simian or human cells.

The term “genetically modified” includes a cell containing and/or expressing a foreign gene or nucleic acid sequence which in turn modifies the genotype or phenotype of the cell or its progeny. This term includes any addition, deletion, or disruption to a cell's endogenous nucleotides.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

“Differentially expressed”, as applied to a gene, includes the differential production of mRNA transcribed from a gene or a protein product encoded by the gene. A differentially expressed gene may be overexpressed or underexpressed as compared to the expression level of a normal or control cell. In one aspect, it includes a differential that is 2 times, preferably 5 times or preferably 10 times higher or lower than the expression level detected in a control sample. The term “differentially expressed” also includes nucleotide sequences in a cell or tissue which are expressed where silent in a control cell or not expressed where expressed in a control cell.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, there or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table A below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE A
Stringency Conditions

| Strin- | | | | |
|---|---|---|---|---|
| gency | Poly- | Hybrid | Hybridization | |
| Con- | nucleotide | Length | Temperature and | Wash Tempera- |
| dition | Hybrid | (bp)¹ | Buffer^{H} | ture and Buffer^{H} |
| A | DNA:DNA | >50 | 65° C.; 1 × SSC -or- | 65° C., |
| | | | 42° C.; 1 × SSC, | 0.3 × SSC |
| | | | 50% formamide | |
| B | DNA:DNA | <50 | T_{B}*; 1 × SSC | T_{B}*; 1 × SSC |
| C | DNA:RNA | >50 | 67° C.; 1 × SSC -or- | 67° C.; |
| | | | 45° C.; 1 × SSC, | 0.3 × SSC |
| | | | 50% formamide | |
| D | DNA:RNA | <50 | T_{D}*; 1 × SSC | T_{D}*; 1 × SSC |
| E | RNA:RNA | >50 | 70° C.; 1 × SSC -or- | 70° C.; |
| | | | 50° C.; 1 × SSC, | 0 3 × SSC |
| | | | 50% formamide | |
| F | RNA:RNA | <50 | T_{F}*; 1 × SSC | T_{f}*, 1 × SSC |
| G | DNA:DNA | >50 | 65° C.; 4 × SSC -or- | 65° C.; 1 × SSC |
| | | | 42° C.; 4 × SSC, | |
| | | | 50% formamide | |
| H | DNA:DNA | <50 | T_{H}*; 4 × SSC | T_{H}*; 4 × SSC |
| I | DNA:RNA | >50 | 67° C.; 4 × SSC -or- | 67° C.; 1 × SSC |
| | | | 45° C.; 4 × SSC, | |
| | | | 50% formamide | |
| J | DNA:RNA | <50 | T_{J}*; 4 × SSC | T_{J}*; 4 × SSC |
| K | RNA:RNA | >50 | 70° C.; 4 × SSC -or- | 67° C., 1 × SSC |
| | | | 50° C.; 4 × SSC, | |
| | | | 50% formamide | |
| L | RNA:RNA | <50 | T_{L}*; 2 × SSC | T_{L}*; 2 × SSC |
| M | DNA:DNA | >50 | 50° C.; 4 × SSC -or- | 50° C.; 2 × SSC |
| | | | 40° C.; 6 × SSC, | |
| | | | 50% formamide | |
| N | DNA:DNA | <50 | T_{N}*; 6 × SSC | T_{N}*, 6 × SSC |
| O | DNA:RNA | >50 | 55° C.; 4 × SSC -or- | 55° C.; 2 × SSC |
| | | | 42° C.; 6 × SSC, | |
| | | | 50% formamide | |
| P | DNA:RNA | <50 | T_{P}*; 6 × SSC | T_{P}*; 6 × SSC |
| Q | RNA:RNA | >50 | 60° C.; 4 × SSC -or- | 60° C.; 2 × SSC |
| | | | 45° C.; 6 × SSC, | |
| | | | 50% formamide | |
| R | RNA:RNA | <50 | T_{R}*, 4 × SSC | T_{R}*; 4 × SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| _{H}*SSPE (1 × SSPE is 0.15M NaCl, 10 mM NaH₂PO₄, and 1.25 mM EDTA, pH 7.4) can be substituted for SSC (1 × SSC is 0.15M NaCl and 15 mM sodium citrate) in the hybridization and wash buffers; washes are performed for 15 minutes after hybridization is complete. | | | | |
| T_{B}*-T_{R}*The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1 × SSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprises an antigen recognition site of the required specificity.

As used herein, the term “diseased” refers to cells, tissues or samples from a subject afflicted with systemic lupus erythematosus, wherein the cell, tissue or sample has been affected by systemic lupus erythematosus (i.e. from facial lesions or kidney cells of a patient suffering from nephritis). As used herein, the term “non-diseased” refers to cells, tissues or other such samples taken from a subject who is not afflicted with systemic lupus erythematosus. As used herein, “non-involved” refers to cells, tissues, or samples wherein the tissue is from a subjected afflicted with SLE, but wherein the cells, tissues or samples are believed to be unaffected by systemic lupus erythematosus. Preferred tissue (and cell) samples are from kidney, skin, blood, sera, lymph, thymus, spleen, bone marrow or pus. For those patients suffering from facial lesions, the samples are preferably from skin. Most preferred samples are kidney tissues.

As used herein, the term “marker” includes a polynucleotide or polypeptide molecule which is present or absent, or increased or decreased in quantity or activity in subjects afflicted with systemic lupus erythematosus, or in SLE-associated cells. The relative change in quantity or activity of the marker is correlated with the incidence or risk of incidence of systemic lupus erythematosus.

As used herein, the term “panel of markers” includes a group of markers, the quantity or activity of each member of which is correlated with the incidence or risk of incidence of a SLE-associated condition. In certain embodiments, a panel of markers may include only those markers which are either increased or decreased in quantity or activity in subjects afflicted with or cells involved in a SLE-associated condition. In a preferred embodiment, the panel of markers comprises at least 5 markers, and most preferably, the panel comprises markers listed in Table 8. In other embodiments, a panel of markers may include only those markers present in a specific tissue type which are correlated with the incidence of risk of incidence of a SLE-associated condition.

Various aspects of the invention are described in further detail in the following subsections:

I. Isolated Nucleic Acid Molecules

One aspect of the invention pertains to isolated nucleic acid molecules that either themselves are the genetic markers (e.g., MRNA) of the invention, or which encode the polypeptide markers of the invention, or fragments thereof. Another aspect of the invention pertains to isolated nucleic acid fragments sufficient for sue as hybridization probes to identify the nucleic acid molecules encoding the markers for the invention in a sample, as well as nucleotide fragments for use as PCR primers of the amplification or mutation of the nucleic acid molecules which encode the markers of the invention. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated marker nucleic acid molecule of the invention, or nucleic acid molecule encoding a polypeptide marker of the invention, can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of one of the genes set forth in Tables 1 and 3-8, or a portion thereof, can be isolated using standard molecular biology techniques and the sequence information provided herein. Using all or portion of the nucleic acid sequence of one of the genes set forth in Tables 1 and 3-8 as a hybridization probe, a marker gene of the invention or a nucleic acid molecule encoding a polypeptide marker of the invention can be isolated using standard hybridization and cloning techniques (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold spring Harbor, N.Y., 1989).

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to marker nucleotide sequences, or nucleotide sequences encoding a marker of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of a marker of the invention (e.g., a gene set forth in Tables 1 and 3-8), or a portion of any of these nucleotide sequences. A nucleic acid molecule which is complementary to such a nucleotide sequence is one which is sufficiently complementary t the nucleotide sequence such that it can hybridize to the nucleotide sequence, thereby forming a stable duplex.

The nucleic acid molecule of the invention, moreover, can comprise only a portion of the nucleic acid sequence of a marker nucleic acid of the invention, or a gene encoding a marker polypeptide of the invention, for example, a fragment which can be used as a probe or primer. The probe/primer typically comprises substantially purified oligonucleotide. The oligonucleotide typically comprises a region of nucleotide sequence that hybridizes under stringent conditions to at least about 7 or 15, preferably about 20 or 25, more preferably about 50, 75, 100, 125, 150, 175, 200, 225, 250, 275, 300, 325, 350, 400 or more consecutive nucleotides of a marker nucleic acid, or a nucleic acid encoding a marker polypeptide of the invention.

Probes based on the nucleotide sequence of a marker gene or of a nucleic acid molecule encoding a marker polypeptide of the invention can be used to detect transcripts or genomic sequences corresponding to the marker gene(s) and/or marker polypeptide(s) of the invention. In preferred embodiments, the probe comprises a label group attached thereto, e.g., the label group can be a radioisotope, a fluorescent compound, an enzyme, or an enzyme co-factor. Such probes can be used as a part of a diagnostic test kit for identifying cells or tissue which misexpress (e.g., over- or under-express) a marker polypeptide of the invention, or which have greater or fewer copies of a marker gene of the invention. For example, a level of a marker polypeptide-encoding nucleic acid in a sample of cells from a subject may be detected, the amount of mRNA transcript of a gene encoding a marker polypeptide may be determined, or the presence of mutations or deletions of a marker gene of the invention may be assessed.

The invention further encompasses nucleic acid molecules that differ from the nucleic acid sequences of the genes set forth in Tables 1 and 3-8, due to degeneracy of the genetic code and which thus encode the same proteins as those encoded by the genes shown in Tables 1 and 3-8.

In addition to the nucleotide sequences of the genes set forth in Tables 1 and 3-8, it will be appreciated by those skilled in the art that DNA sequence polymorphisms that lead to changes in the amino acid sequences of the proteins encoded by the genes set forth in Tables 1 and 3-8 may exist within a population e.g., the human population). Such genetic polymorphism in the genes set forth in Tables 1 and 3-8 may exist among individuals within a population due to natural allelic variation. An allele is one of a group of genes which occur alternatively at a given genetic locus. In addition it will be appreciated that DNA polymorphisms that affect RNA expression levels can also exist that may affect the overall expression level of that gene e.g., by affecting regulation or degradation). As used herein, the phrase “allelic variant” includes a nucleotide sequence which occurs ta a given locus or to a polypeptide encoded by the nucleotide sequence. As used herein, the terms “gene” and “recombinant gene” refer to nucleic acid molecules which include an open reading frame encoding a marker polypeptide of the invention.

Nucleic acid molecules corresponding to natural allelic variants and homologues of the marker genes, or genes encoding the marker proteins of the invention can be isolated based on their homology to the genes set forth in Tables 1 and 3-8, using the cDNAs disclosed herein, or a portion thereof, as a hybridization probe according to standard hybridization techniques under stringent hybridization conditions. Nucleic acid molecules corresponding to natural allelic variants and homologues of the marker genes of the invention can further be isolated by mapping to the same chromosome or locus as the marker genes or genes encoding the marker proteins of the invention.

In another embodiment, an isolated nucleic acid molecule of the invention is at least 15, 20, 25, 30, 50, 100, 150, 200, 250, 300, 350, 400, 450, 500, 550, 600, 650,700,750,800,850, 900,950, 1000, 1100, 1200, 1300, 1400, 1500, 1600, 1700, 1800, 1900, 2000 or more nucleotides in length and hybridizes under stringent conditions to a nucleic acid molecule corresponding to a nucleotide sequence of a marker gene or gene encoding a marker protein of the invention. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. Preferably, an isolated nucleic acid molecule of the invention that hybridizes under stringent conditions to the sequence of one of the genes set forth in Tables 1 and 3-8 corresponds to a naturally-occurring nucleic acid molecule. As used herein, a “naturally-occurring” nucleic acid molecule includes an RNA or DNA molecule having a nucleotide sequence that occurs in nature (e.g., encodes a natural protein).

In addition to naturally-occurring allelic variants of the marker gene and gene encoding a marker protein of the invention sequences that may exist in the population, the skilled artisan will further appreciate that changes can be introduced by mutation into the nucleotide sequences of the marker genes or genes encoding the marker proteins of the invention, thereby leading to changes in the amino acid sequence of the encoded proteins, without altering the functional activity of these proteins. For example, nucleotide substitutions leading to amino acid substitutions at “non-essential” amino acid residues can be made. A “non-essential” amino acid residue is a residue that can be altered from the wild-type sequence of a protein without altering the biological activity, whereas an “essential” amino acid residue is required for biological activity. For example, amino acid residues that are conserved among allelic variants or homologs of a gene (e.g., among homologs of a gene from different species) are predicted to be particularly unamenable to alteration.

Accordingly, another aspect of the invention pertains to nucleic acid molecules encoding a marker protein of the invention that contain changes in amino acid residues that are not essential for activity. Such proteins differ in amino acid sequence from the marker proteins encoded by the genes set forth in Tables 1 and 3-8, yet retain biological activity. In one embodiment, the protein comprises an amino acid sequence at least about 60%, 65%, 70%, 75%, 80%, 85%, 90%, 95%, 98% or more homologous to a marker protein of the invention.

An isolated nucleic acid molecule encoding a protein homologous to a marker protein of the invention can be created by introducing one or more nucleotide substitutions, additions or deletions into the nucleotide sequence of the gene encoding the marker protein, such that one or more amino acid substitutions, additions or deletions are introduced into the encoded protein. Mutations can be introduced into the genes of the invention (e.g., a gene set forth in Tables 3-8) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of a coding sequence of a gene of the invention, such as by saturation mutagenesis, and the resultant mutants can be screened for biological activity to identify mutants that retain activity. Following mutagenesis, the encoded protein can be expressed recombinantly and the activity of the protein can be determined.

Another aspect of the invention pertains to isolated nucleic acid molecules which are antisense to the marker genes and genes encoding marker proteins of the invention. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand of a gene of the invention (e.g., a gene set forth in Tables 1 and 3-8), or to only a portion thereof. In one embodiment, an antisense nucleic acid molecule is antisense to a “coding region” of the coding strand of a nucleotide sequence of the invention. The term “coding region” includes the region of the nucleotide sequence comprising codons which are translated into amino acid. In another embodiment, the antisense nucleic acid molecule is antisense to a “noncoding region” of the coding strand of a nucleotide sequence of the invention.

The term “noncoding region” includes 5′ and 3′ sequences which flank the coding region that are not translated into amino acids (i.e., also referred to as 5′ and 3′ untranslated regions).

Antisense nucleic acids of the invention can be designed according to the rules of Watson and Crick base pairing. The antisense nucleic acid molecule can be complementary to the entire coding region of an MRNA corresponding to a gene of the invention, but more preferably is an oligonucleotide which is antisense to only a portion of the coding or noncoding region. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioatc derivatives and acridine substituted nucleotides can be used. Examples of modified nucleotides which can be used to generate the antisense nucleic acid include 5-fluorouracil, 5-bromouracil, 5-chlorouracil, 5-idodouracil, hypoxanthine, xantine, 4-acetylcytosine, 5-(carboxyhydroxyhnethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladen4exine, unacil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5- oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a marker protein of the invention to thereby inhibit expression of the protein, e.g., by inhibiting transcription and/or translation. The hybridization can be by conventional nucleotide complementary to form a stable duplex, or, for example, in the cases of an antisense nucleic acid molecule which binds to DNA duplexes, through specific interactions in the major groove of the double helix. An example of a route of administration of antisense nucleic acid molecules of the invention include direct injection at a tissue site (e.g., in kidney). Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual α-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoif and Gerlach (1988) Nature 334:585-591)) can be used to catalytically cleave mRNA transcripts of the genes of the invention (e.g., a gene set forth in Tables 1 and 3-8) to thereby inhibit translation of this mRNA. A ribozyme having specificity for a marker protein-encoding nucleic acid can be designed based upon the nucleotide sequence of a gene of the invention, disclosed herein. For example, a derivative of a Tetrahymena L-19 IVS RNA can be constructed in which the nucleotide sequence of the active site is complementary to the nucleotide sequence to be cleaved in a marker protein-encoding mRNA. See, e.g., Cech et al U.S. Pat. No. 4,987,071; and Cech et al. U.S. Pat. No. 5,116,742. Alternatively, mRNA transcribed from a gene of the invention can be used to select a catalytic RNA having a specific ribonuclease activity from a pool of RNA molecules. See, e.g., Bartel, D. and Szostak, J. W. (1993) Science 261:1411-1418.

Alternatively, expression of a gene of the invention (e.g., a gene set forth in Tables 1 and 3-8) can be inhibited by targeting nucleotide sequences complementary to the regulatory region of these genes (e.g., the promoter and/or enhancers) to form triple helical structures that prevent transcription of the gene in target cells. See generally, Helene, C. (1991) Anticancer Drug Des. 6(6):569-84; Helene, C. et al. (1992) Ann. N. Y. Acad Sci. 660:27-36; and Maher, L. J. (1992) Bioassays 14(12):807-15.

Expression of the marker genes, and genes encoding marker proteins of the invention, can also be inhibited using RNA interference (“RNAᵢ”). This is a technique for post transcriptional gene silencing (“PTGS”), in which target gene activity is specifically abolished with cognate double-stranded RNA (“dsRNA”). RNA₁ resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNAᵢ in mammalian systems is disclosed in PCT application WO 00/63364 which is incorporated by reference herein in its entirety. Basically, dsRNA of at least about 600 nucleotides, homologous to the target marker is introduced into the cell and a sequence specific reduction in gene activity is observed. See generally, Ui-Teia, K. et al. FEBS Letters 479: 79-82.

In yet another embodiment, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4(1):5 23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs can be used in therapeutic and diagnostic applications. For example, PNAs can be used as antisense or antigene agents for sequence-specific modulation of gene expression by, for example, inducing transcription or translation arrest or inhibiting replication. PNAs of the nucleic acid molecules of the invention (e.g., a gene set forth in Tables 1 and 3-8) can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of the nucleic acid molecules of the invention can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example; a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Pros. Natl. Acad Sci. USA 84:648-652; PCT Publication No. W088/09810) or the blood-kidney barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent). Finally, the oligonucleotide may be detectably labeled, either such that the label is detected by the addition of another reagent (e.g., a substrate for an enzymatic label), or is detectable immediately upon hybridization of the nucleotide (e.g., a radioactive label or a fluorescent label (e.g., a molecular beacon, as described in U.S. Pat. No. 5,876,930).

II. Isolated Proteins and Antibodies One aspect of the invention pertains to isolated marker proteins, and biologically active portions thereof, as well as polypeptide fragments suitable for use as immunogens to raise anti-marker protein antibodies. In one embodiment, native marker proteins can be isolated from cells or tissue sources by an appropriate purification scheme using standard protein purification techniques. In another embodiment, marker proteins are produced by recombinant DNA techniques. Alternative to recombinant expression, a marker protein or polypeptide can be synthesized chemically using standard peptide synthesis techniques.

An “isolated” or “purified” protein or biologically active portion thereof is substantially free of cellular material or other contaminating proteins from the cell or tissue source from which the marker protein is derived, or substantially free from chemical precursors or other chemicals when chemically synthesized. The language “substantially free of cellular material” includes preparations of marker protein in which the protein is separated from cellular components of the cells from which it is isolated or recombinantly produced. In one embodiment, the language “substantially free of cellular material” includes preparations of marker protein having less than about 30% (by dry weight) of non-marker protein (also referred to herein as a “contaminating protein”), more preferably less than about 20% of non-marker protein, still more preferably less than about 10% of non-marker protein, and most preferably less than about 5% non-marker protein. When the marker protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of marker protein in which the protein is separated from chemical precursors or other chemicals which are involved in the synthesis of the protein. In one embodiment, the language “substantially free of chemical precursors or other chemicals” includes preparations of protein having less than about 30% (by dry weight) of chemical precursors or non-protein chemicals, more preferably less than about 20% chemical precursors or non-protein chemicals, still more preferably less than about 10% chemical precursors or non-protein chemicals, and most preferably less than about 5% chemical precursors or non-protein chemicals.

As used herein, a “biologically active portion” of a marker protein includes a fragment of a marker protein comprising amino acid sequences sufficiently homologous to or derived from the amino acid sequence of the marker protein, which include fewer amino acids than the fill length marker proteins, and exhibit at least one activity of a marker protein. Typically, biologically active portions comprise a domain or motif with at least one activity of the marker protein. A biologically active portion of a marker protein can be a polypeptide which is, for example, 10, 25, 50, 100, 200 or more amino acids in length. Biologically active portions of a marker protein can be used as targets for developing agents which modulate a marker protein-mediated activity.

In a preferred embodiment, marker protein is encoded by a gene set forth in Tables 1 and 3-8. In other embodiments, the marker protein is substantially homologous to a marker protein encoded by a gene set forth in Tables 1 and 3-8, and retains the functional activity of the marker protein, yet differs in amino acid sequence due to natural allelic variation or mutagenesis, as described in detail in subsection I above. Accordingly, in another embodiment, the marker protein is a protein which comprises an amino acid sequence at least about 60%, 65%, 70%, 75%, 80%, 85%, 90%, 95%, 98% or more homologous to the amino acid sequence encoded by a gene set forth in Tables 1 and 3-8.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mot. Biol. (48):444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM 120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to marker protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nim.nih.gov.

The invention also provides chimeric or fusion marker proteins. As used herein, a marker “chimeric protein” or “fusion protein” comprises a marker polypeptide operatively linked to a non-marker polypeptide. An “marker polypeptide” includes a polypeptide having an amino acid sequence encoded by a gene set forth in Tables 1 and 3-8, whereas a “non-marker polypeptide” includes a polypeptide having an amino acid sequence corresponding to a protein which is not substantially homologous to the marker protein, e.g., a protein which is different from marker protein and which is derived from the same or a different organism. Within a marker fusion protein the polypeptide can correspond to all or a portion of a marker protein. In a preferred embodiment, a marker fusion protein comprises at least one biologically active portion of a marker protein. Within the fusion protein, the term “operatively linked” is intended to indicate that the marker polypeptide and the non-marker polypeptide are fused in-frame to each other. The non-marker polypeptide can be fused to the N-terminus or C-terminus of the marker polypeptide.

For example, in one embodiment, the fusion protein is a GST-marker fusion protein in which the marker sequences are fused to the C-terminus of the GST sequences. Such fusion proteins can facilitate the purification of recombinant marker proteins.

In another embodiment, the fusion protein is a marker protein containing a heterologous signal sequence at its N-terminus. In certain host cells (e.g., mammalian host cells), expression and/or secretion of marker proteins can be increased through use of a heterologous signal sequence. Such signal sequences are well known in the art.

The marker fusion proteins of the invention can be incorporated into pharmaceutical compositions and administered to a subject in vivo, as described herein. The marker fusion proteins can be used to affect the bioavailability of a marker protein substrate. Use of marker fusion proteins may be useful therapeutically for the treatment of disorders (e.g., systemic lupus erythematosus) caused by, for example, (i) aberrant modification or mutation of a gene encoding a marker protein; (ii) mis-regulation of the marker protein-encoding gene; and (iii) aberrant post-translational modification of a marker protein.

Moreover, the marker-fusion proteins of the invention can be used as immunogens to produce anti-marker protein antibodies in a subject, to purify marker protein ligands and in screening assays to identify molecules which inhibit the interaction of a marker protein with a marker protein substrate.

Preferably, a marker chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols In Molecular Biology, eds. Ausubel et al. John Wiley & Sons: 1992). Moreover, many expression vectors are commercially available that already encode a fusion moiety (e.g., a GST polypeptide). A marker protein-encoding nucleic acid can be cloned into such an expression vector such that the fusion moiety is linked in-frame to the marker protein.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods.

Alternatively, the signal sequence can be linked to the protein of interest using a sequence which facilitates purification, such as with a GST domain.

The present invention also pertains to variants of the marker proteins of the invention which function as either agonists (mimetics) or as antagonists to the marker proteins. Variants of the marker proteins can be generated by mutagenesis, e.g., discrete point mutation or truncation of a marker protein. An agonist of the marker proteins can retain substantially the same, or a subset, of the biological activities of the naturally occurring form of a marker protein. An antagonist of a marker protein can inhibit one or more of the activities of the naturally occurring form of the marker protein by, for example, competitively modulating an activity of a marker protein. Thus, specific biological effects can be elicited by treatment with a variant of limited function. In one embodiment, treatment of a subject with a variant having a subset of the biological activities of the naturally occurring forth of the protein has fewer side effects in a subject relative to treatment with the naturally occurring form of the marker protein.

Variants of a marker protein which function as either marker protein agonists (mimetics) or as marker protein antagonists can be identified by screening combinatorial libraries of mutants, e.g., truncation mutants, of a marker protein for marker protein agonist or antagonist activity. In one embodiment, a variegated library of marker protein variants is generated by combinatorial mutagenesis at the nucleic acid level and is encoded by a variegated gene library. A variegated library of marker protein variants can be produced by, for example, enzymatically ligating a mixture of synthetic oligonucleotides into gene sequences such that a degenerate set of potential marker protein sequences is expressible as individual polypeptides, or alternatively, as a set of larger fusion proteins (e.g., for phage display) containing the set of marker protein sequences therein. There are a variety of methods which can be used to produce libraries of potential marker protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential marker protein sequences. Methods for synthesizing degenerate oligonucleotides are known in the art (see, e.g., Narang, S. A. (1983) Tetrahedron 39:3; Itakura et al (1984) Annu. Rev. Biochem. 53:323; Itakura et al. (1984) Science 198:1055; Ike et al. (1983) Nucleic Acid Res. 11:477).

In addition, libraries of fragments of a protein coding sequence corresponding to a marker protein of the invention can be used to generate a variegated population of marker protein fragments for screening and subsequent selection of variants of a marker protein. In one embodiment, a library of coding sequence fragments can be generated by treating a double stranded PCR fragment of a marker protein coding sequence with a nucleus under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nucleus, and ligating the resulting fragment library into an expression vector. By this method, an expression library can be derived which encodes N-terminal, C-terminal and internal fragments of various sizes of the marker protein.

Several techniques are known in the art for screening gene products of combinatorial libraries made by point mutations or truncation, and for screening cDNA libraries for gene products having a selected property. The most widely used techniques, which are amenable to high-throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify marker variants (Arkin and Yourvan (1992) Proc. Natl. Acad. Sci. USA 89:7811-7815; Delgrave et al. (1993) Protein Engineering 6(3):327-331).

An isolated marker protein, or a portion or fragment thereof, can be used as an immunogen to generate antibodies that bind marker proteins using standard techniques for polyclonal and monoclonal antibody preparation. A full-length marker protein can be used or, alternatively, the invention provides antigenic peptide fragments of these proteins for use as immunogens. The antigenic peptide of a marker protein comprises at least 8 amino acid residues of an amino acid sequence encoded by a gene set forth in Tables 1 and 3-8, and encompasses an epitope of a marker protein such that an antibody raised against the peptide forms a specific immune complex with the marker protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of the marker protein that are located on the surface of the protein, e.g., hydrophilic regions, as well as regions with high antigenicity.

A marker protein immunogen typically is used to prepare antibodies by immunizing a suitable subject, (e.g., rabbit, goat, mouse or other mammal) with the immunogen. An appropriate immunogenic preparation can contain, for example, recombinantly expressed marker protein or a chemically synthesized marker polypeptide. The preparation can further include an adjuvant, such as Freund's complete or incomplete adjuvant, or similar immunostimulatory agent. Immunization of a suitable subject with an immunogenic marker protein preparation induces a polyclonal anti-marker protein antibody response.

Accordingly, another aspect of the invention pertains to anti-marker protein antibodies. The term “antibody” as used herein includes immunoglobulin molecules and immunologically active portions of immunoglobulin molecules, i.e., molecules that contain an antigen binding site which specifically binds (immunoreacts with) an antigen, such as a marker protein. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to marker proteins. The term “monoclonal antibody” or “monoclonal antibody composition”, as used herein, includes a population of antibody molecules that contain only one species of an antigen binding site capable of immunoreacting with a particular epitope. A monoclonal antibody composition thus typically displays a single binding affinity for a particular marker protein with which it immunoreacts.

Polyclonal anti-marker protein antibodies can be prepared as described above by immunizing a suitable subject with a marker protein of the invention. The anti-marker protein antibody titer in the immunized subject can be monitored over time by standard techniques, such as with an enzyme linked immunosorbent assay (ELISA) using immobilized marker protein. If desired, the antibody molecules directed against marker proteins can be isolated from the mammal (e.g., from the blood) and further purified by well known techniques, such as protein A chromatography, to obtain the IgG fraction. At an appropriate time after immunization, e.g., when the anti-marker protein antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad, Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a marker protein immunogen as described above, and the culture supernatants of the resulting hybridoma cells are screened to identify a hybridoma producing a monoclonal antibody that binds to a marker protein of the invention.

Any of the many well known protocols used for fusing lymphocytes and immortalized cell lines can be applied for the purpose of generating an anti-marker protein monoclonal antibody (see, e.g., G. Galfre et al. (1977) Nature 266:SSOS2; Gefter et al. Somatic Cell Genet., cited supra; Letter, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, axninopterin and thymidine (“HAT medium”). Any of a number of myeloma cell lines can be used as a fusion partner according to standard techniques, e.g., the P3-NS1/1-Ag4-1, P3-x63-Ag8.653 or Sp2 10-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind to a marker protein, e.g., using a standard ELISA assay.

Alternative to preparing monoclonal antibody-secreting hybridomas, a monoclonal anti-marker protein antibody can be identified and isolated by screening a recombinant combinatorial immunoglobulin library (e.g., an antibody phase display library) with marker protein to thereby isolate immunoglobulin library members that bind to a marker protein. Kits for generating and screening phage display libraries are commercially available (e.g., the Pharmacia Recombinant Phage Antibody System, Catalog No. 27-9400-01; and the Stratagene SurfZA™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92115679; Breitling et al. PCT International Publication WO 93/01288; McCafferty et al PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al..(1992) Proc. Natl. Acad Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acacl. Sci. USA 88:7978-7982; and McCafferty et al. Nature (1990) 348:552-554.

Additionally, recombinant anti-marker protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad Sci. USA 84:3439-3443; Liu et al. (1987) J. Immunol. 139:3521 3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. 5,225,539; Jones et al. (1986) Nature 321:552-525; Verhoeyan et al. (1988) Science 239:1534; and Beidler et al. (1988) J. Immunol. 141:4053-4060.

Humanized antibodies are particularly desirable for therapeutic treatment of human subjects. Humanized forms of non-human (e.g. murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues forming a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immmunoglobulin and all or substantially all of the constant regions being those of a human immunoglobulin consensus sequence. The humanized antibody will preferably also comprise at least a portion of an immunoglobulin constant region (Fe), typically that of a human immunoglobulin (Jones et al. Nature 321: 522-525 (1986); Riechmann et al, Nature 323: 323-329 (1988); and Presta Curr.Op.Struct.Biol. 2: 594-596 (1992).

Such humanized antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing humanized antibodies, see Lonberg and Huszar (1995) Int. Rev. Immunol. 13:65-93). For a detailed discussion of this technology for producing humanized antibodies and humanized monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide humanized antibodies directed against a selected antigen using technology similar to that described above.

Humanized antibodies which recognize a selected epitope can be generated using a technique referred to as “guided selection.” In this approach a selected non-human monoclonal antibody, e.g., a murine antibody, is used to guide the selection of a humanized antibody recognizing the same epitope (Jespers et al., 1994, Bio/technology 12:899-903).

An anti-marker protein antibody (e.g., monoclonal antibody) can be used to isolate a marker protein of the invention by standard techniques, such as affinity chromatography or immunoprecipitation. An anti-marker protein antibody can facilitate the purification of natural marker proteins from cells and of recombinantly produced marker proteins expressed in host cells. Moreover, an anti-marker protein antibody can be used to detect marker protein (e.g., in a cellular lysate or cell supernatant) in order to evaluate the abundance and pattern of expression of the marker protein. Anti-marker protein antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatasc, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a marker protein of the invention (or a portion thereof). As used herein, the term “vector” includes a nucleic acid molecule capable of transporting another nucleic acid to which it has been linked. One type of vector is a “plasmid”, which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequences) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., marker proteins, mutant forms of marker proteins, fusion proteins, and the like).

The recombinant expression vectors of the invention can be designed for expression of marker proteins in prokaryotic or eukaryotic cells. For example, marker proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D, B. and Johnson, K. S. (1988) Gene 67:31-40), pMAL (New England Biolabs, Beverly, Mass.) and pRITS (Pharmacia, Piscataway, N.J.) which fuse glutathione S transferase (GST), maltose E binding protein, or protein A, respectively, to the target recombinant protein.

Purified fusion proteins can be utilized in marker activity assays, (e.g., direct assays or competitive assays described in detail below), or to generate antibodies specific for marker proteins, for example.

Examples of suitable inducible non-fusion E. coli expression vectors include pTrc (Hmann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn 1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wade et al., (1992) Nucleic Acids Res. 20:2111-2118). Such alteration of nucleic acid sequences of the invention can be carried out by standard DNA synthesis techniques.

In another embodiment, the marker protein expression vector is a yeast expression vector. Examples of vectors for expression in yeast S. cerevisiae include pYepSec1 (Baldari, et al., (1987) Embo J. 6:229-234), pMFa (Kurjan and Herskowitz, (1982) Cell 30:933-943), pJRY88 (Schultz et al., 21987) Gene 54:113-123), pYES2 (In Vitrogen Corporation, San Diego, Calif.), and picZ (Invitrogen Corp, San Diego, Calif.).

Alternatively, marker proteins of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed.. Cold Spring Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HSLE174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the marker protein expression vector is a yeast expression vector. Examples of vectors for expression in yeast S. cerevisiae include pYepSecl (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (In Vitrogen Corp, San Diego, Calif.).

Alternatively, marker proteins of the invention can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerij et al. (1983) Cell 33:729-740; Queen and Baltimore (1983) Cell 33:741-748), neuron-specific promoters (e.g., the neurofilament promoter, Byrne and R.aaddle (1989) Proc. Nall. Acad Sci. USA 86:5473-5477), pancreas-specific promoters (Edlund et al. (1985) Science 230:912-916), and mammary gland-specific promoters (e.g., milk whey promoter, U.S. Pat. No. 4,873,316 and European Application Publication No. 264,166). Developmentally-regulated promoters are also encompassed, for example the marine hox promoters (Kessel and Grass (1990) Science 249:374-379) and the ÿ×-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to mRNA corresponding to a gene of the invention (e.g., a gene set forth in Tables 1 and 3-8). Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1)1986.

Another aspect of the invention pertains to host cells into which a nucleic acid molecule of the invention is introduced, e.g., a gene set forth in Tables 1 and 3-8 within a recombinant expression vector or a nucleic acid molecule of the invention containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a marker protein of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DAKD-dextran-mediated transfection, lipofection, or electmporation. Suitable methods for transforming or transferring host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable flag (e.g., resistance to antibiotics) is generally introduced into the host cells along with the gene of interest. Preferred selectable flags include those which confer resistance to drugs, such as G418, hygromycin and methotrexate. Nucleic acid encoding a selectable flag can be introduced into a host cell on the same vector as that encoding a marker protein or can be introduced on a separate vector. Cells stably transfected with the introduced nucleic acid can be identified by drug selection (e.g., cells that have incorporated the selectable flag gene will survive, while the other cells die).

A host cell of the invention, such as a prokaryotic or eukaryotic host cell in culture, can be used to produce (i.e., express) a marker protein. Accordingly, the invention further provides methods for producing a marker protein using the host cells of the invention. In one embodiment, the method comprises culturing the host cell of invention (into which a recombinant expression vector encoding a marker protein has been introduced) in a suitable medium such that a marker protein of the invention is produced. In another embodiment, the method further comprises isolating a marker protein from the medium or the host cell.

The host cells of the invention can also be used to produce non-human transgenic animals. For example, in one embodiment, a host cell of the invention is a fertilized oocyte or an embryonic stem cell into which marker-protein-coding sequences have been introduced. Such host cells can then be used to create non-human transgenic animals in which exogenous sequences encoding a marker protein of the invention have been introduced into their genome or homologous recombinant animals in which endogenous sequences encoding the marker proteins of the invention have been altered. Such animals are useful for studying the function and/or activity of a marker protein and for identifying and/or evaluating modulators of marker protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the tike. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene of the invention (e.g., a gene set forth in Tables 1 and 3-8) has been altered by homologous recombination between the endogenous gene and an exogenous DNA molecule introduced into a cell of the animal, e.g., an embryonic cell of the animal, prior to development of the animal.

A transgenic animal of the invention can be created by introducing a marker-encoding nucleic acid into the mate pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a transgene to direct expression of a marker protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a transgene of the invention in its genome and/or expression of mRNA corresponding to a gene of the invention in tissues or cells of the animals. A transgenic founder animal can then be used to breed additional animals carrying the transgene. Moreover, transgenic animals carrying a transgene encoding a marker protein can further be bred to other transgenic animals carrying other transgenes.

To create a homologous recombinant animal, a vector is prepared which contains at least a portion of a gene of the invention into which a deletion, addition or substitution has been introduced to thereby alter, e.g., functionally disrupt, the gene. The gene can be a human gene, but more preferably, is a non-human homologue of a human gene of the invention (e.g., a gene set forth in Tables 1 and 3-8). For example, a mouse gene can be used to construct a homologous recombination nucleic acid molecule, e.g., a vector, suitable far altering an endogenous gene of the invention in the mouse genome. In a preferred embodiment, the homologous recombination nucleic acid molecule is designed such that, upon homologous recombination, the endogenous gene of the invention is functionally disrupted (i.e., no longer encodes a functional protein; also referred to as a “knock out” vector). Alternatively, the homologous recombination nucleic acid molecule can be designed such that, upon homologous recombination, the endogenous gene is mutated or otherwise altered but still encodes functional protein (e.g., the upstream regulatory region can be altered to thereby alter the expression of the endogenous marker protein). In the homologous recombination nucleic acid molecule, the altered portion of the gene of the invention is flanked at its 5′ and 3′ ends by additional nucleic acid sequence of the gene of the invention to allow for homologous recombination to occur between the exogenous gene carried by the homologous recombination nucleic acid molecule and an endogenous gene in a cell, e.g., an embryonic stem cell. The additional flanking nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced gene has homologously recombined with the endogenous gene are selected (see e.g., Li, E. et al. (1992) Cell 69:915). The selected cells can then injected into a blastocyst of an animal (e.g., a mouse) to form aggregation chimeras (see e.g. Bradley, S A. in Teratocareirtomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Laksa et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The nucleic acid molecules of the invention (e.g., the genes set forth in Tables 1 and 3-8), fragments of marker proteins, and anti-marker protein antibodies of the invention can be incorporated into pharmaceutical compositions suitable for administration. Such compositions (also referred to herein as “bioactive agents or compounds”) typically comprise the nucleic acid molecule, protein, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary bioactive agents can also be incorporated into the compositions.

The invention includes methods for preparing pharmaceutical compositions for modulating the expression or activity of a polypeptide or nucleic acid corresponding to a marker of the invention. Such methods comprise formulating a pharmaceutically acceptable carrier with an agent which modulates expression or activity of a polypeptide or nucleic acid corresponding to a marker of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid corresponding to a marker of the invention and one or more additional bioactive agents.

The invention also provides methods (also referred to herein as “screening assays”) for identifying modulators, i.e., candidate or test compounds or agents comprising therapeutic moieties (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormnone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

The test compounds of the present invention may be bioactive agents, i.e. protein, oligopeptide, molecule, polysaccharide, polynucleotides. In a preferred embodiment the bioactive agents are proteins, in particular naturally occurring proteins or fragments thereof.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckennann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The earner can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a fragment of a marker protein or an anti-marker protein antibody) in the required amount in an appropriate solvent with one or a combination of ingredients enmnerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bioactive compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the therapeutic moieties, which may contain a bioactive compound, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on-the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Computer Readable Means and Arrays

Computer readable media comprising a marker(s) of the present invention is also provided. As used herein, “computer readable media” includes a medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a marker of the present invention.

As used herein, “recorded” includes a process for storing information on computer readable medium. Those skilled in the art can readily adopt any of the presently known methods for recording information on computer readable medium to generate manufactures comprising the markers of the present invention.

A variety of data processor programs and formats can be used to store the marker information of the present invention on computer readable medium. For example, the nucleic acid sequence corresponding to the markers can be represented in a word processing text file, formatted in commercially-available software such as WordPerfect and Microsoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the markers of the present invention.

By providing the markers of the invention in computer readable form, one can routinely access the marker sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

The invention also includes an array comprising a marker(s) of the present invention, i.e. a biochip. The array can be used to assay expression of one or more genes in the array. In one embodiment, the array can be used to assay gene expression in a tissue to ascertain tissue specificity of genes in the array. In this manner, up to about 8600 genes can be simultaneously assayed for expression. This allows an expression profile to be developed showing a battery of genes specifically expressed in one or more tissues at a given point in time.

In addition to such qualitative determination, the invention allows the quantitation of gene expression in the biochip. Thus, not only tissue specificity, but also the level of expression of a battery of genes in the tissue is ascertainable. Thus, genes can be grouped on the basis of their tissue expression per se and level of expression in that tissue. As used herein, a “normal level of expression” refers to the level of expression of a gene provided in a control sample, typically the control is from non-involved cells or tissues, or from a non-diseased subject. Furthermore, as used herein, a “normalized” expression level is where the expression level of an otherwise diseased or involved sample is rendered the same or similar to a control sample. In Examples 1 and 2 below, strict standards were applied by which a gene was said to have “normalized” expression, the difference in expression was required to be less than five. The determination of normal levels of expression is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the arrays can be used to monitor the time course of expression of one or more genes in the array. This can occur in various biological contexts, as disclosed herein, for example development and differentiation, disease progression, in vitro processes, such a cellular transformation and senescence, autonomic neural and neurological processes, such as, for example, pain and appetite, and cognitive functions, such as learning or memory.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in non-involved or diseased cells. This provides a battery of genes that could serve as a molecular target for diagnosis or therapeutic intervention. In particular, biochips can be made comprising arrays not only of the differentially expressed markers listed in Tables 1 and 3-8, but of markers specific to subjects suffering from specific manifestations or degrees of the disease (i.e. facial lesions, nephritis, endocarditis, hemolytic anemia and leukopenia).

VI. Predictive Medicine

The present invention pertains to the field of predictive medicine in which diagnostic assays, prognostic assays, pharmacogenetics and monitoring clinical trials are used for prognostic (predictive) purposes to thereby treat an individual prophylactically. Accordingly, one aspect of the present invention relates to diagnostic assays for determining marker protein and/or nucleic acid expression as welt as marker protein activity, in the context of a biological sample (e.g., blood, serum, cells, tissue) to thereby determine whether an individual is afflicted with a disease or disorder, or is at risk of developing a disorder, associated with increased or decreased marker protein expression or activity. The invention also provides for prognostic (or predictive) assays for determining whether an individual is at risk of developing a disorder associated with marker protein, nucleic acid expression or activity. For example, the number of copies of a marker gene can be assayed in a biological sample. Such assays can be used for prognostic or predictive purposes to thereby phophylactically treat an individual prior to the onset of a disorder (e.g., systemic lupus erythematosus)characterized by or associated with marker protein, nucleic acid expression or activity.

Another aspect of the invention pertains to monitoring the influence of agents (e.g., drugs, compounds) on the expression or activity of marker in clinical trials.

These and other agents are described in further detail in the following sections.

1. Diagnostic Assays

An exemplary method for detecting the presence or absence of marker protein or nucleic acid of the invention in a biological sample involves obtaining a biological sample from a test subject and contacting the biological sample with a compound or an agent capable of detecting the protein or nucleic acid (e.g., MRNA, genomic DNA) that encodes the marker protein such that the presence of the marker protein or nucleic acid is detected in the biological sample. A preferred agent for detecting mRNA or genomic DNA corresponding to a marker gene or protein of the invention is a labeled nucleic acid probe capable of hybridizing to a mRNA or genomic DNA of the invention. Suitable probes for use in the diagnostic assays of the invention are described herein.

A preferred agent for detecting marker protein is an antibody capable of binding to marker protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to defeat marker mRNA, protein, or genomic DNA in a biological sample in vitro as well as in vivo. For example, in vitro techniques for detection of marker mRNA include Northern hybridizations and in situ hybridizations. In vitro techniques for detection of marker protein include enzyme linked immunosorbent assays (ELISAs), Western blots, immunoprecipitations and immunofluoresCence. In vitro techniques for detection of marker genomic DNA include Southern hybridizations. Furthermore, in vivo techniques for detection of marker protein include introducing into a subject a labeled anti-marker antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample (e.g. , noninvolved tissue or from a non-diseased subject) from a control subject, contacting the control sample with a compound or agent capable of detecting marker protein, mRNA, or genomic DNA, such that the presence of marker protein, mRNA or genomic DNA is detected in the biological sample, and comparing the presence of marker protein, mRNA or genomic DNA in the control sample with the presence of marker protein, mRNA or genomic DNA in the test sample.

The invention also encompasses kits for detecting the presence of marker in a biological sample. For example, the kit can comprise a labeled compound or agent capable of detecting marker protein or mRNA in a biological sample; means for determining the amount of marker in the sample; and means for comparing the amount of marker in the sample with a standard. The compound or agent can be packaged in a suitable container. The kit can further comprise instructions for using the kit to detect marker protein or nucleic acid.

2. Prognostic Assays

The diagnostic methods, described herein can furthermore be utilized to identify subjects having or at risk of developing a disease or disorder associated with aberrant marker expression or activity. As used herein, the term “aberrant” includes a marker expression or activity which deviates from the wild type marker expression or activity. Aberrant expression or activity includes increases or decreased expression or activity, as well as expression or activity which does not follow the wild type developmental pattern of expression or the subcellular pattern of expression. For example, aberrant marker expression or activity is intended to include the cases in which a mutation in the marker gene causes the marker gene to be under-expressed or over-expressed and situations in which such mutations result in a non-functional marker protein or a protein which does not function in a wild-type fashion, e.g., a protein which does not interact with a marker ligand or one which interacts with a non marker protein ligand.

The assays described herein, such as the preceding diagnostic assays or the following assays, can be utilized to identify a subject having or at risk of developing a disorder associated with a misregulation in marker protein activity or nucleic acid expression, such as systemic lupus erythematosus. Alternatively, the prognostic assays can be utilized to identify a subject having or at risk for developing a, disorder associated with a misregulation in marker protein activity or nucleic acid expression, such as systemic lupus erythematosus. Thus, the present invention provides a method for identifying a disease or disorder associated with aberrant marker expression or activity in which a test sample is obtained from a subject and marker protein or nucleic acid (e.g., mRNA or genomie DNA) is detected, wherein the presence of marker protein or nucleic acid is diagnostic for a subject having or at risk of developing a disease or disorder associated with aberrant marker expression or activity. As used herein, a “test sample” includes a biological sample obtained from a subject of interest. For example, a test sample can be a biological fluid (e.g., blood PBMCs), cell sample, or tissue (e.g., kidney).

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with increased or degreased marker expression or activity. For example, such methods can be used to determine whether a subject can be effectively treated with an agent for a disorder such as systemic lupus erythematosus. Thus, the present invention provides methods for determining whether a subject can be effectively treated with an agent for a disorder associated with increased or decreased marker expression or activity in which a test sample is obtained and marker protein or nucleic acid expression or activity is detected (e.g., wherein the abundance of marker protein or nucleic acid expression or activity is diagnostic for a subject that can be administered the agent to treat a disorder associated with increased or decreased marker expression or activity).

The methods of the invention can also be used to detect genetic alterations in a marker gene, thereby determining if a subject with the altered gene is at risk for a disorder characterized by misregulation in marker protein activity or nucleic acid expression, such as systemic lupus erythematosus. In preferred embodiments, the methods include detecting, in a sample of cells from the subject, the presence or absence of a genetic alteration characterized by at least one of an alteration affecting the integrity of a gene encoding a marker-protein, or the mis-expression of the marker gene. For example, such genetic alterations can be detected by ascertaining the existence of at least one of 1) a deletion of one or more nucleotides from a marker gene; 2) an addition of one or more nucleotides to a marker gene; 3) a substitution of one or more nucleotides of a marker gene, 4) a chromosomal rearrangement of a marker gene; 5) an alteration in the level of a messenger RNA transcript of a marker gene, 6) aberrant modification of a ma=ker gene, such as of the methylation pattern of the genomic DNA, 7) the presence of a non-wild type splicing pattern of a messenger RNA transcript of a marker gene, 8) a non-wild type level of a marker-protein, 9) allelic loss of a marker gene, and 10) inappropriate post-translational modification of a marker-protein. As described herein, there are a large number of assays known in the art which can be used for detecting alterations in a marker gene. A preferred biological sample is a tissue (e.g., kidney) or blood sample isolated by conventional means from a subject.

In certain embodiments, detection of the alteration involves the use of a probe/primer in a polymerase chain reaction (PCR) (see, e.g., U.S. Pat. Nos. 4,683,(95 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Mail. Acad. Sci. USA 91:360-364), the latter of which can be particularly useful for detecting point mutations in the marker-gene (see Abravaya et al. (1995) Nucleic Acids Res. 23:675-682). This method can include the steps of collecting a sample of cells from a subject, isolating nucleic acid (e.g., genomic, MRNA or both) from the cells of the sample, contacting the nucleic acid sample with one or more primers which specifically hybridize to a marker gene under conditions such that hybridization and amplification of the marker-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a marker gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in a marker gene or a gene encoding a marker protein of the invention can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in marker can be identified in two dimensional arrays containing light generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the marker gene and detect mutations by comparing the sequence of the sample marker with the corresponding wild-type (control) sequence. Examples of sequencing reactions include those based on techniques developed by Maxam and Gilbert ((1977) Proc. Natl. Acad. Scl. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94116101; Cohen et al. (1996) Adv. Chromatogr. 36:127-162; and Griffin et al. (1993) Appl. Biochem. Biotechnol. 38:147-159).

Other methods for detecting mutations in the marker gene or gene encoding a marker protein of the invention include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes by hybridizing (labeled) RNA or DNA containing the wild-type marker sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nucleus to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 517:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in marker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1652). According to an exemplary embodiment, a probe based on a marker sequence, e.g., a wild-type marker sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in marker genes or genes encoding a marker protein of the invention. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech Appl. 9:73-79). Single-stranded DNA fragments of sample and control marker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in elecrtophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing prepackaged diagnostic kits comprising at least one probe nucleic acid or antibody reagent described herein, which may be conveniently used, e.g., in clinical settings to diagnose subjects exhibiting symptoms or family history of a disease or illness involving a marker gene.

Furthermore, any cell type or tissue in which marker is expressed may be utilized in the prognostic assays described herein.

3. Monitoring of Effects During Clinical Trials

Monitoring the influence of agents (e.g., drugs) on the expression or activity of a marker protein (e.g., the modulation of systemic lupus erythematosus) can be applied not only in basic drug screening, but also in clinical trials. For example, the effectiveness of an agent determined by a screening assay as described herein to increase marker gene expression, protein levels, or upregulate marker activity, can be monitored in clinical trials of subjects exhibiting decreased marker gene expression, protein levels, or downruegulated marker activity. Alternatively, the effectiveness of an agent determined by a screening assay to decrease marker gene expression, protein levels, or downregulate marker activity, can be monitored in clinical trials of subjects exhibiting increased marker gene expression, protein levels, or upregulated marker activity. In such clinical trials, the expression or activity of a marker gene, and preferably, other genes that have been implicated in, for example, a marker-associated disorder (e.g., systemic lupus erythematosus) can be used as a “read out” or markers of the phenotype of a particular cell.

For example, and not by way of limitation, genes, including marker genes and genes encoding a marker protein of the invention, that are modulated in cells by treatment with an agent (e.g., compound, drug or small molecule) which modulates marker activity (e.g., identified in a screening assay as described herein) can be identified. Thus, to study the effect of agents on marker-associated disorders (e.g., systemic lupus erythematosus), for example, in a clinical trial, cells can be isolated and RNA prepared and analyzed for the levels of expression of marker and other genes implicated in the marker-associated disorder, respectively. The levels of gene expression (e.g., a gene expression pattern) can be quantified by northern blot analysis or RT-PCR, as described herein, or alternatively by measuring the amount of protein produced, by one of the methods as described herein, or by measuring the levels of activity of marker or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of a marker protein, mRNA, or genomic DNA in the pre-administration sample; (iii) obtaining one or more post-administration samples from the subject; (iv) detecting the level of expression or activity of the marker protein, mRNA, or genomic DNA in the post-administration samples; (v) comparing the level of expression or activity of the marker protein, mRNA, or genomic DNA in the pre-administration sample with the marker protein, mRNA, or genomic DNA in the post administration sample or samples; and (vi) altering the administration of the agent to the subject accordingly. For example, increased administration of the agent may be desirable to increase the expression or activity of marker to higher levels than detected, i.e., to increase the effectiveness of the agent. Alternatively, decreased administration of the agent may be desirable to decrease expression or activity of marker to lower levels than detected, i.e. to decrease the effectiveness of the agent. According to such an embodiment, marker expression or activity may be used as an indicator of the effectiveness of an agent, even in the absence of an observable phenotypic response.

C. Methods of Treatment

The present invention provides for both prophylactic and therapeutic methods of treating a subject at risk for (or susceptible to) a disorder or having a disorder associated with aberrant marker expression or activity. With regards to both prophylactic and therapeutic methods of treatment, such treatments may be specifically tailored or modified, based on knowledge obtained from the field of pharmacogenomics. “Pharmacogenomics”, as used herein, includes the application of genomics technologies such as gene sequencing, statistical genetics, and gene expression analysis to drugs in clinical development and on the market. More specifically, the term refers the study of how a subject's genes determine his or her response to a drug (e.g., a subject's “drug response phenotype”, or “drug response genotype”.) Thus, another aspect of the invention provides methods for tailoring an individual's prophylactic or therapeutic treatment with either the marker molecules of the present invention or marker modulators according to that individual's drug response genotype. Pharmacogenomics allows a clinician or physician to target prophylactic or therapeutic treatments to subjects who will most benefit from the treatment and to avoid treatment of subjects who will experience toxic drug-related side effects.

1. Prophylactic Methods

In one aspect, the invention provides a method for preventing in a subject, a disease or condition (e.g., systemic lupus erythematosus) associated with increased or decreased marker expression or activity, by administering to the subject a marker protein or an agent which modulates marker protein expression or at least one marker protein activity. Subjects at risk for a disease which is caused or contributed to by increased or decreased marker expression or activity can be identified by, for example, any or a combination of diagnostic or prognostic assays as described herein. Administration of a prophylactic agent can occur prior to the manifestation of symptoms characteristic of the differential marker protein expression, such that a disease or disorder is prevented or, alternatively, delayed in its progression. Depending on the type of marker aberrancy (e.g., increase or decrease in expression level), for example, a marker protein, marker protein agonist or marker protein antagonist agent can be used for treating the subject. The appropriate agent can be determined based on screening assays described herein.

2. Therapeutic Methods

Another aspect of the invention pertains to methods of modulating marker protein expression or activity for therapeutic purposes. Accordingly, in an exemplary embodiment, the modulatory method of the invention involves contacting a cell with a marker protein or agent that modulates one or more of the activities of a marker protein activity associated with the cell. An agent that modulates marker protein activity can be an agent as described herein, such as a nucleic acid or a protein, a naturally-occurring target molecule of a marker protein (e.g., a marker protein substrate), a marker protein antibody, a marker protein agonist or antagonist, a peptidomimetic of a marker protein agonist or antagonist, or other small molecule. In one embodiment, the agent stimulates one or more marker protein activities. Examples of such stimulatory agents include active marker protein and a nucleic acid molecule encoding marker protein that has been introduced into the cell. In another embodiment, the agent inhibits one or more marker protein activities. Examples of such inhibitory agents include antisense marker protein nucleic said molecules, anti-marker protein antibodies, and marker protein inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a marker protein or nucleic acid molecule. In one embodiment, the method involves administering an agent (e.g., an agent identified by a screening assay described herein), or combination of agents that modulates (e.g., upregulates or downregulates) marker protein expression or activity. In another embodiment, the method involves administering a marker protein or nucleic acid molecule as therapy to compensate for reduced or aberrant marker protein expression or activity.

Stimulation of marker protein activity is desirable in situations in which marker protein is abnormally downregulated and/or in which increased marker protein activity is likely to have a beneficial effect. For example, stimulation of marker protein activity is desirable in situations in which a marker is downregulated and/or in which increased marker protein activity is likely to have a beneficial erect. Likewise, inhibition of marker protein activity is desirable in situations in which marker protein is abnormally upregulated and/or in which decreased marker protein activity is likely to have a beneficial effect.

3. Pharmacogenomics

The marker protein and nucleic acid molecules of the present invention, as well as agents, inhibitors or modulators which have a stimulatory or inhibitory effect on marker protein activity (e.g., marker gene expression) as identified by a screening assay described herein can be administered to individuals to treat (prophylactically or therapeutically) marker-associated disorders (e.g., systemic lupus erythematosus) associated with aberrant marker protein activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a marker molecule or marker modulator as well as tailoring the dosage and/or therapeutic regimen of treatment with a marker molecule or marker modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11) :983-985 and Linden, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically substantial number of subjects taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a marker protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYPZC 19) has provided an explanation as to why some subjects do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a marker molecule or marker modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a marker molecule or marker modulator, such as a modulator identified by one of the exemplary screening assays described herein.

This invention is further illustrated by the following examples which should not be construed as limiting. The contents of all references, patents and published patent applications cited throughout this application, as well as the Figures and Tables are incorporated herein by reference.

### EXAMPLES

### EXAMPLE 1

### IDENTIFICATION AND CHARACTERIZATION OF MARKER cDNA IN MURINE MODEL OF SEVERE AUTOIMMUNE KIDNEY DISEASE

A. Development of Autoimmune Kidney Disease and Isolation of Immune Cells

NZB/NZW F1 (B/W) mice develop an autoimmune kidney disease that is analogous to human systemic erythematosus. Mice, aged at intervals from 12 weeks (asymptomatic) to 42 weeks (diseased) were chosen to mimic the clinical presentation of patients with established SLE. By seven months of age, mice begin to develop nephritis characterized by proteinuria, anti-DNA antibody production and histopathologic changes in the kidney.

Whole kidney samples from six groups of mice were harvested: from young, generally asymptomatic mice (12 weeks), from mice evidencing onset at 25 weeks and from older, diseased mice (36 and 42 weeks), as well as from two groups of disease-free mice aged at 3 months (C57/3m) and 8 months (C57/8m).

B. Isolation of RNA

Total RNA was isolated using the RNeasy mini kit (Quiagen, Hilden, Germany). To prepare cRNA for hybridization, 5 μg of total RNA was denatured at 70° C. with T7-tagged oligo-dT primer, cooled on ice, then reverse transcribed with 200 units Superscript RT II at 50° C. for 1 hour in 1x first strand buffer, 10 mM DTT and 0.5 mM of each dNTP (Gibco BRL, Gaiethersburg, Md.). Second strand cDNA was synthesized by adding 40 units DNA pol I, 10 units E. coli DNA ligase, 2 units Rnase H, 30 μL second strand buffer, 3 μl 10 mM each dNTP, and water to 150 μL final volume and incubating at 15.8° C. for 2 hours. The resulting cDNA was extracted once with phenol/chloroform/isoamylalcohol. cDNA was separated on a Phase Lock Gel tube at maximum speed for 2 min and precipitated with sodium acetate and 100 ethanol. The resulting pellet was washed with 80% ethanol, was dried and was resuspended in diethylpyrocarbonate-treated (DEPC-treated) water.

Labeled RNA was prepared from clones containing a T7 RNA polymerase promoter site by incorporating labeled ribonucleotides in an in vitro transcription (IVT) reaction. Half of the purified cDNA was used for in vitro transcription with a T7 RNA polymerase kit, following manufacturer instructions and using an overnight 37 ° C. incubation, thereby incorporating biotinylated CTP and UTP. Labeled RNA was purified using RNeasy columns (Quiagen). RNA was concentrated and then quantitated by spectrophotometry. Labeled RNA (13-15 μg) was fragmented in 40 mM Tris-acetate 8.0, 100 mM potassium acetate, 30 mM magnesium acetate for 35 min at 94 C. in a total volume of 40 μL.

C. Array Hybridization and Detection of Fluorescence

The labeled and fragmented RNA probes were diluted in 1× MES buffer, B10948, Bio C, B cre, 100 μg/ml herring sperm DNA, and 50 μg/ml acetylated BSA. New probes were pre-hybridized in a microfuge tube with glass beads at 45° C. overnight to remove debris. Oligonucleotide arrays composed of approximately 11,000 murine genes (Microarray, Affymetrix, Cat Nos. SubA #510243, SubB #510244) were pre-hybridized with 1× MES hybridization buffer at 45° C. for 5 min and then insoluble material was removed by centrifugation. Pre-hybridization buffer was removed from oligo array cartridges, 200 μL probe added and cartridges were hybridized for 16 hours at 45° C. at 60 rpm. After hybridization, probes were removed and the cartridges washed extensively with 6× SSPET using a fluidics station (Affymetrix). Following hybridization, the solutions were removed, the arrays were washed with 6× SSPE-T at 22° C. for 7 min, and then washed with 0.5× SSPE-T at 40° C. for 15 minutes. When biotin-labeled RNA was used, the hybridized RNA was stained with a streptavidin-phycoerythrin conjugate (Molecule Probes, Eugene, Oreg.) prior to reading. Hybridized arrays were stained with 2 μg/ml streptavidin-phycoerythrin in 6× SSPE-T at 40° C. for 5 minutes and subsequently stained with goat antibody against streptavidin-biotin. The arrays were again washed and stained with streptavidin SSPE-T prior to being reading. The arrays were read using a scanning confocal microscope made for Affymetrix by Molecular Dynamics (commercially available through Affymetrix, Santa Clara, Calif.). The scanner uses an argon ion laser as the excitation source, with the emission detected by a photomultiplier tube through either a 530 nm bandpass filter (fluorescein), or a 560 nm longpass filter (phycoerythrin). Nucleic acids of either sense or antisense orientations were used in hybridization experiments. Arrays with probes for either orientation (reverse complements of each other) are made using the same set of photolithographic masks by reversing the order of the photochemical steps and incorporating the complementary nucleotide.

D. Quantitative Analysis of Hybridization Patterns and Insensitivities

Following a quantitative scan of an array, or biochip, a grid is aligned to the image using the known dimensions of the array and the comer control regions as markers. The image is reduced to a simple text file containing position and intensity information using software developed at Affymetrix (GENECHIP 3.0 software). This information is merged with another text file that contains information relating physical position on the array to probe sequence and the identity of the RNA and the specific part of the RNA for which the oligonucleotide probe is designed. The quantitative analysis of the hybridization results involves a simple form of pattern recognition based on the assumption that, in the presence of a specific RNA, the PM probes will hybridize more strongly on average than their MM partners. The number of instances in which the PM hybridization signal is larger than the MM signal is computed along with the average of the logarithm of the PM/MM ratios for each probe set. These values are used to make a decision (using a predefined decision matrix) concerning the presence or absence of an RNA. To determine the quantitative RNA abundance, the average of the differences (PM minus MM) for each probe family is calculated. The advantage of the difference method is that signals from random cross-hybridization contribute equally, on average, to the PM and MM probes, while specific hybridization contributes more to the PM probes. By averaging the pairwise differences, the real signals add constructively while the contributions from cross-hybridization tend to cancel. When assessing the differences between two different RNA samples, the hybridization signals from side-by-side experiments on identically synthesized arrays are compared directly. The magnitude of the changes in the average of the difference (PM−MM) values is interpreted by comparison with the results of spiking experiments as well as the signals observed for the internal standard bacterial and phage RNAs spiked into each sample at a known amount. Data analysis programs developed at Affymetrix, such as the GENECHIP 3.0 software, perform these operation automatically.

Distinct gene expression patterns emerged between the young, asymptomatic mice and the older diseased mice. In order to identify the most active genes in SLE development, genes were sought which revealed a pattern of deregulation in the older mice, as compared to the younger mice (asymptomatic, or onset stage). The asymptomatic and onset stages had similar expression levels when compared to control samples from disease-free C57 mice). The genes demonstrating differential expression between the younger and older mice are set forth in Tables 1 and 3-4. Moreover, as validation, there are several genes which were previously known to be associated with SLE in which are provided separately in Table 2. The vast majority (95%) of the genes however, were not significantly different between young and old C57BL/6 mice, a disease free strain, indicating that most of the expression differences observed in the disease strain were not due to normal age-related changes in the kidney.

To identify genes that were differentially regulated between the early stage of the disease and peak stages, the average fold change between the younger and older genes were calculated. Table 1 indicates the average fold change between unreated, diseased mice at 36 weeks versus 12 weeks, and the undiseased C57 mice at 8 months versus 3 months. Moreover, Table 3 provides a list of genes which were up-regulated in the diseased stage versus the asymptomatic or onset stage; while Table 4 provides a list of genes which were down-regulated in the diseased stage. In addition, Table 8 provides a list of genes which are differentially expressed at 25 weeks or earlier, as compared to the expression levels of the asymptomatic 12 week old mice. Many of the genes listed in Table 8 are retroviral in nature.

### EXAMPLE 2

### METHOD OF ASSESSING EFFICACY OF RAPAMYCIN TREATMENT IN MURINE MODEL OF AUTOIMMUNE KIDNEY DISEASE

A. Treatment with Rapamycin

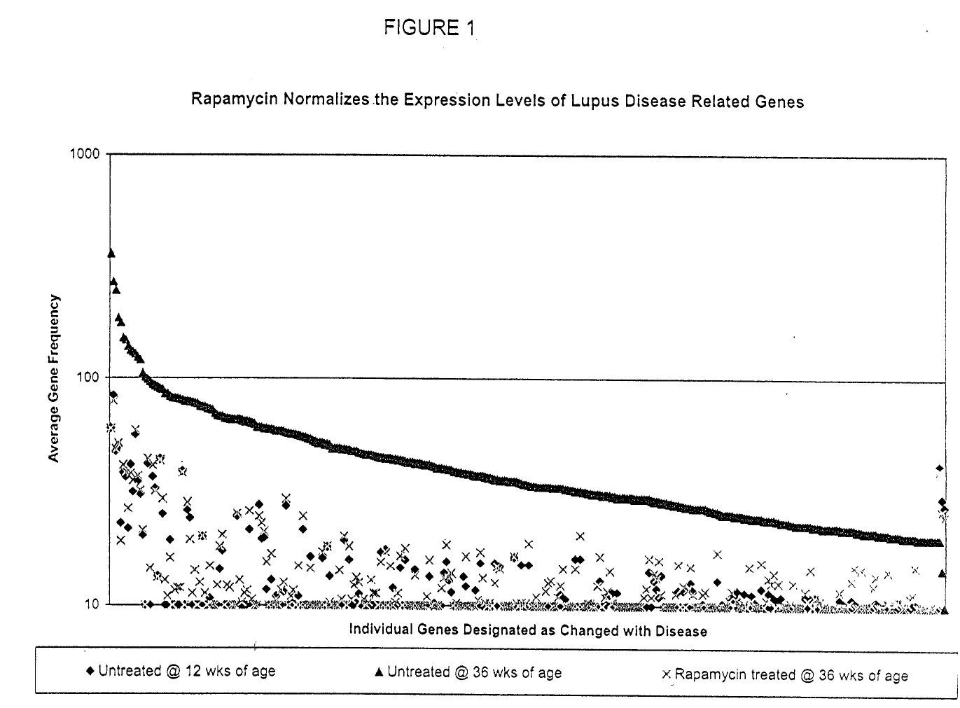

In addition, to further identify disease-related and to assess the efficacy of treatment, rapamycin was administered to NZB/NZW F1 mice starting at 25 weeks old. Rapamycin protocol included 3 doses a week at 5 mg/kg subcutaneously for 8 weeks. The 25 week old mice were selected for onset of nephritis by monitoring for signs of proteinuria (kidney damage can be measured by the amount of albumin excreted). After an 8 week course of treatment, the kidneys were harvested and isolation of RNA was performed as described above, with the rapamycin-treated samples being compared to the untreated samples at 12 and 36 weeks. Table 5 identifies genes which were up-regulated in Example 1 but were reduced in expression level upon treatment of rapamycin at 36 weeks, as compared untreated mice at 36 weeks. In particular, by indicating “yes”, Table 5 identifies those up-regulated genes which were ‘normalized’ by rapamycin treatment (having a difference in expression of less than five as compared to untreated, asymptomatic mice of 5 weeks). As shown in FIG. 1, treatment with rapamycin reduced expression levels of the indicated genes from diseased levels to nondiseased levels, thus suggesting that rapamycin may be efficacious in treating SLE. These results were confirmed by prolonged survival and decreasing anti-DNA antibody production.

The genes were also clustered hierarchically into groups on the basis of similarity of function to evaluate similarities or trends in up- or down-regulation. These genes and their groupings are listed in Table 6.

B. Treatment with Anti-B7

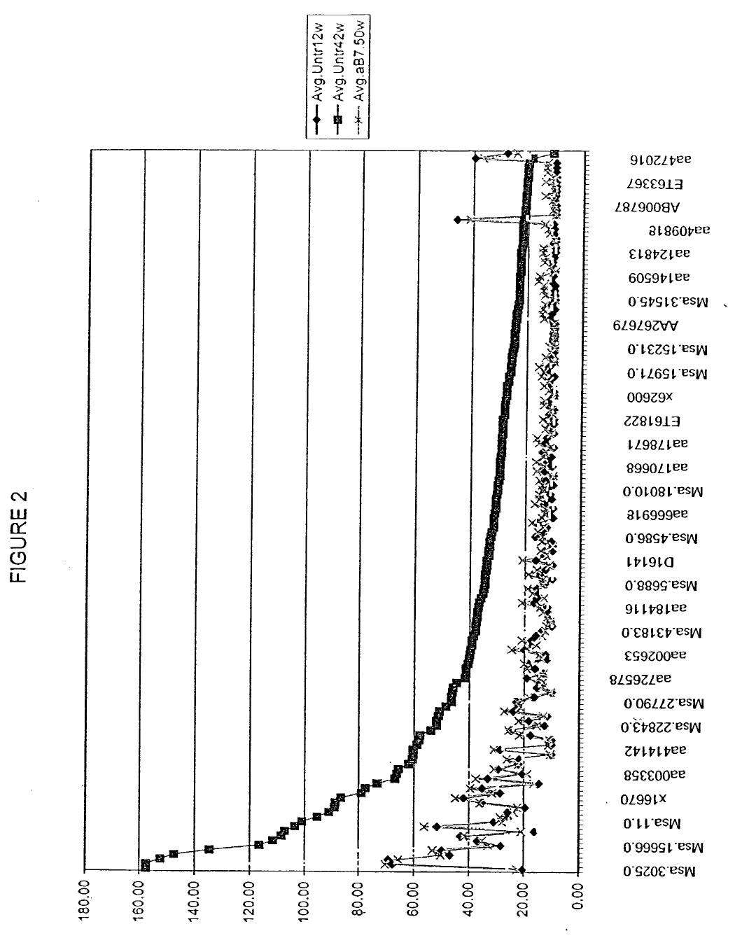

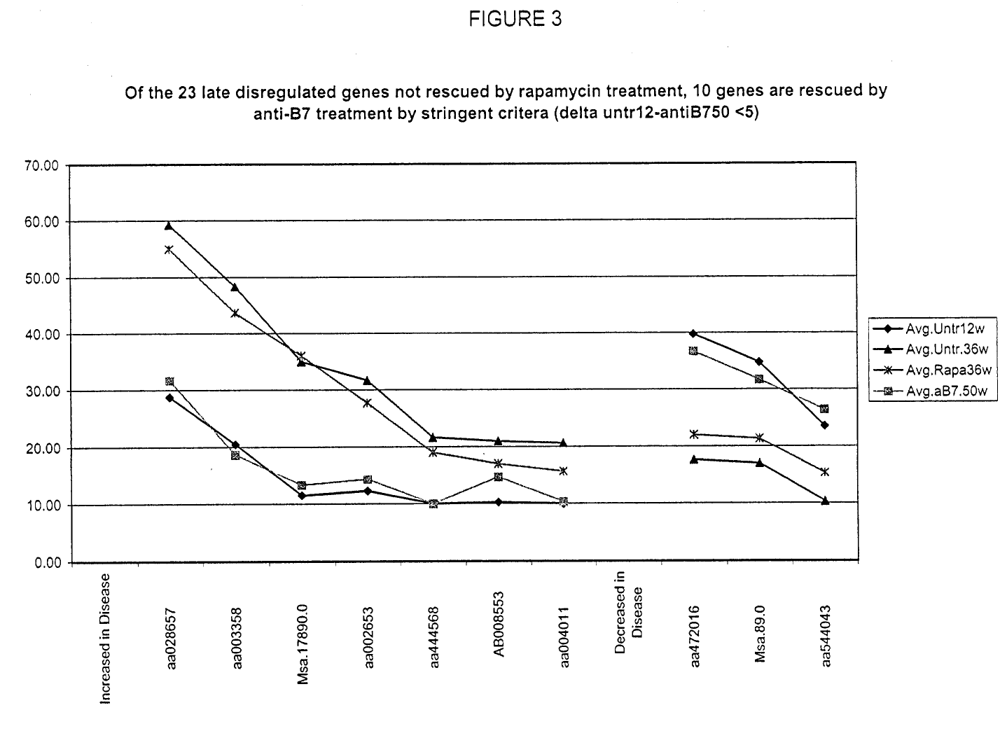

In addition to rapamycin, the efficacy of anti-B7 treatment was also assessed in NZB/NZW F1 mice. Anti-B7 200 μg anti-murine B7-1 and 200 μg anti-murine B7-2 were administered three times a week subcutaneously for two weeks, starting at onset of the disease (25 weeks). As with rapamycin, the mice were selected at onset by monitoring for signs of proteinuria. After the anti-B7 treated mice were about 50 weeks old, the kidneys were harvested and isolation of RNA, was performed as described above, with the anti-B7 treated mice being compared to the untreated samples at 12 weeks and 42 weeks. The genes which were normalized (expressing a difference of less than five as compared to untreated 12 week old mice) are listed in Table 7. As shown in FIG. 2, treatment with anti-B7 reduced expression levels of the indicated genes from diseased levels to nondiseased levels, and was in some cases more efficacious than rapamycin in treating SLE. Again, these results were confirmed by prolonged survival (untreated, diseased mice did not survive to 50 weeks) and decreasing grade of anti-DNA antibody production. Furthermore, as shown in FIG. 3, of the 23 genes which were not normalized by rapamycin treatment in Example 2(A) above, 10 genes were normalized by anti-B7 treatment.

Other variations and modifications of this invention will be obvious to those skilled in the art. This invention is not limited except as set forth in the claims.

TABLE 1
Differentially-regulated genes

| | | | | | | | | Untr | C57 | |
|---|---|---|---|---|---|---|---|---|---|---|
| | | | | | | | | 36w/12w | 8m/3m | |
| | | | | | | | | (fold | (fold | |
| Name | Accession# | Avg Untr12w | Avg.Untr25w | Avg Untr.36w | Avg Untr42w | Avg C57/3m | Avg C57/8m | change) | change) | Description |
| E_TC19066_s | AA444568 | 10.00 | 17.33 | 21.67 | 33.00 | 12.00 | 13.00 | 2.17 | 1.08 | vf79g11.r1 Soares mouse mammary gland NbMMG |
| | | | | | | | | | | Mus musculus cDNA clone 850052 5′ |
| ADAMTS1_s | D67076 | 10.00 | 10.00 | 36.00 | 46.33 | 10.00 | 10.33 | 3.60 | 1.03 | Mouse mRNA for secretory protein containing |
| | | | | | | | | | | thrombospondin motifs, complete cds |
| LPC1_s | X07486 | 15.00 | 12.67 | 36.00 | 42.67 | 10.00 | 14.67 | 2.40 | 1.47 | Mouse mRNA for lipocortin l |
| CAL1H_f | D10024 | 20.50 | 18.00 | 105.67 | 106.00 | 42.50 | 45.00 | 5.15 | 1.06 | D10024 Mouse mRNA for protein-tyrosine kinase |
| | | | | | | | | | | substrate p36 (calpactin l heavy chain), complete cds |
| CAL1H_f | M14044 | 22.00 | 17.33 | 139.67 | 159.00 | 47.50 | 50.33 | 6.35 | 1.06 | Mouse calpactin l heavy chain (p36) mRNA, complete |
| | | | | | | | | | | cds |
| W98864_f | W98864 | 12.00 | 15.00 | 29.33 | 30.33 | 13.00 | 20.00 | 2.44 | 1.54 | W98864 mg11h11.r1 Mus musculus cDNA, 5′ end |
| ANX5_s | U29396 | 13.00 | 13.00 | 40.00 | 38.00 | 22.00 | 29.67 | 3.08 | 1.35 | Mus musculus annexin V (Anx5) mRNA, complete cds |
| AF032466_s | AF032466 | 10.25 | 10.33 | 21.33 | 36.00 | 10.00 | 16.67 | 2.08 | 1.67 | Mus musculus arginase II mRNA, complete cds |
| ARH9_s | X80638 | 47.00 | 43.33 | 104.67 | 147.33 | 48.50 | 51.33 | 2.23 | 1.06 | M. musculus rhoC mRNA |
| ARHGDIB_s | L07918 | 10.00 | 10.00 | 26.00 | 32.00 | 12.50 | 14.33 | 2.60 | 1.15 | Mus musculus GDP-dissociation inhibitor mRNA, |
| | | | | | | | | | | preferentially expressed in hematopoietic cells, |
| | | | | | | | | | | complete cds |
| AF004591_s | AF004591 | 44.25 | 41.33 | 90.00 | 94.33 | 149.50 | 178.00 | 2.03 | 1.19 | Mus musculus copper transport protein Atox1 (ATOX1) |
| | | | | | | | | | | mRNA, complete cds |
| ATPA_s | U13837 | 47.50 | 26.67 | 22.67 | 28.33 | 19.00 | 17.67 | 0.48 | 0.93 | U13837 Mus musculus vacuolar adenosine |
| | | | | | | | | | | triphosphatase subunit A gene, complete cds |
| BGN_s | L20276 | 71.25 | 54.67 | 169.33 | 134.33 | 73.50 | 107.00 | 2.38 | 1.46 | Mouse biglycan (Bgn) mRNA, complete cds |
| CALB1_s | M21531 | 76.75 | 57.00 | 26.67 | 37.00 | 138.00 | 105.67 | 0.35 | 0.77 | Mus musculus calbindin (PCD-29) mRNA, complete cds |
| CAPPB1_f | U10406 | 35.75 | 37.00 | 72.67 | 90.33 | 40.50 | 52.33 | 2.03 | 1.29 | Mus musculus capping protein beta-subunit isoform |
| CCR4_f | X04120 | 50.00 | 72.33 | 112.33 | 134.67 | 35.50 | 50.33 | 2.25 | 1.42 | M. musculus intracisternal A-particle IAP-IL3 genome |
| | | | | | | | | | | deleted type l element inserted 5′ to the interleukin-3 |
| | | | | | | | | | | gene |
| CD14_s | X13333 | 25.50 | 28.67 | 89.33 | 95.33 | 21.50 | 27.33 | 3.50 | 1.27 | Mouse CD14 mRNA for myelid cell-specific leucine-rich |
| | | | | | | | | | | glycoprotein |
| AB008553 | AB008553 | 10.25 | 12.67 | 21.00 | 21.00 | 10.00 | 15.33 | 2.05 | 1.53 | Mus musculus mRNA for mLGP85/LIMP II, complete |
| | | | | | | | | | | cds |
| CD80_s | M55561 | 10.00 | 10.00 | 31.33 | 34.00 | 10.00 | 15.33 | 3.13 | 1.53 | Mouse phosphatidylinositol-linked antigen (pB7) mR |
| AB009287_s | AB009287 | 10.00 | 11.33 | 23.33 | 29.00 | 10.00 | 12.33 | 2.33 | 1.23 | Mus musculus gene for Macrosialin, complete cds |
| TESK1_s | J04170 | 10.00 | 10.00 | 22.67 | 36.33 | 10.00 | 10.00 | 2.27 | 1.00 | Mouse B-cell differentiation antigen Lyb-2.1 protein, |
| | | | | | | | | | | complete cds |
| CEBPB_s | X62600 | 10.00 | 10.00 | 22.33 | 27.33 | 10.50 | 10.00 | 2.23 | 0.95 | M. musculus mRNA for C/EBP beta |
| AB000713 | AB000713 | 10.00 | 10.00 | 23.00 | 50.00 | 10.00 | 10.00 | 2.30 | 1.00 | Mus musculus mCPE-R mRNA for CPE-receptor, |
| | | | | | | | | | | complete cds |
| AB000713_g | AB000713 | 16.00 | 13.33 | 48.67 | 107.33 | 10.00 | 12.00 | 3.04 | 1.20 | Mus musculus mCPE-R mRNA for CPE-receptor, |
| | | | | | | | | | | complete cds |
| CLU_s | L08235 | 163.25 | 115.00 | 415.33 | 608.00 | 270.00 | 362.00 | 2.54 | 1.34 | Mus musculus clusterin mRNA, complete cds |
| CNN2_f | Z19543 | 15.25 | 16.67 | 34.33 | 35.33 | 16.50 | 22.00 | 2.25 | 1.33 | Z19543 M. musculus h2-calponin cDNA |
| COL6A2_s | X65582 | 11.25 | 12.00 | 33.33 | 25.00 | 13.50 | 17.33 | 2.96 | 1.28 | M. musculus mRNA for alpha-2 collagen VI |
| CP_s | U49430 | 20.50 | 15.67 | 69.00 | 157.67 | 22.50 | 28.33 | 3.37 | 1.26 | U49430 Mus musculus ceruloplasmin mRNA, complete |
| | | | | | | | | | | cds |
| CRIP_f | M13018 | 10.25 | 11.33 | 48.00 | 49.67 | 14.00 | 25.67 | 4.68 | 1.83 | M13018 Mouse cysteine-rich intestinal protein (CRIP) |
| | | | | | | | | | | mRNA, complete cds |
| CRIP_f | M13018 | 10.00 | 10.67 | 49.33 | 55.33 | 14.00 | 18.67 | 4.93 | 1.33 | Mouse cysteine-rich intestinal protein (CRIP) mRNA, |
| | | | | | | | | | | complete cds |
| CSTB_f | U59807 | 14.50 | 15.33 | 68.00 | 71.67 | 24.00 | 27.33 | 4.69 | 1.14 | Mus musculus cystatin B (Stfb) gene, complete cds |
| FISP12_s | M70642 | 19.50 | 20.00 | 83.00 | 79.33 | 30.50 | 24.33 | 4.26 | 0.80 | Mouse FISP-12 protein (fisp-12) mRNA, complete cds |
| CTSC_s | AA144887 | 10.00 | 10.00 | 26.33 | 27.67 | 10.00 | 10.00 | 2.63 | 1.00 | AA144887 mr11d06.r1 Mus musculus cDNA, 5′ end |
| CTSC_s | U89269 | 16.50 | 12.33 | 54.00 | 71.67 | 11.00 | 11.33 | 3.27 | 1.03 | Mus musculus preprodipeptidyl peptidase l mRNA, |
| | | | | | | | | | | complete cds |
| E_CTSS_s | AA146437 | 10.00 | 10.00 | 42.67 | 53.00 | 11.00 | 16.67 | 4.27 | 1.52 | AA146437 mr05a08.r1 Mus musculus cDNA, 5′ end |
| E_CTSS_s | AA089333 | 10.00 | 10.00 | 45.33 | 41.67 | 10.00 | 15.33 | 4.53 | 1.53 | AA089333 mo60e02.r1 Mus musculus cDNA, 5′ end |
| E_TC26364_s | AA014563 | 38.25 | 47.00 | 77.33 | 101.67 | 43.00 | 57.67 | 2.02 | 1.34 | mi67c05.r1 Soares mouse embryo NbME13.5 14.5 Mus |
| | | | | | | | | | | musculus cDNA clone 468584 5′. |
| E_G1P3_s | AA120109 | 26.50 | 27.67 | 79.00 | 82.33 | 53.00 | 50.67 | 2.98 | 0.96 | AA120109 mq09a11.r1 Mus musculus cDNA, 5′ end |
| E_1193052_s | AA711625 | 102.00 | 109.67 | 317.67 | 430.00 | 145.50 | 221.67 | 3.11 | 1.52 | vu31g07.r1 Stratagene mouse Tcell 937311 Mus |
| | | | | | | | | | | musculus cDNA clone 1193052 5′similar to |
| | | | | | | | | | | SW IN17_HUMAN P40305 INTERFERON-ALPHA |
| | | | | | | | | | | INDUCED 11.5 KD PROTEIN,, mRNA sequence. |
| C80103_rc_s | C80103 | 10.00 | 10.00 | 31.67 | 36.67 | 13.00 | 15.33 | 3.17 | 1.18 | Mouse 3.5-dpc blastocyst cDNA Mus musculus cDNA |
| | | | | | | | | | | clone J0076E08 3′, mRNA sequence. |
| POU2F2_s | AA674986 | 11.75 | 10.00 | 37.67 | 21.67 | 10.00 | 10.67 | 3.21 | 1.07 | vq57g08.r1 Barstead mouse proximal colon MPLRB6 |
| | | | | | | | | | | Mus musculus cDNA clone 1106462 5′, mRNA |
| | | | | | | | | | | sequence. |
| D17NK17_s | U69488 | 10.00 | 10.00 | 22.33 | 35.67 | 10.00 | 10.00 | 2.23 | 1.00 | Mus musculus viral envelope like protein (G7e) gene, |
| | | | | | | | | | | complete cds |
| AA727845_s | AA727845 | 84.50 | 80.33 | 189.67 | 262.00 | 93.00 | 121.33 | 2.24 | 1.30 | vp33f01.r1 Barstead mouse proximal colon MPLRB6 |
| | | | | | | | | | | Mus musculus cDNA clone 1078489 5′, mRNA |
| | | | | | | | | | | sequence. |
| E_D90239_s | AA246000 | 32.75 | 48.33 | 12.67 | 78.33 | 43.00 | 26.00 | 0.39 | 0.60 | mx04h05.r1 Soares mouse NML Mus musculus cDNA |
| | | | | | | | | | | clone 679257 5′ similar to gb D90239 GLYCINE |
| | | | | | | | | | | DEHYDROGENASE (HUMAN), |
| AA409826_rc_s | AA409826 | 34.50 | 20.67 | 78.00 | 105.00 | 30.00 | 33.00 | 2.26 | 1.10 | EST01599 Mouse 7.5 dpc embryo ectoplacental cone |
| | | | | | | | | | | cDNA library Mus musculus cDNA clone C0012A02 3′, |
| | | | | | | | | | | mRNA sequence. |
| AA638539_s | AA638539 | 11.25 | 10.33 | 47.33 | 63.33 | 10.00 | 15.33 | 4.21 | 1.53 | vo54d12.r1 Barstead mouse irradiated colon MPLRB7 |
| | | | | | | | | | | Mus musculus cDNA clone 1053719 5′, mRNA |
| | | | | | | | | | | sequence. |
| E_TC36937_s | AA472016 | 39.75 | 21.33 | 17.67 | 18.67 | 33.00 | 38.67 | 0.44 | 1.17 | vh09f02.r1 Soares mouse mammary gland NbMMG |
| | | | | | | | | | | Mus musculus cDNA clone 874971.5′ |
| AA666918_g | AA666918 | 11.75 | 10.00 | 25.33 | 31.33 | 10.50 | 10.00 | 2.16 | 0.95 | vq87c07.r1 Knowles Solter mouse blastocyst B3 Mus |
| | | | | | | | | | | musculus cDNA clone 1109292 5′, mRNA sequence. |
| X04097_s | X04097 | 102.50 | 68.00 | 35.33 | 40.33 | 184.00 | 185.00 | 0.34 | 1.01 | Mouse kidney testosterone-regulated RP2 mRNA |
| E_TC39388_s | AA028770 | 10.00 | 10.00 | 20.00 | 28.00 | 19.50 | 35.33 | 2.00 | 1.81 | mi15h02.r1 Soares mouse p3NMF19.5 Mus musculus |
| | | | | | | | | | | cDNA clone 463635.5′ |
| E_TC39388_i | AA028770 | 36.50 | 45.00 | 81.33 | 101.00 | 65.50 | 107.67 | 2.23 | 1.64 | mi15ho2.r1 Soares mouse p3NMF19.5 Mus musculus |
| | | | | | | | | | | cDNA clone 463635.5′ |
| E_EEF2_f | W98531 | 11.50 | 20.67 | 35.00 | 37.33 | 16.50 | 30.00 | 3.04 | 1.82 | W98531 mg21e05.r1 Mus musculus cDNA, 5′end |
| CD39L1_s | W10995 | 11.00 | 17.00 | 23.00 | 22.67 | 12.00 | 16.67 | 2.09 | 1.39 | ma41d10.r1 Soares mouse p3NMF19.5 Mus musculus |
| | | | | | | | | | | cDNA clone 313267.5′, mRNA sequence. |
| E_TC27896_s | AA059883 | 10.50 | 10.00 | 21.33 | 23.67 | 10.00 | 10.00 | 2.03 | 1.00 | mj76a06.r1 Soares mouse p3NMF19.5 Mus musculus |
| | | | | | | | | | | cDNA clone 482002 5′ |
| E_PFKFB1 | AA109491 | 71.25 | 41.00 | 26.67 | 33.00 | 53.50 | 62.67 | 0.37 | 1.17 | AA109491 ml92d01.r1 Mus musculus cDNA, 5′end |
| E_TC39517_g | AA451220 | 10.00 | 12.00 | 22.00 | 28.33 | 10.50 | 15.33 | 2.20 | 1.46 | vf83b09.r1 Soares mouse mammary gland NbMMG |
| | | | | | | | | | | Mus musculus cDNA clone 850361 5′ similar to |
| | | | | | | | | | | WP:C14B1.3 CE00900; |
| FKBP5_s | U36220 | 14.25 | 24.33 | 28.33 | 60.33 | 15.50 | 16.00 | 1.99 | 1.03 | Mus musculus FK506 binding protein 51 mRNA, |
| | | | | | | | | | | complete cds |
| U87456_s | U87456 | 128.50 | 78.00 | 44.67 | 57.00 | 97.50 | 86.33 | 0.35 | 0.89 | Mus musculus flavin-containing monooxygenase 1 |
| | | | | | | | | | | (FMO1) mRNA, complete cds |
| E_TC14259_f | AA268913 | 51.25 | 21.33 | 25.00 | 35.33 | 19.50 | 28.67 | 0.49 | 1.47 | va44h06.r1 Soares mouse 3NME12.5 Mus musculus |
| | | | | | | | | | | cDNA clone 734267 5′ |
| FSTL_s | M91380 | 10.00 | 10.00 | 20.00 | 13.00 | 10.00 | 10.00 | 2.00 | 1.00 | Mus musculus TGF-beta-inducible protein (TSC-36) |
| | | | | | | | | | | mRNA, complete cds |
| U72680_s | U72680 | 10.25 | 10.00 | 31.00 | 29.67 | 10.00 | 14.00 | 3.02 | 1.40 | Mus musculus ion channel homolog RIC mRNA, |
| | | | | | | | | | | complete cds |
| GAS5_f | X59728 | 16.00 | 18.00 | 32.00 | 47.00 | 43.00 | 54.00 | 2.00 | 1.26 | X59728 M. musculus mRNA for gas5 growth arrest |
| | | | | | | | | | | specific protein |
| GAS5_f | X59728 | 14.00 | 19.00 | 36.33 | 45.33 | 38.50 | 45.00 | 2.60 | 1.17 | M. musculus mRNA for gas5 growth arrest specific |
| | | | | | | | | | | protein |
| GLUD_f | X57024 | 66.25 | 38.33 | 33.00 | 49.00 | 57.50 | 65.00 | 0.50 | 1.13 | X57024 Murine GLUD mRNA for glutamate |
| | | | | | | | | | | dehydrogenase |
| GNB1_f | U29055 | 11.75 | 11.33 | 28.33 | 37.33 | 12.00 | 14.33 | 2.41 | 1.19 | Mus musculus G protein beta 36 subunit mRNA, compl |
| GP49A_s | M65027 | 14.00 | 18.67 | 28.33 | 32.00 | 10.00 | 11.67 | 2.02 | 1.17 | Mouse cell surface antigen gp49 mRNA, complete cds |
| GRN_f | M86736 | 56.25 | 51.67 | 129.00 | 159.67 | 55.50 | 81.00 | 2.29 | 1.46 | Mouse acrogranin mRNA, complete cds |
| HMOX1_s | M33203 | 10.00 | 10.00 | 20.00 | 28.33 | 10.00 | 10.00 | 2.00 | 1.00 | Mouse tumor-induced 32 kD protein (p32) mRNA, |
| | | | | | | | | | | complete cds |
| HN1_s | U90123 | 10.00 | 10.00 | 23.67 | 25.00 | 12.50 | 14.00 | 2.37 | 1.12 | Mus musculus HN1 (Hn1) mRNA, complete cds |
| E_HSPB1_f | AA034638 | 10.00 | 10.00 | 20.00 | 29.67 | 10.00 | 10.00 | 2.00 | 1.00 | AA034638 mh17a07.r1 Mus musculus cDNA, 5′ end |
| E_HSPB1_f | AA015458 | 10.50 | 10.00 | 24.67 | 20.67 | 12.00 | 11.00 | 2.35 | 0.92 | AA015458 mh22b09.r1 Mus musculus cDNA, 5′ end |
| E_HSPB1_f | AA015026 | 12.25 | 14.33 | 38.67 | 44.33 | 15.00 | 11.00 | 3.16 | 0.73 | AA015026 mh26f03.r1 Mus musculus cDNA, 5′ end |
| HSP25_s | L07577 | 31.75 | 35.00 | 131.67 | 191.00 | 56.00 | 50.67 | 4.15 | 0.90 | Mus musculus small heat shock protein (HSP25) gene |
| HSP25_f | AA015057 | 18.75 | 26.33 | 51.33 | 70.67 | 24.50 | 20.67 | 2.74 | 0.84 | AA015057 mh14d03.r1 Mus musculus cDNA, 5′ end |
| E_HSPB1_f | AA038607 | 12.50 | 14.00 | 37.67 | 52.00 | 21.00 | 19.00 | 3.01 | 0.90 | AA038607 mi88e06.r1 Mus musculus cDNA, 5′ end |
| E_U27830_s | AA038775 | 13.75 | 24.33 | 43.00 | 45.00 | 10.50 | 22.67 | 3.13 | 2.16 | mi95f04.r1 Soares mouse p3NMF19.5 Mus musculus |
| | | | | | | | | | | cDNA clone 474367 5′ similar to gb U27830 Mus |
| | | | | | | | | | | musculus extendin mRNA, complete cds (MOUSE), |
| IDB4 | X75018 | 43.00 | 26.67 | 16.67 | 25.67 | 20.50 | 16.67 | 0.39 | 0.81 | X75018 M.Musculus mRNA for Id4 helix-loop-helix |
| | | | | | | | | | | protein |
| IFI49_s | L32974 | 13.75 | 10.00 | 29.00 | 33.00 | 14.50 | 14.67 | 2.11 | 1.01 | Mouse interferon-inducible protein homologue mRNA, |
| | | | | | | | | | | complete cds |
| IFNGR_s | J05265 | 12.75 | 11.00 | 27.67 | 40.67 | 15.00 | 16.00 | 2.17 | 1.07 | Mouse interferon gamma receptor mRNA, complete cds |
| IGK_V20_i | X16678 | 10.00 | 10.00 | 36.33 | 24.00 | 10.00 | 10.00 | 3.63 | 1.00 | Mouse VK gene for kappa light chain variable region |
| | | | | | | | | | | and J4 sequence. |
| IGK_V20_s | X16678 | 20.00 | 29.67 | 141.33 | 99.67 | 15.50 | 21.67 | 7.07 | 1.40 | Mouse VK gene for kappa light chain variable region |
| | | | | | | | | | | and J4 sequence. |
| IRF1_s | M21065 | 12.00 | 15.00 | 40.67 | 43.67 | 16.00 | 19.33 | 3.39 | 1.21 | Mouse interferon regulatory factor 1 mRNA, complete |
| | | | | | | | | | | cds |
| MIRF7_s | U73037 | 10.00 | 10.67 | 27.33 | 33.33 | 11.50 | 11.33 | 2.73 | 0.99 | Mus musculus interferon regulatory factor 7 (mirf7) |
| | | | | | | | | | | mRNA, complete cds |
| E_TC15056_s | AA122622 | 11.25 | 10.00 | 25.33 | 16.33 | 10.00 | 10.00 | 2.25 | 1.00 | mn33e03.r1 Beddington mouse embryonic region Mus |
| | | | | | | | | | | musculus cDNA clone 539740 5′ similar to TR E236822 |
| | | | | | | | | | | E236822 HYPOTHETICAL 26.5 KD PROTEIN |
| X15373-2_s | X15373 | 42.25 | 30.00 | 20.00 | 24.67 | 30.50 | 28.67 | 0.47 | 0.94 | Mouse cerebellum mRNA for P400 protein |
| E_JUN_s | W09701 | 16.25 | 16.33 | 32.33 | 32.67 | 18.00 | 13.00 | 1.99 | 0.72 | W09701 ma56e02.r1 Mus musculus cDNA, 5′ end |
| JUND1_f | X15358 | 53.75 | 73.00 | 109.33 | 135.00 | 82.00 | 85.67 | 2.03 | 1.04 | Mouse mRNA for junD proto-oncogene |
| D50581 | D50581 | 10.50 | 14.67 | 23.00 | 31.00 | 10.00 | 16.00 | 2.19 | 1.60 | Mouse mRNA for inward rectifier K+ channel |
| KPNA2_rc_s | C79184 | 33.75 | 41.33 | 101.33 | 109.00 | 50.00 | 43.67 | 3.00 | 0.87 | Mouse 3.5-dpc blastocyst cDNA Mus musculus cDNA |
| | | | | | | | | | | clone J0062A04 3′ similar to Mouse mRNA for nuclear |
| | | | | | | | | | | pore-targeting complex, mRNA sequence. |
| KRT2_1_f | AA541913 | 126.50 | 150.33 | 265.67 | 356.33 | 249.50 | 327.00 | 2.10 | 1.31 | vj02d01.r1 Barstead mosue pooled organs MPLRB4 |
| | | | | | | | | | | Mus musculus cDNA clone 920545 5′ similar to |
| | | | | | | | | | | gb.M17887 60S ACIDIC RIBOSOMAL PROTEIN P2 |
| | | | | | | | | | | (HUMAN); gb:U29402 Mus musculus acidic ribosomal |
| | | | | | | | | | | phosphoprotein P1 mRNA, complete (MOU. |
| KRT2_8_s | D90360 | 19.50 | 18.00 | 49.00 | 92.67 | 23.50 | 30.67 | 2.51 | 1.30 | Mouse gene for cytokeratin endo A |
| D84391_f | D84391 | 14.00 | 36.67 | 43.33 | 53.33 | 11.00 | 10.00 | 3.10 | 0.91 | Mouse L1 repetitive element, complete sequence. |
| E_LAP18_f | AA117100 | 11.50 | 11.67 | 24.33 | 19.67 | 19.00 | 14.00 | 2.12 | 0.74 | AA117100 mo60a10.r1 Mus musculus cDNA, 5′ end |
| LAPTM5_s | U29539 | 10.25 | 11.00 | 27.33 | 34.00 | 10.00 | 16.33 | 2.67 | 1.63 | Mus musculus retinoic acid-inducible E3 protein mR |
| LGALS1_f | X66532 | 34.75 | 46.67 | 190.33 | 133.67 | 101.50 | 109.33 | 5.48 | 1.08 | M. musculus mRNA for L14 lectin. |
| LGALS1_f | W13002 | 26.00 | 30.33 | 151.67 | 101.67 | 81.50 | 81.67 | 5.83 | 1.00 | W13002 mb21e10.r1 Mus musculus cDNA, 5′ end |
| E_LGALS3_f | W10936 | 10.00 | 10.00 | 27.33 | 28.33 | 14.00 | 12.67 | 2.73 | 0.90 | W10936 ma03e09.r1 Mus musculus cDNA, 5′ end |
| LGALS3_f | X16834 | 29.00 | 36.67 | 116.67 | 134.00 | 44.50 | 44.00 | 4.02 | 0.99 | X16834 Mouse mRNA for Mac-2 antigen |
| E_TC39260_s | AA542220 | 14.50 | 11.33 | 42.67 | 64.33 | 11.00 | 17.67 | 2.94 | 1.61 | vk43h10.r1 Soares mouse mammary gland NbMMG |
| | | | | | | | | | | Mus musculus cDNA clone 949411 5′ |
| LST1_s | U72643 | 11.00 | 13.00 | 29.33 | 29.67 | 17.00 | 14.33 | 2.67 | 0.84 | Mus musculus lymphocyte specific transcript (LST) |
| | | | | | | | | | | mRNA, partial cds |
| LYN_f | M57696 | 14.25 | 13.67 | 30.00 | 43.33 | 20.50 | 21.00 | 2.11 | 1.02 | Mouse lyn A protein tyrosine kinase (lynA) mRNA, |
| | | | | | | | | | | complete cds |
| E_PRKM1_s | AA104744 | 10.00 | 10.00 | 28.67 | 23.00 | 10.00 | 10.00 | 2.87 | 1.00 | AA104744 mo56d02.r1 Mus musculus cDNA, 5′ end |
| MDK_f | AA072643 | 15.50 | 25.00 | 37.67 | 28.00 | 16.00 | 18.00 | 2.43 | 1.13 | AA072643 mm75a09.r1 Mus musculus cDNA, 5′ end |
| MDK_f | M34094 | 30.25 | 38.00 | 90.67 | 49.67 | 25.50 | 28.00 | 3.00 | 1.10 | M34094 Mouse retinoic acid-responsive protein (MK) |
| | | | | | | | | | | gene, complete cds |
| MDK_f | M35833 | 33.25 | 42.33 | 105.67 | 112.00 | 27.00 | 30.00 | 3.18 | 1.11 | Mouse retinoic acid-responsive protein (MK) mRNA, |
| | | | | | | | | | | complete cds |
| ETV6_f | D00613 | 47.75 | 44.33 | 249.67 | 132.33 | 113.00 | 190.00 | 5.23 | 1.68 | D00613 Mouse mRNA for matrix Gla protein (MGP) |
| E_X61399_s | AA245242 | 11.25 | 11.00 | 31.00 | 32.33 | 11.50 | 17.00 | 2.76 | 1.48 | mw28h11.r1 Soares mouse 3NME12.5 Mus musculus |
| | | | | | | | | | | cDNA clone 672069 5′ similar to gb.X61399 Mouse F52 |
| | | | | | | | | | | mRNA for a novel protein (MOUSE); |
| MPS1_s | L20315 | 10.00 | 10.00 | 30.00 | 37.67 | 10.00 | 12.33 | 3.00 | 1.23 | L20315 Mus musculus MPS1 gene and mRNA, 3′end |
| NFKBIA | U36277 | 17.75 | 18.33 | 44.67 | 47.00 | 29.50 | 19.33 | 2.52 | 0.66 | U36277 Mus musculus l-kappa B alpha chain mRNA, |
| | | | | | | | | | | complete cds |
| NFKBIA_g | U36277 | 14.75 | 17.67 | 44.00 | 42.00 | 23.00 | 18.00 | 2.98 | 0.78 | U36277 Mus musculus l-kappa B alpha chain mRNA, |
| | | | | | | | | | | complete cds |
| AA607353 | AA607353 | 37.75 | 28.67 | 12.67 | 19.67 | 24.50 | 21.00 | 0.34 | 0.86 | v039d02.r1 Barstead mouse irradiated colon MPLRB7 |
| | | | | | | | | | | Mus musculus cDNA clone 1052259 5′, mRNA |
| | | | | | | | | | | sequence. |
| M33863_s | M33863 | 11.50 | 10.00 | 25.00 | 28.33 | 12.00 | 12.67 | 2.17 | 1.06 | Mouse 2′-5′ oligo A synthetase mRNA, complete cds |
| E_TC32548_rc_s | AA408672 | 39.25 | 36.00 | 80.00 | 75.67 | 50.00 | 42.00 | 2.04 | 0.84 | EST03133 Mouse 7.5 dpc embryo ectoplacental cone |
| | | | | | | | | | | cDNA library Mus musculus cDNA clone C0031D07 3′ |
| E_PEA15_s | AA108330 | 11.50 | 10.00 | 40.00 | 51.33 | 10.00 | 10.00 | 3.48 | 1.00 | AA108330 mp28b03.r1 Mus musculus cDNA, 5′ end |
| MAT1_s | L31958 | 34.25 | 16.67 | 77.67 | 111.33 | 16.00 | 29.00 | 2.27 | 1.81 | Mus musculus (clone pMAT1) mRNA, complete cds |
| PPICAP_s | X67809 | 15.25 | 10.00 | 78.00 | 84.00 | 21.50 | 32.67 | 51.11 | 1.52 | M. musculus mama mRNA |
| SERGLYCIN_s | X16133 | 18.25 | 14.00 | 51.33 | 43.67 | 33.00 | 35.00 | 2.81 | 1.06 | Mouse mRNA for mastocytoma proteoglycan core |
| | | | | | | | | | | protein, serglycin |
| PSME1_s | D87910 | 21.75 | 24.67 | 64.00 | 74.00 | 41.00 | 38.33 | 2.94 | 0.93 | Mus musculus mRNA for PA28 alpha subunit, complete |
| | | | | | | | | | | cds |
| PVA | X67141 | 28.00 | 22.33 | 10.00 | 11.00 | 22.00 | 24.33 | 0.36 | 1.11 | M. musculus Pva mRNA for parvalbumin |
| D50500_f | D50500 | 18.00 | 22.33 | 41.67 | 49.00 | 28.00 | 31.67 | 2.31 | 1.13 | Mouse mRNA for Rab 11, partial sequence. |
| RAC2_s | X53247 | 13.00 | 17.67 | 59.67 | 59.67 | 16.50 | 25.67 | 4.59 | 1.56 | M. musculus EN-7 mRNA. |
| E_TC31065_g | AA538285 | 13.50 | 10.67 | 42.00 | 82.33 | 10.50 | 13.67 | 3.11 | 1.30 | vj03d05.r1 Barstead mouse pooled organs MPLRB4 |
| | | | | | | | | | | Mus musculus cDNA clone 920649 5′ similar to |
| | | | | | | | | | | TR.G881954 G881954 RNPL; |
| C77421_rc_f | C77421 | 88.25 | 81.00 | 197.00 | 306.00 | 109.00 | 80.33 | 2.23 | 0.74 | Mouse 3.5-dpc blastocyst cDNA Mus musculus cDNA |
| | | | | | | | | | | clone J0030G04 3′ similar to Mouse B10.VL30LTR |
| | | | | | | | | | | gene, 5′ flank, mRNA sequence. |
| U67187_s | U67187 | 10.00 | 14.67 | 24.33 | 41.33 | 12.00 | 10.00 | 2.43 | 0.83 | Mus musculus G protein signaling regulator RGS2 |
| | | | | | | | | | | (rgs2) mRNA, complete cds |
| TSTAP198_7_rc_s | AA408475 | 11.00 | 11.33 | 24.33 | 21.67 | 20.00 | 19.33 | 2.21 | 0.97 | EST02956 Mouse 7.5 dpc embryo ectoplacental cone |
| | | | | | | | | | | cDNA library Mus musculus cDNA clone C0028E12 3′, |
| | | | | | | | | | | mRNA sequence. |
| E_RPL44_f | W30137 | 45.75 | 70.00 | 122.33 | 113.67 | 132.00 | 129.00 | 2.67 | 0.98 | mc27f10.r1 Soares mouse pdNMF19.5 Mus musculus |
| | | | | | | | | | | cDNA clone 349771 5′ similar to gb.M15661 60S |
| | | | | | | | | | | RIBOSOMAL PROTEIN L44 (HUMAN);, mRNA |
| | | | | | | | | | | sequence. |
| X15962_f | X15962 | 191.25 | 211.00 | 428.00 | 404.00 | 350.00 | 437.00 | 2.24 | 1.25 | Mouse mRNA for ribosomal protein S12. |
| C76830_rc_f | C76830 | 11.75 | 10.00 | 27.33 | 34.67 | 37.00 | 37.33 | 2.33 | 1.01 | Mouse 3.5-dpc blastocyst cDNA Mus musculus cDNA |
| | | | | | | | | | | clone J0020H05 3′ similar to Mus musculus ribosomal |
| | | | | | | | | | | protein S26 (RPS26) mRNA, mRNA sequence. |
| RRAS_s | W41501 | 10.25 | 10.00 | 21.67 | 25.67 | 10.00 | 10.00 | 2.11 | 1.00 | W41501 mc43d11.r1 Mus musculus cDNA, 5′ end |
| RRAS_s | M21019 | 16.00 | 12.00 | 43.33 | 53.33 | 18.00 | 28.00 | 2.71 | 1.56 | Mouse R-ras mRNA, complete cds |
| RRM2_rc_f | C81593 | 10.00 | 10.00 | 23.00 | 17.67 | 10.00 | 10.00 | 2.30 | 1.00 | Mouse 3.5-dpc blastocyst cDNA Mus musculus cDNA |
| | | | | | | | | | | clone J0101H11 3′ similar to Mouse ribonucleotide |
| | | | | | | | | | | reductase M2 subunit mRNA, mRNA sequence. |
| CAL1L_f | M16465 | 40.00 | 29.67 | 96.67 | 137.67 | 48.50 | 58.67 | 2.42 | 1.21 | Mouse calpactin I light chain (p11) mRNA, complete cds |
| U41341_s | U41341 | 24.25 | 24.67 | 120.67 | 171.33 | 65.50 | 78.67 | 4.98 | 1.20 | Mus musculus endothelial monocyte-activating |
| | | | | | | | | | | polypeptide I mRNA, complete cds |
| S100A4_s | D00208 | 14.50 | 19.33 | 35.33 | 38.33 | 13.00 | 20.33 | 2.44 | 1.56 | Mouse pEL98 protein mRNA which is enhanced in |
| | | | | | | | | | | established cells, Balb/c373 |
| CACY_s | X66449 | 10.00 | 10.00 | 23.67 | 34.00 | 13.50 | 19.67 | 2.37 | 1.46 | X66449 M. musculus mRNA for calcyclin |
| CACY_s | M37761 | 21.50 | 33.33 | 161.67 | 178.00 | 58.50 | 95.00 | 7.52 | 1.62 | Mouse calcyclin mRNA, complete cds |
| E_TC17285_s | AA137292 | 16.25 | 22.00 | 32.33 | 46.67 | 14.00 | 22.33 | 1.99 | 1.60 | mq98h01.r1 Soares mouse 3NbMS Mus musculus |
| | | | | | | | | | | cDNA clone 596017 5′ |
| SCYA5_s | U02298 | 10.00 | 10.00 | 22.33 | 13.67 | 10.00 | 10.00 | 2.23 | 1.00 | Mus musculus NIH 3T3 chemokine rantes (Scya5) |
| | | | | | | | | | | gene, complete cds |
| SCYD1_g | U92565 | 11.50 | 10.00 | 30.33 | 25.67 | 10.00 | 13.00 | 2.64 | 1.30 | Mus musculus fractalkine mRNA, complete cds |
| U11027_s | U11027 | 37.00 | 42.00 | 75.67 | 96.33 | 99.50 | 116.67 | 2.05 | 1.17 | Mus musculus C57BL/6J Sec61 protein complex |
| | | | | | | | | | | gamma subunit mRNA, complete cds |
| AF015284_s | AF015284 | 24.75 | 26.33 | 50.67 | 65.00 | 56.50 | 92.00 | 2.05 | 1.63 | Mus musculus selenoprotein W (mSelW) mRNA, |
| | | | | | | | | | | complete cds |
| GLVR1_s | M73696 | 10.00 | 10.00 | 20.67 | 31.67 | 10.00 | 10.00 | 2.07 | 1.00 | Murine Glvr-1 mRNA, complete cds |
| SLPI_s | U73004 | 10.00 | 10.00 | 24.00 | 26.67 | 10.00 | 11.33 | 2.40 | 1.13 | Mus musculus secretory leukocyte protease inhibitor |
| | | | | | | | | | | mRNA, complete cds |
| SNRPD1_s | M58558 | 10.00 | 10.33 | 20.33 | 24.33 | 16.50 | 15.33 | 2.03 | 0.93 | Murine sm D small nuclear ribonucleoprotein sequence. |
| SPARC_f | X04017 | 24.50 | 22.00 | 78.67 | 54.67 | 32.00 | 37.00 | 3.21 | 1.16 | X04017 Mouse mRNA for cysteine-rich glycoprotein |
| | | | | | | | | | | SPARC |
| SPP1_f | X51834 | 331.25 | 271.33 | 682.00 | 658.33 | 334.00 | 376.33 | 2.06 | 1.13 | Murine gene for osteopontin |
| SPP1_f | X16151 | 238.00 | 183.00 | 596.00 | 571.00 | 270.00 | 281.33 | 2.50 | 1.04 | X16151 Mouse mRNA for early T-lymphocyte activation |
| | | | | | | | | | | 1 protein (ETa-1) |
| E_SPP1_f | AA123395 | 116.00 | 72.67 | 307.33 | 282.00 | 122.50 | 90.67 | 2.65 | 0.74 | AA123395 mq74h12.r1 Mus musculus cDNA, 5′ end |
| E_SPP1_f | AA066782 | 61.75 | 63.33 | 376.67 | 316.67 | 93.00 | 78.00 | 6.10 | 0.84 | AA066782 mm16f08.r1 Mus musculus cDNA, 5′ end |
| SPRR1A_s | X91824 | 11.25 | 11.33 | 68.67 | 40.67 | 16.50 | 23.00 | 6.10 | 1.39 | M. musculus mRNA for SPRR1a protein |
| E_TC33582_s | AA396029 | 10.00 | 10.00 | 20.67 | 34.00 | 11.00 | 20.00 | 2.07 | 1.82 | vb41e05.r1 Soares mouse lymph node NbMLN Mus |
| | | | | | | | | | | musculus cDNA clone 751520 5′ |
| STAT3_s | U06922 | 42.25 | 37.67 | 99.33 | 152.33 | 26.00 | 16.67 | 2.35 | 0.64 | Mus musculus signal transducer and activator of |
| | | | | | | | | | | transcription (Stat3) mRNA, complete cds |
| STAT5A_s | U21103 | 10.75 | 20.33 | 26.33 | 32.67 | 10.00 | 13.00 | 2.45 | 1.30 | Mus musculus mammary gland factor (Stat5a) mRNA, c |
| E_TC28792_s | AA108677 | 10.00 | 11.00 | 21.00 | 24.33 | 10.00 | 12.00 | 2.10 | 1.20 | mp39a05.r1 Barstead MPLRB1 Mus musculus cDNA |
| | | | | | | | | | | clone 571568 5′ |
| TAGLN_s | L41154 | 20.50 | 20.33 | 79.67 | 50.67 | 30.00 | 34.67 | 3.89 | 1.16 | Mus musculus SM22 alpha mRNA, complete cds |
| E_D21261_s | AA120653 | 35.25 | 34.00 | 124.67 | 148.33 | 51.50 | 82.67 | 3.54 | 1.61 | mp71g11.r1 Soares 2NbMT Mus musculus cDNA clone |
| | | | | | | | | | | 574724 5′ similar to gb D21261 SM22-ALPHA |
| | | | | | | | | | | HOMOLOG (HUMAN), |
| TGFB1I4_s | X62940 | 137.50 | 123.67 | 306.33 | 424.33 | 179.50 | 176.33 | 2.23 | 0.98 | M. musculus TSC-22 mRNA. |
| TGFBI_s | L19932 | 10.00 | 10.00 | 30.33 | 25.67 | 10.00 | 11.33 | 3.03 | 1.13 | Mouse (beta ig-h3) mRNA, complete cds |
| L38444_s | L38444 | 10.00 | 10.00 | 20.00 | 20.33 | 13.00 | 19.33 | 2.00 | 1.49 | Mus musculus (clone U2) T-cell specific protein mRNA, |
| | | | | | | | | | | complete cds |
| UCP2_s | U69135 | 14.50 | 15.33 | 75.33 | 148.33 | 28.50 | 30.67 | 5.20 | 1.08 | Mus musculus UCP2 mRNA, complete cds) |
| AA000380_s | AA000380 | 28.00 | 40.00 | 63.00 | 72.00 | 30.00 | 25.00 | 2.25 | 0.83 | mg24e05.r1 Soares mouse embryo NbME13.5 14 5 |
| | | | | | | | | | | Mus musculus cDNA clone 424736 5′ |
| E_TC22765_s | AA002653 | 12.25 | 19.67 | 31.67 | 40.00 | 11.00 | 13.33 | 2.59 | 1.21 | mg38h07.r1 Soares mouse embryo NbME13.5 14 5 |
| | | | | | | | | | | Mus musculus cDNA clone 424736 5′ |
| E_TC18790_s | AA002761 | 10.00 | 10.00 | 22.67 | 24.00 | 10.00 | 11.67 | 2.27 | 1.17 | mg45b10.r1 Soares mouse embryo NbME13.5 14 5 |
| | | | | | | | | | | Mus musculus cDNA clone 426715 5′. |
| E_TC31090_s | AA003358 | 20.50 | 38.33 | 48.33 | 66.33 | 17.50 | 29.67 | 2.36 | 1.70 | mg49h01.r1 Soares mouse embryo NbME 13.5 14 5 |
| | | | | | | | | | | Mus musculus cDNA clone 427153 5′. |
| E_TC18985_s | AA004011 | 10.00 | 16.67 | 20.67 | 24.33 | 10.00 | 10.67 | 2.07 | 1.07 | mg80f01.r1 Soares mouse embryo NbME13.5 14 5 Mus |
| | | | | | | | | | | musculus cDNA clone 439321 5′. |
| E_455906 | AA023065 | 26.00 | 13.67 | 11.67 | 18.33 | 10.00 | 10.00 | 0.45 | 1.00 | AA023065 mh66c02.r1 Mus musculus cDNA, 5′ end |
| E_ABP1_s | AA023491 | 10.00 | 10.00 | 38.33 | 20.33 | 10.00 | 10.00 | 3.83 | 1.00 | AA023491 mh74e11.r1 Mus musculus cDNA, 5′ end |
| E_TC22882_s | AA028657 | 28.75 | 37.67 | 59.33 | 79.00 | 31.00 | 42.00 | 2.06 | 1.35 | mi14h12.r1 Soares mouse p3NMF19 5 Mus musculus |
| | | | | | | | | | | cDNA clone 463559 5′ |
| E_TC23744_s | AA030688 | 10.25 | 10.00 | 25.67 | 36.33 | 10.00 | 10.00 | 2.50 | 1.00 | mi22g02.r1 Soares mouse embryo NbME13 5 14 5 Mus |
| | | | | | | | | | | musculus cDNA clone 464306 5′ |
| E_K_ALPHA_1_f | AA068158 | 26.25 | 29.67 | 81.00 | 71.67 | 25.00 | 32.00 | 3.09 | 1.28 | AA068158 mm56e10.r1 Mus musculus cDNA, 5′ end |
| E_POL_s | AA087673 | 10.00 | 22.33 | 81.67 | 245.33 | 13.00 | 11.00 | 8.17 | 0.85 | AA087673 mm27b09.r1 Mus musculus cDNA, 5′ end |
| E_ABP1_s | AA104688 | 10.00 | 10.00 | 42.67 | 27.33 | 10.00 | 10.00 | 4.27 | 1.00 | AA104688 mo55c10.r1 Mus musculus cDNA, 5′ end |
| E_ABP1_s | AA107847 | 10.00 | 10.00 | 34.67 | 16.00 | 10.00 | 10.00 | 3.47 | 1.00 | AA107847 mo49d08.r1 Mus musculus cDNA, 5′ end |
| E_ABP1_s | AA109909 | 10.00 | 10.00 | 28.67 | 17.00 | 10.00 | 10.00 | 2.87 | 1.00 | AA109909 mp10d09.r1 Mus musculus cDNA, 5′ end |
| E_TC17629_s | AA165775 | 30.25 | 25.00 | 14.67 | 23.67 | 23.50 | 36.33 | 0.48 | 1.55 | mt74d01.r1 Soares mouse lymph node NbMLN Mus |
| | | | | | | | | | | musculus cDNA clone 635617 5′ |
| AA168865_f | AA168865 | 11.25 | 15.33 | 35.67 | 37.33 | 13.00 | 11.67 | 3.17 | 0.90 | AA168865 ms38c08.r1 Mus musculus cDNA, 5′ end |
| E_TC37973_s | AA172851 | 10.00 | 11.33 | 21.67 | 58.33 | 10.00 | 11.67 | 2.17 | 1.17 | mr31f05.r1 Soares mouse 3NbMS Mus musculus cDNA |
| | | | | | | | | | | clone 599073 5′ |
| E_TC27387_f | AA174883 | 25.00 | 32.00 | 65.67 | 109.67 | 10.00 | 10.00 | 2.63 | 1.00 | ms77e07.r1 Soares mouse 3NbMS Mus musculus |
| | | | | | | | | | | cDNA clone 617604 5′ |
| E_TC19964 | AA184455 | 10.00 | 13.67 | 21.00 | 25.33 | 11.00 | 10.00 | 2.10 | 0.91 | mt58c09.r1 Soares 2NbMT Mus musculus cDNA clone |
| | | | | | | | | | | 634096 5′ |
| E_TC32253_s | AA197973 | 46.00 | 26.67 | 20.67 | 21.67 | 31.00 | 40.33 | 0.45 | 1.30 | mv12g09.r1 GuayWoodford Beier mouse kidney day 0 |
| | | | | | | | | | | Mus musculus cDNA clone 654880 5′ similar to |
| | | | | | | | | | | SW BCCP_PROFR P02904 BIOTIN CARBOXYL |
| | | | | | | | | | | CARRIER PROTEIN OF METHYLMALONYL-COA |
| | | | | | | | | | | CARBOXYL-TRANSFERASE, |
| E_TC27481_s | AA210359 | 13.00 | 11.00 | 29.33 | 37.33 | 13.00 | 13.00 | 2.26 | 1.00 | mu72h03.r1 Soares mouse lymph node NbMLN Mus |
| | | | | | | | | | | musculus cDNA clone 644981 5′ |
| E_TC30948_s | AA245784 | 65.00 | 41.00 | 29.67 | 45.33 | 35.50 | 41.00 | 0.46 | 1.15 | mx03b10.r1 Soares mouse NML Mus musculus cDNA |
| | | | | | | | | | | clone 679099 5′ |
| E_TC35691_f | AA538477 | 11.00 | 11.67 | 22.67 | 42.67 | 10.00 | 10.00 | 2.06 | 1.00 | vj53e12.r1 Knowles Solter mouse blastocyst B1 Mus |
| | | | | | | | | | | musculus cDNA clone 932782 5′ |
| E_COLA1_f | AA562685 | 11.50 | 10.00 | 58.33 | 28.67 | 11.00 | 14.33 | 5.07 | 1.30 | vl56h09.r1 Stratagene mouse skin (#937313) Mus |
| | | | | | | | | | | musculus cDNA clone 976289 5′ similar to gb X06753 |
| | | | | | | | | | | Mouse pro-alpha1 (MOUSE), |
| AA563404 | AA563404 | 86.75 | 42.00 | 31.00 | 36.33 | 52.50 | 56.33 | 0.36 | 1.07 | vl75d10.r1 Knowles Solter mouse blastocyst B1 Mus |
| | | | | | | | | | | musculus cDNA clone 978067 5′ |
| AA606926_s | AA606926 | 15.25 | 10.33 | 35.00 | 46.00 | 13.00 | 23.67 | 2.30 | 1.82 | vm91d04.r1 Knowles Solter mouse blastocyst B1 Mus |
| | | | | | | | | | | musculus cDNA clone 1005607 5′ similar to |
| | | | | | | | | | | TR.G497940 G497940 MAJOR VAULT PROTEIN. ,, |
| | | | | | | | | | | mRNA sequence. |
| AA616243_s | AA616243 | 10.00 | 10.00 | 21.33 | 37.67 | 10.00 | 10.00 | 2.13 | 1.00 | vo50d04.r1 Barstead mouse irradiated colon MPLRB7 |
| | | | | | | | | | | Mus musculus cDNA clone 1053319 5′, mRNA |
| | | | | | | | | | | sequence. |
| AA617093 | AA617093 | 10.75 | 16.67 | 21.33 | 39.33 | 10.50 | 12.67 | 1.98 | 1.21 | vi21f09.r1 Barstead mouse proximal colon MPLRB6 |
| | | | | | | | | | | Mus musculus cDNA clone 904457 5′, mRNA sequence. |
| AA690738_s | AA690738 | 15.50 | 12.00 | 36.33 | 49.67 | 12.50 | 15.33 | 2.34 | 1.23 | vu57b03.r1 Soares mouse mammary gland NbMMG |
| | | | | | | | | | | Mus musculus cDNA clone 1195469 5′, mRNA |
| | | | | | | | | | | sequence. |
| AA710451_s | AA710451 | 10.00 | 10.00 | 46.33 | 31.67 | 10.00 | 10.00 | 4.63 | 1.00 | vt42f07.r1 Barstead mouse proximal colon MPLRB6 |
| | | | | | | | | | | Mus musculus cDNA clone 1165765 5′, mRNA |
| | | | | | | | | | | sequence. |
| AA711130_f | AA711130 | 149.75 | 166.33 | 321.33 | 525.33 | 142.00 | 210.67 | 2.15 | 1.48 | vt56c05.r2 Barstead mouse irradiated colon MPLRB7 |
| | | | | | | | | | | Mus musculus cDNA clone 1167080 5′, mRNA |
| | | | | | | | | | | sequence. |
| HCPH_genePTPN6_s | AC002397 | 10.00 | 13.00 | 25.00 | 33.33 | 10.00 | 14.00 | 2.50 | 1.40 | Mouse chromosome 6 BAC-284H12 (Research |
| | | | | | | | | | | Genetics mouse BAC library) complete sequence. |
| C75983_rc_f | C75983 | 14.50 | 51.00 | 60.33 | 73.33 | 10.00 | 10.00 | 4.16 | 1.00 | Mouse 3 5-dpc blastocyst cDNA Mus musculus cDNA |
| | | | | | | | | | | clone J0001E09 3′ similar to Unannotatable data, |
| | | | | | | | | | | mRNA sequence. |
| C76162_rc_f | C76162 | 11.50 | 35.00 | 42.33 | 48.67 | 10.00 | 10.00 | 3.68 | 1.00 | Mouse 3 5-dpc blastocyst cDNA Mus musculus cDNA |
| | | | | | | | | | | clone J0004G06 3′ similar to Rat insulin-I (ins-1) gene, |
| | | | | | | | | | | mRNA sequence. |
| C76523_rc_g | C76523 | 10.00 | 10.00 | 23.00 | 19.67 | 10.00 | 10.00 | 2.30 | 1.00 | Mouse 3 5-dpc blastocyst cDNA Mus musculus cDNA |
| | | | | | | | | | | clone J0012E07 3′, mRNA sequence. |
| C76523_rc | C76523 | 11.50 | 10.00 | 30.67 | 40.33 | 10.00 | 11.00 | 2.67 | 1.10 | Mouse 3 5-dpc blastocyst cDNA Mus musculus cDNA |
| | | | | | | | | | | clone J0012E07 3′, mRNA sequence. |
| C77514_rc_s | C77514 | 90.75 | 112.33 | 190.67 | 223.67 | 139.00 | 201.33 | 2.10 | 1.45 | Mouse 3 5-dpc blastocyst cDNA Mus musculus cDNA |
| | | | | | | | | | | clone J0032G04 3′ similar to Rat G protein gamma-5 |
| | | | | | | | | | | subunit, mRNA sequence. |
| C77861_rc_s | C77861 | 16.50 | 13.33 | 35.67 | 42.67 | 17.50 | 17.67 | 2.16 | 1.01 | Mouse 3 5-dpc blastocyst cDNA Mus musculus cDNA |
| | | | | | | | | | | clone J0038G08 3′ similar to Rattus norvegicus major |
| | | | | | | | | | | vault protein mRNA, mRNA sequence. |
| C78546_rc_s | C78546 | 40.25 | 40.67 | 87.33 | 103.67 | 33.00 | 52.00 | 2.17 | 1.58 | Mouse 3 5-dpc blastocyst cDNA Mus musculus cDNA |
| | | | | | | | | | | clone J0051B02 3′ similar to moesin homolog [mice, |
| | | | | | | | | | | teratocarcinoma F9 cells, mRNA, mRNA sequence. |
| C80574_rc_s | C80574 | 28.00 | 21.67 | 60.67 | 83.00 | 36.00 | 42.00 | 2.17 | 1.17 | Mouse 3 5-dpc blastocyst cDNA Mus musculus cDNA |
| | | | | | | | | | | clone J0084D04 3′ similar to Human clone 23665 |
| | | | | | | | | | | mRNA sequence. |
| ET61420_f | ET61420 | 10.00 | 10.00 | 65.67 | 86.33 | 10.0 | 10.67 | 6.57 | 1.07 | Mus musculus anti-glycoprotein-B of human |
| | | | | | | | | | | Cytomegalovirus immunoglobulin Vh chain gene, partial |
| | | | | | | | | | | cds |
| ET61464_f | ET61464 | 10.00 | 10.00 | 23.00 | 34.67 | 10.00 | 10.00 | 2.30 | 1.00 | Mus musculus immunoglobulin heavy chain mRNA, V, |
| | | | | | | | | | | D, and J segments, partial cds |
| ET61520_f | ET61520 | 10.00 | 10.00 | 45.00 | 47.00 | 10.00 | 10.00 | 4.50 | 1.00 | Mus musculus IgG rearranged heavy chain mRNA, |
| | | | | | | | | | | variable region partial cds |
| ET61599_f | ET61599 | 10.00 | 11.00 | 42.00 | 57.33 | 10.00 | 10.33 | 4.20 | 1.03 | Mus musculus monoclonal antibody against hepatitis B |
| | | | | | | | | | | surface antigen, IgG light chain variable region gene, |
| | | | | | | | | | | partial cds |
| ET61727_f | ET61727 | 10.00 | 10.00 | 29.00 | 36.67 | 10.00 | 10.00 | 2.90 | 1.00 | Mus musculus Ig 2G11 E2 heavy chain mRNA, specific |
| | | | | | | | | | | for rat (mouse) cytochrome c, partial cds |
| ET61730_f | ET61730 | 10.00 | 10.00 | 37.67 | 60.00 | 10.00 | 10.00 | 3.77 | 1.00 | Mus musculus Ig 2G3 H5 heavy chain mRNA, specific |
| | | | | | | | | | | for rat (mouse) cytochrome c, partial cds |
| ET61732_f | ET61732 | 10.00 | 10.00 | 30.33 | 36.00 | 10.00 | 10.00 | 3.03 | 1.00 | Mus musculus Ig 5C12.A4 heavy chain mRNA, specific |
| | | | | | | | | | | for rat (mouse) cytochrome c, partial cds |
| ET61733_f | ET61733 | 10.00 | 10.00 | 32.67 | 36.00 | 10.00 | 10.00 | 3.27 | 1.00 | Mus musculus Ig 7A12.A2 heavy chain mRNA, specific |
| | | | | | | | | | | for rat (mouse) cytochrome c, partial cds |
| ET61736_f | ET61736 | 10.00 | 10.00 | 44.67 | 50.00 | 10.00 | 10.00 | 4.47 | 1.00 | Mus musculus Ig 9G7.A10 heavy chain mRNA, specific |
| | | | | | | | | | | for rat (mouse) cytochrome c, partial cds |
| ET61737_f | ET61737 | 10.00 | 10.00 | 30.33 | 37.67 | 10.00 | 10.00 | 3.03 | 1.00 | Mus musculus Ig 3A6.A5 heavy chain mRNA, specific |
| | | | | | | | | | | for rat (mouse) cytochrome c, partial cds |
| ET61739_f | ET61739 | 10.00 | 10.00 | 23.67 | 28.33 | 10.00 | 10.00 | 2.37 | 1.00 | Mus musculus Ig 7D1.B8 heavy chain mRNA, specific |
| | | | | | | | | | | for rat (mouse) cytochrome c, partial cds |
| ET61741_f | ET61741 | 10.00 | 10.00 | 31.33 | 45.00 | 10.00 | 10.00 | 3.13 | 1.00 | Mus musculus Ig 2C9.B12 heavy chain mRNA, specific |
| | | | | | | | | | | for rat (mouse) cytochrome c, partial cds |
| ET61744_f | ET61744 | 10.00 | 10.00 | 20.00 | 24.00 | 10.00 | 10.00 | 2.00 | 1.00 | Mus musculus Ig 3F10.C9 heavy chain mRNA, specific |
| | | | | | | | | | | for rat (mouse) cytochrome c, partial cds |
| ET61746_f | ET61746 | 10.00 | 10.00 | 43.00 | 40.67 | 10.00 | 10.00 | 4.30 | 1.00 | Mus musculus Ig 4A6.A8 heavy chain mRNA, specific |
| | | | | | | | | | | for rat (mouse) cytochrome c, partial cds |
| ET61747_f | ET61747 | 10.00 | 10.00 | 40.67 | 39.00 | 10.00 | 10.00 | 4.07 | 1.00 | Mus musculus Ig 4C4.A10 heavy chain mRNA, specific |
| | | | | | | | | | | for rat (mouse) cytochrome c, partial cds |
| ET61748_f | ET61748 | 10.00 | 10.00 | 35.67 | 40.33 | 10.00 | 10.00 | 3.57 | 1.00 | Mus musculus Ig 4C5.A11 heavy chain mRNA, specific |
| | | | | | | | | | | for rat (mouse) cytochrome c, partial cds |
| ET61749_f | ET61749 | 10.00 | 10.00 | 21.00 | 23.33 | 10.00 | 10.00 | 2.10 | 1.00 | Mus musculus Ig 6C3.B8 heavy chain mRNA, specific |
| | | | | | | | | | | for rat (mouse) cytochrome c, partial cds |
| ET62172_f | ET62172 | 10.00 | 10.33 | 61.00 | 72.67 | 10.00 | 11.33 | 6.10 | 1.13 | Mus musculus anti-PAH immunoglobulin Fab 10C10 |
| | | | | | | | | | | heavy chain V and CH1 regions gene, partial cds |
| ET62206_f | ET62206 | 10.00 | 12.67 | 27.67 | 38.00 | 10.50 | 10.67 | 2.77 | 1.02 | Mus musculus anti-digoxin immunoglobulin heavy chain |
| | | | | | | | | | | variable region precursor mRNA, partial cds |
| ET62224_f | ET62224 | 10.00 | 10.00 | 31.33 | 26.33 | 10.00 | 10.00 | 3.13 | 1.00 | Mus musculus immunoglobulin heavy chain variable |
| | | | | | | | | | | region mRNA, partial cds |
| ET62233_f | ET62233 | 10.00 | 10.00 | 32.00 | 50.33 | 10.00 | 10.00 | 3.20 | 1.00 | Mus musculus polyreactive autoantibody, |
| | | | | | | | | | | immunoglobulin IgM heavy chain mRNA, partial cds |
| ET62234_f | ET62234 | 10.00 | 10.00 | 26.67 | 47.33 | 10.00 | 10.00 | 2.67 | 1.00 | Mus musculus polyreactive autoantibody, |
| | | | | | | | | | | immunoglobulin IgM heavy chain mRNA, partial cds |
| ET62256_f | ET62256 | 10.00 | 10.00 | 36.00 | 46.33 | 10.00 | 10.00 | 3.60 | 1.00 | Mus musculus anti-PAH immunoglobulin Fab 4D5 |
| | | | | | | | | | | heavy chain V and CH1 regions mRNA, partial cds |
| ET62260_f | ET62260 | 10.00 | 11.67 | 37.67 | 51.33 | 10.00 | 12.00 | 3.77 | 1.20 | Mus musculus immunoglobulin light chain variable |
| | | | | | | | | | | region mRNA, partial cds |
| ET62430_f | ET62430 | 10.00 | 10.00 | 21.33 | 22.33 | 10.00 | 10.00 | 2.13 | 1.00 | Mus musculus Ig heavy chain Fv fragment mRNA, |
| | | | | | | | | | | partial cds |
| ET62779_f | ET62779 | 10.00 | 10.00 | 65.67 | 76.67 | 10.00 | 10.00 | 6.57 | 1.00 | Mus musculus IgM heavy chain variable region mRNA, |
| | | | | | | | | | | partial cds |
| ET62868_f | ET62868 | 10.00 | 10.00 | 33.67 | 40.33 | 10.00 | 10.00 | 3.37 | 1.00 | Mus musculus anti-CD8 immunoglobulin heavy chain V |
| | | | | | | | | | | region mRNA, partial cds |
| ET62923_f | ET62923 | 10.00 | 10.00 | 56.67 | 65.00 | 10.00 | 10.00 | 5.67 | 1.00 | M. musculus antibody heavy chain variable region |
| | | | | | | | | | | (354bp). |
| ET62924_f | ET62924 | 10.00 | 10.00 | 59.67 | 54.00 | 10.00 | 10.00 | 5.97 | 1.00 | M. musculus antibody heavy chain variable region |
| | | | | | | | | | | (363bp). |
| ET62925_f | ET62925 | 10.00 | 10.33 | 74.67 | 76.67 | 10.00 | 13.00 | 7.47 | 1.30 | M. musculus antibody heavy chain variable region |
| | | | | | | | | | | (372bp). |
| ET62926_f | ET62926 | 10.00 | 10.00 | 30.00 | 26.67 | 10.00 | 10.00 | 3.00 | 1.00 | M. musculus antibody heavy chain variable region |
| | | | | | | | | | | (354bp). |
| ET62928_f | ET62928 | 11.00 | 10.33 | 23.00 | 30.67 | 10.00 | 10.00 | 2.09 | 1.00 | M. musculus antibody heavy chain variable region |
| | | | | | | | | | | (366bp) |
| ET62932_f | ET62932 | 10.00 | 10.00 | 22.00 | 34.00 | 10.00 | 10.00 | 2.20 | 1.00 | M. musculus antibody heavy chain variable region |
| | | | | | | | | | | (372bp) |
| ET62933_f | ET62933 | 10.00 | 10.00 | 25.67 | 34.33 | 10.00 | 10.00 | 2.57 | 1.00 | M. musculus antibody heavy chain variable region |
| | | | | | | | | | | (360bp) |
| ET62934_f | ET62934 | 10.00 | 10.00 | 30.33 | 37.00 | 10.00 | 10.00 | 3.03 | 1.00 | M. musculus antibody heavy chain variable region |
| | | | | | | | | | | (348bp) |
| ET62936_f | ET62936 | 10.00 | 10.00 | 24.67 | 38.00 | 10.00 | 10.00 | 2.47 | 1.00 | M. musculus antibody heavy chain variable region |
| | | | | | | | | | | (375bp). |
| ET62941_f | ET62941 | 10.00 | 10.00 | 37.33 | 45.00 | 10.00 | 10.00 | 3.73 | 1.00 | M. musculus antibody light chain variable region |
| | | | | | | | | | | (318bp). |
| ET62942_f | ET62942 | 10.00 | 10.00 | 44.00 | 49.33 | 10.00 | 10.33 | 4.40 | 1.03 | M. musculus antibody light chain variable region |
| | | | | | | | | | | (324bp) |
| ET62983_f | ET62983 | 11.00 | 13.00 | 56.00 | 70.67 | 10.00 | 15.67 | 5.09 | 1.57 | M. musculus mRNA (2F7) for IgA V-D-J-heavy chain |
| ET62984_f | ET62984 | 10.00 | 10.33 | 66.00 | 69.67 | 10.00 | 15.33 | 6.60 | 1.53 | M. musculus mRNA (3C10) for IgA V-D-J-heavy chain. |
| ET63027_f | ET63027 | 10.00 | 10.00 | 24.33 | 18.67 | 10.00 | 10.00 | 2.43 | 1.00 | M. musculus mRNA for immunoglobulin variable region, |
| | | | | | | | | | | heavy chain |
| ET63041_f | ET63041 | 10.00 | 10.00 | 55.00 | 60.00 | 10.00 | 10.67 | 5.50 | 1.07 | M. musculus mRNA for immunoglobulin heavy variable |
| | | | | | | | | | | region. |
| ET63042_f | ET63042 | 10.00 | 10.00 | 29.00 | 34.00 | 10.00 | 10.00 | 2.90 | 1.00 | M. musculus mRNA for immunoglobulin kappa variable |
| | | | | | | | | | | region |
| ET63085_f | ET63085 | 10.00 | 10.00 | 49.33 | 57.33 | 10.00 | 10.00 | 4.93 | 1.00 | M. musculus mRNA for monoclonal antibody heavy |
| | | | | | | | | | | chain variable region. |
| ET63093_f | ET63093 | 10.00 | 10.00 | 34.00 | 46.00 | 10.00 | 11.67 | 3.40 | 1.17 | M. musculus mRNA for immunoglobulin heavy chain |
| | | | | | | | | | | variable domain, subgroup IIb |
| ET63106_f | ET63106 | 10.00 | 10.00 | 22.33 | 32.67 | 10.00 | 10.00 | 2.23 | 1.00 | M. musculus mRNA for immunoglobulin heavy chain |
| | | | | | | | | | | variable region, isolate 205. |
| ET63107_f | ET63107 | 10.00 | 10.00 | 32.67 | 21.67 | 10.00 | 10.00 | 3.27 | 1.00 | M. musculus mRNA for immunoglobulin kappa light |
| | | | | | | | | | | chain variable region |
| ET63126_f | ET63126 | 10.00 | 11.67 | 30.00 | 40.33 | 10.00 | 11.00 | 3.00 | 1.10 | M. musculus mRNA for anti folate binding protein, |
| | | | | | | | | | | MOv19 Vkappa. |
| ET63271_f | ET63271 | 11.00 | 10.33 | 23.67 | 32.00 | 10.00 | 10.00 | 2.15 | 1.00 | M. domesticus IgG variable region.)PIR.PH1015 (Ig |
| | | | | | | | | | | heavy chain V region (clone 111.55) - mouse (fragment) |
| ET63274_f | ET63274 | 10.00 | 10.00 | 51.33 | 61.33 | 10.00 | 11.00 | 5.13 | 1.10 | M. domesticus IgG variable region.)PIR.PH1001 (Ig |
| | | | | | | | | | | heavy chain V region (clone 111.68) - mouse (fragment) |
| ET63276_f | ET63276 | 10.00 | 10.00 | 85.67 | 93.33 | 10.00 | 16.00 | 8.57 | 1.60 | M. domesticus IgM variable region.)PIR:S26746 (Ig |
| | | | | | | | | | | heavy chain J region JH3 - mouse)PIR:PH0985 (Ig |
| | | | | | | | | | | heavy chain V region (clone 163.100) - mouse |
| | | | | | | | | | | (fragment) |
| ET63278_f | ET63278 | 10.00 | 10.00 | 38.33 | 51.67 | 10.00 | 10.00 | 3.83 | 1.00 | M. domesticus IgG variable region.)PIR:PH1007 (Ig |
| | | | | | | | | | | heavy chain V region (clone 163-c1) - mouse (fragment) |
| ET63288_f | ET63288 | 10.00 | 10.00 | 40.67 | 46.33 | 10.00 | 10.00 | 4.07 | 1.00 | M. domesticus IgM variable region.)PIR:PH0975 (Ig |
| | | | | | | | | | | heavy chain V region (clone 163.72) - mouse (fragment) |
| ET63290_f | ET63290 | 10.00 | 10.00 | 40.67 | 26.00 | 10.00 | 10.00 | 4.07 | 1.00 | M. domesticus IgK variable region.)PIR:PH1006 (Ig light |
| | | | | | | | | | | chain V region (clone 165.14) - mouse (fragment) |
| ET63295_f | ET6295 | 10.00 | 10.67 | 75.33 | 79.67 | 10.00 | 11.33 | 7.53 | 1.13 | M. domesticus IgM variable region.)PIR:S26747 (Ig |
| | | | | | | | | | | heavy chain J region JH4 - mouse |
| ET63300_f | ET63300 | 10.00 | 10.00 | 63.00 | 81.00 | 10.00 | 11.33 | 6.30 | 1.13 | M. domesticus IgG variable region.)PIR:PH0983 (Ig |
| | | | | | | | | | | heavy chain V region (clone 165.49) - mouse (fragment) |
| ET63314_f | ET63314 | 10.00 | 10.00 | 45.67 | 50.00 | 10.00 | 10.00 | 4.57 | 1.00 | M. domesticus IgM variable region.)PIR:S26747 (Ig |
| | | | | | | | | | | heavy chain J region JH4 - mouse)PIR:PH1012 (Ig |
| | | | | | | | | | | heavy chain V region (clone 17p.73) - mouse (fragment) |
| ET63320_f | ET63320 | 10.00 | 10.33 | 57.00 | 81.33 | 10.00 | 10.00 | 5.70 | 1.00 | M. domesticus IgM variable region.)PIR:PH0972 (Ig |
| | | | | | | | | | | heavy chain V region (clone 17s.128) - mouse |
| | | | | | | | | | | (fragment) |
| ET63322_f | ET63322 | 10.00 | 10.00 | 27.00 | 33.33 | 10.00 | 10.00 | 2.70 | 1.00 | M. domesticus IgK variable region.)PIR:PH1073 (Ig light |
| | | | | | | | | | | chain V region (clone 17s.130) - mouse (fragment) |
| ET63324_f | ET63324 | 10.00 | 10.00 | 35.67 | 46.67 | 10.00 | 10.00 | 3.57 | 1.00 | M. domesticus IgM variable region.)PIR:PH0980 (Ig |
| | | | | | | | | | | heavy chain V region (clone 17s.13) - mouse (fragment) |
| ET63328_f | ET63328 | 10.00 | 10.00 | 55.67 | 67.67 | 10.00 | 10.00 | 5.57 | 1.00 | M. domesticus IgM variable region.)PIR:PH0978 (Ig |
| | | | | | | | | | | heavy chain V region (clone 17s.166) - mouse |
| | | | | | | | | | | (fragment) |
| ET63331 _f | ET63331 | 10.00 | 10.00 | 33.33 | 42.00 | 10.00 | 10.67 | 33.33 | 1.07 | M. domesticus IgG variable region.)PIR:PH0988 (Ig |
| | | | | | | | | | | heavy chain V region (clone 17s-c3) - mouse (fragment) |
| ET63333_f | ET63333 | 10.00 | 10.67 | 78.33 | 97.33 | 10.00 | 11.67 | 7.83 | 1.17 | M. domesticus IgG variable region. |
| ET63337_f | ET63337 | 10.00 | 10.00 | 22.33 | 32.33 | 10.00 | 10.00 | 2.23 | 1.00 | M. domesticus IgG variable region.)PIR:PH1009 (Ig |
| | | | | | | | | | | heavy chain V region (clone 17s.5) - mouse (fragment) |
| ET63339_f | ET63339 | 10.00 | 10.00 | 42.33 | 50.67 | 10.00 | 10.00 | 4.23 | 1.00 | M. domesticus IgM variable region.)PIR:PH0986 (Ig |
| | | | | | | | | | | heavy chain V region (clone 17s-c6) - mouse (fragment) |
| ET63341 _f | ET63341 | 10.00 | 10.00 | 54.33 | 72.00 | 10.00 | 10.67 | 5.43 | 1.07 | M. domesticus IgG variable region.)PIR:PH0984 (Ig |
| | | | | | | | | | | heavy chain V region (clone 17s.83) - mouse (fragment) |
| ET63348_f | ET63348 | 10.00 | 10.00 | 46.33 | 59.67 | 10.00 | 10.00 | 4.63 | 1.00 | M. domesticus IgG variable region.)PIR:S26747 (Ig |
| | | | | | | | | | | heavy chain J region JH4 - mouse)PIR:PH1000 (Ig |
| | | | | | | | | | | heavy chain V region (clone 202.105) - mouse |
| | | | | | | | | | | (fragment) |
| ET63351 _f | ET63351 | 10.00 | 10.00 | 34.00 | 47.33 | 10.00 | 10.00 | 3.40 | 1.00 | M. domesticus IgM variable region.)PIR:PH1006 (Ig |
| | | | | | | | | | | heavy chain V region (clone 202.33) - mouse (fragment) |
| ET63354_f | ET63354 | 10.00 | 11.00 | 64.33 | 75.00 | 10.00 | 10.00 | 6.43 | 1.00 | M. domesticus IgM variable region.)PIR:PH0995 (Ig |
| | | | | | | | | | | heavy chain V region (clone 202.61) - mouse (fragment) |
| ET63358_f | ET63358 | 10.00 | 10.33 | 42.00 | 46.33 | 10.00 | 10.00 | 4.20 | 1.00 | M. domesticus IgK variable region.)PIR:PH1046 (Ig light |
| | | | | | | | | | | chain V region (clone 202.9) - mouse |
| | | | | | | | | | | (fragment))PIR:PH1048 (Ig light chain V region (clone |
| | | | | | | | | | | 165.49) - mouse (fragment))PIR:PH1047 (Ig light chain |
| | | | | | | | | | | V region (clones 165.45 and 163-c1) - mouse |
| ET63359_f | ET63359 | 10.00 | 10.00 | 35.67 | 56.33 | 10.00 | 10.00 | 3.57 | 1.00 | M. domesticus IgM variable region.)PIR:PH1011 (Ig |
| | | | | | | | | | | heavy chain V region (clone 202.38m) - mouse |
| | | | | | | | | | | (fragment) |
| ET63363_f | ET63363 | 10.00 | 10.00 | 43.00 | 56.00 | 10.00 | 10.00 | 4.30 | 1.00 | M. domesticus IgM variable region.)PIR:PH0976 (Ig |
| | | | | | | | | | | heavy chain V region (clone 25.12m) - mouse |
| | | | | | | | | | | (fragment) |
| ET63365_f | ET63365 | 10.00 | 11.67 | 64.33 | 75.00 | 10.00 | 10.67 | 6.43 | 1.07 | M. domesticus IgG variable region |
| ET63368_f | ET63368 | 10.00 | 11.33 | 30.00 | 47.33 | 10.00 | 10.00 | 3.00 | 1.00 | M. domesticus IgK variable region.)PIR:PH1076 (Ig light |
| | | | | | | | | | | chain V region (clone 74-c2) - mouse (fragment) |
| ET63369_f | ET63369 | 10.00 | 10.00 | 24.33 | 38.33 | 10.00 | 10.00 | 2.43 | 1.00 | M. domesticus IgG variable region. |
| ET63387_f | ET63387 | 10.00 | 10.33 | 48.67 | 66.00 | 10.00 | 10.00 | 4.87 | 1.00 | Artificial mRNA for single chain antibody scFv |
| | | | | | | | | | | (scFvP25). |
| ET63415_f | ET63415 | 10.00 | 10.00 | 34.67 | 38.00 | 10.00 | 10.00 | 3.47 | 1.00 | Mus musculus mRNA for IgG1/kappa antibody, |
| | | | | | | | | | | scFv(glyc)-CK.)PIR:PH1043 (Ig light chain V region |
| | | | | | | | | | | (clone 111.68) - mouse (fragment))PIR:PH1042 (Ig light |
| | | | | | | | | | | chain V region (clone 202.s38) - mouse (fragment) |
| IGBCR1 _f | L28060 | 10.00 | 10.00 | 21.00 | 20.33 | 10.00 | 10.00 | 2.10 | 1.00 | L28060 Mus musculus Ig B cell antigen receptor gene, |
| | | | | | | | | | | complete cds |
| IGH_VH10 | M12813 | 10.00 | 10.33 | 33.33 | 36.00 | 10.00 | 10.00 | 3.33 | 1.00 | M12813 Mouse Ig germline H-chain gene H10 V-region |
| | | | | | | | | | | (V), exons 1 and 2 |
| GAG_f | M26005 | 12.25 | 24.00 | 61.67 | 128.00 | 10.00 | 10.00 | 5.03 | 1.00 | M26005 Mouse endogenous retrovirus truncated gag |
| | | | | | | | | | | protein, complete cds, clone del env-1 3 1 |
| R74638_rc | R74638 | 13.00 | 27.00 | 27.00 | 37.33 | 14.00 | 27.00 | 2.08 | 1.93 | MDB0793 Mouse brain, Stratagene Mus musculus |
| | | | | | | | | | | cDNA 3′end. |
| U23089_f | U23089 | 10.00 | 10.67 | 30.67 | 60.67 | 10.00 | 10.00 | 3.07 | 1.00 | Mus musculus CB17 SCID immunoglobulin heavy chain |
| | | | | | | | | | | V region mRNA, clone 58-53, partial cds |
| E_HSPB1 _f | W08057 | 10.00 | 11.00 | 48.00 | 59.00 | 13.50 | 14.67 | 4.80 | 1.09 | W08057 mb37e05.r1 Mus musculus cDNA, 5′ end |
| E_TC22922_g | W11156 | 27.75 | 31.00 | 57.67 | 51.33 | 28.00 | 40.33 | 2.08 | 1.44 | ma74d01.r1 Soares mouse p3NMF 19.5 Mus musculus |
| | | | | | | | | | | cDNA clone 316417 5′ similar to gb.J03909 GAMMA- |
| | | | | | | | | | | INTERFERON-INDUCIBLE PROTEIN IP-30 |
| | | | | | | | | | | PRECURSOR (HUMAN),, mRNA sequence. |
| E_DNCH1_s | W11954 | 12.75 | 20.33 | 30.67 | 34.67 | 11.00 | 12.67 | 2.41 | 1.15 | W11954 ma79e11.r1 Mus musculus cDNA, 5′ end |
| E_DNCH1_s | W18503 | 12.25 | 14.67 | 25.00 | 31.67 | 16.50 | 16.33 | 2.04 | 0.99 | W18503 mb88b08.r1 Mus musculus cDNA, 5′ end |
| E_1_8D_f | W20873 | 10.00 | 10.00 | 32.00 | 34.67 | 12.00 | 18.67 | 3.20 | 1.56 | W20873 mb92c11.r1 Mus musculus cDNA, 5′ end |
| E_FLNA_s | W29429 | 10.00 | 13.33 | 33.67 | 29.33 | 11.00 | 14.67 | 3.37 | 1.33 | W29429 mb99d03.r1 Mus musculus cDNA, 5′ end |
| E_W48951 _i | W48951 | 10.00 | 10.00 | 20.00 | 10.00 | 10.00 | 10.00 | 2.00 | 1.00 | W48951 md24g11.r1 Mus musculus cDNA, 5′ end |
| E_W50888_f | W50888 | 12.00 | 21.67 | 24.67 | 27.67 | 16.00 | 10.00 | 2.06 | 0.63 | W50888 ma23e03.r1 Mus musculus cDNA, 5′ end |
| W50898_i | W50898 | 15.75 | 18.67 | 40.33 | 31.67 | 13.00 | 14.33 | 2.56 | 1.10 | W50898 ma23g03.r1 Mus musculus cDNA, 5′ end |
| W57485_f | W57485 | 10.00 | 10.00 | 23.67 | 21.33 | 11.50 | 10.00 | 2.37 | 0.87 | W57485 ma34h02.r1 Mus musculus cDNA, 5′ end |
| IN | X52622 | 10.25 | 10.00 | 20.33 | 57.00 | 10.00 | 10.00 | 1.98 | 1.00 | X52622 Mouse IN gene for the integrase of an |
| | | | | | | | | | | endogenous retrovirus |
| IGA_VDJ_f | X94418 | 11.75 | 14.33 | 60.00 | 71.00 | 10.00 | 15.33 | 5.11 | 1.53 | X94418 M. musculus mRNA (2F7) for IgA V-D-J-heavy |
| | | | | | | | | | | chain |
| IGH_4_f | Z70662 | 10.00 | 10.00 | 39.00 | 60.67 | 10.00 | 10.00 | 3.90 | 1.00 | Z70662 Artificial mRNA for single chain antibody scFv |
| | | | | | | | | | | (scFvP25) |
| E_TC22736_s | W12941 | 31.00 | 27.33 | 121.33 | 91.00 | 49.00 | 82.33 | 3.91 | 1.68 | ma89d07.r1 Soares mouse p3NMF 19.5 Mus musculus |
| | | | | | | | | | | cDNA clone 317869 5′ similar to gb.X57352 |
| | | | | | | | | | | INTERFERON-INDUCIBLE PROTEIN 1-8U (HUMAN),, |
| | | | | | | | | | | mRNA sequence. |
| YWHAH_s | D87661 | 10.50 | 10.00 | 22.00 | 27.33 | 10.00 | 10.00 | 2.10 | 1.00 | House mouse; Musculus domesticus mRNA for 14-3-3 |
| | | | | | | | | | | eta, complete cds |

TABLE 1A
Differentially-regulated genes

| | Accession | Untr12w | Untr.25w | Untr.36w | Untr42w | C57/3m | C57/8m | Untr 36w/12w | C57 8m/3m | | |
|---|---|---|---|---|---|---|---|---|---|---|---|
| Name | No. | (avg) | (avg) | (avg) | (avg) | (avg) | (avg) | (fold change) | (fold change) | p value | Description |
| YWHAH | D87661 | 10.50 | 10.00 | 22.00 | 27.33 | 10.00 | 10.00 | 2.10 | 1.00 | 0.02 | House mouse, Musculus domesticus mRNA for 14-3-3 eta, complete |
| | | | | | | | | | | | cds |
| VIM | X51438 | 20.25 | 16.67 | 75.00 | 53.33 | 20.00 | 23.00 | 3.70 | 1.15 | 0.02 | Mouse mRNA for vimentin. |
| VCP | W12941 | 31.00 | 27.33 | 121.33 | 91.00 | 49.00 | 82.33 | 3.91 | 1.68 | 0.02 | ma89d07.r1 Soares mouse p3NMF19 5 Mus musculus cDNA clone |
| | | | | | | | | | | | 317869 5′ similar to gb.X57352 INTERFERON-INDUCIBLE PROTEIN |
| | | | | | | | | | | | 1-8U (HUMAN),, mRNA sequence. |
| UNK_Z70662 | Z70662 | 10.00 | 10.00 | 39.00 | 60.67 | 10.00 | 10.00 | 3.90 | 1.00 | 0.06 | Z70662 Artificial mRNA for single chain antibody scFv (scFvP25) |
| UNK_X94418 | X94418 | 11.75 | 14.33 | 60.00 | 71.00 | 10.00 | 15.33 | 5.11 | 1.53 | 0.01 | X94418 M. musculus mRNA (2F7) for IgA V-D-J-heavy chain |
| UNK_X52622 | X52622 | 10.25 | 10.00 | 20.33 | 57.00 | 10.00 | 10.00 | 1.98 | 1.00 | 0.10 | X52622 Mouse IN gene for the integrase of an endogenous retrovirus |
| UNK_W57485 | W57485 | 10.00 | 10.00 | 23.67 | 21.33 | 11.50 | 10.00 | 2.37 | 0.87 | 0.07 | W57485 ma34h02.r1 Mus musculus cDNA, 5′ end |
| UNK_W50898 | W50898 | 15.75 | 18.67 | 40.33 | 31.67 | 13.00 | 14.33 | 2.56 | 1.10 | 0.06 | W50898 ma23g03.r1 Mus musculus cDNA, 5′ end |
| UNK_W50888 | W50888 | 12.00 | 21.67 | 24.67 | 27.67 | 16.00 | 10.00 | 2.06 | 0.63 | 0.01 | W50888 ma23e03.r1 Mus musculus cDNa, 5′ end |
| UNK_W48951 | W48951 | 10.00 | 10.00 | 20.00 | 10.00 | 10.00 | 10.00 | 2.00 | 1.00 | 0.36 | W48951 md24g11.r1 Mus musculus cDNA, 5′ end |
| UNK_W29429 | W29429 | 10.00 | 13.33 | 33.67 | 29.33 | 11.00 | 14.67 | 3.37 | 1.33 | 0.03 | W29429 mb99d03.r1 Mus musculus cDNA, 5′ end |
| UNK_W20873 | W20873 | 10.00 | 10.00 | 32.00 | 34.67 | 12.00 | 18.67 | 3.20 | 1.56 | 0.00 | W20873 mb92c11.r1 Mus musculus cDNA, 5′ end |
| UNK_W11156 | W11156 | 27.75 | 31.00 | 57.67 | 51.33 | 28.00 | 40.33 | 2.08 | 1.44 | 0.00 | ma74d01.r1 Soares mouse p3NMF19 5 Mus musculus cDNA clone |
| | | | | | | | | | | | 316417 5′ similar to gb.J03909 GAMMA-INTERFERON-INDUCIBLE |
| | | | | | | | | | | | PROTEIN IP-30 PRECURSOR (HUMAN),, mRNA sequence. |
| UNK_W08057 | W08057 | 10.00 | 11.00 | 48.00 | 59.00 | 13.50 | 14.67 | 4.80 | 1.09 | 0.05 | W08057 mb37e05.r1 Mus musculus cDNA, 5′ end |
| UNK_U23089 | U23089 | 10.00 | 10.67 | 30.67 | 60.67 | 10.00 | 10.00 | 3.07 | 1.00 | 0.06 | Mus musculus CB17 SCID immunoglobulin heavy chain V region |
| | | | | | | | | | | | mRNA, clone 58-53, partial cds |
| UNK_M12813 | M12813 | 10.00 | 10.33 | 33.33 | 36.00 | 10.00 | 10.00 | 3.33 | 1.00 | 0.07 | M12813 Mouse Ig germline H-chain gene H10 V-region (V), exons 1 |
| | | | | | | | | | | | and 2 |
| UNK_L28060 | L28060 | 10.00 | 10.00 | 21.00 | 20.33 | 10.00 | 10.00 | 2.10 | 1.00 | 0.12 | L28060 Mus musculus Ig B cell antigen receptor gene, complete cds |
| UNK_ET63415 | ET63415 | 10.00 | 10.00 | 34.67 | 38.00 | 10.00 | 10.00 | 3.47 | 1.00 | 0.07 | Mus musculus mRNA for IgG1/kappa antibody, scFv(glyc)- |
| | | | | | | | | | | | CK)PIR:PH1043 (Ig light chain V region (clone 111.68) - mouse |
| | | | | | | | | | | | (fragment))PIR:PH1042 (Ig light chain V region (clone 202.s38) - |
| | | | | | | | | | | | mouse (fragment) |
| UNK_ET63387 | ET63387 | 10.00 | 10.33 | 48.67 | 66.00 | 10.00 | 10.00 | 4.87 | 1.00 | 0.05 | Artificial mRNA for single chain antibody scFv (scFvP25). |
| UNK_ET63369 | ET63369 | 10.00 | 10.00 | 24.33 | 38.33 | 10.00 | 10.00 | 2.43 | 1.00 | 0.07 | M. domesticus IgG variable region. |
| UNK_ET63368 | ET63368 | 10.00 | 11.33 | 30.00 | 47.33 | 10.00 | 10.00 | 3.00 | 1.00 | 0.04 | M. domesticus IgK variable region.)PIR:PH1076 (Ig light chain V region |
| | | | | | | | | | | | (clone 74-c2) - mouse (fragment) |
| UNK_ET63365 | ET63365 | 10.00 | 11.67 | 64.33 | 75.00 | 10.00 | 10.67 | 6.43 | 1.07 | 0.04 | M. domesticus IgG variable region. |
| UNK_ET63363 | ET63363 | 10.00 | 10.00 | 43.00 | 56.00 | 10.00 | 10.00 | 4.30 | 1.00 | 0.06 | M. domesticus IgM variable region.)PIR:PH0976 (Ig heavy chain V |
| | | | | | | | | | | | region (clone 25.12m) - mouse (fragment) |
| UNK_ET63359 | ET63359 | 10.00 | 10.00 | 35.67 | 56.33 | 10.00 | 10.00 | 3.57 | 1.00 | 0.06 | M. domesticus IgM variable region.)PIR:PH1011 (Ig heavy chain V |
| | | | | | | | | | | | region (clone 202.38m) - mouse (fragment) |
| UNK_ET63358 | ET63358 | 10.00 | 10.33 | 42.00 | 46.33 | 10.00 | 10.00 | 4.20 | 1.00 | 0.06 | M. domesticus IgK variable region.)PIR:PH1046 (Ig light chain V region |
| | | | | | | | | | | | (clone 202.9) - mouse (fragment))PIR:PH1048 (Ig light chain V region |
| | | | | | | | | | | | (clone 165.49) - mouse (fragment))PIR:PH1047 (Ig light chain V region |
| | | | | | | | | | | | (clones 165.45 and 163-c1) - mouse |
| UNK_ET63354 | ET63354 | 10.00 | 11.00 | 64.33 | 75.00 | 10.00 | 10.00 | 6.43 | 1.00 | 0.06 | M. domesticus IgM variable region.)PIR:PH0995 (Ig heavy chain V |
| | | | | | | | | | | | region (clone 202.61) - mouse (fragment) |
| UNK_ET63351 | ET63351 | 10.00 | 10.00 | 34.00 | 47.33 | 10.00 | 10.00 | 3.40 | 1.00 | 0.07 | M. domesticus IgM variable region.)PIR:PH1006 (Ig heavy chain V |
| | | | | | | | | | | | region (clone 202.33) - mouse (fragment) |
| UNK_ET63348 | ET63348 | 10.00 | 10.00 | 46.33 | 59.67 | 10.00 | 10.00 | 4.63 | 1.00 | 0.07 | M. domesticus IgG variable region.)PIR:S26747 (Ig heavy chain J region |
| | | | | | | | | | | | JH4 - mouse)PIR:PH1000 (Ig heavy chain V region (clone 202.105) - |
| | | | | | | | | | | | mouse (fragment) |
| UNK_ET63341 | ET63341 | 10.00 | 10.00 | 54.33 | 72.00 | 10.00 | 10.67 | 5.43 | 1.07 | 0.04 | M. domesticus IgG variable region.)PIR:PH0984 (Ig heavy chain V |
| | | | | | | | | | | | region (clone 17s.83) - mouse (fragment) |
| UNK_ET63339 | ET63339 | 10.00 | 10.00 | 42.33 | 50.67 | 10.00 | 10.00 | 4.23 | 1.00 | 0.07 | M. domesticus IgM variable region.)PIR:PH0986 (Ig heavy chain V |
| | | | | | | | | | | | region (clone 17s-c6) - mouse (fragment) |
| UNK_ET63337 | ET63337 | 10.00 | 10.00 | 22.33 | 32.33 | 10.00 | 10.00 | 2.23 | 1.00 | 0.08 | M. domesticus IgG variable region.)PIR:PH1009 (Ig heavy chain V |
| | | | | | | | | | | | region (clone 17s.5) - mouse (fragment) |
| UNK_ET63333 | ET63333 | 10.00 | 10.67 | 78.33 | 97.33 | 10.00 | 11.67 | 7.83 | 1.17 | 0.05 | M. domesticus IgG variable region |
| UNK_ET63331 | ET63331 | 10.00 | 10.00 | 33.33 | 42.00 | 10.00 | 10.67 | 3.33 | 1.07 | 0.06 | M. domesticus IgG variable region.)PIR:PH0988 (Ig heavy chain V |
| | | | | | | | | | | | region (clone 17s-c3) - mouse (fragment) |
| UNK_ET63328 | ET63328 | 10.00 | 10.00 | 55.67 | 67.67 | 10.00 | 10.00 | 5.57 | 1.00 | 0.05 | M. domesticus IgM variable region.)PIR:PH0978 (Ig heavy chain V |
| | | | | | | | | | | | region (clone 17s.166) - mouse (fragment) |
| UNK_ET63324 | ET63324 | 10.00 | 10.00 | 35.67 | 46.67 | 10.00 | 10.00 | 3.57 | 1.00 | 0.06 | M. domesticus IgM variable region.)PIR:PH0980 (Ig heavy chain V |
| | | | | | | | | | | | region (clone 17s.13) - mouse (fragment) |
| UNK_ET63322 | ET63322 | 10.00 | 10.00 | 27.00 | 33.33 | 10.00 | 10.00 | 2.70 | 1.00 | 0.09 | M. domesticus IgK variable region.)PIR:PH1073 (Ig light chain V region |
| | | | | | | | | | | | (clone 17s.130) - mouse (fragment) |
| UNK_ET63320 | ET63320 | 10.00 | 10.33 | 57.00 | 81.33 | 10.00 | 10.00 | 5.70 | 1.00 | 0.06 | M. domesticus IgM variable region.)PIR:PH0972 (Ig heavy chain V |
| | | | | | | | | | | | region (clone 17s.128) - mouse (fragment) |
| UNK_ET63314 | ET63314 | 10.00 | 10.00 | 45.67 | 50.00 | 10.00 | 10.00 | 4.57 | 1.00 | 0.07 | M. domesticus IgM variable region.)PIR:S26747 (Ig heavy chain J region |
| | | | | | | | | | | | JH4 - mouse)PIR:PH1012 (Ig heavy chain V region (clone 17p.73) - |
| | | | | | | | | | | | mouse (fragment) |
| UNK_ET63300 | ET63300 | 10.00 | 10.00 | 63.00 | 81.00 | 10.00 | 11.33 | 6.30 | 1.13 | 0.04 | M. domesticus IgG variable region.)PIR:PH0983 (Ig heavy chain V |
| | | | | | | | | | | | region (clone 165.49) - mouse (fragment) |
| UNK_ET63295 | ET63295 | 10.00 | 10.67 | 75.33 | 79.67 | 10.00 | 11.33 | 7.53 | 1.13 | 0.06 | M. domesticus IgM variable region.)PIR.S26747 (Ig heavy chain J region |
| | | | | | | | | | | | JH4 - mouse |
| UNK_ET63290 | ET63290 | 10.00 | 10.00 | 40.67 | 26.00 | 10.00 | 10.00 | 4.07 | 1.00 | 0.17 | M. domesticus IgK variable region.)PIR.PH1066 (Ig light chain V region |
| | | | | | | | | | | | (clone 165.14) - mouse (fragment) |
| UNK_ET63288 | ET63288 | 10.00 | 10.00 | 40.67 | 46.33 | 10.00 | 10.00 | 4.07 | 1.00 | 0.06 | M. domesticus IgM variable region.)PIR.PH0975 (Ig heavy chain V |
| | | | | | | | | | | | region (clone 163.72) - mouse (fragment) |
| UNK_ET63278 | ET63278 | 10.00 | 10.00 | 38.33 | 51.67 | 10.00 | 10.00 | 3.83 | 1.00 | 0.06 | M. domesticus IgG variable region.)PIR.PH1007 (Ig heavy chain V |
| | | | | | | | | | | | region (clone 163-c1) - mouse (fragment) |
| UNK_ET63276 | ET63276 | 10.00 | 10.00 | 85.67 | 93.33 | 10.00 | 16.00 | 8.57 | 1.60 | 0.04 | M. domesticus IgM variable region.)PIR.S26746 (Ig heavy chain J region |
| | | | | | | | | | | | JH3 - mouse)PIR:PH0985 (Ig heavy chain V region (clone 163.100) - |
| | | | | | | | | | | | mouse (fragment) |
| UNK_ET63274 | ET63274 | 10.00 | 10.00 | 51.33 | 61.33 | 10.00 | 11.00 | 5.13 | 1.10 | 0.06 | M. domesticus IgG variable region.)PIR.PH1001 (Ig heavy chain V |
| | | | | | | | | | | | region (clone 111.68) - mouse (fragment) |
| UNK_ET63271 | ET63271 | 11.00 | 10.33 | 23.67 | 32.00 | 10.00 | 10.00 | 2.15 | 1.00 | 0.07 | M. domesticus IgG variable region.)PIR.PH1015 (Ig heavy chain V |
| | | | | | | | | | | | region (clone 111.55) - mouse (fragment) |
| UNK_ET63126 | ET63126 | 10.00 | 11.67 | 30.00 | 40.33 | 10.00 | 11.00 | 3.00 | 1.10 | 0.03 | M. musculus mRNA for anti folate binding protein, MOv19 Vkappa |
| UNK_ET63107 | ET63107 | 10.00 | 10.00 | 32.67 | 21.67 | 10.00 | 10.00 | 3.27 | 1.00 | 0.15 | M. musculus mRNA for immunoglobulin kappa light chain variable |
| | | | | | | | | | | | region. |
| UNK_ET63106 | ET63106 | 10.00 | 10.00 | 22.33 | 32.67 | 10.00 | 10.00 | 2.23 | 1.00 | 0.07 | M. musculus mRNA for immunoglobulin heavy chain variable region, |
| | | | | | | | | | | | isloate 205 |
| UNK_ET63093 | ET63093 | 10.00 | 10.00 | 34.00 | 46.00 | 10.00 | 11.67 | 3.40 | 1.17 | 0.07 | M. musculus mRNA for immunoglobulin heavy chain variable domain, |
| | | | | | | | | | | | subgroup llb |
| UNK_ET63085 | ET63085 | 10.00 | 10.00 | 49.33 | 57.33 | 10.00 | 10.00 | 4.93 | 1.00 | 0.07 | M. musculus mRNA for monoclonal antibody heavy chain variable |
| | | | | | | | | | | | region |
| UNK_ET63042 | ET63042 | 10.00 | 10.00 | 29.00 | 34.00 | 10.00 | 10.00 | 2.90 | 1.00 | 0.11 | M. musculus mRNA for immunoglobulin kappa variable region. |
| UNK_ET63041 | ET63041 | 10.00 | 10.00 | 55.00 | 60.00 | 10.00 | 10.67 | 5.50 | 1.07 | 0.06 | M. musculus mRNA for immunoglobulin heavy variable region. |
| UNK_ET63027 | ET63027 | 10.00 | 10.00 | 24.33 | 18.67 | 10.00 | 10.00 | 2.43 | 1.00 | 0.14 | M. musculus mRNA for immunoglobulin variable region, heavy chain. |
| UNK_ET62984 | ET62984 | 10.00 | 10.33 | 66.00 | 69.67 | 10.00 | 15.33 | 6.60 | 1.53 | 0.02 | M. musculus mRNA (3C10) for IgA V-D-J-heavy chain. |
| UNK_ET62983 | ET62983 | 11.00 | 13.00 | 56.00 | 70.67 | 10.00 | 15.67 | 5.09 | 1.57 | 0.01 | M. musculus mRNA (2F7) for IgA V-D-J-heavy chain. |
| UNK_ET62942 | ET62942 | 10.00 | 10.00 | 44.00 | 49.33 | 10.00 | 10.33 | 4.40 | 1.03 | 0.04 | M. musculus antibody light chain variable region (324bp). |
| UNK_ET62941 | ET62941 | 10.00 | 10.00 | 37.33 | 45.00 | 10.00 | 10.00 | 3.73 | 1.00 | 0.06 | M. musculus antibody light chain variable region (318bp). |
| UNK_ET62936 | ET62936 | 10.00 | 10.00 | 24.67 | 38.00 | 10.00 | 10.00 | 2.47 | 1.00 | 0.07 | M. musculus antibody heavy chain variable region (375bp). |
| UNK_ET62934 | ET62934 | 10.00 | 10.00 | 30.33 | 37.00 | 10.00 | 10.00 | 3.03 | 1.00 | 0.09 | M. musculus antibody heavy chain variable region (348bp). |
| UNK_ET62933 | ET62933 | 10.00 | 10.00 | 25.67 | 34.33 | 10.00 | 10.00 | 2.57 | 1.00 | 0.08 | M. musculus antibody heavy chain variable region (360bp). |
| UNK_ET62932 | ET62932 | 10.00 | 10.00 | 22.00 | 34.00 | 10.00 | 10.00 | 2.20 | 1.00 | 0.08 | M. musculus antibody heavy chain variable region (372bp). |
| UNK_ET62928 | ET62928 | 11.00 | 10.33 | 23.00 | 30.67 | 10.00 | 10.00 | 2.09 | 1.00 | 0.05 | M. musculus antibody heavy chain variable region (366bp). |
| UNK_ET62926 | ET62926 | 10.00 | 10.00 | 30.00 | 26.67 | 10.00 | 10.00 | 3.00 | 1.00 | 0.12 | M. musculus antibody heavy chain variable region (354bp). |
| UNK_ET62925 | ET62925 | 10.00 | 10.33 | 74.67 | 76.67 | 10.00 | 13.00 | 7.47 | 1.30 | 0.06 | M. musculus antibody heavy chain variable region (372bp). |
| UNK_ET62924 | ET62924 | 10.00 | 10.00 | 59.67 | 54.00 | 10.00 | 10.00 | 5.97 | 1.00 | 0.09 | M. musculus antibody heavy chain variable region (363bp). |
| UNK_ET62923 | ET62923 | 10.00 | 10.00 | 56.67 | 65.00 | 10.00 | 10.00 | 5.67 | 1.00 | 0.06 | M. musculus antibody heavy chain variable region (354bp). |
| UNK_ET62868 | ET62868 | 10.00 | 10.00 | 33.67 | 40.33 | 10.00 | 10.00 | 3.37 | 1.00 | 0.06 | Mus musculus anti-CD8 immunoglobulin heavy chain V region mRNA, |
| | | | | | | | | | | | partial cds |
| UNK_ET62779 | ET62779 | 10.00 | 10.00 | 65.67 | 76.67 | 10.00 | 10.00 | 6.57 | 1.00 | 0.06 | Mus musculus IgM heavy chain variable region mRNA, partial cds |
| UNK_ET62430 | ET62430 | 10.00 | 10.00 | 21.33 | 22.33 | 10.00 | 10.00 | 2.13 | 1.00 | 0.10 | Mus musculus Ig heavy chain Fv fragment mRNA, partial cds |
| UNK_ET62260 | ET62260 | 10.00 | 11.67 | 37.67 | 51.33 | 10.00 | 12.00 | 3.77 | 1.20 | 0.03 | Mus musculus immunoglobulin light chain variable region mRNA, |
| | | | | | | | | | | | partial cds |
| UNK_ET62256 | ET62256 | 10.00 | 10.00 | 36.00 | 46.33 | 10.00 | 10.00 | 3.60 | 1.00 | 0.08 | Mus musculus anti-PAH immunoglobulin Fab 4D5 heavy chain V and |
| | | | | | | | | | | | CH1 regions mRNA, partial cds |
| UNK_ET62234 | ET62234 | 10.00 | 10.00 | 26.67 | 47.33 | 10.00 | 10.00 | 2.67 | 1.00 | 0.06 | Mus musculus polyreactive autoantibody, immunoglobulin IgM heavy |
| | | | | | | | | | | | chain mRNA, partial cds |
| UNK_ET62233 | ET62233 | 10.00 | 10.00 | 32.00 | 50.33 | 10.00 | 10.00 | 3.20 | 1.00 | 0.07 | Mus musculus polyreactive autoantibody, immunoglobulin IgM heavy |
| | | | | | | | | | | | chain mRNA, partial cds |
| UNK_ET62224 | ET62224 | 10.00 | 10.00 | 31.33 | 26.33 | 10.00 | 10.00 | 3.13 | 1.00 | 0.10 | Mus musculus immunoglobulin heavy chain variable region mRNA, |
| | | | | | | | | | | | partial cds |
| UNK_ET62206 | ET62206 | 10.00 | 12.67 | 27.67 | 38.00 | 10.50 | 10.67 | 2.77 | 1.02 | 0.02 | Mus musculus anti-digoxin immunoglobulin heavy chain variable region |
| | | | | | | | | | | | precursor mRNA, partial cds |
| UNK_ET62172 | ET62172 | 10.00 | 10.33 | 61.00 | 72.67 | 10.00 | 11.33 | 6.10 | 1.13 | 0.06 | Mus musculus anti-PAH immunoglobulin Fab 10C10 heavy chain V and |
| | | | | | | | | | | | CH1 regions gene, partial cds |
| UNK_ET61749 | ET61749 | 10.00 | 10.00 | 21.00 | 23.33 | 10.00 | 10.00 | 2.10 | 1.00 | 0.09 | Mus musculus Ig 6C3.B8 heavy chain mRNA, specific for rat (mouse) |
| | | | | | | | | | | | cytochrome c, partial cds |
| UNK_ET61748 | ET61748 | 10.00 | 10.00 | 35.67 | 40.33 | 10.00 | 10.00 | 3.57 | 1.00 | 0.06 | Mus musculus Ig 4C5.A11 heavy chain mRNA, specific for rat (mouse) |
| | | | | | | | | | | | cytochrome c, partial cds |
| UNK_ET61747 | ET61747 | 10.00 | 10.00 | 40.67 | 39.00 | 10.00 | 10.00 | 4.07 | 1.00 | 0.07 | Mus musculus Ig 4C4.A10 heavy chain mRNA, specific for rat (mouse) |
| | | | | | | | | | | | cytochrome c, partial cds |
| UNK_ET61746 | ET61746 | 10.00 | 10.00 | 43.00 | 40.67 | 10.00 | 10.00 | 4.30 | 1.00 | 0.08 | Mus musculus Ig 4A6.A8 heavy chain mRNA, specific for rat (mouse) |
| | | | | | | | | | | | cytochrome c, partial cds |
| UNK_ET61744 | ET61744 | 10.00 | 10.00 | 20.00 | 24.00 | 10.00 | 10.00 | 2.00 | 1.00 | 0.08 | Mus musculus Ig 3F10.C9 heavy chain mRNA, specific for rat (mouse) |
| | | | | | | | | | | | cytochrome c, partial cds |
| UNK_ET61741 | ET61741 | 10.00 | 10.00 | 31.33 | 45.00 | 10.00 | 10.00 | 3.13 | 1.00 | 0.07 | Mus musculus Ig 2C9.B12 heavy chain mRNA, specific for rat (mouse) |
| | | | | | | | | | | | cytochrome c, partial cds |
| UNK_ET61739 | ET61739 | 10.00 | 10.00 | 23.67 | 28.33 | 10.00 | 10.00 | 2.37 | 1.00 | 0.08 | Mus musculus Ig 7D1.B8 heavy chain mRNA, specific for rat (mouse) |
| | | | | | | | | | | | cytochrome c, partial cds |
| UNK_ET61737 | ET61737 | 10.00 | 10.00 | 30.33 | 37.67 | 10.00 | 10.00 | 3.03 | 1.00 | 0.08 | Mus musculus Ig 3A6.A5 heavy chain mRNA, specific for rat (mouse) |
| | | | | | | | | | | | cytochrome c, partial cds |
| UNK_ET61736 | ET61736 | 10.00 | 10.00 | 44.67 | 50.00 | 10.00 | 10.00 | 4.47 | 1.00 | 0.05 | Mus musculus Ig 9G7.A10 heavy chain mRNA, specific for rat (mouse) |
| | | | | | | | | | | | cytochrome c, partial cds |
| UNK_ET61733 | ET61733 | 10.00 | 10.00 | 32.67 | 36.00 | 10.00 | 10.00 | 3.27 | 1.00 | 0.08 | Mus musculus Ig 7A12.A2 heavy chain mRNA, specific for rat (mouse) |
| | | | | | | | | | | | cytochrome c, partial cds |
| UNK_ET61732 | ET61732 | 10.00 | 10.00 | 30.33 | 36.00 | 10.00 | 10.00 | 3.03 | 1.00 | 0.07 | Mus musculus Ig 5C12.A4 heavy chain mRNA, specific for rat (mouse) |
| | | | | | | | | | | | cytochrome c, partial cds |
| UNK_ET61730 | ET61730 | 10.00 | 10.00 | 37.67 | 60.00 | 10.00 | 10.00 | 3.77 | 1.00 | 0.05 | Mus musculus Ig 2G3.H5 heavy chain mRNA, specific for rat (mouse) |
| | | | | | | | | | | | cytochrome c, partial cds |
| UNK_ET61727 | ET61727 | 10.00 | 10.00 | 29.00 | 36.67 | 10.00 | 10.00 | 2.90 | 1.00 | 0.08 | Mus musculus Ig 2G11.E2 heavy chain mRNA, specific for rat (mouse) |
| | | | | | | | | | | | cytochrome c, partial cds |
| UNK_ET61599 | ET61599 | 10.00 | 11.00 | 42.00 | 57.33 | 10.00 | 10.33 | 4.20 | 1.03 | 0.03 | Mus musculus monoclonal antibody against hepatitis B surface antigen, |
| | | | | | | | | | | | IgG light chain variable region gene, partial cds |
| UNK_ET61520 | ET61520 | 10.00 | 10.00 | 45.00 | 47.00 | 10.00 | 10.00 | 4.50 | 1.00 | 0.09 | Mus musculus IgG rearranged heavy chain mRNA, variable region |
| | | | | | | | | | | | partial cds |
| UNK_ET61464 | ET61464 | 10.00 | 10.00 | 23.00 | 34.67 | 10.00 | 10.00 | 2.30 | 1.00 | 0.07 | Mus musculus immunoglobulin heavy chain mRNA, V, D, and J |
| | | | | | | | | | | | segments, partial cds |
| UNK_ET61420 | ET61420 | 10.00 | 10.00 | 65.67 | 86.33 | 10.00 | 10.67 | 6.57 | 1.07 | 0.04 | Mus musculus anti-glycoprotein-B of human Cytomegalovirus |
| | | | | | | | | | | | immunoglobulin Vh chain gene, partial cds |
| UNK_C80574 | C80574 | 28.00 | 21.67 | 60.67 | 83.00 | 36.00 | 42.00 | 2.17 | 1.17 | 0.02 | Mouse 3.5-dpc blastocyst cDNA musculus cDNA clone J0084D04 |
| | | | | | | | | | | | 3′ similar to Human clone 23665 mRNA sequence. |
| UNK_C77861 | C77861 | 16.50 | 13.33 | 35.67 | 42.67 | 17.50 | 17.67 | 2.16 | 1.01 | 0.01 | Mouse 3.5-dpc blastocyst cDNA Mus musculus cDNA clone J0038G08 |
| | | | | | | | | | | | 3′ similar to Rattus norvegicus major vault protein mRNA, mRNA |
| | | | | | | | | | | | sequence. |
| UNK_C76523 | C76523 | 11.50 | 10.00 | 30.67 | 40.33 | 10.00 | 11.00 | 2.67 | 1.10 | 0.03 | Mouse 3.5-dpc blastocyst cDNA Mus musculus cDNA clone J0012E07 |
| | | | | | | | | | | | 3′, mRNA sequence. |
| UNK_C76523 | C76523 | 10.00 | 10.00 | 23.00 | 19.67 | 10.00 | 10.00 | 2.30 | 1.00 | 0.06 | Mouse 3.5-dpc blastocyst cDNA Mus musculus cDNA clone J0012E07 |
| | | | | | | | | | | | 3′, mRNA sequence. |
| UNK_AC002397 | AC002397 | 10.00 | 13.00 | 25.00 | 33.33 | 10.00 | 14.00 | 2.50 | 1.40 | 0.01 | Mouse chromosome 6 BAC-284H12 (Research Genetics mouse BAC |
| | | | | | | | | | | | library) complete sequence. |
| UNK_AA710451 | AA710451 | 10.00 | 10.00 | 46.33 | 31.67 | 10.00 | 10.00 | 4.63 | 1.00 | 0.10 | vt42f07.r1 Barstead mouse proximal colon MPLRB6 Mus musculus |
| | | | | | | | | | | | cDNA clone 1165765 5′, mRNA sequence. |
| UNK_AA690738 | AA690738 | 15.50 | 12.00 | 36.33 | 49.67 | 12.50 | 15.33 | 2.34 | 1.23 | 0.01 | vu57b03.r1 Soares mouse mammary gland NbMMG Mus musculus |
| | | | | | | | | | | | cDNA clone 1195469 5′, mRNA sequence. |
| UNK_AA616243 | AA616243 | 10.00 | 10.00 | 21.33 | 37.67 | 10.00 | 10.00 | 2.13 | 1.00 | 0.08 | vo50d04.r1 Barstead mouse irradiated colon MPLRB7 Mus musculus |
| | | | | | | | | | | | cDNA clone 1053319 5′, mRNA sequence. |
| UNK_AA606926 | AA606926 | 15.25 | 10.33 | 35.00 | 46.00 | 13.00 | 23.67 | 2.30 | 1.82 | 0.03 | vm91d04.r1 Knowles Solter mouse blastocyst B1 Mus musculus cDNA |
| | | | | | | | | | | | clone 1005607 5′ similar to TR.G497940 G497940 MAJOR VAULT |
| | | | | | | | | | | | PROTEIN ,, mRNA sequence. |
| UNK_AA562685 | AA562685 | 11.50 | 10.00 | 58.33 | 28.67 | 11.00 | 14.33 | 5.07 | 1.30 | 0.04 | vl56h09.r1 Stratagene mouse skin (#937313) Mus musculus cDNA |
| | | | | | | | | | | | clone 976289 5′ similar to gb X06753 Mouse pro-alpha1 (MOUSE), |
| UNK_AA538477 | AA538477 | 11.00 | 11.67 | 22.67 | 42.67 | 10.00 | 10.00 | 2.06 | 1.00 | 0.08 | vj53e12.r1 Knowles Solter mouse blastocyst B1 Mus musculus cDNA |
| | | | | | | | | | | | clone 932782 5′ |
| UNK_AA210359 | AA210359 | 13.00 | 11.00 | 29.33 | 37.33 | 13.00 | 13.00 | 2.26 | 1.00 | 0.01 | mu72h03.r1 Soares mouse lymph node NbMLN Mus musculus cDNA |
| | | | | | | | | | | | clone 644981 5′ |
| UNK_AA174883 | AA174883 | 25.00 | 32.00 | 65.67 | 109.67 | 10.00 | 10.00 | 2.63 | 1.00 | 0.05 | ms77e07.r1 Soares mouse 3NbMS Mus musculus cDNA clone 617604 5′ |
| UNK_AA172851 | AA172851 | 10.00 | 11.33 | 21.67 | 58.33 | 10.00 | 11.67 | 2.17 | 1.17 | 0.07 | mr31f05.r1 Soares mouse 3NbMS Mus musculus cDNA clone 599073 5′ |
| UNK_AA165775 | AA165775 | 30.25 | 25.00 | 14.67 | 23.67 | 23.50 | 36.33 | 0.48 | 1.55 | 0.01 | mt74d01.r1 Soares mouse lymph node NbMLN Mus musculus cDNA |
| | | | | | | | | | | | clone 635617 5′ |
| UNK_AA109909 | AA109909 | 10.00 | 10.00 | 28.67 | 17.00 | 10.00 | 10.00 | 2.87 | 1.00 | 0.21 | AA109909 mp10d09.r1 Mus musculus cDNA, 5′ end |
| UNK_AA107847 | AA107847 | 10.00 | 10.00 | 34.67 | 16.00 | 10.00 | 10.00 | 3.47 | 1.00 | 0.26 | AA107847 mo49d08.r1 Mus musculus cDNA, 5′ end |
| UNK_AA104688 | AA104688 | 10.00 | 10.00 | 42.67 | 27.33 | 10.00 | 10.00 | 4.27 | 1.00 | 0.12 | AA104688 mo55c10.r1 Mus musculus cDNA, 5′ end |
| UNK_AA087673 | AA087673 | 10.00 | 22.33 | 81.67 | 245.33 | 13.00 | 11.00 | 8.17 | 0.85 | 0.04 | AA087673 mm27b09.r1 Mus musculus cDNA, 5′ end |
| UNK_AA030688 | AA030688 | 10.25 | 10.00 | 25.67 | 36.33 | 10.00 | 10.00 | 2.50 | 1.00 | 0.06 | mi22g02.r1 Soares mouse embryo NbME13.5 14.5 Mus musculus |
| | | | | | | | | | | | cDNA clone 464306 5′ |
| UNK_AA023491 | AA023491 | 10.00 | 10.00 | 38.33 | 20.33 | 10.00 | 10.00 | 3.83 | 1.00 | 0.22 | AA023491 mh74e11.r1 Mus musculus cDNA, 5′ end |
| UNK_AA002761 | AA002761 | 10.00 | 10.00 | 22.67 | 24.00 | 10.00 | 11.67 | 2.27 | 1.17 | 0.07 | mg45b10.r1 Soares mouse embryo NbME13.5 14.5 Mus musculus |
| | | | | | | | | | | | cDNA clone 426715 5′ |
| TGTP | L38444 | 10.00 | 10.00 | 20.00 | 20.33 | 13.00 | 19.33 | 2.00 | 1.49 | 0.07 | Mus musculus (clone U2) T-cell specific protein mRNA, complete cds |
| TGFBI | L19932 | 10.00 | 10.00 | 30.33 | 25.67 | 10.00 | 11.33 | 3.03 | 1.13 | 0.03 | Mouse (beta ig-h3) mRNA, complete cds |
| TAGLN2 | AA120653 | 35.25 | 34.00 | 124.67 | 148.33 | 51.50 | 82.67 | 3.54 | 1.61 | 0.02 | mp71g11.r1 Soares 2NbMT Mus musculus cDNA clone 574724 5′ |
| | | | | | | | | | | | similar to gb D21261 SM22-ALPHA HOMOLOG (HUMAN), |
| STK2 | AA108677 | 10.00 | 11.00 | 21.00 | 24.33 | 10.00 | 12.00 | 2.10 | 1.20 | 0.02 | mp39a05.r1 Barstead MPLRB1 Mus musculus cDNA clone 571568 5′ |
| STAT3 | AA396029 | 10.00 | 10.00 | 20.67 | 34.00 | 11.00 | 20.00 | 2.07 | 1.82 | 0.01 | vb41e05.r1 Soares mouse lymph node NbMLN Mus musculus cDNA |
| | | | | | | | | | | | clone 751520 5′ |
| STAT3 | U06922 | 42.25 | 37.67 | 99.33 | 152.33 | 26.00 | 16.67 | 2.35 | 0.64 | 0.01 | Mus musculus signal transducer and activator of transcription (Stat3) |
| | | | | | | | | | | | mRNA, complete cds |
| SPARC | X04017 | 24.50 | 22.00 | 78.67 | 54.67 | 32.00 | 37.00 | 3.21 | 1.16 | 0.02 | X04017 Mouse mRNA for cysteine-rich glycoprotein SPARC |
| SNRPD1 | M58558 | 10.00 | 10.33 | 20.33 | 24.33 | 16.50 | 15.33 | 2.03 | 0.93 | 0.02 | Murine sm D small nuclear ribonucleoprotein sequence. |
| SLPI | U73004 | 10.00 | 10.00 | 24.00 | 26.67 | 10.00 | 11.33 | 2.40 | 1.13 | 0.05 | Mus musculus secretory leukocyte protease inhibitor mRNA, complete |
| | | | | | | | | | | | cds |
| SLC20A1 | M73696 | 10.00 | 10.00 | 20.67 | 31.67 | 10.00 | 10.00 | 2.07 | 1.00 | 0.03 | Munne Glvr-1 mRNA, complete cds |
| SCYD1 | U92565 | 11.50 | 10.00 | 30.33 | 25.67 | 10.00 | 13.00 | 2.64 | 1.30 | 0.14 | Mus musculus fractalkine mRNA, complete cds |
| SCYA5 | U02298 | 10.00 | 10.00 | 22.33 | 13.67 | 10.00 | 10.00 | 2.23 | 1.00 | 0.24 | Mus musculus NIH.3T3 chemokine rantes (Scya5) gene, complete cds |
| SCYA19 | AA137292 | 16.25 | 22.00 | 32.33 | 46.67 | 14.00 | 22.33 | 1.99 | 1.60 | 0.04 | mq98h01.r1 Soares mouse 3NbMS Mus musculus cDNA clone 596017 5′ |
| RRM2 | C81593 | 10.00 | 10.00 | 23.00 | 17.67 | 10.00 | 10.00 | 2.30 | 1.00 | 0.02 | Mouse 3.5-dpc blastocyst cDNA Mus musculus cDNA clone J0101H11 |
| | | | | | | | | | | | 3′ similar to Mouse ribonucleotide reductase M2 subunit mRNA, mRNA |
| | | | | | | | | | | | sequence. |
| RRAS | W41501 | 10.25 | 10.00 | 21.67 | 25.67 | 10.00 | 10.00 | 2.11 | 1.00 | 0.02 | W41501 mc43d11.r1 Mus musculus cDNA, 5′ end |
| RRAS | M21019 | 16.00 | 12.00 | 43.33 | 53.33 | 18.00 | 28.00 | 2.71 | 1.56 | 0.06 | Mouse R-ras mRNA, complete cds |
| RPS26 | C76830 | 11.75 | 10.00 | 27.33 | 34.67 | 37.00 | 37.33 | 2.33 | 1.01 | 0.05 | Mouse 3.5-dpc blastocyst cDNA Mus musculus cDNA clone J0020H05 |
| | | | | | | | | | | | 3′ similar to Mus musculus ribosomal protein S26 (RPS26) mRNA, |
| | | | | | | | | | | | mRNA sequence. |
| RPL13A | AA408475 | 11.00 | 11.33 | 24.33 | 21.67 | 20.00 | 19.33 | 2.21 | 0.97 | 0.02 | Mus musculus cDNA clone C0028E12 3′, mRNA sequence. |
| RGS2 | U67187 | 10.00 | 14.67 | 24.33 | 41.33 | 12.00 | 10.00 | 2.43 | 0.83 | 0.02 | Mus musculus G protein signaling regulator RGS2 (rgs2) mRNA, |
| | | | | | | | | | | | complete cds |
| RBM3 | AA538285 | 13.50 | 10.67 | 42.00 | 82.33 | 10.50 | 13.67 | 3.11 | 1.30 | 0.02 | vj03d05.r1 Barstead mouse pooled organs MPLRB4 Mus musculus |
| | | | | | | | | | | | cDNA clone 920649 5′ similar to TR.G881954 G881954 RNPL , |
| RAC2 | X53247 | 13.00 | 17.67 | 59.67 | 59.67 | 16.50 | 25.67 | 4.59 | 1.56 | 0.02 | M. musculus EN-7 mRNA |
| PVA | X67141 | 28.00 | 22.33 | 10.00 | 11.00 | 22.00 | 24.33 | 0.36 | 1.11 | 0.00 | M. musculus Pva mRNA for parvalbumin |
| PTPN1 | U24700 | 10.00 | 11.67 | 22.00 | 42.33 | 17.50 | 14.33 | 2.20 | 0.82 | 0.03 | Mus musculus protein tyrosine phosphatase (HA2) mR |
| PSME2 | D87910 | 21.75 | 24.67 | 64.00 | 74.00 | 41.00 | 38.33 | 2.94 | 0.93 | 0.01 | Mus musculus mRNA for PA28 beta subunit, complete cds |
| PRG | X16133 | 18.25 | 14.00 | 51.33 | 43.67 | 33.00 | 35.00 | 2.81 | 1.06 | 0.03 | Mouse mRNA for mastocytoma proteoglycan core protein, serglycin |
| PEA15 | AA108330 | 11.50 | 10.00 | 40.00 | 51.33 | 10.00 | 10.00 | 3.48 | 1.00 | 0.03 | AA108330 mp28b03.r1 Mus musculus cDNA, 5′ end |
| P21ARC | AA408672 | 39.25 | 36.00 | 80.00 | 75.67 | 50.00 | 42.00 | 2.04 | 0.84 | 0.02 | EST03133 Mouse 7.5 dpc embryo ectoplacental cone cDNA library |
| | | | | | | | | | | | Mus musculus cDNA clone C0031D07 3′ |
| OAS1A | M33863 | 11.50 | 10.00 | 25.00 | 28.33 | 12.00 | 12.67 | 2.17 | 1.06 | 0.09 | Mouse 2′-5′ oligo A synthetase mRNA, complete cds |
| NFKBIA | U36277 | 17.75 | 18.33 | 44.67 | 47.00 | 29.50 | 19.33 | 2.52 | 0.66 | 0.00 | U36277 Mus musculus I-kappa B alpha chain mRNA, complete cds |
| NFKBIA | U36277 | 14.75 | 17.67 | 44.00 | 42.00 | 23.00 | 18.00 | 2.98 | 0.78 | 0.00 | U36277 Mus musculus I-kappa B alpha chain mRNA, complete cds |
| MPEG1 | L20315 | 10.00 | 10.00 | 30.00 | 37.67 | 10.00 | 12.33 | 3.00 | 1.23 | 0.02 | L20315 Mus musculus MPS1 gene and mRNA, 3′ end |
| MLP | AA245242 | 11.25 | 11.00 | 31.00 | 32.33 | 11.50 | 17.00 | 2.76 | 1.48 | 0.01 | mw28h11.r1 Soares mouse 3NME12.5 Mus musculus cDNA clone |
| | | | | | | | | | | | 672069.5′ similar to gb:x61399 Mouse F52 mRNA for a novel protein |
| | | | | | | | | | | | (MOUSE), |
| MGLAP | D00613 | 47.75 | 44.33 | 249.67 | 132.33 | 113.00 | 190.00 | 5.23 | 1.68 | 0.01 | D00613 Mouse mRNA for matrix Gla protein (MGP) |
| MDK | AA072643 | 15.50 | 25.00 | 37.67 | 28.00 | 16.00 | 18.00 | 2.43 | 1.13 | 0.01 | AA072643 mm75a09.r1 Mus musculus cDNA, 5′ end |
| MAPK1 | AA104744 | 10.00 | 10.00 | 28.67 | 23.00 | 10.00 | 10.00 | 2.87 | 1.00 | 0.04 | AA104744 mo56d02.r1 Mus musculus cDNA, 5′ end |
| LYN | M57696 | 14.25 | 13.67 | 30.00 | 43.33 | 20.50 | 21.00 | 2.11 | 1.02 | 0.01 | Mouse lyn A protein tyrosine kinase (lynA) mRNA, complete cds |
| LST1 | U72643 | 11.00 | 13.00 | 29.33 | 29.67 | 17.00 | 14.33 | 2.67 | 0.84 | 0.02 | Mus musculus lymphocyte specific transcript (LST) mRNA, partial cds |
| LOC56722 | AA542220 | 14.50 | 11.33 | 42.67 | 64.33 | 11.00 | 17.67 | 2.94 | 1.61 | 0.03 | vk43h10.r1 Soares mouse mammary gland NbMMG Mus musculus |
| | | | | | | | | | | | cDNA clone 949411 5′ |
| LGALS3 | W10936 | 10.00 | 10.00 | 27.33 | 28.33 | 14.00 | 12.67 | 2.73 | 0.90 | 0.03 | W10936 ma03e09.r1 Mus musculus cDNA, 5′ end |
| LAPTM5 | U29539 | 10.25 | 11.00 | 27.33 | 34.00 | 10.00 | 16.33 | 2.67 | 1.63 | 0.02 | Mus musculus retinoic acid-inducible E3 protein mR |
| LAG | AA117100 | 11.50 | 11.67 | 24.33 | 19.67 | 19.00 | 14.00 | 2.12 | 0.74 | 0.06 | AA117100 mo60a10.r1 Mus musculus cDNA, 5′ end |
| KRT2-8 | D90360 | 19.50 | 18.00 | 49.00 | 92.67 | 23.50 | 30.67 | 2.51 | 1.30 | 0.04 | Mouse gene for cytokeratin endo A |
| JUN | W09701 | 16.25 | 16.33 | 32.33 | 32.67 | 18.00 | 13.00 | 1.99 | 0.72 | 0.00 | W09701 ma56e02.r1 Mus musculus cDNA, 5′ end |
| ITPR1 | X15373 | 42.25 | 30.00 | 20.00 | 24.67 | 30.50 | 28.67 | 0.47 | 0.94 | 0.00 | Mouse cerebellum mRNA for P400 protein |
| | | | | | | | | | | | mn33e03.r1 Beddington mouse embryonic region Mus musculus cDNA |
| | | | | | | | | | | | clone 539740 5′ similar to TR. E236822 E236822 HYPOTHETICAL 26.5 |
| ITGB4BP | AA122622 | 11.25 | 10.00 | 25.33 | 16.33 | 10.00 | 10.00 | 2.25 | 1.00 | 0.25 | KD PROTEIN; |
| IRF7 | U73037 | 10.00 | 10.67 | 27.33 | 33.33 | 11.50 | 11.33 | 2.73 | 0.99 | 0.03 | Mus musculus interferon regulatory factor 7 (mirf7) mRNA, complete |
| | | | | | | | | | | | cds |
| IGK-V20 | X16678 | 10.00 | 10.00 | 36.33 | 24.00 | 10.00 | 10.00 | 3.63 | 1.00 | 0.17 | Mouse VK gene for kappa light chain variable region and J4 sequence. |
| IFNGR | J05265 | 12.75 | 11.00 | 27.67 | 40.67 | 15.00 | 16.00 | 2.17 | 1.07 | 0.02 | Mouse interferon gamma receptor mRNA, complete cds |
| IFIT3 | L32974 | 13.75 | 10.00 | 29.00 | 33.00 | 14.50 | 14.67 | 2.11 | 1.01 | 0.04 | Mouse interferon-inducible protein homologue mRNA, complete cds |
| HSP25 | AA015458 | 10.50 | 10.00 | 24.67 | 20.67 | 12.00 | 11.00 | 2.35 | 0.92 | 0.17 | AA015458 mh22b09.r1 Mus musculus cDNA, 5′ end |
| HSP25 | AA034638 | 10.00 | 10.00 | 20.00 | 29.67 | 10.00 | 10.00 | 2.00 | 1.00 | 0.06 | AA034638 mh17a07.r1 Mus musculus cDNA, 5′ end |
| HSP25 | L07577 | 31.75 | 35.00 | 131.67 | 191.00 | 56.00 | 50.67 | 4.15 | 0.90 | 0.03 | Mus musculus small heat shock protein (HSP25) gene |
| HSP25 | AA015026 | 12.25 | 14.33 | 38.67 | 44.33 | 15.00 | 11.00 | 3.16 | 0.73 | 0.04 | AA015026 mh26f03.r1 Mus musculus cDNA, 5′ end |
| HN1 | U90123 | 10.00 | 10.00 | 23.67 | 25.00 | 12.50 | 14.00 | 2.37 | 1.12 | 0.05 | Mus musculus HN1 (Hn1) mRNA, complete cds |
| HMOX1 | M33203 | 10.00 | 10.00 | 20.00 | 28.33 | 10.00 | 10.00 | 2.00 | 1.00 | 0.07 | Mouse tumor-induced 32 kD protein (p32) mRNA, complete cds |
| GRN | M86736 | 56.25 | 51.67 | 129.00 | 159.67 | 55.50 | 81.00 | 2.29 | 1.46 | 0.01 | Mouse acrogranin mRNA, complete cds |
| GNB1 | U29055 | 11.75 | 11.33 | 28.33 | 37.33 | 12.00 | 14.33 | 2.41 | 1.19 | 0.02 | Mus musculus G protein beta 36 subunit mRNA, compl |
| FXYD5 | U72680 | 10.25 | 10.00 | 31.00 | 29.67 | 10.00 | 14.00 | 3.02 | 1.40 | 0.03 | Mus musculus ion channel homolog RIC mRNA, complete cds |
| FSTL | M91380 | 10.00 | 10.00 | 20.00 | 13.00 | 10.00 | 10.00 | 2.00 | 1.00 | 0.16 | Mus musculus TGF-beta-inducible protein (TSC-36) mRNA, complete |
| | | | | | | | | | | | cds |
| FBXO6B | AA451220 | 10.00 | 12.00 | 22.00 | 28.33 | 10.50 | 15.33 | 2.20 | 1.46 | 0.01 | vf83b09.r1 Soares mouse mammary gland NbMMG Mus musculus |
| | | | | | | | | | | | cDNA clone 850361 5′ similar to WP C14B1.3 CE00900 |
| FARP-PENDING | AA059883 | 10.50 | 10.00 | 21.33 | 23.67 | 10.00 | 10.00 | 2.03 | 1.00 | 0.07 | mj76a06.r1 Soares mouse p3NMF19 5 Mus musculus cDNA clone |
| | | | | | | | | | | | 482002 5′ |
| ENTPD2 | W10995 | 11.00 | 17.00 | 23.00 | 22.67 | 12.00 | 16.67 | 2.09 | 1.39 | 0.00 | ma41d10.r1 Soares mouse p3NMF 19.5 Mus musculus cDNA clone |
| | | | | | | | | | | | 313267 5′, mRNA sequence. |
| DIPP | AA028770 | 10.00 | 10.00 | 20.00 | 28.00 | 19.50 | 35.33 | 2.00 | 1.81 | 0.07 | mi15h02.r1 Soares mouse p3NMF 19.5 Mus musculus cDNA clone |
| | | | | | | | | | | | 463635 5′ |
| D7ERTD237E | AA666918 | 11.75 | 10.00 | 25.33 | 31.33 | 10.50 | 10.00 | 2.16 | 0.95 | 0.01 | vq87c07.r1 Knowles Solter mouse blastocyst B3 Mut musculus cDNA |
| | | | | | | | | | | | clone 1109292 5′, mRNA sequence. |
| D5WSU111E | AA638539 | 11.25 | 10.33 | 47.33 | 63.33 | 10.00 | 15.33 | 4.21 | 1.53 | 0.02 | vo54d12.r1 Barstead mouse irradiated colon MPLRB7 Mus musculus |
| | | | | | | | | | | | cDNA clone 1053719 5′, mRNA sequence. |
| D17H6S56E-5 | U69488 | 10.00 | 10.00 | 22.33 | 35.67 | 10.00 | 10.00 | 2.23 | 1.00 | 0.10 | Mus musculus viral envelope like protein (G7e) gene, complete cds |
| D16WSU103E | AA674986 | 11.75 | 10.00 | 37.67 | 21.67 | 10.00 | 10.67 | 3.21 | 1.07 | 0.07 | vq57g08.r1 Barstead mouse proximal colon MPLRB6 Mus musculus |
| | | | | | | | | | | | cDNA clone 1106462 5′, mRNA sequence. |
| D14ERTD310E | C80103 | 10.00 | 10.00 | 31.67 | 36.67 | 13.00 | 15.33 | 3.17 | 1.18 | 0.02 | Mouse 3.5-dpc blastocyst cDNA Mus musculus cDNA clone J0076E08 |
| | | | | | | | | | | | 3′, mRNA sequence. |
| D12ERTD647E | AA120109 | 26.50 | 27.67 | 79.00 | 82.33 | 53.00 | 50.67 | 2.98 | 0.96 | 0.02 | AA120109 mq09a11.r1 Mus musculus cDNA, 5′ end |
| CTSS | AA089333 | 10.00 | 10.00 | 45.33 | 41.67 | 10.00 | 15.33 | 4.53 | 1.53 | 0.01 | AA089333 mo60e02.r1 Mus musculus cDNA, 5′ end |
| CTSS | AA146437 | 10.00 | 10.00 | 42.67 | 53.00 | 11.00 | 16.67 | 4.27 | 1.52 | 0.02 | AA146437 mr05a08.r1 Mus musculus cDNA, 5′ end |
| CTSC | U89269 | 16.50 | 12.33 | 54.00 | 71.67 | 11.00 | 11.33 | 3.27 | 1.03 | 0.01 | Mus musculus preprodipeptidyl peptidase 1 mRNA, complete cds |
| CTSC | AA144887 | 10.00 | 10.00 | 26.33 | 27.67 | 10.00 | 10.00 | 2.63 | 1.00 | 0.02 | AA144887 mr11d06.r1 Mus musculus cDNA, 5′ end |
| CTGF | M70642 | 19.50 | 20.00 | 83.00 | 79.33 | 30.50 | 24.33 | 4.26 | 0.80 | 0.01 | Mouse FISP-12 protein (fisp-12) mRNA, complete cds |
| CSTB | U59807 | 14.50 | 15.33 | 68.00 | 71.67 | 24.00 | 27.33 | 4.69 | 1.14 | 0.02 | Mus musculus cystatin B (Stfb) gene, complete cds |
| CRIP | M13018 | 10.25 | 11.33 | 48.00 | 49.67 | 14.00 | 25.67 | 4.68 | 1.83 | 0.01 | M13018 Mouse cysteine-rich intestinal protein (CRIP) mRNA, complete |
| | | | | | | | | | | | cds |
| CRIP | M13018 | 10.00 | 10.67 | 49.33 | 55.33 | 14.00 | 18.67 | 4.93 | 1.33 | 0.01 | Mouse cysteine-rich intestinal protein (CRIP) mRNA, complete cds |
| COL6A2 | X65582 | 11.25 | 12.00 | 33.33 | 25.00 | 13.50 | 17.33 | 2.96 | 1.28 | 0.02 | M. musculus mRNA for alpha-2 collagen VI |
| COL6A1 | X66405 | 11.25 | 10.33 | 24.67 | 18.00 | 11.50 | 12.33 | 2.19 | 1.07 | 0.04 | M. musculus mRNA for collagen alpha1(VI)-collagen |
| CNN2 | Z19543 | 15.25 | 16.67 | 34.33 | 35.33 | 16.50 | 22.00 | 2.25 | 1.33 | 0.01 | Z19543 M. musculus h2-calponin cDNA |
| CLDN4 | AB000713 | 10.00 | 10.00 | 23.00 | 50.00 | 10.00 | 10.00 | 2.30 | 1.00 | 0.07 | Mus musculus mCPE-R mRNA for CPE-receptor, complete cds |
| CLDN4 | AB000713 | 16.00 | 13.33 | 48.67 | 107.33 | 10.00 | 12.00 | 3.04 | 1.20 | 0.05 | Mus musculus mCPE-R mRNA for CPE-receptor, complete cds |
| CEBPB | X62600 | 10.00 | 10.00 | 22.33 | 27.33 | 10.50 | 10.00 | 2.23 | 0.95 | 0.01 | M. musculus mRNA for C/EBP beta. |
| CD72 | J04170 | 10.00 | 10.00 | 22.67 | 36.33 | 10.00 | 10.00 | 2.27 | 1.00 | 0.08 | Mouse B-cell differentiation antigen Lyb-2.1 protein, complete cds |
| CD68 | AB009287 | 10.00 | 11.33 | 23.33 | 29.00 | 10.00 | 12.33 | 2.33 | 1.23 | 0.01 | Mus musculus gene for Macrosialin, complete cds |
| CD52 | M55561 | 10.00 | 10.00 | 31.33 | 34.00 | 10.00 | 15.33 | 3.13 | 1.53 | 0.03 | Mouse phosphatidylinositol-linked antigen (pB7) mR |
| CD14 | X13333 | 25.50 | 28.67 | 89.33 | 95.33 | 21.50 | 27.33 | 3.50 | 1.27 | 0.01 | Mouse CD14 mRNA for myelid cell-specific leucine-rich glycoprotein |
| ATOX1 | AF004591 | 44.25 | 41.33 | 90.00 | 94.33 | 149.50 | 178.00 | 2.03 | 1.19 | 0.03 | Mus musculus copper transport protein Atox1 (ATOX1) mRNA, |
| | | | | | | | | | | | complete cds |
| ARHGDIB | L07918 | 10.00 | 10.00 | 26.00 | 32.00 | 12.50 | 14.33 | 2.60 | 1.15 | 0.06 | Mus musculus GDP-dissociation inhibitor mRNA, preferentially |
| | | | | | | | | | | | expressed in hematopoietic cells, complete cds |
| ARG2 | AF032466 | 10.25 | 10.33 | 21.33 | 36.00 | 10.00 | 16.67 | 2.08 | 1.67 | 0.03 | Mus musculus arginase II mRNA, complete cds |
| ANXA5 | U29396 | 13.00 | 13.00 | 40.00 | 38.00 | 22.00 | 29.67 | 3.08 | 1.35 | 0.00 | Mus musculus annexin V (Anx5) mRNA, complete cds |
| ANXA5 | W98864 | 12.00 | 15.00 | 29.33 | 30.33 | 13.00 | 20.00 | 2.44 | 1.54 | 0.01 | W98864 mg11h11.r1 Mus musculus cDNA, 5′ end |
| ANXA2 | D10024 | 20.50 | 18.00 | 105.67 | 106.00 | 42.50 | 45.00 | 5.15 | 1.06 | 0.02 | D10024 Mouse mRNA for protein-tyrosine kinase substrate p36 |
| | | | | | | | | | | | (calpactin I heavy chain), complete cds |
| ANXA2 | M14044 | 22.00 | 17.33 | 139.67 | 159.00 | 47.50 | 50.33 | 6.35 | 1.06 | 0.02 | Moeuse calpactin I heavy chain (p36) mRNA, complete cds |
| ANXA1 | X07486 | 15.00 | 12.67 | 36.00 | 42.67 | 10.00 | 14.67 | 2.40 | 1.47 | 0.01 | Mouse mRNA for lipocortin I. |
| ADAMTS1 | D67076 | 10.00 | 10.00 | 36.00 | 46.33 | 10.00 | 10.33 | 3.60 | 1.03 | 0.03 | Mouse mRNA for secretory protein containing thrombospondin motifs, |
| | | | | | | | | | | | complete cds |

TABLE 2
Genes with a known Link to Lupus Nephritis

| | Accession | Avg | Avg | Avg | Avg | Avg | Avg | | |
|---|---|---|---|---|---|---|---|---|---|
| Name | No | Untr12w | Untr25w | Untr36w | Untr42w | C57/3m | C57/8m | p value | Description |
| C3 | K02782 | 23.25 | 13.67 | 178.67 | 361.33 | 38.50 | 27.67 | 0.02 | Mouse complement component C3 mRNA, alpha and beta subunits, complete cds |
| FN1 | M18194 | 13.50 | 10.00 | 38.67 | 28.00 | 14.00 | 16.33 | 0.06 | M18194 Mouse fibronectin (FN) mRNA |
| H2-AA | V00832 | 41.75 | 36.67 | 134.00 | 138.67 | 43.00 | 79.33 | 0.00 | V00832 Mouse fragment of mRNA encoding for the la antigen (heavy chain) from major histocompatibility |
| | | | | | | | | | complex (A-k-alpha). This is coded by the I-A region of the MHC and corresponds to the k haplotype |
| FN1 | M18194 | 13.50 | 10.00 | 51.00 | 38.33 | 15.00 | 17.33 | 0.04 | Mouse fibronectin (FN) mRNA |
| COLA1 | U08020 | 12.00 | 13.67 | 44.33 | 18.33 | 12.00 | 18.33 | 0.08 | U08020 Mus musculus FVB/N collagen pro-alpha-1 type I chain mRNA, complete cds |
| UNK_AA163096 | AA163096 | 17.25 | 13.67 | 45.00 | 43.33 | 25.50 | 25.67 | 0.02 | mt65a03.r1 Soares mouse lymph node NbMLN Mus musculus cDNA clone 634732 5′ |
| UNK_AA596794 | AA596794 | 33.00 | 21.00 | 92.67 | 93.00 | 51.00 | 61.67 | 0.02 | vo16a05.r1 Barstead mouse myotubes MPLRB5 Mus musculus cDNA clone 1050032 5′, mRNA |
| | | | | | | | | | sequence. |
| UNK_W90837 | W90837 | 10.75 | 10.00 | 33.00 | 27.33 | 10.00 | 12.00 | 0.05 | W90837 mf78g07.r1 Mus musculus cDNA, 5′ end |
| TUBA2 | AA030759 | 14.00 | 10.00 | 40.33 | 44.67 | 23.50 | 27.00 | 0.01 | AA030759 mi32e11.r1 Mus musculus cDNA, 5′ end |
| COL6A1 | X66405 | 11.25 | 10.33 | 24.67 | 18.00 | 11.50 | 12.33 | 0.04 | M. musculus mRNA for collagen alpha1(VI)-collagen |
| II | X00496 | 60.00 | 64.67 | 363.33 | 343.67 | 98.50 | 183.67 | 0.00 | Mouse la-associated invariant chain (li) mRNA fragment. |
| C1CB | M22531 | 11.00 | 11.33 | 58.67 | 64.00 | 23.00 | 37.67 | 0.00 | M22531 Mouse complement C1q B chain mRNA, complete cds |
| H2-BA | U13648 | 13.50 | 13.67 | 91.67 | 92.33 | 10.00 | 10.00 | 0.01 | Mus musculus domesticus MHC class II antigen H-2E alpha precursor (allele w29) mRNA, complete cds |
| UNK_ET62052 | ET62052 | 10.00 | 10.00 | 101.33 | 94.33 | 10.00 | 10.67 | 0.07 | Mus musculus immunoglobulin rearranged gamma-1 chain mRNA, partial cds |
| LCN2 | X81627 | 10.00 | 10.00 | 81.67 | 194.33 | 10.00 | 11.33 | 0.06 | M. musculus 24p3 gene |
| IGH-4 | M60429 | 10.00 | 10.00 | 79.00 | 83.00 | 10.00 | 10.00 | 0.07 | Mouse Ig rearranged H-chain mRNA constant region |
| UNK_ET63039 | ET63039 | 10.00 | 10.00 | 77.33 | 77.67 | 10.00 | 12.33 | 0.05 | M. musculus mRNA for variable heavy chain. |
| UNK_ET61876 | ET61876 | 10.00 | 10.00 | 73.33 | 87.67 | 10.00 | 12.67 | 0.05 | Mus musculus anti-DNA immunoglobulin heavy chain IgM mRNA, antibody 452p.70, partial cds |
| UNK_ET61918 | ET61918 | 10.00 | 10.00 | 72.33 | 64.67 | 10.00 | 10.00 | 0.08 | Mus musculus anti-DNA immunoglobulin light chain IgM mRNA, antibody 363p.202, partial cds |
| C1CA | X58861 | 10.00 | 10.00 | 66.67 | 86.00 | 12.50 | 20.67 | 0.02 | Mouse mRNA for complement subcomponent C1Q alpha-chain |
| UNK_J00475 | J00475 | 10.00 | 10.00 | 59.33 | 59.00 | 10.00 | 10.00 | 0.09 | Mouse germline IgH chain gene, DJC region segment D-FL16 1 |
| UNK_ET61788 | ET61788 | 10.00 | 10.00 | 58.67 | 65.33 | 10.00 | 10.00 | 0.06 | Mus musculus anti-DNA immunoglobulin heavy chain IgM mRNA, antibody 363p.197, partial cds |
| UNK_ET61857 | ET61857 | 10.00 | 10.00 | 57.33 | 66.33 | 10.00 | 10.00 | 0.06 | Mus musculus anti-DNA immunoglobulin heavy chain IgG mRNA, antibody 423p.195, partial cds |
| UNK_ET61660 | ET61660 | 10.00 | 11.00 | 53.33 | 54.67 | 10.00 | 10.00 | 0.07 | Mus musculus clone 1G2 IgG anti-nucleosome heavy chain variable region mRNA, partial cds |
| COLA2 | X58251 | 10.00 | 10.00 | 52.67 | 15.67 | 10.00 | 12.33 | 0.22 | Mouse COL1A2 mRNA for pro-alpha-2(I) collagen. |
| VCAM1 | X67783 | 10.00 | 10.00 | 52.00 | 40.00 | 10.00 | 12.33 | 0.03 | M. musculus VCAM-1 mRNA |
| UNK_ET61286 | ET61286 | 10.00 | 10.00 | 49.33 | 62.00 | 10.00 | 10.00 | 0.07 | Mus musculus anti-DNA immunoglobulin heavy chain variable region, clone 20F4, partial cds |
| UNK_ET61798 | ET61798 | 10.00 | 10.00 | 49.33 | 70.00 | 10.00 | 10.00 | 0.07 | Mus musculus anti-DNA immunoglobulin heavy chain IgG mRNA, antibody 363s.68, partial cds |
| UNK_ET61285 | ET61285 | 10.00 | 10.00 | 52.00 | 65.33 | 10.00 | 10.00 | 0.05 | Mus musculus anti-DNA immunoglobulin heavy chain variable region, clone 4B2, partial cds |
| UNK_ET61845 | ET61845 | 10.00 | 10.00 | 43.00 | 48.67 | 10.00 | 10.00 | 0.08 | Mus musculus anti-DNA immunoglobulin heavy chain IgG mRNA, antibody 384s.14, partial cds |
| UNK_ET61814 | ET61814 | 10.00 | 10.00 | 41.67 | 54.67 | 10.00 | 10.00 | 0.05 | Mus musculus anti-DNA immunoglobulin heavy chain IgG mRNA, antibody 373s.5, partial cds |
| UNK_ET61870 | ET61870 | 10.00 | 10.00 | 39.33 | 59.67 | 10.00 | 10.00 | 0.07 | Mus musculus anti-DNA immunoglobulin heavy chain IgM mRNA, antibody 452p.17, partial cds |
| UNK_ET62985 | ET62985 | 10.00 | 11.00 | 39.00 | 45.67 | 10.00 | 13.00 | 0.04 | M. musculus mRNA (1B5) for IgA V-D-J-heavy chain |
| COLA2 | X58251 | 10.00 | 10.00 | 38.00 | 13.00 | 10.00 | 11.33 | 0.18 | X58251 Mouse COL1A2 mRNA for pro-alpha-2(l) collagen |
| UNK_ET61854 | ET61854 | 10.00 | 10.00 | 37.00 | 43.33 | 10.00 | 10.00 | 0.06 | Mus musculus anti-DNA immunoglobulin heavy chain IgG mRNA, antibody 423p.107, partial cds |
| UNK_M35667 | M35667 | 10.00 | 10.00 | 36.67 | 44.33 | 10.00 | 14.00 | 0.02 | Mouse lysozyme-binding Ig kappa chain (HyHEL-10) V23-J2 region mRNA, partial cds |
| UNK_ET61801 | ET61801 | 10.00 | 10.00 | 36.00 | 49.33 | 10.00 | 10.33 | 0.06 | Mus musculus anti-DNA immunoglobulin heavy chain IgM mRNA, antibody 373p.95, partial cds |
| UNK_ET61800 | ET61800 | 10.00 | 10.00 | 35.33 | 64.67 | 10.00 | 10.00 | 0.07 | Mus musculus anti-DNA immunoglobulin heavy chain IgG mRNA, antibody 363s.73, partial cds |
| UNK_ET62188 | ET62188 | 10.00 | 10.00 | 34.00 | 41.00 | 10.00 | 10.00 | 0.08 | Mus musculus Ig anti-DNA heavy chain VDJ (J558) mRNA, partial cds |
| UNK_ET61809 | ET61809 | 10.00 | 10.00 | 33.67 | 37.00 | 10.00 | 10.00 | 0.09 | Mus musculus anti-DNA immunoglobulin heavy chain IgM mRNA, antibody 373s.83, partial cds |
| UNK_ET61859 | ET61859 | 10.00 | 10.00 | 33.67 | 41.33 | 10.00 | 10.00 | 0.06 | Mus musculus anti-DNA immunoglobulin heavy chain IgG mRNA, antibody 423p.226, partial cds |
| UNK_ET61792 | ET61792 | 10.00 | 10.00 | 33.00 | 56.33 | 10.00 | 10.00 | 0.07 | Mus musculus anti-DNA immunoglobulin heavy chain IgG mRNA, antibody 363p.8, partial cds |
| UNK_ET61821 | ET61821 | 10.00 | 10.00 | 32.33 | 44.33 | 10.00 | 10.00 | 0.06 | Mus musculus anti-DNA immunoglobulin heavy chain IgG mRNA, antibody 373s.32, partial cds |
| PSMB8 | L11145 | 10.00 | 10.00 | 31.00 | 37.33 | 12.00 | 16.00 | 0.03 | Mus musculus Balb/c proteasome subunit (Imp7) gene, complete cds and intergenic region. |
| COLA1 | U08020 | 10.00 | 10.00 | 30.00 | 15.33 | 10.00 | 10.00 | 0.12 | Mus musculus FVB/N collagen pro-alpha-1 type I chain mRNA, complete cds |
| UNK_ET61846 | ET61846 | 10.00 | 10.00 | 28.33 | 37.67 | 10.00 | 10.00 | 0.08 | Mus musculus anti-DNA immunoglobulin heavy chain IgG mRNA, antibody 384s.15, partial cds |
| UNK_ET62192 | ET62192 | 10.00 | 10.00 | 27.67 | 34.67 | 10.00 | 10.00 | 0.08 | Mus musculus Ig anti-DNA heavy chain VDJ (J558) mRNA, partial cds |
| UNK_ET61837 | ET61837 | 10.00 | 10.00 | 27.33 | 36.00 | 10.00 | 10.00 | 0.08 | Mus musculus anti-DNA immunoglobulin heavy chain IgG mRNA, antibody 384s.73, partial cds |
| UNK_ET61851 | ET61851 | 10.00 | 10.00 | 27.33 | 49.00 | 10.00 | 10.00 | 0.09 | Mus musculus anti-DNA immunoglobulin heavy chain IgG mRNA, antibody 423p.78, partial cds |
| UNK_ET61863 | ET61863 | 10.00 | 10.00 | 26.67 | 36.33 | 10.00 | 10.00 | 0.08 | Mus musculus anti-DNA immunoglobulin heavy chain IgG mRNA, antibody 423s.38, partial cds |
| UNK_ET61947 | ET61947 | 10.00 | 10.00 | 25.33 | 31.00 | 10.00 | 10.00 | 0.06 | Mus musculus anti-DNA immunoglobulin light chain IgG, antibody 373s.20, partial cds |
| UNK_ET62717 | ET62717 | 10.00 | 10.00 | 25.33 | 32.67 | 10.00 | 10.00 | 0.08 | Mus musculus anti-DNA antibody heavy chain variable region mRNA, partial cds |
| UNK_ET62026 | ET62026 | 10.00 | 10.00 | 25.00 | 22.67 | 10.00 | 10.00 | 0.12 | Mus musculus anti-DNA immunoglobulin light chain IgG, antibody 452s.88, partial cds |
| UNK_ET61937 | ET61937 | 10.00 | 10.00 | 24.67 | 22.33 | 10.00 | 10.00 | 0.06 | Mus musculus anti-DNA immunoglobulin light chain IgM mRNA, antibody 373s.70, partial cds |
| UNK_ET61832 | ET61832 | 10.00 | 10.00 | 22.67 | 25.67 | 10.00 | 10.00 | 0.08 | Mus musculus anti-DNA immunoglobulin heavy chain IgG mRNA, antibody 384p.113, partial cds |
| UNK_ET61955 | ET61955 | 10.00 | 10.00 | 22.33 | 16.33 | 10.00 | 10.00 | 0.20 | Mus musculus anti-DNA immunoglobulin light chain IgG, antibody 373s.116, partial cds |
| UNK_ET62112 | ET62112 | 10.00 | 10.00 | 22.33 | 31.67 | 10.00 | 10.00 | 0.06 | Mus musculus J558+ IgM heavy chain mRNA, partial cds |
| UNK_ET62725 | ET62725 | 10.00 | 11.00 | 81.33 | 92.00 | 10.00 | 14.33 | 0.04 | Mus musculus anti-DNA antibody heavy chain variable region mRNA, partial cds |
| UNK_ET62707 | ET62707 | 10.00 | 10.00 | 25.67 | 24.33 | 10.00 | 10.00 | 0.12 | Mus musculus anti-DNA antibody heavy chain variable region mRNA, partial cds |
| UNK_ET62705 | ET62705 | 10.00 | 10.00 | 65.00 | 80.00 | 10.00 | 12.00 | 0.05 | Mus musculus anti-DNA antibody heavy chain variable region mRNA, partial cds |
| UNK_ET62459 | ET62459 | 10.00 | 10.00 | 20.33 | 22.00 | 10.00 | 10.00 | 0.10 | Mus musculus Ig light chain Fv fragment specific for human apolipoprotein A-I, mRNA, partial cds |
| UNK_ET62422 | ET62422 | 10.00 | 10.00 | 22.00 | 24.67 | 10.00 | 10.00 | 0.09 | Mus musculus type II collagen antibody heavy chain variable region mRNA, partial cds |
| UNK_ET62199 | ET62199 | 10.00 | 10.00 | 46.00 | 63.33 | 10.00 | 13.00 | 0.05 | Mus musculus Ig anti-DNA light chain (Vk4/5) mRNA, partial cds |
| UNK_ET62191 | ET62191 | 10.00 | 10.33 | 58.67 | 67.00 | 10.00 | 12.00 | 0.05 | Mus musculus Ig anti-DNA heavy chain VDJ (J558) mRNA, partial cds |
| UNK_ET62039 | ET62039 | 10.00 | 10.00 | 46.33 | 52.67 | 10.00 | 13.33 | 0.04 | Mus musculus anti-DNA immunoglobulin light chain IgG, antibody 452s.61, partial cds |
| UNK_ET62023 | ET62023 | 10.00 | 10.00 | 20.00 | 18.67 | 10.00 | 10.00 | 0.11 | Mus musculus anti-DNA immunoglobulin light chain IgG, antibody 452s.36, partial cds |
| UNK_ET62015 | ET62015 | 10.00 | 10.00 | 20.00 | 30.67 | 10.00 | 10.00 | 0.08 | Mus musculus anti-DNA immunoglobulin light chain IgG, antibody 452p.151, partial cds |
| UNK_ET61984 | ET61984 | 10.00 | 10.33 | 33.33 | 55.33 | 10.00 | 11.00 | 0.05 | Mus musculus anti-DNA immunoglobulin light chain IgG, antibody 423p.195, partial cds |
| UNK_ET61976 | ET61976 | 10.00 | 10.00 | 29.67 | 30.33 | 10.00 | 10.00 | 0.03 | Mus musculus anti-DNA immunoglobulin light chain IgG, antibody 384s.89, partial cds |
| UNK_ET61970 | ET61970 | 10.00 | 12.67 | 33.67 | 51.67 | 10.00 | 12.33 | 0.02 | Mus musculus anti-DNA immunoglobulin light chain IgG, antibody 384s.63, partial cds |
| UNK_ET61965 | ET61965 | 10.00 | 10.00 | 20.67 | 26.67 | 10.00 | 10.00 | 0.08 | Mus musculus anti-DNA immunoglobulin light chain IgG, antibody 384s.80, partial cds |
| UNK_ET61957 | ET61957 | 10.75 | 16.00 | 73.00 | 110.67 | 10.00 | 15.67 | 0.03 | Mus musculus anti-DNA immunoglobulin light chain IgG, antibody 384p.41, partial cds |
| UNK_ET61942 | ET61942 | 10.00 | 13.33 | 77.67 | 106.00 | 10.00 | 14.33 | 0.04 | Mus musculus anti-DNA immunoglobulin light chain IgG, antibody 373s.51, partial cds |
| UNK_ET61925 | ET61925 | 10.00 | 10.33 | 65.67 | 73.67 | 10.00 | 14.33 | 0.03 | Mus musculus anti-DNA immunoglobulin light chain IgG, antibody 363s.71, partial cds |
| UNK_ET61921 | ET61921 | 10.00 | 12.67 | 33.67 | 42.00 | 10.00 | 13.33 | 0.02 | Mus musculus anti-DNA immunoglobulin light chain IgG, antibody 363p.8, partial cds |
| UNK_ET61919 | ET61919 | 10.00 | 10.00 | 30.33 | 39.33 | 10.00 | 10.00 | 0.06 | Mus musculus anti-DNA immunogolobulin light chain IgM mRNA, antibody 363s.57, partial cds |
| UNK_ET61916 | ET61916 | 10.00 | 16.00 | 44.67 | 53.67 | 10.00 | 14.00 | 0.05 | Mus musculus anti-DNA immunoglobulin light chain IgM mRNA, antibody 363p.193, partial cds |
| UNK_ET61909 | ET61909 | 10.00 | 10.00 | 31.67 | 41.00 | 10.00 | 10.00 | 0.07 | Mus musculus anti-DNA immunoglobulin heavy chain IgG mRNA, antibody 452s.43, partial cds |
| UNK_ET61908 | ET61908 | 10.00 | 10.00 | 49.00 | 52.00 | 10.00 | 10.00 | 0.09 | Mus musculus anti-DNA immunoglobulin heavy chain IgG mRNA, antibody 452s.5, partial cds |
| UNK_ET61885 | ET61885 | 10.00 | 11.00 | 66.33 | 83.67 | 10.00 | 10.00 | 0.05 | Mus musculus anti-DNA immunoglobulin heavy chain IgG mRNA, antibody 452p.33, partial cds |
| UNK_ET61874 | ET61874 | 10.00 | 10.00 | 21.67 | 32.00 | 10.00 | 10.00 | 0.05 | Mus musculus anti-DNA immunoglobulin heavy chain IgM mRNA, antibody 452p.71 m, partial cds |
| UNK_ET61873 | ET61873 | 10.00 | 10.00 | 31.00 | 34.67 | 10.00 | 10.00 | 0.09 | Mus musculus anti-DNA immunoglobulin heavy chain IgM mRNA, antibody 452p.53, partial cds |
| UNK_ET61871 | ET61871 | 10.00 | 10.00 | 20.67 | 36.67 | 10.00 | 10.00 | 0.07 | Mus musculus anti-DNA immunoglobulin heavy chain IgM mRNA, antibody 452p.18, partial cds |
| UNK_ET61855 | ET61855 | 10.00 | 10.00 | 46.67 | 51.33 | 10.00 | 10.00 | 0.06 | Mus musculus anti-DNA immunoglobulin heavy chain IgG mRNA, antibody 423p.135, partial cds |
| UNK_ET61853 | ET61853 | 10.00 | 10.33 | 48.00 | 53.00 | 10.00 | 10.00 | 0.06 | Mus musculus anti-DNA immunoglobulin heavy chain IgG mRNA, antibody 423p.83, partial cds |
| UNK_ET61841 | ET61841 | 10.00 | 10.67 | 28.33 | 46.33 | 10.00 | 10.00 | 0.05 | Mus musculus anti-DNA immunoglobulin heavy chain IgG mRNA, antibody 384s.17, partial cds |
| UNK_ET61839 | ET61839 | 10.00 | 10.33 | 68.33 | 90.33 | 10.0 | 10.67 | 0.03 | Mus musculus anti-DNA immunoglobulin heavy chain IgG mRNA, antibody 384s.95, partial cds |
| UNK_ET61838 | ET61838 | 10.00 | 10.00 | 20.33 | 30.33 | 10.00 | 10.00 | 0.08 | Mus musculus anti-DNA immunoglobulin heavy chain IgG mRNA, antibody 384s.80, partial cds |
| UNK_ET61833 | ET61833 | 10.00 | 17.67 | 96.67 | 118.67 | 10.00 | 13.00 | 0.05 | Mus musculus anti-DNA immunoglobulin heavy chain IgG mRNA, antibody 384p.20, partial cds |
| UNK_ET61815 | ET61815 | 10.00 | 10.67 | 81.00 | 92.00 | 10.00 | 14.67 | 0.04 | Mus musculus anti-DNA immunoglobulin heavy chain IgG mRNA, antibody 373s.51, partial cds |
| UNK_ET61810 | ET61810 | 10.00 | 10.00 | 39.33 | 55.67 | 10.00 | 10.00 | 0.04 | Mus musculus anti-DNA immunoglobulin heavy chain IgM mRNA, antibody 373s.70, partial cds |
| UNK_ET61802 | ET61802 | 10.75 | 14.33 | 22.00 | 37.33 | 10.50 | 10.00 | 0.02 | Mus musculus anti-DNA immunoglobulin heavy chain IgM mRNA, antibody 373p.72, partial cds |
| UNK_ET61791 | ET61791 | 10.00 | 10.00 | 21.00 | 31.67 | 10.00 | 10.00 | 0.08 | Mus musculus anti-DNA immunoglobulin heavy chain IgG mRNA, antibody 363p.24, partial cds |
| UNK_ET61785 | ET61785 | 10.00 | 10.00 | 85.33 | 92.67 | 10.00 | 15.67 | 0.04 | Mus musculus anti-DNA immunoglobulin heavy chain IgM mRNA, antibody 363p.168, partial cds |
| UNK_ET61783 | ET61783 | 10.00 | 10.67 | 63.33 | 62.67 | 10.00 | 10.00 | 0.06 | Mus musculus anti-DNA immunoglobulin heavy chain IgM mRNA, antibody 363p.138, partial cds |
| UNK_ET61753 | ET61753 | 10.00 | 10.00 | 25.67 | 27.00 | 10.00 | 10.00 | 0.09 | Mus musculus Ig 10B7 A1 heavy chain mRNA, specific for rat (mouse) cytochrome c, partial cds |
| UNK_ET61296 | ET61296 | 10.00 | 10.67 | 33.33 | 46.00 | 10.00 | 14.00 | 0.05 | Mus musculus anti-DNA immunoglobulin light chain variable region, clone 22F8, partial cds |
| UNK_ET61288 | ET61288 | 10.00 | 10.00 | 23.00 | 37.67 | 10.00 | 10.00 | 0.06 | Mus musculus anti-DNA immunoglobulin heavy chain variable region, clone 22F8, partial cds |
| UNK_ET61287 | ET61287 | 10.00 | 10.00 | 44.33 | 52.67 | 10.00 | 10.00 | 0.06 | Mus musculus anti-DNA immunoglobulin heavy chain variable region, clone 8D8, partial cds |
| UNK_AA244836 | AA244836 | 10.00 | 10.00 | 37.00 | 60.33 | 10.00 | 10.00 | 0.02 | mx25h11.r1 Soares mouse NML Mus musculus cDNA clone 681285 5′ similar to gb X02415_rna3 |
| | | | | | | | | | FIBRINOGEN GAMMA-A CHAIN PRECURSOR (HUMAN); |
| C1QC | X66295 | 10.25 | 14.00 | 57.00 | 64.00 | 14.00 | 22.00 | 0.01 | M. musculus mRNA for C1q C-chain. |
| C1QB | X16874 | 10.00 | 10.00 | 47.00 | 50.33 | 15.00 | 19.33 | 0.01 | Mouse mRNA for complement protein C1q B-chain |
| C1NH | Y10386 | 37.00 | 37.00 | 94.00 | 63.33 | 42.50 | 60.00 | 0.03 | M. musculus mRNA for C1 inhibitor. |
| UNK_ET61662 | ET61662 | 10.00 | 10.00 | 21.33 | 23.67 | 10.00 | 10.00 | 0.09 | Mus musculus clone 4F7 IgG anti-nucleosome heavy chain variable region mRNA, partial cds |
| UNK_AA238483 | AA238483 | 13.00 | 15.00 | 31.33 | 34.33 | 26.00 | 35.00 | 0.02 | mx94f04.r1 Soares mouse NML Mus musculus cDNA clone 694015 5′ similar to TR G806566 G806566 |
| | | | | | | | | | SM PROTEIN G; |
| UNK_AA184116 | AA184116 | 11.75 | 11.00 | 28.00 | 37.00 | 10.00 | 11.00 | 0.02 | mt22f04.r1 Soares mouse 3NbMS Mus musculus cDNA clone 621823 5′ |
| UNK_AA011784 | AA011784 | 17.50 | 16.00 | 67.67 | 60.00 | 25.50 | 30.00 | 0.02 | AA011784 mg92b08.r1 Mus musculus cDNA, 5′ end |
| TUBB5 | W12548 | 16.25 | 15.67 | 52.00 | 53.33 | 11.00 | 15.67 | 0.00 | W12548 ma59d04.r1 Mus musculus cDNA, 5′ end |
| TUBB5 | X04663 | 21.75 | 22.00 | 55.33 | 60.67 | 21.50 | 23.00 | 0.00 | X04663 Mouse mRNA for beta-tubulin (Isotype Mbeta 5) |
| TUBB5 | X04663 | 19.75 | 18.33 | 60.67 | 57.33 | 20.00 | 20.67 | 0.00 | Mouse mRNA for beta-tubulin (Isotype Mbeta 5) |
| TPM2 | M22479 | 20.00 | 17.33 | 60.00 | 63.33 | 23.00 | 32.00 | 0.02 | Mouse tropomyosin isoform 2 mRNA, complete cds |
| TLN | X56123 | 10.00 | 10.00 | 28.00 | 11.00 | 13.50 | 15.67 | 0.23 | Mouse mRNA for talin |
| UNK_Z22111 | Z22111 | 10.00 | 10.00 | 43.67 | 58.67 | 10.00 | 10.00 | 0.07 | Z22111 M domesticus IgG variable region |
| UNK_M86751 | M86751 | 10.00 | 13.00 | 30.00 | 66.67 | 10.00 | 11.00 | 0.07 | Mouse Ig L-chain gene variable region, complete cds |
| PTMB4 | W41883 | 83.75 | 78.33 | 272.00 | 194.67 | 180.00 | 200.67 | 0.00 | W41883 mc64g08.r1 Mus musculus cDNA, 5′ end |
| SPI6 | AA108054 | 10.00 | 10.00 | 23.33 | 28.67 | 11.00 | 16.67 | 0.02 | mp09d07.r1 Life Tech mouse embryo 8.5dpc 10664019 Mus musculus cDNA clone 568717 5′ |
| SPI3 | U25844 | 10.75 | 10.00 | 25.67 | 45.33 | 14.00 | 21.00 | 0.05 | Mus musculus serine proteinase inhibitor (SPI3) mR |
| SPI2-1 | M64085 | 10.00 | 12.67 | 20.67 | 28.33 | 10.00 | 13.00 | 0.03 | M64085 Mouse spi2 proteinase inhibitor (spi2/eb1) mRNA, 3′ end |
| MKI67 | X82786 | 10.00 | 10.00 | 21.33 | 19.00 | 10.00 | 10.00 | 0.03 | M. musculus mRNA for Ki-67 |
| LCN2 | W13166 | 10.00 | 10.00 | 70.00 | 192.33 | 10.00 | 10.00 | 0.08 | W13166 ma93f11.r1 Mus musculus cDNA, 5′ end |
| H2-DMB1 | X62743 | 10.25 | 11.00 | 20.33 | 22.67 | 10.50 | 12.33 | 0.01 | M. musculus Mb mRNA |
| H2-D | M69069 | 10.00 | 10.00 | 21.00 | 27.67 | 10.00 | 10.00 | 0.05 | Mus musculus mRNA, complete cds |
| H2-AA | K01923 | 50.00 | 50.33 | 187.67 | 177.33 | 58.00 | 84.67 | 0.00 | K01923 Mouse MHC class II H2-IA-alpha gene (d haplotype) mRNA, complete cds |
| H2-AA | K01923 | 38.50 | 41.00 | 152.33 | 172.00 | 32.50 | 63.00 | 0.01 | Mouse MHC class II H2-IA-alpha gene (d haplotype) mRNA, complete cds |
| FBN1 | L29454 | 10.00 | 10.00 | 20.33 | 14.67 | 10.00 | 10.00 | 0.11 | Mouse fibrillin (Fbn-1) mRNA, complete cds |
| ACTVS | X13297 | 37.00 | 30.67 | 148.67 | 55.67 | 65.00 | 52.67 | 0.11 | Mouse mRNA for vascular smooth muscle alpha-actin |
| ACTG2 | U20365 | 13.00 | 18.00 | 26.33 | 27.67 | 11.00 | 10.00 | 0.03 | Mus musculus smooth muscle gamma-actin gene |
| ACTC1 | AA117701 | 10.75 | 10.67 | 22.33 | 19.67 | 12.50 | 10.00 | 0.02 | AA117701 mo64d03.r1 Mus musculus cDNA, 5′ end |

TABLE 3
Genes Increased in Disease

| | | | Untreated | Untreated | Untreated | Untreated |
|---|---|---|---|---|---|---|
| Gene | Accession | | @ 12 wks | @ 25 wks | @ 36 wks | @ 42 wks |
| name | number | Description | of age | of age | of age | of age |
| ACTC1 | AA117701 | mo64d03.r1 Mus musculus cDNA, 5′ end | 10.75 | 10.67 | 22.33 | 19.67 |
| | | Mouse mRNA for secretory protein containing. | | | | |
| ADAMTS1 | D67076 | thrombospondin motifs, complete cds. | 10.00 | 10.00 | 36.00 | 46.33 |
| ANXA1 | X07486 | Mouse mRNA for lipocortin I. | 15.00 | 12.67 | 36.00 | 42.67 |
| ANXA2 | M14044 | Mouse calpactin I heavy chain (p36) mRNA, complete cds | 22.00 | 17.33 | 139.67 | 159.00 |
| | | Mouse mRNA for protein-tyrosine kinase substrate p36 | | | | |
| ANXA2 | D10024 | (calpactin I heavy chain), complete cds | 20.50 | 18.00 | 105.67 | 106.00 |
| ANXA5 | W98864 | mg11h11.r1 Mus musculus cDNA, 5′ end | 12.00 | 15.00 | 29.33 | 30.33 |
| ARG2 | AF032466 | Mus musculus arginase II mRNA, complete cds. | 10.25 | 10.33 | 21.33 | 36.00 |
| | | Mus musculus copper transport protein Atox1 (ATOX1) | | | | |
| ATOX1 | AF004591 | mRNA, complete cds. | 44.25 | 41.33 | 90.00 | 94.33 |
| C1NH | Y10386 | M. musculus mRNA for C1 inhibitor. | 37.00 | 37.00 | 94.00 | 63.33 |
| | | Mouse CD14 mRNA for myelid cell-specific leucine-rich | | | | |
| CD14 | X13333 | glycoprotein. | 25.50 | 28.67 | 89.33 | 95.33 |
| CD52 | M55561 | Mouse phosphatidylinositol-linked antigen (pB7) mR | 10.00 | 10.00 | 31.33 | 34.00 |
| CD68 | AB009287 | Mus musculus gene for Macrosialin, complete cds. | 10.00 | 11.33 | 23.33 | 29.00 |
| | | Mouse B-cell differentiation antigen Lyb-2.1 protein, complete | | | | |
| CD72 | J04170 | cds | 10.00 | 10.00 | 22.67 | 36.33 |
| CEBPB | X62600 | M. musculus mRNA for C/EBP beta. | 10.00 | 10.00 | 22.33 | 27.33 |
| | | Mus musculus mCPE-R mRNA for CPE-receptor, complete | | | | |
| CLDN4 | AB000713 | cds. | 16.00 | 13.33 | 48.67 | 107.33 |
| CNN2 | Z19543 | M. musculus h2-calponin cDNA | 15.25 | 16.67 | 34.33 | 35.33 |
| | | Mouse cysteine-rich intestinal protein (CRIP) mRNA, | | | | |
| CRIP | M13018 | complete cds | 10.00 | 10.67 | 49.33 | 55.33 |
| CSTB | U59807 | Mus musculus cystatin B (Stfb) gene, complete cds. | 14.50 | 15.33 | 68.00 | 71.67 |
| CTGF | M70642 | Mouse FISP-12 protein (fisp-12) mRNA, complete cds | 19.50 | 20.00 | 83.00 | 79.33 |
| | | Mus musculus preprodipeptidyl peptidase I mRNA, complete | | | | |
| CTSC | U89269 | cds. | 16.50 | 12.33 | 54.00 | 71.67 |
| CTSC | AA144887 | mr11d06.r1 Mus musculus cDNA, 5′ end | 10.00 | 10.00 | 26.33 | 27.67 |
| CTSS | AA089333 | mo60e02.r1 Mus musculus cDNA, 5′ end | 10.00 | 10.00 | 45.33 | 41.67 |
| CTSS | AA146437 | mr05a08.r1 Mus musculus cDNA, 5′ end | 10.00 | 10.00 | 42.67 | 53.00 |
| D12ERTD647E | AA120109 | mq09a11.r1 Mus musculus cDNA, 5′ end | 26.50 | 27.67 | 79.00 | 82.33 |
| | | Mouse 3.5-dpc-blastocyst cDNA Mus musculus cDNA clone | | | | |
| D14ERTD310E | C80103 | J0076E08 3′, mRNA sequence. | 10.00 | 10.00 | 31.67 | 36.67 |
| | | vq57g08.r1 Barstead mouse proximal colon MPLRB6 Mus | | | | |
| D16WSU103E | AA674986 | musculus cDNA clone 1106462 5′, mRNA sequence. | 11.75 | 10.00 | 37.67 | 21.67 |
| | | Mus musculus viral envelope like protein (G7e) gene, | | | | |
| D17H6S56E5 | U69488 | complete cds | 10.00 | 10.00 | 22.33 | 35.67 |
| | | vo54d12.r1 Barstead mouse irradiated colon MPLRB7 Mus | | | | |
| D5WSU111E | AA638539 | musculus cDNA clone 1053719 5′, mRNA sequence. | 11.25 | 10.33 | 47.33 | 63.33 |
| | | vq87c07.r1 Knowles Solter mouse blastocyst B3 Mus | | | | |
| D7ERTD237E | AA666918 | musculus cDNA clone 1109292 5′, mRNA sequence. | 11.75 | 10.00 | 25.33 | 31.33 |
| | | mi15h02.r1 Soares mouse p3NMF19.5 Mus musculus cDNA | | | | |
| DIPP | AA028770 | clone 463635 5′ | 36.50 | 45.00 | 81.33 | 101.00 |
| | | ma41d10.r1 Soares mouse p3NMF19.5 Mus musculus cDNA | | | | |
| ENTPD2 | W10995 | clone 313267 5′, mRNA sequence. | 11.00 | 17.00 | 23.00 | 22.67 |
| FARP- | | mj76a06.r1 Soares mouse p3NMF19.5 Mus musculus cDNA | | | | |
| PENDING | AA059883 | clone 482002 5′ | 10.50 | 10.00 | 21.33 | 23.67 |
| | | vf83b09.r1 Soares mouse mammary gland NbMMG Mus | | | | |
| | | musculus cDNA clone 850361 5′ similar to WP:C14B1.3 | | | | |
| FBXO6B | AA451220 | CE00900; | 10.00 | 12.00 | 22.00 | 28.33 |
| | | Mus musculus TGF-beta-inducible protein (TSC-36) mRNA, | | | | |
| FSTL | M91380 | complete cds | 10.00 | 10.00 | 20.00 | 13.00 |
| | | Mus musculus ion channel homolog RIC mRNA, complete | | | | |
| FXYD5 | U72680 | cds. | 10.25 | 10.00 | 31.00 | 29.67 |
| GNB1 | U29055 | Mus musculus G protein beta 36 subunit mRNA, compl | 11.75 | 11.33 | 28.33 | 37.33 |
| GRN | M86736 | Mouse acrogranin mRNA, complete cds | 56.25 | 51.67 | 129.00 | 159.67 |
| | | Mouse tumor-induced 32 kD protein (p32) mRNA, complete | | | | |
| HMOX1 | M33203 | cds | 10.00 | 10.00 | 20.00 | 28.33 |
| HN1 | U90123 | Mus musculus HN1 (Hn1) mRNA, complete cds. | 10.00 | 10.00 | 23.67 | 25.00 |
| HSP25 | L07577 | Mus musculus small heat shock protein (HSP25) gene | 31.75 | 35.00 | 131.67 | 191.00 |
| | | Mouse interferon-inducible protein homologue mRNA, | | | | |
| IFIT3 | L32974 | complete cds | 13.75 | 10.00 | 29.00 | 33.00 |
| | | Mus musculus interferon regulatory factor 7 (mirf7) mRNA, | | | | |
| IRF7 | U73037 | complete cds | 10.00 | 10.67 | 27.33 | 33.33 |
| ITGB4BP | AA122622 | B integrin interactor homolog | 11.25 | 10.00 | 25.33 | 16.33 |
| JUN | W09701 | ma56e02.r1 Mus musculus cDNA, 5′ end | 16.25 | 16.33 | 32.33 | 32.67 |
| KRT2-8 | D90360 | Mouse gene for cytokeratin endo A | 19.50 | 18.00 | 49.00 | 92.67 |
| LAPTM5 | U29539 | Mus musculus retinoic acid-inducible E3 protein mR | 10.25 | 11.00 | 27.33 | 34.00 |
| LCN2 | X81627 | M. musculus 24p3 gene. | 10.00 | 10.00 | 81.67 | 194.33 |
| LGALS3 | W10936 | ma03e09.r1 Mus musculus cDNA, 5′ end | 10.00 | 10.00 | 27.33 | 28.33 |
| LOC56722 | AA542220 | TBX1 protein (novel) | 14.50 | 11.33 | 42.67 | 64.33 |
| | | Mus musculus lymphocyte specific transcript (LST) mRNA, | | | | |
| LST1 | U72643 | partial cds. | 11.00 | 13.00 | 29.33 | 29.67 |
| | | Mouse lyn A protein tyrosine kinase (lynA) mRNA, complete | | | | |
| LYN | M57696 | cds | 14.25 | 13.67 | 30.00 | 43.33 |
| MAPK1 | AA104744 | MAP kinase | 10.00 | 10.00 | 28.67 | 23.00 |
| MGLAP | D00613 | Mouse mRNA for matrix Gla protein (MGP) | 47.75 | 44.33 | 249.67 | 132.33 |
| MKI67 | X82786 | M. musculus mRNA for Ki-67. | 10.00 | 10.00 | 21.33 | 19.00 |
| | | mw28h11.r1 Soares mouse 3NME12 5 Mus musculus cDNA | | | | |
| | | clone 672069 5′ similar to gb:X61399 Mouse F52 mRNA for a | | | | |
| MLP | AA245242 | novel protein (MOUSE); | 11.25 | 11.00 | 31.00 | 32.33 |
| MPEG1 | L20315 | Mus musculus MPS1 gene and mRNA, 3′end | 10.00 | 10.00 | 30.00 | 37.67 |
| NFKBIA | U36277 | Mus musculus I-kappa B alpha chain mRNA, complete cds | 14.75 | 17.67 | 44.00 | 42.00 |
| OAS1A | M33863 | Mouse 2′-5′ oligo A synthetase mRNA, complete cds. | 11.50 | 10.00 | 25.00 | 28.33 |
| | | EST03133 Mouse 7.5 dpc embryo ectoplacental cone cDNA | | | | |
| P21ARC | AA408672 | library Mus musculus cDNA clone C0031D07 3′ | 39.25 | 36.00 | 80.00 | 75.67 |
| PEA15 | AA108330 | mp28b03.r1 Mus musculus cDNA, 5′ end | 11.50 | 10.00 | 40.00 | 51.33 |
| | | Mouse mRNA for mastocytoma proteoglycan core protein, | | | | |
| PRG | X16133 | serglycin. | 18.25 | 14.00 | 51.33 | 43.67 |
| | | Mus musculus 20S proteasome subunit Lmp7 (Lmp7d allele) | | | | |
| PSMB8 | U22031 | mRNA, complete cds | 10.25 | 10.00 | 41.33 | 38.67 |
| PSME2 | D87910 | Mus musculus mRNA for PA28 beta subunit, complete cds. | 21.75 | 24.67 | 64.00 | 74.00 |
| PTMB4 | W41883 | mc64g08.r1 Mus musculus cDNA 5′ end | 83.75 | 78.33 | 272.00 | 194.67 |
| PTPN1 | U24700 | Mus musculus protein tyrosine phosphatase (HA2) mR | 10.00 | 11.67 | 22.00 | 42.33 |
| RAC2 | X53247 | Mus musculus EN-7 mRNA. | 13.00 | 17.67 | 59.67 | 59.67 |
| | | vj03d05.r1 Barstead mouse pooled organs MPLRB4 Mus | | | | |
| | | musculus cDNA clone 920649 5′ similar to TR:G881954 | | | | |
| RBM3 | AA538285 | G881954 RNPL.; | 13.50 | 10.67 | 42.00 | 82.33 |
| | | Mus musculus G protein signaling regulator RGS2 (rgs2) | | | | |
| RGS2 | U67187 | mRNA, complete cds. | 10.00 | 14.67 | 24.33 | 41.33 |
| | | EST02956 Mouse 7.5 dpc embryo ectoplacental cone cDNA | | | | |
| | | library Mus musculus cDNA clone C0028E12 3′, mRNA | | | | |
| RPL13A | AA408475 | sequence. | 11.00 | 11.33 | 24.33 | 21.67 |
| RRAS | M21019 | Mouse R-ras mRNA, complete cds | 16.00 | 12.00 | 43.33 | 53.33 |
| RRAS | W41501 | mc43d11.r1 Mus musculus cDNA, 5′ end | 10.25 | 10.00 | 21.67 | 25.67 |
| | | Mouse 3.5-dpc blastocyst cDNA Mus musculus cDNA clone | | | | |
| | | J0101H11 3′ similar to Mouse ribonucleotide reductase M2 | | | | |
| RRM2 | C81593 | subunit mRNA, mRNA sequence. | 10.00 | 10.00 | 23.00 | 17.67 |
| | | mq98h01.r1 Soares mouse 3NbMS Mus musculus cDNA | | | | |
| SCYA19 | AA137292 | clone 596017 5′ | 16.25 | 22.00 | 32.33 | 46.67 |
| | | Mus musculus NIH 3T3 chemokine rantes (Scya5) gene, | | | | |
| SCYA5 | U02298 | complete cds | 10.00 | 10.00 | 22.33 | 13.67 |
| SCYD1 | U92565 | Mus musculus fractalkine mRNA, complete cds. | 11.50 | 10.00 | 30.33 | 25.67 |
| SLC20A1 | M73696 | Murine Glvr-1 mRNA, complete cds | 10.00 | 10.00 | 20.67 | 31.67 |
| | | Mus musculus secretory leukocyte protease inhibitor mRNA, | | | | |
| SLPI | U73004 | complete cds. | 10.00 | 10.00 | 24.00 | 26.67 |
| SNRPD1 | M58558 | Murine sm D small nuclear ribonucleoprotein sequence. | 10.00 | 10.33 | 20.33 | 24.33 |
| SPI2-1 | M64085 | Mouse spi2 proteinase inhibitor (spi2/eb1) mRNA, 3 | 10.00 | 12.67 | 20.67 | 28.33 |
| | | mp09d07.r1 Life Tech mouse embryo 8 5dpc 10664019 Mus | | | | |
| SPI6 | AA108054 | musculus cDNA clone 568717 5′ | 10.00 | 10.00 | 23.33 | 28.67 |
| | | Mus musculus signal transducer and activator of transcription | | | | |
| STAT3 | U06922 | (Stat3) mRNA, complete cds | 42.25 | 37.67 | 99.33 | 152.33 |
| | | vb41e05.r1 Soares mouse lymph node NbMLN Mus | | | | |
| STAT3 | AA396029 | musculus cDNA clone 751520 5′ | 10.00 | 10.00 | 20.67 | 34.00 |
| | | mp39a05.r1 Barstead MPLRB1 Mus musculus cDNA clone | | | | |
| STK2 | AA108677 | 571568 5′ | 10.00 | 11.00 | 21.00 | 24.33 |
| | | Mus musculus (clone U2) T-cell specific protein mRNA, | | | | |
| TGTP | L38444 | complete cds | 10.00 | 10.00 | 20.00 | 20.33 |
| TLN | X56123 | Mouse mRNA for talin | 10.00 | 10.00 | 28.00 | 11.00 |
| UNK_AA011 | | | | | | |
| 784 | AA011784 | mg92b08.r1 Mus musculus cDNA, 5′ end | 17.50 | 16.00 | 67.67 | 60.00 |
| UNK_AA023 | | | | | | |
| 491 | AA023491 | mh74e11.r1 Mus musculus cDNA, 5′ end | 10.00 | 10.00 | 38.33 | 20.33 |
| UNK_AA030 | | mi22g02.r1 Soares mouse embryo NbME13.5 14.5 Mus | | | | |
| 688 | AA030688 | musculus cDNA clone 464306 5′ | 10.25 | 10.00 | 25.67 | 36.33 |
| UNK_AA087 | | | | | | |
| 673 | AA087673 | mm27b09.r1 Mus musculus cDNA, 5′ end | 10.00 | 22.33 | 81.67 | 245.33 |
| UNK_AA104 | | | | | | |
| 688 | AA104688 | mo55c10.r1 Mus musculus cDNA, 5′ end | 10.00 | 10.00 | 42.67 | 27.33 |
| UNK_AA107 | | | | | | |
| 847 | AA107847 | mo49d08.r1 Mus musculus cDNA, 5′ end | 10.00 | 10.00 | 34.67 | 16.00 |
| UNK_AA109 | | | | | | |
| 909 | AA109909 | mp10d09.r1 Mus musculus cDNA, 5′ end | 10.00 | 10.00 | 28.67 | 17.00 |
| UNK_AA163 | | mt65a03.r1 Soares mouse lymph node NbMLN Mus | | | | |
| 096 | AA163096 | musculus cDNA clone 634732 5′ | 17.25 | 13.67 | 45.00 | 43.33 |
| UNK_AA172 | | mr31f05.r1 Soares mouse 3NbMS Mus musculus cDNA | | | | |
| 851 | AA172851 | clone 599073 5′ | 10.00 | 11.33 | 21.67 | 58.33 |
| UNK_AA174 | | ms77e07.r1 Soares mouse 3NbMS Mus musculus cDNA | | | | |
| 883 | AA174883 | clone 617604 5′ | 25.00 | 32.00 | 65.67 | 109.67 |
| UNK_AA184 | | mt22f04.r1 Soares mouse 3NbMS Mus musculus cDNA | | | | |
| 116 | AA184116 | clone 621823 5′ | 11.75 | 11.00 | 28.00 | 37.00 |
| UNK_AA210 | | mu72h03.r1 Soares mouse lymph node NbMLN Mus | | | | |
| 359 | AA210359 | musculus cDNA clone 644981 5′ | 13.00 | 11.00 | 29.33 | 37.33 |
| | | mx94f04.r1 Soares mouse NML Mus musculus cDNA clone | | | | |
| UNK_AA238 | | 694015 5′ similar to TR:G806566 G806566 SM PROTEIN G. | | | | |
| 483 | AA238483 | ; | 13.00 | 15.00 | 31.33 | 34.33 |
| UNK_AA538 | | vj53e12.r1 Knowles Solter mouse blastocyst B1 Mus | | | | |
| 477 | AA538477 | musculus cDNA clone 932782 5′ | 11.00 | 11.67 | 22.67 | 42.67 |
| | | vl56h09.r1 Stratagene mouse skin (#937313) Mus musculus | | | | |
| UNK_AA562 | | cDNA clone 976289 5′ similar to gb:X06753 Mouse pro- | | | | |
| 685 | AA562685 | alpha1 (MOUSE); | 11.50 | 10.00 | 58.33 | 28.67 |
| | | vm91d04.r1 Knowles Solter mouse blastocyst B1 Mus | | | | |
| UNK_AA606 | | musculus cDNA clone 1005607 5′ similar to TR:G497940 | | | | |
| 926 | AA606926 | G497940 MAJOR VAULT PROTEIN.;, mRNA sequence. | 15.25 | 10.33 | 35.00 | 46.00 |
| UNK_AA616 | | vo50d04.r1 Barstead mouse irradiated colon MPLRB7 Mus | | | | |
| 243 | AA616243 | musculus cDNA clone 1053319 5′, mRNA sequence. | 10.00 | 10.00 | 21.33 | 37.67 |
| UNK_AA690 | | vu57b03.r1 Soares mouse mammary gland NbMMG Mus | | | | |
| 738 | AA690738 | musculus cDNA clone 1195469 5′, mRNA sequence. | 15.50 | 12.00 | 36.33 | 49.67 |
| UNK_AA710 | | vt42f07.r1 Barstead mouse proximal colon MPLRB6 Mus | | | | |
| 451 | AA710451 | musculus cDNA clone 1165765 5′, mRNA sequence. | 10.00 | 10.00 | 46.33 | 31.67 |
| UNK_AC002 | | Mouse chromosome 6 BAC-284H12 (Research Genetics | | | | |
| 397 | AC002397 | mouse BAC library) complete sequence. | 10.00 | 13.00 | 25.00 | 33.33 |
| | | Mouse 3.5-dpc blastocyst cDNA Mus musculus cDNA clone | | | | |
| UNK_C76523 | C76523 | J0012E07 3′, mRNA sequence. | 11.50 | 10.00 | 30.67 | 40.33 |
| | | Mouse 3.5-dpc blastocyst cDNA Mus musculus cDNA clone | | | | |
| UNK_C76523 | C76523 | J0012E07 3′, mRNA sequence. | 10.00 | 10.00 | 23.00 | 19.67 |
| | | Mouse 3.5-dpc blastocyst cDNA Mus musculus cDNA clone | | | | |
| | | J0038G08 3′ similar to Rattus norvegicus major vault protein | | | | |
| UNK_C77861 | C77861 | mRNA, mRNA sequence. | 16.50 | 13.33 | 35.67 | 42.67 |
| | | Mouse 3.5-dpc blastocyst cDNA Mus musculus cDNA clone | | | | |
| UNK_C80574 | C80574 | J0084D04 3′ similar to Human clone 23665 mRNA sequence. | 28.00 | 21.67 | 60.67 | 83.00 |
| UNK_ET61420 | | Mus musculus anti-glycoprotein-B of human Cytomegalovirus | | | | |
| | ET61420 | immunoglobulin Vh chain gene, partial cds. | 10.00 | 10.00 | 65.67 | 86.33 |
| UNK_ET63106 | | M. musculus mRNA for immunoglobulin heavy chain variable | | | | |
| | ET63106 | region, isolate 205. | 10.00 | 10.00 | 22.33 | 32.67 |
| UNK_ET63126 | | M. musculus mRNA for anti folate binding protein, MOv19 | | | | |
| | ET63126 | Vkappa. | 10.00 | 11.67 | 30.00 | 40.33 |
| UNK_W08057 | | | | | | |
| | W08057 | mb37e05.r1 Mus musculus cDNA, 5′ end | 10.00 | 11.00 | 48.00 | 59.00 |
| | | ma74d01.r1 Soares mouse p3NMF19.5 Mus musculus cDNA | | | | |
| | | clone 316417 5′ similar to gb:J03909 GAMMA-INTERFERON. | | | | |
| UNK_W11156 | | INDUCIBLE PROTEIN IP-30 PRECURSOR (HUMAN);, | | | | |
| | W11156 | mRNA sequence. | 27.75 | 31.00 | 57.67 | 51.33 |
| UNK_W20873 | W20873 | mb92c11.r1 Mus musculus cDNA, 5′ end | 10.00 | 10.00 | 32.00 | 34.67 |
| UNK_W29429 | W29429 | mb99d03.r1 Mus musculus cDNA, 5′ end | 10.00 | 13.33 | 33.67 | 29.33 |
| UNK_W48951 | W48951 | md24g11.r1 Mus musculus cDNA, 5′ end | 10.00 | 10.00 | 20.00 | 10.00 |
| UNK_W50888 | W50888 | ma23e03.r1 Mus musculus cDNA, 5′ end | 12.00 | 21.67 | 24.67 | 27.67 |
| UNK_W50898 | W50898 | ma23g03.r1 Mus musculus cDNA, 5′ end | 15.75 | 18.67 | 40.33 | 31.67 |
| UNK_W57485 | W57485 | ma34h02.r1 Mus musculus cDNA, 5′ end | 10.00 | 10.00 | 23.67 | 21.33 |
| UNK_W90837 | W90837 | mf78g07.r1 Mus musculus cDNA, 5′ end | 10.75 | 10.00 | 33.00 | 27.33 |
| UNK_X52622 | X52622 | Mouse IN gene for the integrase of an endogenous retrovirus | 10.25 | 10.00 | 20.33 | 57.00 |
| | | ma89d07.r1 Soares mouse p3NMF19.5 Mus musculus cDNA | | | | |
| | | clone 317869 5′ similar to gb:X57352 INTERFERON- | | | | |
| VCP | W12941 | INDUCIBLE PROTEIN 1-8U (HUMAN);, mRNA sequence. | 31.00 | 27.33 | 121.33 | 91.00 |
| | | House mouse; Musculus domesticus mRNA for 14-3-3 eta, | | | | |
| YWHAH | D87661 | complete cds | 10.50 | 10.00 | 22.00 | 27.33 |
| ACINUS- | | vf79g11.r1 Soares mouse mammary gland NbMMG Mus | | | | |
| PENDING | AA444568 | musculus cDNA clone 850052 5′ | 10.00 | 17.33 | 21.67 | 33.00 |
| APOE | AA048604 | mj32g02.r1 Mus musculus cDNA, 5′ end | 70.75 | 86.33 | 236.67 | 301.33 |
| ARHC | X80638 | M. musculus rhoC mRNA. | 47.00 | 43.33 | 104.67 | 147.33 |
| BGN | L20276 | Mouse biglycan (Bgn) mRNA, complete cds | 71.25 | 54.67 | 169.33 | 134.33 |
| CAPPB1 | U10406 | Mus musculus capping protein beta-subunit isoform | 35.75 | 37.00 | 72.67 | 90.33 |
| | | M. musculus intracisternal A-particle IAP-IL3 genome deleted | | | | |
| CCR4 | X04120 | type I element inserted 5′ to the interleukin-3 gene. | 50.00 | 72.33 | 112.33 | 134.67 |
| CD36L2 | AB008553 | Mus musculus mRNA for mLGP85/LIMP II, complete cds. | 10.25 | 12.67 | 21.00 | 21.00 |
| CFL1 | D00472 | Mouse mRNA for cofilin, complete cds and flanks | 28.25 | 37.67 | 81.00 | 108.33 |
| CLU | L08235 | Mus musculus clusterin mRNA, complete cds | 163.25 | 115.00 | 415.33 | 608.00 |
| CP | U49430 | Mus musculus ceruloplasmin mRNA, complete cds | 20.50 | 15.67 | 69.00 | 157.67 |
| | | mi67c05.r1 Soares mouse embryo NbME13.5 14.5 Mus | | | | |
| D11ERTD172E | AA014563 | musculus cDNA clone 468584 5′. | 38.25 | 47.00 | 77.33 | 101.67 |
| | | vu31g07.r1 Stratagene mouse Tcell 937311 Mus musculus | | | | |
| | | cDNA clone 1193052 5′ similar to SW:INI7_HUMAN P40305 | | | | |
| | | INTERFERON-ALPHA INDUCED 11.5 KD PROTEIN;, | | | | |
| D12ERTD647E | AA711625 | mRNA sequence. | 102.00 | 109.67 | 317.67 | 430.00 |
| | | vp33f01.r1 Barstead mouse proximal colon MPLRB6 Mus | | | | |
| D17WSU91E | AA727845 | musculus cDNA clone 1078489 5′, mRNA sequence. | 84.50 | 80.33 | 189.67 | 262.00 |
| | | EST01599 Mouse 7.5 dpc embryo ectoplacental cone cDNA | | | | |
| | | library Mus musculus cDNA clone C0012A02 3′, mRNA | | | | |
| D4WSU27E | AA409826 | sequence. | 34.50 | 20.67 | 78.00 | 105.00 |
| EEF2 | W98531 | elongation factor 2 (ef-2) | 11.50 | 20.67 | 35.00 | 37.33 |
| | | Mus musculus FK506 binding protein 51 mRNA, complete | | | | |
| FKBP5 | U36220 | cds | 14.25 | 24.33 | 28.33 | 60.33 |
| FTH | W18308 | mb68h11.r1 Mus musculus cDNA, 5′ end | 331.75 | 595.00 | 695.33 | 621.33 |
| GAS5 | X59728 | M. musculus mRNA for gas5 growth arrest specific protein | 14.00 | 19.00 | 36.33 | 45.33 |
| GP49A | M65027 | Mouse cell surface antigen gp49 mRNA, complete cds | 14.00 | 18.67 | 28.33 | 32.00 |
| | | mi95f04.r1 Soares mouse p3NMF19.5 Mus musculus cDNA | | | | |
| HZF- | | clone 474367 5′ similar to gb:U27830 Mus musculus extendin | | | | |
| PENDING | AA038775 | mRNA, complete cds (MOUSE); | 13.75 | 24.33 | 43.00 | 45.00 |
| IRF1 | M21065 | Mouse interferon regulatory factor 1 mRNA, complete cds | 12.00 | 15.00 | 40.67 | 43.67 |
| JUND1 | X15358 | Mouse mRNA for junD proto-oncogene. | 53.75 | 73.00 | 109.33 | 135.00 |
| KCNJ11 | D50581 | Mouse mRNA for inward rectifier K+ channel | 10.50 | 14.67 | 23.00 | 31.00 |
| KLF2 | U25096 | Mus musculus Kruppel-like factor LKLF mRNA, complete cds | 10.00 | 12.67 | 26.67 | 33.33 |
| KPNA2 | C79184 | nuclear pore-targeting complex, mRNA sequence. | 33.75 | 41.33 | 101.33 | 109.00 |
| L1MD-TF14 | D84391 | Mouse L1 repetitive element, complete sequence. | 14.00 | 36.67 | 43.33 | 53.33 |
| LGALS1 | X66532 | M. musculus mRNA for L14 lectin. | 34.75 | 46.67 | 190.33 | 133.67 |
| LGALS1 | W13002 | mb21e10.r1 Mus musculus cDNA, 5′ end | 26.00 | 30.33 | 151.67 | 101.67 |
| LGALS3 | X16834 | Mouse mRNA for Mac-2 antigen | 29.00 | 36.67 | 116.67 | 134.00 |
| | | Mus musculus C57BL/6 Sca-2 precursor mRNA, complete | | | | |
| LY6E | U04268 | cds. | 91.00 | 82.67 | 362.67 | 585.67 |
| | | mg36a03.r1 Soares mouse embryo NbME13.5 14.5 Mus | | | | |
| LY6E | AA000467 | musculus cDNA clone 425836 5′. | 26.75 | 54.67 | 73.00 | 90.00 |
| | | Mouse retinoic acid-responsive protein (MK) mRNA, | | | | |
| MDK | M35833 | complete cds | 33.25 | 42.33 | 105.67 | 112.00 |
| | | Mouse retinoic acid-responsive protein (MK) gene, complete | | | | |
| MDK | M34094 | cds | 30.25 | 38.00 | 90.67 | 49.67 |
| | | Mouse microfibril-associated glycoprotein (Magp) mRNA, | | | | |
| MFAP2 | L23769 | complete cds | 10.50 | 11.00 | 21.67 | 16.67 |
| MT2 | AA109597 | metallothione 2 | 24.25 | 86.33 | 69.00 | 84.33 |
| PEA15 | L31958 | Mus musculus (clone: pMAT1) mRNA, complete cds | 34.25 | 16.67 | 77.67 | 111.33 |
| PPICAP | X67809 | M. musculus mama mRNA. | 15.25 | 10.00 | 78.00 | 84.00 |
| | | Mouse 3.5-dpc blastocyst cDNA Mus musculus cDNA clone | | | | |
| | | J0087G04 3′ similar to Rat mRNA for proteasome subunit | | | | |
| PSMA3 | C80757 | RC8, mRNA sequence. | 12.25 | 18.33 | 27.00 | 29.67 |
| | | mg75a06.r1 Soares mouse embryo NbME13.5 14.5 Mus | | | | |
| | | musculus cDNA clone 438802 5′ similar to gb:D00763 | | | | |
| PSMA4 | AA008321 | PROTEASOME COMPONENT C9 (HUMAN);. | 41.50 | 71.67 | 88.00 | 89.00 |
| RAB11A | D50500 | Mouse mRNA for Rab 11, partial sequence. | 18.00 | 22.33 | 41.67 | 49.00 |
| | | Mouse 3.5-dpc blastocyst cDNA Mus musculus cDNA clone | | | | |
| | | J0030G04 3′ similar to Mouse B10.VL30LTR gene, 5′ flank, | | | | |
| RBPSUH | C77421 | mRNA sequence. | 88.25 | 81.00 | 197.00 | 306.00 |
| RPL22 | D17653 | Mouse mRNA for HBp15/L22, complete cds | 71.50 | 73.33 | 147.33 | 171.67 |
| S100A10 | M16465 | Mouse calpactin I light chain (p11) mRNA, complete cds | 40.00 | 29.67 | 96.67 | 137.67 |
| | | Mus musculus endothelial monocyte-activating polypeptide I | | | | |
| S100A11 | U41341 | mRNA, complete cds. | 24.25 | 24.67 | 120.67 | 171.33 |
| | | Mouse pEL98 protein mRNA which is enhanced in | | | | |
| S100A4 | D00208 | established cells, Balb/c373 | 14.50 | 19.33 | 35.33 | 38.33 |
| S100A6 | M37761 | Mouse calcyclin mRNA, complete cds | 21.50 | 33.33 | 161.67 | 178.00 |
| S100A6 | X66449 | M. musculus mRNA for calcyclin | 10.00 | 10.00 | 23.67 | 34.00 |
| | | Mus musculus C57BL/6J Sec61 protein complex gamma | | | | |
| SEC61G | U11027 | subunit mRNA, complete cds | 37.00 | 42.00 | 75.67 | 96.33 |
| | | Mus musculus selenoprotein W (mSelW) mRNA, complete | | | | |
| SEPW1 | AF015284 | cds. | 24.75 | 26.33 | 50.67 | 65.00 |
| SPRR1A | X91824 | M. musculus mRNA for SPRR1a protein. | 11.25 | 11.33 | 68.67 | 40.67 |
| STAT5A | U21103 | Mus musculus mammary gland factor (Stat5a) mRNA, c | 10.75 | 20.33 | 26.33 | 32.67 |
| TAGLN | L41154 | Mus musculus SM22 alpha mRNA, complete cds | 20.50 | 20.33 | 79.67 | 50.67 |
| TGFB1I4 | X62940 | M. musculus TSC-22 mRNA. | 137.50 | 123.67 | 306.33 | 424.33 |
| UCP2 | U69135 | Mus musculus UCP2 mRNA, complete cds.) | 14.50 | 15.33 | 75.33 | 148.33 |
| UNK_AA000 | | mg24e05.r1 Soares mouse embryo NbME13.5 14.5 Mus | | | | |
| 380 | AA000380 | musculus cDNA clone 424736 5′. | 28.00 | 40.00 | 63.00 | 72.00 |
| UNK_AA002 | | mg38h07.r1 Soares mouse embryo NbME13.5 14.5 Mus | | | | |
| 653 | AA002653 | musculus cDNA clone 426109 5′. | 12.25 | 19.67 | 31.67 | 40.00 |
| UNK_AA004 | | mg80f01.r1 Soares mouse embryo NbME13.5 14.5 Mus | | | | |
| 011 | AA004011 | musculus cDNA clone 439321 5′. | 10.00 | 16.67 | 20.67 | 24.33 |
| UNK_AA028 | | mi14h12.r1 Soares mouse p3NMF19.5 Mus musculus cDNA | | | | |
| 657 | AA028657 | clone 463559 5′ | 28.75 | 37.67 | 59.33 | 79.00 |
| UNK_AA038 | | | | | | |
| 347 | AA038347 | mi84d05.r1 Mus musculus cDNA, 5′ end | 10.50 | 12.33 | 38.33 | 37.67 |
| UNK_AA038 | | | | | | |
| 511 | AA038511 | mi85h01.r1 Mus musculus cDNA, 5′ end | 28.50 | 41.33 | 113.00 | 116.67 |
| UNK_AA068 | | | | | | |
| 158 | AA068158 | mm56e10.r1 Mus musculus cDNA, 5′ end | 26.25 | 29.67 | 81.00 | 71.67 |
| UNK_AA097 | | | | | | |
| 626 | AA097626 | mo08g01.r1 Mus musculus cDNA, 5′ end | 29.50 | 73.67 | 176.33 | 409.33 |
| UNK_AA168 | | | | | | |
| 865 | AA168865 | ms38c08.r1 Mus musculus cDNA, 5′ end | 11.25 | 15.33 | 35.67 | 37.33 |
| UNK_AA184 | | mt58c09.r1 Soares 2NbMT Mus musculus cDNA clone | | | | |
| 455 | AA184455 | 634096 5′ | 10.00 | 13.67 | 21.00 | 25.33 |
| UNK_AA617 | | vi21f09.r1 Barstead mouse proximal colon MPLRB6 Mus | | | | |
| 093 | AA617093 | musculus cDNA clone 904457 5′, mRNA sequence. | 10.75 | 16.67 | 21.33 | 39.33 |
| UNK_AA711 | | vt56c05.r2 Barstead mouse irradiated colon MPLRB7 Mus | | | | |
| 130 | AA711130 | musculus cDNA clone 1167080 5′, mRNA sequence. | 149.75 | 166.33 | 321.33 | 525.33 |
| | | Mouse 3.5-dpc blastocyst cDNA Mus musculus cDNA clone | | | | |
| | | J0004G06 3′ similar to Rat insulin-I (ins-1) gene, mRNA | | | | |
| UNK_C76162 | C76162 | sequence. | 11.50 | 35.00 | 42.33 | 48.67 |
| | | Mouse 3.5-dpc blastocyst cDNA Mus musculus cDNA clone | | | | |
| | | J0032G04 3′ similar to Rat G protein gamma-5 subunit, | | | | |
| UNK_C77514 | C77514 | mRNA sequence. | 90.75 | 112.33 | 190.67 | 223.67 |
| | | Mouse 3.5-dpc blastocyst cDNA Mus musculus cDNA clone | | | | |
| | | J0051B02 3′ similar to moesin homolog [mice, | | | | |
| UNK_C78546 | C78546 | teratocarcinoma F9 cells, mRNA, mRNA sequence. | 40.25 | 40.67 | 87.33 | 103.67 |
| UNK_M26005 | | Mouse endogenous retrovirus truncated gag protein, | | | | |
| | M26005 | complete cds, clone del env-1 3.1 | 12.25 | 24.00 | 61.67 | 128.00 |
| UNK_M29325 | | | | | | |
| | M29325 | Mouse L1Md-9 repetitive sequence (EXTRACTED 3′UTR) | 13.75 | 39.67 | 36.67 | 38.00 |
| | | MDB1515 Mouse brain, Stratagene Mus musculus cDNA | | | | |
| UNK_N28179 | N28179 | 3′ end. | 12.75 | 27.67 | 32.33 | 49.33 |
| | | MDB0793 Mouse brain, Stratagene Mus musculus cDNA | | | | |
| UNK_R74638 | R74638 | 3′end. | 13.00 | 27.00 | 27.00 | 37.33 |
| UNK_W11954 | | | | | | |
| | W11954 | ma79e11.r1 Mus musculus cDNA, 5′ end | 12.75 | 20.33 | 30.67 | 34.67 |
| UNK_W18503 | | | | | | |
| | W18503 | mb88b08.r1 Mus musculus cDNA, 5′ end | 12.25 | 14.67 | 25.00 | 31.67 |
| VCP | AA002526 | mg54a04.r1 Mus musculus cDNA, 5′ end | 18.50 | 21.33 | 84.33 | 84.00 |

TABLE 4
Genes Decreased in Disease

| | | | Untreated | Untreated | Untreated | Untreated |
|---|---|---|---|---|---|---|
| Gene | Accession | | @ 12 wks | @ 25 wks | @ 36 wks | @ 42 wks |
| name | number | Function | of age | of age | of age | of age |
| LPL | AA683731 | lipoprotein lipase | 207.00 | 136.00 | 85.00 | 79.33 |
| FMO1 | U87456 | flavin containing monooxygenase 1 | 128.50 | 78.00 | 44.67 | 57.00 |
| D7RP2E | X04097 | DNA segment, Chr 7, Roswell Park 2 complex, expressed | 102.50 | 68.00 | 35.33 | 40.33 |
| GLUD | X57024 | glutamate dehydrogenase | 66.25 | 38.33 | 33.00 | 49.00 |
| | | ESTs, Highly similar to sodium-dependent multi-vitamin | | | | |
| UNK_AA5634 | AA563404 | transporter [R. norvegicus] | 86.75 | 42.00 | 31.00 | 36.33 |
| | | mx03b10.r1 Soares mouse NML Mus musculus cDNA clone | | | | |
| UNK_AA2457 | AA245784 | 679099 5′ | 65.00 | 41.00 | 29.67 | 45.33 |
| DNASE1 | AA109013 | deoxyribonuclease I | 163.25 | 168.00 | 29.00 | 44.67 |
| CALB1 | M21531 | calbindin-28K | 76.75 | 57.00 | 26.67 | 37.00 |
| FBP1 | AA109491 | fructose bisphosphatase 1 | 71.25 | 41.00 | 26.67 | 33.00 |
| FMO5 | AA268913 | flavin containing monooxygenase 5 | 51.25 | 21.33 | 25.00 | 35.33 |
| | | ATPase, H+ transporting, lysosomal (vacuolar proton pump), | | | | |
| ATP6A2 | U13837 | alpha 70 kDa, isoform 2 | 47.50 | 26.67 | 22.67 | 28.33 |
| | | ESTs, Weakly similar to A34337 propionyl-CoA carboxylase | | | | |
| UNK_AA1979 | AA197973 | [R. norvegicus] | 46.00 | 26.67 | 20.67 | 21.67 |
| ITPR1 | X15373 | inositol 1,4,5-triphosphate receptor 1 | 42.25 | 30.00 | 20.00 | 24.67 |
| IDB4 | X75018 | inhibitor of DNA binding 4 | 43.00 | 26.67 | 16.67 | 25.67 |
| | | ESTs, Moderately similar to multidrug resistance protein | | | | |
| UNK_AA1657 | AA165775 | [M. musculus] | 30.25 | 25.00 | 14.67 | 23.67 |
| | | DNA segment, Chr 19, Wayne State University 57, | | | | |
| D19WSU57E | AA246000 | expressed | 32.75 | 48.33 | 12.67 | 78.33 |
| NGEF | AA607353 | neuronal guanine nucleotide exchange factor | 37.75 | 28.67 | 12.67 | 19.67 |
| UNK_AA0230 | AA023065 | ESTs, Weakly similar to SIG41 [M. musculus] | 26.00 | 13.67 | 11.67 | 18.33 |
| PVA | X67141 | parvalbumin | 28.00 | 22.33 | 10.00 | 11.00 |

TABLE 5
Genes Increased in Disease and Treated with Rapamycin

| | | | Normal- | | | |
|---|---|---|---|---|---|---|
| | | | ized by | Untreated | Untreated | Rapa |
| Gene | Accession | | Rapamy | @ 12 wks | @ 36 wks | @ 36 |
| name | number | Description | cin | of age | of age | weeks |
| ACTC1 | AA117701 | mo64d03.r1 Mus musculus cDNA, 5′ end | YES | 10.75 | 22.33 | 11.67 |
| | | Mouse mRNA for secretory protein containing. | | | | |
| ADAMTS1 | D67076 | thrombospondin motifs, complete cds. | YES | 10.00 | 36.00 | 10.00 |
| ANXA1 | X07486 | Mouse mRNA for lipocortin I. | YES | 15.00 | 36.00 | 14.67 |
| ANXA2 | M14044 | Mouse calpactin I heavy chain (p36) mRNA, complete cds | YES | 22.00 | 139.67 | 27.00 |
| | | Mouse mRNA for protein-tyrosine kinase substrate p36 | | | | |
| ANXA2 | D10024 | (calpactin I heavy chain), complete cds | YES | 20.50 | 105.67 | 21.67 |
| ANXA5 | W98864 | mg11h11.r1 Mus musculus cDNA, 5′ end | YES | 12.00 | 29.33 | 13.33 |
| ARG2 | AF032466 | Mus musculus arginase II mRNA, complete cds. | YES | 10.25 | 21.33 | 12.33 |
| | | Mus musculus copper transport protein Atox1 (ATOX1) | | | | |
| ATOX1 | AF004591 | mRNA, complete cds. | YES | 44.25 | 90.00 | 43.67 |
| C1NH | Y10386 | M. musculus mRNA for C1 inhibitor. | YES | 37.00 | 94.00 | 41.67 |
| | | Mouse CD14 mRNA for myelid cell-specific leucine-rich | | | | |
| CD14 | X13333 | glycoprotein. | YES | 25.50 | 89.33 | 29.67 |
| CD52 | M55561 | Mouse phosphatidylinositol-linked antigen (pB7) mR | YES | 10.00 | 31.33 | 10.00 |
| CD68 | AB009287 | Mus musculus gene for Macrosialin, complete cds. | YES | 10.00 | 23.33 | 12.67 |
| | | Mouse B-cell differentiation antigen Lyb-2.1 protein, complete | | | | |
| CD72 | J04170 | cds | YES | 10.00 | 22.67 | 10.67 |
| CEBPB | X62600 | M. musculus mRNA for C/EBP beta. | YES | 10.00 | 23.33 | 10.00 |
| | | Mus musculus mCPE-R mRNA for CPE-receptor, complete | | | | |
| CLDN4 | AB000713 | cds. | YES | 16.00 | 48.67 | 18.33 |
| CNN2 | Z19543 | M. musculus h2-calponin cDNA | YES | 15.25 | 34.33 | 19.00 |
| | | Mouse cysteine-rich intestinal protein (CRIP) mRNA, | | | | |
| CRIP | M13018 | complete cds | YES | 10.00 | 49.33 | 14.33 |
| CSTB | U59807 | Mus musculus cystatin B (Stfb) gene, complete cds. | YES | 14.50 | 68.00 | 18.33 |
| CTGF | M70642 | Mouse FISP-12 protein (fisp-12) mRNA, complete cds | YES | 19.50 | 83.00 | 16.33 |
| | | Mus musculus preprodipeptidyl peptidase I mRNA, complete | | | | |
| CTSC | U89269 | cds. | YES | 16.50 | 54.00 | 14.67 |
| CTSC | AA144887 | mr11d06.r1 Mus musculus cDNA, 5′ end | YES | 10.00 | 26.33 | 10.00 |
| CTSS | AA089333 | mo60e02.r1 Mus musculus cDNA, 5′ end | YES | 10.00 | 45.33 | 10.00 |
| CTSS | AA146437 | mr05a08.r1 Mus musculus cDNA, 5′ end | YES | 10.00 | 42.67 | 10.00 |
| D12ERTD647E | AA120109 | mq09a11.r1 Mus musculus cDNA, 5′ end | YES | 26.50 | 79.00 | 28.67 |
| | | Mouse 3.5-dpc-blastocyst cDNA Mus musculus cDNA clone | | | | |
| D14ERTD310E | C80103 | J0076E08 3′, mRNA sequence. | YES | 10.00 | 31.67 | 10.33 |
| | | vq57g08.r1 Barstead mouse proximal colon MPLRB6 Mus | | | | |
| D16WSU103E | AA674986 | musculus cDNA clone 1106462 5′, mRNA sequence. | YES | 11.75 | 37.67 | 10.00 |
| | | Mus musculus viral envelope like protein (G7e) gene, | | | | |
| D17H6S56E5 | U69488 | complete cds | YES | 10.00 | 22.33 | 10.00 |
| | | vo54d12.r1 Barstead mouse irradiated colon MPLRB7 Mus | | | | |
| D5WSU111E | AA638539 | musculus cDNA clone 1053719 5′, mRNA sequence. | YES | 11.25 | 47.33 | 12.67 |
| | | vq87c07.r1 Knowles Solter mouse blastocyst B3 Mus | | | | |
| D7ERTD237E | AA666918 | musculus cDNA clone 1109292 5′, mRNA sequence. | YES | 11.75 | 25.33 | 10.67 |
| | | mi15h02.r1 Soares mouse p3NMF19.5 Mus musculus cDNA | | | | |
| DIPP | AA028770 | clone 463635 5′ | YES | 36.50 | 81.33 | 57.67 |
| | | ma41d10.r1 Soares mouse p3NMF19.5 Mus musculus cDNA | | | | |
| ENTPD2 | W10995 | clone 313267 5′, mRNA sequence. | YES | 11.00 | 23.00 | 14.67 |
| FARP- | | mj76a06.r1 Soares mouse p3NMF19.5 Mus musculus cDNA | | | | |
| PENDING | AA059883 | clone 482002 5′ | YES | 10.50 | 21.33 | 13.67 |
| | | vf83b09.r1 Soares mouse mammary gland NbMMG Mus | | | | |
| | | musculus cDNA clone 850361 5′ similar to WP:C14B1.3 | | | | |
| FBXO6B | AA451220 | CE00900; | YES | 10.00 | 22.00 | 15.00 |
| | | Mus musculus TGF-beta-inducible protein (TSC-36) mRNA, | | | | |
| FSTL | M91380 | complete cds | YES | 10.00 | 20.00 | 10.00 |
| | | Mus musculus ion channel homolog RIC mRNA, complete | | | | |
| FXYD5 | U72680 | cds. | YES | 10.25 | 31.00 | 11.67 |
| GNB1 | U29055 | Mus musculus G protein beta 36 subunit mRNA, compl | YES | 11.75 | 28.33 | 15.33 |
| GRN | M86736 | Mouse acrogranin mRNA, complete cds | YES | 56.25 | 129.00 | 59.00 |
| | | Mouse tumor-induced 32 kD protein (p32) mRNA, complete | | | | |
| HMOX1 | M33203 | cds | YES | 10.00 | 20.00 | 10.33 |
| HN1 | U90123 | Mus musculus HN1 (Hn1) mRNA, complete cds. | YES | 10.00 | 23.67 | 10.00 |
| HSP25 | L07577 | Mus musculus small heat shock protein (HSP25) gene | YES | 31.75 | 131.67 | 35.67 |
| | | Mouse interferon-inducible protein homologue mRNA, | | | | |
| IFIT3 | L32974 | complete cds | YES | 13.75 | 29.00 | 11.33 |
| | | Mus musculus interferon regulatory factor 7 (mirf7) mRNA, | | | | |
| IRF7 | U73037 | complete cds | YES | 10.00 | 27.33 | 11.67 |
| ITGB4BP | AA122622 | B integrin interactor homolog | YES | 11.25 | 25.33 | #N/A |
| JUN | W09701 | ma56e02.r1 Mus musculus cDNA, 5′ end | YES | 16.25 | 32.33 | 14.67 |
| KRT2-8 | D90360 | Mouse gene for cytokeratin endo A | YES | 19.50 | 49.00 | 20.33 |
| LAPTM5 | U29539 | Mus musculus retinoic acid-inducible E3 protein mR | YES | 10.25 | 27.33 | 11.33 |
| LCN2 | X81627 | M. musculus 24p3 gene. | YES | 10.00 | 81.67 | 10.00 |
| LGALS3 | W10936 | ma03e09.r1 Mus musculus cDNA, 5′ end | YES | 10.00 | 27.33 | 10.00 |
| LOC56722 | AA542220 | TBX1 protein (novel) | YES | 14.50 | 42.67 | 13.67 |
| | | Mus musculus lymphocyte specific transcript (LST) mRNA, | | | | |
| LST1 | U72643 | partial cds. | YES | 11.00 | 29.33 | 16.00 |
| | | Mouse lyn A protein tyrosine kinase (lynA) mRNA, complete | | | | |
| LYN | M57696 | cds | YES | 14.25 | 30.00 | 16.33 |
| MAPK1 | AA104744 | MAP kinase | YES | 10.00 | 28.67 | 10.00 |
| MGLAP | D00613 | Mouse mRNA for matrix Gla protein (MGP) | YES | 47.75 | 249.67 | 48.67 |
| MKI67 | X82786 | M. musculus mRNA for Ki-67. | YES | 10.00 | 21.33 | 10.00 |
| | | mw28h11.r1 Soares mouse 3NME12 5 Mus musculus cDNA | | | | |
| | | clone 672069 5′ similar to gb:X61399 Mouse F52 mRNA for a | | | | |
| MLP | AA245242 | novel protein (MOUSE); | YES | 11.25 | 31.00 | 14.33 |
| MPEG1 | L20315 | Mus musculus MPS1 gene and mRNA, 3′end | YES | 10.00 | 30.00 | 10.00 |
| NFKBIA | U36277 | Mus musculus I-kappa B alpha chain mRNA, complete cds | YES | 14.75 | 44.00 | 16.67 |
| OAS1A | M33863 | Mouse 2′-5′ oligo A synthetase mRNA, complete cds. | YES | 11.50 | 25.00 | 10.33 |
| | | EST03133 Mouse 7.5 dpc embryo ectoplacental cone cDNA | | | | |
| P21ARC | AA408672 | library Mus musculus cDNA clone C0031D07 3′ | YES | 39.25 | 80.00 | 38.67 |
| PEA15 | AA108330 | mp28b03.r1 Mus musculus cDNA, 5′ end | YES | 11.50 | 40.00 | 14.00 |
| | | Mouse mRNA for mastocytoma proteoglycan core protein, | | | | |
| PRG | X16133 | serglycin. | YES | 18.25 | 51.33 | 18.33 |
| | | Mus musculus 20S proteasome subunit Lmp7 (Lmp7d allele) | | | | |
| PSMB8 | U22031 | mRNA, complete cds | YES | 10.25 | 41.33 | 10.00 |
| PSME2 | D87910 | Mus musculus mRNA for PA28 beta subunit, complete cds. | YES | 21.75 | 64.00 | 26.33 |
| PTMB4 | W41883 | mc64g08.r1 Mus musculus cDNA 5′ end | YES | 83.75 | 272.00 | 79.00 |
| PTPN1 | U24700 | Mus musculus protein tyrosine phosphatase (HA2) mR | YES | 10.00 | 22.00 | 10.33 |
| RAC2 | X53247 | Mus musculus EN-7 mRNA. | YES | 13.00 | 59.67 | 17.00 |
| | | vj03d05.r1 Barstead mouse pooled organs MPLRB4 Mus | | | | |
| | | musculus cDNA clone 920649 5′ similar to TR:G881954 | | | | |
| RBM3 | AA538285 | G881954 RNPL.; | YES | 13.50 | 42.00 | 16.00 |
| | | Mus musculus G protein signaling regulator RGS2 (rgs2) | | | | |
| RGS2 | U67187 | mRNA, complete cds. | YES | 10.00 | 24.33 | 12.67 |
| | | EST02956 Mouse 7.5 dpc embryo ectoplacental cone cDNA | | | | |
| | | library Mus musculus cDNA clone C0028E12 3′, mRNA | | | | |
| RPL13A | AA408475 | sequence. | YES | 11.00 | 24.33 | 14.00 |
| RRAS | M21019 | Mouse R-ras mRNA, complete cds | YES | 16.00 | 43.33 | 18.00 |
| RRAS | W41501 | mc43d11.r1 Mus musculus cDNA, 5′ end | YES | 10.25 | 21.67 | 10.00 |
| | | Mouse 3.5-dpc blastocyst cDNA Mus musculus cDNA clone | | | | |
| | | J0101H11 3′ similar to Mouse ribonucleotide reductase M2 | | | | |
| RRM2 | C81593 | subunit mRNA, mRNA sequence. | YES | 10.00 | 23.00 | 10.00 |
| | | mq98h01.r1 Soares mouse 3NbMS Mus musculus cDNA | | | | |
| SCYA19 | AA137292 | clone 596017 5′ | YES | 16.25 | 32.33 | 20.67 |
| | | Mus musculus NIH 3T3 chemokine rantes (Scya5) gene, | | | | |
| SCYA5 | U02298 | complete cds | YES | 10.00 | 22.33 | 10.00 |
| SCYD1 | U92565 | Mus musculus fractalkine mRNA, complete cds. | YES | 11.50 | 30.33 | 10.00 |
| SLC20A1 | M73696 | Murine Glvr-1 mRNA, complete cds | YES | 10.00 | 20.67 | 10.67 |
| | | Mus musculus secretory leukocyte protease inhibitor mRNA, | | | | |
| SLPI | U73004 | complete cds. | YES | 10.00 | 24.00 | 10.00 |
| SNRPD1 | M58558 | Murine sm D small nuclear ribonucleoprotein sequence. | YES | 10.00 | 20.33 | 11.00 |
| SPI2-1 | M64085 | Mouse spi2 proteinase inhibitor (spi2/eb1) mRNA, 3 | YES | 10.00 | 20.67 | 10.33 |
| | | mp09d07.r1 Life Tech mouse embryo 8 5dpc 10664019 Mus | | | | |
| SPI6 | AA108054 | musculus cDNA clone 568717 5′ | YES | 10.00 | 23.33 | 10.33 |
| | | Mus musculus signal transducer and activator of transcription | | | | |
| STAT3 | U06922 | (Stat3) mRNA, complete cds | YES | 42.25 | 99.33 | 44.33 |
| | | vb41e05.r1 Soares mouse lymph node NbMLN Mus | | | | |
| STAT3 | AA396029 | musculus cDNA clone 751520 5′ | YES | 10.00 | 20.67 | 10.67 |
| | | mp39a05.r1 Barstead MPLRB1 Mus musculus cDNA clone | | | | |
| STK2 | AA108677 | 571568 5′ | YES | 10.00 | 21.00 | 14.33 |
| | | Mus musculus (clone U2) T-cell specific protein mRNA, | | | | |
| TGTP | L38444 | complete cds | YES | 10.00 | 20.00 | 10.00 |
| TLN | X56123 | Mouse mRNA for talin | YES | 10.00 | 28.00 | 10.00 |
| UNK_AA011 | | | | | | |
| 784 | AA011784 | mg92b08.r1 Mus musculus cDNA, 5′ end | YES | 17.50 | 67.67 | 20.67 |
| UNK_AA023 | | | | | | |
| 491 | AA023491 | mh74e11.r1 Mus musculus cDNA, 5′ end | YES | 10.00 | 38.33 | 10.00 |
| UNK_AA030 | | mi22g02.r1 Soares mouse embryo NbME13.5 14.5 Mus | | | | |
| 688 | AA030688 | musculus cDNA clone 464306 5′ | YES | 10.25 | 25.67 | 10.00 |
| UNK_AA087 | | | | | | |
| 673 | AA087673 | mm27b09.r1 Mus musculus cDNA, 5′ end | YES | 10.00 | 81.67 | 11.67 |
| UNK_AA104 | | | | | | |
| 688 | AA104688 | mo55c10.r1 Mus musculus cDNA, 5′ end | YES | 10.00 | 42.67 | 10.00 |
| UNK_AA107 | | | | | | |
| 847 | AA107847 | mo49d08.r1 Mus musculus cDNA, 5′ end | YES | 10.00 | 34.67 | 10.00 |
| UNK_AA109 | | | | | | |
| 909 | AA109909 | mp10d09.r1 Mus musculus cDNA, 5′ end | YES | 10.00 | 28.67 | 10.00 |
| UNK_AA163 | | mt65a03.r1 Soares mouse lymph node NbMLN Mus | | | | |
| 096 | AA163096 | musculus cDNA clone 634732 5′ | YES | 17.25 | 45.00 | 15.67 |
| UNK_AA172 | | mr31f05.r1 Soares mouse 3NbMS Mus musculus cDNA | | | | |
| 851 | AA172851 | clone 599073 5′ | YES | 10.00 | 21.67 | 14.67 |
| UNK_AA174 | | ms77e07.r1 Soares mouse 3NbMS Mus musculus cDNA | | | | |
| 883 | AA174883 | clone 617604 5′ | YES | 25.00 | 65.67 | 25.67 |
| UNK_AA184 | | mt22f04.r1 Soares mouse 3NbMS Mus musculus cDNA | | | | |
| 116 | AA184116 | clone 621823 5′ | YES | 11.75 | 28.00 | 12.33 |
| UNK_AA210 | | mu72h03.r1 Soares mouse lymph node NbMLN Mus | | | | |
| 359 | AA210359 | musculus cDNA clone 644981 5′ | YES | 13.00 | 29.33 | 14.00 |
| | | mx94f04.r1 Soares mouse NML Mus musculus cDNA clone | | | | |
| UNK_AA238 | | 694015 5′ similar to TR:G806566 G806566 SM PROTEIN G. | | | | |
| 483 | AA238483 | ; | YES | 13.00 | 31.33 | 16.67 |
| UNK_AA538 | | vj53e12.r1 Knowles Solter mouse blastocyst B1 Mus | | | | |
| 477 | AA538477 | musculus cDNA clone 932782 5′ | YES | 11.00 | 22.67 | #N/A |
| | | vl56h09.r1 Stratagene mouse skin (#937313) Mus musculus | | | | |
| UNK_AA562 | | cDNA clone 976289 5′ similar to gb:X06753 Mouse pro- | | | | |
| 685 | AA562685 | alpha1 (MOUSE); | YES | 11.50 | 58.33 | 12.67 |
| | | vm91d04.r1 Knowles Solter mouse blastocyst B1 Mus | | | | |
| UNK_AA606 | | musculus cDNA clone 1005607 5′ similar to TR:G497940 | | | | |
| 926 | AA606926 | G497940 MAJOR VAULT PROTEIN.;, mRNA sequence. | YES | 15.25 | 35.00 | 10.33 |
| UNK_AA616 | | vo50d04.r1 Barstead mouse irradiated colon MPLRB7 Mus | | | | |
| 243 | AA616243 | musculus cDNA clone 1053319 5′, mRNA sequence. | YES | 10.00 | 21.33 | 10.33 |
| UNK_AA690 | | vu57b03.r1 Soares mouse mammary gland NbMMG Mus | | | | |
| 738 | AA690738 | musculus cDNA clone 1195469 5′, mRNA sequence. | YES | 15.50 | 36.33 | 12.67 |
| UNK_AA710 | | vt42f07.r1 Barstead mouse proximal colon MPLRB6 Mus | | | | |
| 451 | AA710451 | musculus cDNA clone 1165765 5′, mRNA sequence. | YES | 10.00 | 46.33 | 10.33 |
| UNK_AC002 | | Mouse chromosome 6 BAC-284H12 (Research Genetics | | | | |
| 397 | AC002397 | mouse BAC library) complete sequence. | YES | 10.00 | 25.00 | 15.00 |
| | | Mouse 3.5-dpc blastocyst cDNA Mus musculus cDNA clone | | | | |
| UNK_C76523 | C76523 | J0012E07 3′, mRNA sequence. | YES | 11.50 | 30.67 | 10.33 |
| | | Mouse 3.5-dpc blastocyst cDNA Mus musculus cDNA clone | | | | |
| UNK_C76523 | C76523 | J0012E07 3′, mRNA sequence. | YES | 10.00 | 23.00 | 10.00 |
| | | Mouse 3.5-dpc blastocyst cDNA Mus musculus cDNA clone | | | | |
| | | J0038G08 3′ similar to Rattus norvegicus major vault protein | | | | |
| UNK_C77861 | C77861 | mRNA, mRNA sequence. | YES | 16.50 | 35.67 | 16.67 |
| | | Mouse 3.5-dpc blastocyst cDNA Mus musculus cDNA clone | | | | |
| UNK_C80574 | C80574 | J0084D04 3′ similar to Human clone 23665 mRNA sequence. | YES | 28.00 | 60.67 | 25.00 |
| UNK_ET61420 | ET61420 | Mus musculus anti-glycoprotein-B of human Cytomegalovirus | | | | |
| | | immunoglobulin Vh chain gene, partial cds. | YES | 10.00 | 65.67 | 10.00 |
| UNK_ET63106 | ET63106 | M. musculus mRNA for immunoglobulin heavy chain variable | | | | |
| | | region, isolate 205. | YES | 10.00 | 22.33 | 10.00 |
| UNK_ET63126 | ET63126 | M. musculus mRNA for anti folate binding protein, MOv19 | | | | |
| | | Vkappa. | YES | 10.00 | 30.00 | 12.00 |
| UNK_W08057 | W08057 | mb37e05.r1 Mus musculus cDNA, 5′ end | YES | 10.00 | 48.00 | 12.33 |
| | | ma74d01.r1 Soares mouse p3NMF19.5 Mus musculus cDNA | | | | |
| | | clone 316417 5′ similar to gb:J03909 GAMMA-INTERFERON. | | | | |
| UNK_W11156 | W11156 | INDUCIBLE PROTEIN IP-30 PRECURSOR (HUMAN);, | | | | |
| | | mRNA sequence. | YES | 27.75 | 57.67 | 29.67 |
| UNK_W20873 | W20873 | mb92c11.r1 Mus musculus cDNA, 5′ end | YES | 10.00 | 32.00 | 10.00 |
| UNK_W29429 | W29429 | mb99d03.r1 Mus musculus cDNA, 5′ end | YES | 10.00 | 33.67 | 12.67 |
| UNK_W48951 | W48951 | md24g11.r1 Mus musculus cDNA, 5′ end | YES | 10.00 | 20.00 | #N/A |
| UNK_W50888 | W50888 | ma23e03.r1 Mus musculus cDNA, 5′ end | YES | 12.00 | 24.67 | 15.67 |
| UNK_W50898 | W50898 | ma23g03.r1 Mus musculus cDNA, 5′ end | YES | 15.75 | 40.33 | 18.67 |
| UNK_W57485 | W57485 | ma34h02.r1 Mus musculus cDNA, 5′ end | YES | 10.00 | 23.67 | 10.00 |
| UNK_W90837 | W90837 | mf78g07.r1 Mus musculus cDNA, 5′ end | YES | 10.75 | 33.00 | 10.00 |
| UNK_X52622 | X52622 | Mouse IN gene for the integrase of an endogenous retrovirus | YES | 10.25 | 20.33 | 15.00 |
| | | ma89d07.r1 Soares mouse p3NMF19.5 Mus musculus cDNA | | | | |
| | | clone 317869 5′ similar to gb:X57352 INTERFERON- | | | | |
| VCP | W12941 | INDUCIBLE PROTEIN 1-8U (HUMAN);, mRNA sequence. | YES | 31.00 | 121.33 | 32.33 |
| | | House mouse; Musculus domesticus mRNA for 14-3-3 eta, | | | | |
| YWHAH | D87661 | complete cds | YES | 10.50 | 22.00 | 10.00 |
| ACINUS- | | vf79g11.r1 Soares mouse mammary gland NbMMG Mus | | | | |
| PENDING | AA444568 | musculus cDNA clone 850052 5′ | NO | 10.00 | 21.67 | 19.00 |
| APOE | AA048604 | mj32g02.r1 Mus musculus cDNA, 5′ end | NO | 70.75 | 236.67 | 86.00 |
| ARHC | X80638 | M. musculus rhoC mRNA. | NO | 47.00 | 104.67 | 57.67 |
| BGN | L20276 | Mouse biglycan (Bgn) mRNA, complete cds | NO | 71.25 | 169.33 | 64.00 |
| CAPPB1 | U10406 | Mus musculus capping protein beta-subunit isoform | NO | 35.75 | 72.67 | 47.33 |
| | | M. musculus intracisternal A-particle IAP-IL3 genome deleted | | | | |
| CCR4 | X04120 | type I element inserted 5′ to the interleukin-3 gene. | NO | 50.00 | 112.33 | 93.33 |
| CD36L2 | AB008553 | Mus musculus mRNA for mLGP85/LIMP II, complete cds. | NO | 10.25 | 21.00 | 17.00 |
| CFL1 | D00472 | Mouse mRNA for cofilin, complete cds and flanks | NO | 28.25 | 81.00 | 47.67 |
| CLU | L08235 | Mus musculus clusterin mRNA, complete cds | NO | 163.25 | 415.33 | 117.33 |
| CP | U49430 | Mus musculus ceruloplasmin mRNA, complete cds | NO | 20.50 | 69.00 | 30.33 |
| | | mi67c05.r1 Soares mouse embryo NbME13.5 14.5 Mus | | | | |
| D11ERTD172E | AA014563 | musculus cDNA clone 468584 5′. | NO | 38.25 | 77.33 | 56.33 |
| | | vu31g07.r1 Stratagene mouse Tcell 937311 Mus musculus | | | | |
| | | cDNA clone 1193052 5′ similar to SW:INI7_HUMAN P40305 | | | | |
| | | INTERFERON-ALPHA INDUCED 11.5 KD PROTEIN;, | | | | |
| D12ERTD647E | AA711625 | mRNA sequence. | NO | 102.00 | 317.67 | 124.67 |
| | | vp33f01.r1 Barstead mouse proximal colon MPLRB6 Mus | | | | |
| D17WSU91E | AA727845 | musculus cDNA clone 1078489 5′, mRNA sequence. | NO | 84.50 | 189.67 | 96.67 |
| | | EST01599 Mouse 7.5 dpc embryo ectoplacental cone cDNA | | | | |
| | | library Mus musculus cDNA clone C0012A02 3′, mRNA | | | | |
| D4WSU27E | AA409826 | sequence. | NO | 34.50 | 78.00 | 24.33 |
| EEF2 | W98531 | elongation factor 2 (ef-2) | NO | 11.50 | 35.00 | 36.00 |
| | | Mus musculus FK506 binding protein 51 mRNA, complete | | | | |
| FKBP5 | U36220 | cds | NO | 14.25 | 28.33 | 26.67 |
| FTH | W18308 | mb68h11.r1 Mus musculus cDNA, 5′ end | NO | 331.75 | 695.33 | 618.67 |
| GAS5 | X59728 | M. musculus mRNA for gas5 growth arrest specific protein | NO | 14.00 | 36.33 | 24.67 |
| GP49A | M65027 | Mouse cell surface antigen gp49 mRNA, complete cds | NO | 14.00 | 28.33 | 24.67 |
| | | mi95f04.r1 Soares mouse p3NMF19.5 Mus musculus cDNA | | | | |
| HZF- | | clone 474367 5′ similar to gb:U27830 Mus musculus extendin | | | | |
| PENDING | AA038775 | mRNA, complete cds (MOUSE); | NO | 13.75 | 43.00 | #N/A |
| IRF1 | M21065 | Mouse interferon regulatory factor 1 mRNA, complete cds | NO | 12.00 | 40.67 | 20.67 |
| JUND1 | X15358 | Mouse mRNA for junD proto-oncogene. | NO | 53.75 | 109.33 | 87.00 |
| KCNJ11 | D50581 | Mouse mRNA for inward rectifier K+ channel | NO | 10.50 | 23.00 | 17.67 |
| KLF2 | U25096 | Mus musculus Kruppel-like factor LKLF mRNA, complete cds | NO | 10.00 | 26.67 | 15.67 |
| KPNA2 | C79184 | nuclear pore-targeting complex, mRNA sequence. | NO | 33.75 | 101.33 | 48.67 |
| L1MD-TF14 | D84391 | Mouse L1 repetitive element, complete sequence. | NO | 14.00 | 43.33 | 31.67 |
| LGALS1 | X66532 | M. musculus mRNA for L14 lectin. | NO | 34.75 | 190.33 | 49.67 |
| LGALS1 | W13002 | mb21e10.r1 Mus musculus cDNA, 5′ end | NO | 26.00 | 151.67 | 34.00 |
| LGALS3 | X16834 | Mouse mRNA for Mac-2 antigen | NO | 29.00 | 116.67 | 43.33 |
| | | Mus musculus C57BL/6 Sca-2 precursor mRNA, complete | | | | |
| LY6E | U04268 | cds. | NO | 91.00 | 362.67 | 98.33 |
| | | mg36a03.r1 Soares mouse embryo NbME13.5 14.5 Mus | | | | |
| LY6E | AA000467 | musculus cDNA clone 425836 5′. | NO | 26.75 | 73.00 | 65.00 |
| | | Mouse retinoic acid-responsive protein (MK) mRNA, | | | | |
| MDK | M35833 | complete cds | NO | 33.25 | 105.67 | 26.33 |
| | | Mouse retinoic acid-responsive protein (MK) gene, complete | | | | |
| MDK | M34094 | cds | NO | 30.25 | 90.67 | 23.00 |
| | | Mouse microfibril-associated glycoprotein (Magp) mRNA, | | | | |
| MFAP2 | L23769 | complete cds | NO | 10.50 | 21.67 | #N/A |
| MT2 | AA109597 | metallothione 2 | NO | 24.25 | 69.00 | 76.33 |
| PEA15 | L31958 | Mus musculus (clone: pMAT1) mRNA, complete cds | NO | 34.25 | 77.67 | 19.67 |
| PPICAP | X67809 | M. musculus mama mRNA. | NO | 15.25 | 78.00 | 10.00 |
| | | Mouse 3.5-dpc blastocyst cDNA Mus musculus cDNA clone | | | | |
| | | J0087G04 3′ similar to Rat mRNA for proteasome subunit | | | | |
| PSMA3 | C80757 | RC8, mRNA sequence. | NO | 12.25 | 27.00 | 21.00 |
| | | mg75a06.r1 Soares mouse embryo NbME13.5 14.5 Mus | | | | |
| | | musculus cDNA clone 438802 5′ similar to gb:D00763 | | | | |
| PSMA4 | AA008321 | PROTEASOME COMPONENT C9 (HUMAN);. | NO | 41.50 | 88.00 | 74.33 |
| RAB11A | D50500 | Mouse mRNA for Rab 11, partial sequence. | NO | 18.00 | 41.67 | 24.67 |
| | | Mouse 3.5-dpc blastocyst cDNA Mus musculus cDNA clone | | | | |
| | | J0030G04 3′ similar to Mouse B10.VL30LTR gene, 5′ flank, | | | | |
| RBPSUH | C77421 | mRNA sequence. | NO | 88.25 | 197.00 | 102.00 |
| RPL22 | D17653 | Mouse mRNA for HBp15/L22, complete cds | NO | 71.50 | 147.33 | 93.67 |
| S100A10 | M16465 | Mouse calpactin I light chain (p11) mRNA, complete cds | NO | 40.00 | 96.67 | 34.00 |
| | | Mus musculus endothelial monocyte-activating polypeptide I | | | | |
| S100A11 | U41341 | mRNA, complete cds. | NO | 24.25 | 120.67 | 30.00 |
| | | Mouse pEL98 protein mRNA which is enhanced in | | | | |
| S100A4 | D00208 | established cells, Balb/c373 | NO | 14.50 | 35.33 | 24.67 |
| S100A6 | M37761 | Mouse calcyclin mRNA, complete cds | NO | 21.50 | 161.67 | 50.00 |
| S100A6 | X66449 | M. musculus mRNA for calcyclin | NO | 10.00 | 23.67 | 15.33 |
| | | Mus musculus C57BL/6J Sec61 protein complex gamma | | | | |
| SEC61G | U11027 | subunit mRNA, complete cds | NO | 37.00 | 75.67 | 48.67 |
| | | Mus musculus selenoprotein W (mSelW) mRNA, complete | | | | |
| SEPW1 | AF015284 | cds. | NO | 24.75 | 50.67 | 31.67 |
| SPRR1A | X91824 | M. musculus mRNA for SPRR1a protein. | NO | 11.25 | 68.67 | 17.33 |
| STAT5A | U21103 | Mus musculus mammary gland factor (Stat5a) mRNA, c | NO | 10.75 | 26.33 | 26.33 |
| TAGLN | L41154 | Mus musculus SM22 alpha mRNA, complete cds | NO | 20.50 | 79.67 | 26.33 |
| TGFB1I4 | X62940 | M. musculus TSC-22 mRNA. | NO | 137.50 | 306.33 | 168.67 |
| UCP2 | U69135 | Mus musculus UCP2 mRNA, complete cds.) | NO | 14.50 | 75.33 | 21.00 |
| UNK_AA000 | | mg24e05.r1 Soares mouse embryo NbME13.5 14.5 Mus | | | | |
| 380 | AA000380 | musculus cDNA clone 424736 5′. | NO | 28.00 | 63.00 | 42.67 |
| UNK_AA002 | | mg38h07.r1 Soares mouse embryo NbME13.5 14.5 Mus | | | | |
| 653 | AA002653 | musculus cDNA clone 426109 5′. | NO | 12.25 | 31.67 | 27.67 |
| UNK_AA004 | | mg80f01.r1 Soares mouse embryo NbME13.5 14.5 Mus | | | | |
| 011 | AA004011 | musculus cDNA clone 439321 5′. | NO | 10.00 | 20.67 | 15.67 |
| UNK_AA028 | | mi14h12.r1 Soares mouse p3NMF19.5 Mus musculus cDNA | | | | |
| 657 | AA028657 | clone 463559 5′ | NO | 28.75 | 59.33 | 55.00 |
| UNK_AA038 | | | | | | |
| 347 | AA038347 | mi84d05.r1 Mus musculus cDNA, 5′ end | NO | 10.50 | 38.33 | 17.33 |
| UNK_AA038 | | | | | | |
| 511 | AA038511 | mi85h01.r1 Mus musculus cDNA, 5′ end | NO | 28.50 | 113.00 | 47.67 |
| UNK_AA068 | | | | | | |
| 158 | AA068158 | mm56e10.r1 Mus musculus cDNA, 5′ end | NO | 26.25 | 81.00 | 45.33 |
| UNK_AA097 | | | | | | |
| 626 | AA097626 | mo08g01.r1 Mus musculus cDNA, 5′ end | NO | 29.50 | 176.33 | 47.00 |
| UNK_AA168 | | | | | | |
| 865 | AA168865 | ms38c08.r1 Mus musculus cDNA, 5′ end | NO | 11.25 | 35.67 | 18.33 |
| UNK_AA184 | | mt58c09.r1 Soares 2NbMT Mus musculus cDNA clone | | | | |
| 455 | AA184455 | 634096 5′ | NO | 10.00 | 21.00 | 20.33 |
| UNK_AA617 | | vi21f09.r1 Barstead mouse proximal colon MPLRB6 Mus | | | | |
| 093 | AA617093 | musculus cDNA clone 904457 5′, mRNA sequence. | NO | 10.75 | 21.33 | 17.00 |
| UNK_AA711 | | vt56c05.r2 Barstead mouse irradiated colon MPLRB7 Mus | | | | |
| 130 | AA711130 | musculus cDNA clone 1167080 5′, mRNA sequence. | NO | 149.75 | 321.33 | 221.00 |
| | | Mouse 3.5-dpc blastocyst cDNA Mus musculus cDNA clone | | | | |
| | | J0004G06 3′ similar to Rat insulin-I (ins-1) gene, mRNA | | | | |
| UNK_C76162 | C76162 | sequence. | NO | 11.50 | 42.33 | 30.67 |
| | | Mouse 3.5-dpc blastocyst cDNA Mus musculus cDNA clone | | | | |
| | | J0032G04 3′ similar to Rat G protein gamma-5 subunit, | | | | |
| UNK_C77514 | C77514 | mRNA sequence. | NO | 90.75 | 190.67 | 133.00 |
| | | Mouse 3.5-dpc blastocyst cDNA Mus musculus cDNA clone | | | | |
| | | J0051B02 3′ similar to moesin homolog [mice, | | | | |
| UNK_C78546 | C78546 | teratocarcinoma F9 cells, mRNA, mRNA sequence. | NO | 40.25 | 87.33 | 56.33 |
| UNK_M26005 | M26005 | Mouse endogenous retrovirus truncated gag protein, | | | | |
| | | complete cds, clone del env-1 3.1 | NO | 12.25 | 61.67 | 24.33 |
| UNK_M29325 | M29325 | Mouse L1Md-9 repetitive sequence (EXTRACTED 3′UTR) | NO | 13.75 | 36.67 | 29.33 |
| | | MDB1515 Mouse brain, Stratagene Mus musculus cDNA | | | | |
| UNK_N28179 | N28179 | 3′ end. | NO | 12.75 | 32.33 | 37.33 |
| | | MDB0793 Mouse brain, Stratagene Mus musculus cDNA | | | | |
| UNK_R74638 | R74638 | 3′end. | NO | 13.00 | 27.00 | 26.67 |
| UNK_W11954 | W11954 | ma79e11.r1 Mus musculus cDNA, 5′ end | NO | 12.75 | 30.67 | 23.33 |
| UNK_W18503 | W18503 | mb88b08.r1 Mus musculus cDNA, 5′ end | NO | 12.25 | 25.00 | 18.33 |
| VCP | AA002526 | mg54a04.r1 Mus musculus cDNA, 5′ end | NO | 18.50 | 84.33 | 26.00 |

TABLE 6
Rapamycin-Normalized Genes Clustered by Function

| | | delta | Avg. | Avg. | Avg. | |
|---|---|---|---|---|---|---|
| Name | Acc. # | r36-u12 | Untr12w | Untr.36w | Rapa36w | Description |

Antigen Presentation

| | | | | | | |
|---|---|---|---|---|---|---|
| H2 | M69069 | 0.00 | 10.00 | 21.00 | 10.00 | Mus Musculus mRNA, complete cds |
| PSME2 | d87910 | 4.58 | 21.75 | 64.00 | 26.33 | Mus musculus mRNA for PA28 beta subunit, complete cds. |
| | | | | | | Mus musculus Balb/c proteasome subunit (Imp7) gene, |
| LMP7 | I11145 | 0.00 | 10.00 | 31.00 | 10.00 | complete cds and intergenic region. |
| | | | | | | Mus musculus domesticus MHC class II antigen H-2E alpha |
| H2_EA | u13648 | −0.17 | 13.50 | 91.67 | 13.33 | precursor (allele w29) mRNA, complete cds |
| LA_LI | X00496 | −0.33 | 60.00 | 363.33 | 59.67 | Mouse Ia-associated invariant chain (Ii) mRNA fragment. |
| | | | | | | Mouse MHC class II H2-IA-alpha gene (d haplotype) mRNA, |
| H2_AA | k01923 | 3.17 | 38.50 | 152.33 | 41.67 | complete cds |
| | | | | | | K01923 Mouse MHC class II H2-IA-alpha gene (d haplotype) |
| H2_AA | k01923 | 1.33 | 50.00 | 187.67 | 51.33 | mRNA, complete cds |
| | | | | | | V00832 Mouse fragment of mRNA encoding for the Ia antigen |
| | | | | | | (heavy chain) from major histocompatibility complex (A-k- |
| H2_AA | v00832 | −3.42 | 41.75 | 134.00 | 38.33 | alpha). This is coded by the I-A region of the MHC and |

Tissue Remodeling and Repair

| | | | | | | |
|---|---|---|---|---|---|---|
| FISP12 | m70642 | −3.17 | 19.50 | 83.00 | 16.33 | Mouse FISP-12 protein (fisp-12) mRNA, complete cds |
| ETV6 | d00613 | 0.92 | 47.75 | 249.67 | 48.67 | D00613 Mouse mRNA for matrix Gla protein (MGP) |
| TUBB5 | x04663 | 3.58 | 19.75 | 60.67 | 23.33 | Mouse mRNA for beta-tubulin (isotype Mbeta5). |
| TUBB5 | x04663 | 3.25 | 21.75 | 55.33 | 25.00 | X04663 Mouse mRNA for beta-tubulin (isotype Mbeta 5) |
| COL6A2 | x65582 | 1.08 | 11.25 | 33.33 | 12.33 | M. musculus mRNA for alpha-2 collagen VI. |
| | | | | | | Mus musculus FVB/N collagen pro-alpha-1 type I chain |
| COLA1 | u08020 | 0.00 | 10.00 | 30.00 | 10.00 | mRNA, complete cds |
| | | | | | | U08020 Mus musculus FVB/N collagen pro-alpha-1 type I |
| COLA1 | u08020 | −2.00 | 12.00 | 44.33 | 10.00 | chain mRNA, complete cds |
| CNN2 | z19543 | 3.75 | 15.25 | 34.33 | 19.00 | Z19543 M. musculus h2-calponin cDNA |
| COL6A | x66405 | −0.58 | 11.25 | 24.67 | 10.67 | M. musculus mRNA for collagen alpha1 (VI)-collagen. |
| COLA2 | X58251 | 0.00 | 10.00 | 52.67 | 10.00 | Mouse COL1A2 mRNA for pro-alpha-2 (I) collagen. |
| COLA2 | x58251 | 0.00 | 10.00 | 38.00 | 10.00 | X58251 Mouse COL1A2 mRNA for pro-alpha-2(I) collagen. |
| ACTVS | X13297 | 1.67 | 37.00 | 148.67 | 38.67 | Mouse mRNA for vascular smooth muscle alpha-actin. |
| FN | M18194 | −3.17 | 13.50 | 51.00 | 10.33 | Mouse fibronectin (FN) mRNA |
| FN | m18194 | −3.50 | 13.50 | 38.67 | 10.00 | M18194 Mouse fibronectin (FN) mRNA |
| KRT2_8 | d90360 | 0.83 | 19.50 | 49.00 | 20.33 | Mouse gene for cytokeratin endo A |
| FBN1 | I29454 | 0.33 | 10.00 | 20.33 | 10.33 | Mouse fibrillin (Fbn-1) mRNA, complete cds |
| x56123-2 | x56123 | 0.00 | 10.00 | 28.00 | 10.00 | Mouse mRNA for talin. |
| ACTG2 | u20365 | 4.33 | 13.00 | 26.33 | 17.33 | Mus musculus smooth muscle gamma-actin gene |
| GRN | m86736 | 2.75 | 56.25 | 129.00 | 59.00 | Mouse acrogranin mRNA, complete cds |
| SPARC | x04017 | −4.83 | 24.50 | 78.67 | 19.67 | X04017 Mouse mRNA for cysteine-rich glycorotein SPARC |

Complement

| | | | | | | |
|---|---|---|---|---|---|---|
| | | | | | | Mouse complement component C3 mRNA, alpha and beta |
| K02782 | k02782 | −3.92 | 23.25 | 178.67 | 19.33 | subunits, complete cds |
| | | | | | | Mouse mRNA for complement subcomponent C1Q alpha- |
| C1QA | X58861 | 0.00 | 10.00 | 66.67 | 10.00 | chain. |
| C1QC | X66295 | 1.42 | 10.25 | 57.00 | 11.67 | M. musculus mRNA for C1q C-chain. |
| | | | | | | M22531 Mouse complement C1q B chain mRNA, complete |
| C1QB | m22531 | −0.33 | 11.00 | 58.67 | 10.67 | cds |
| X16874 | X16874 | 0.33 | 10.00 | 47.00 | 10.33 | Mouse mRNA for complement protein C1q B-chain. |
| C1NH | Y10386 | 4.67 | 37.00 | 94.00 | 41.67 | M. musculus mRNA for C1 inhibitor. |

Protease Inhibitor

| | | | | | | |
|---|---|---|---|---|---|---|
| | | | | | | M64085 Mouse spi2 proteinase inhibitor (spi2/eb1) mRNA, 3′ |
| SPI2_1 | m64085 | 0.33 | 10.00 | 20.67 | 10.33 | end |
| | | | | | | Mus musculus secretory leukocyte protease inhibitor mRNA, |
| SLPI | u73004 | 0.00 | 10.00 | 24.00 | 10.00 | complete cds. |
| SPI3 | U25844 | 0.92 | 10.75 | 25.67 | 11.67 | Mus musculus serine proteinase inhibitor (SPI3) mR |
| CSTB | U59807 | 3.83 | 14.50 | 68.00 | 18.33 | Mus musculus cystatin B (Stfb) gene, complete cds. |

Transcription Factors

| | | | | | | |
|---|---|---|---|---|---|---|
| | | | | | | U36277 Mus musculus I-kappa B alpha chain mRNA, |
| NFKBIA | u36277 | 1.92 | 14.75 | 44.00 | 16.67 | complete cds |
| | | | | | | U36277 Mus musculus I-kappa B alpha chain mRNA, |
| NFKBIA | u36277 | −0.42 | 17.75 | 44.67 | 17.33 | complete cds |
| | | | | | | Mus musculus signal transducer and activator of transcription |
| STAT3 | u06922 | 2.08 | 42.25 | 99.33 | 44.33 | (Stat3) mRNA, complete cds |
| CEBPB | x62600 | 0.00 | 10.00 | 22.33 | 10.00 | M. musculus mRNA for C/EBP beta |
| | | | | | | Mouse cysteine-rich intestinal protein (CRIP) mRNA, |
| CRIP | M13018 | 4.33 | 10.00 | 49.33 | 14.33 | complete cds |
| | | | | | | M13018 Mouse cysteine-rich intestinal protein (CRIP) mRNA, |
| CRIP | m13018 | 3.08 | 10.25 | 48.00 | 13.33 | complete cds |

Interferon Related

| | | | | | | |
|---|---|---|---|---|---|---|
| IFNGR | j05265 | 2.25 | 12.75 | 27.67 | 15.00 | Mouse interferon gamma receptor mRNA, complete cds |
| | | | | | | Mouse interferon-inducible protein homologue mRNA, |
| IFI49 | I32974 | −2.42 | 13.75 | 29.00 | 11.33 | complete cds |
| | | | | | | Mus musculus interferon regulatory factor 7 (mirf7) mRNA, |
| MIRF7 | U73037 | 1.67 | 10.00 | 27.33 | 11.67 | complete cds |
| | | | | | | Messenger RNA fragment for mouse interferon beta (type 1) |
| IFNB | v00755 | 0.00 | 10.00 | 30.33 | 10.00 | coding for the c-terminal part. |
| | | | | | | ma/74d01.R1 Soares mouse p3NMF19.5 Mus musculus cDNA |
| | | | | | | clone 316417 5′ similar to gb:J03909 GAMMA-INTERFERON- |
| E_TC22922 | w11156 | 1.92 | 27.75 | 57.67 | 29.67 | INDUCIBLE PROTEIN IP-30 PRECURSOR (HUMAN);, |

Protease

| | | | | | | |
|---|---|---|---|---|---|---|
| | | | | | | Mus musculus preprodipeptidyl peptidase I mRNA, complete |
| CTSC | u89269 | −1.83 | 16.50 | 54.00 | 14.67 | cds. |

Heat shock proteins

| | | | | | | |
|---|---|---|---|---|---|---|
| HSP25 | I07577 | 3.92 | 31.75 | 131.67 | 35.67 | Mus musculus small heat shock protein (HSP25) gene |

Phosphatase

| | | | | | | |
|---|---|---|---|---|---|---|
| MBPTP1b | u24700 | 0.33 | 10.00 | 22.00 | 10.33 | Mus musculus protein tyrosine phosphatase (HA2) mR |
| | | | | | | The annexins are a family of proteins that bind anionic |
| | | | | | | phospholipid surfaces in a Ca(2+)-dependent manner |

Calcium binding proteins

| | | | | | | |
|---|---|---|---|---|---|---|
| LPC1 | x07486 | −0.33 | 15.00 | 36.00 | 14.67 | Mouse mRNA for lipocortin I. |
| CAL1H | m14044 | 5.00 | 22.00 | 139.67 | 27.00 | Mouse calpactin I heavy chain (p36) mRNA, complete cds |
| | | | | | | D10024 Mouse mRNA for protein-tyrosine kinase substrate |
| CAL1H | d10024 | 1.17 | 20.50 | 105.67 | 21.67 | p36 (calpactin I heavy chain), complete cds |
| ANX5 | u29396 | 0.33 | 13.00 | 40.00 | 13.33 | Mus musculus annexin V (Anx5) mRNA, complete cds |

Tcell

| | | | | | | |
|---|---|---|---|---|---|---|
| | | | | | | Mus musculus (clone U2) T-cell specific protein mRNA, |
| L38444 | I38444 | 0.00 | 10.00 | 20.00 | 10.00 | complete cds |

Bcell

| | | | | | | |
|---|---|---|---|---|---|---|
| | | | | | | Mouse B-cell differentiation antigen Lyb-2.1 protein, complete |
| TESK1 | J04170 | 0.67 | 10.00 | 22.67 | 10.67 | cds |
| | | | | | | L28060 Ig B cell antigen receptor gene, |
| IGBCR1 | L28060 | 0.00 | 10.00 | 21.00 | 10.00 | complete cds |

Tumor Induced

| | | | | | | |
|---|---|---|---|---|---|---|
| | | | | | | Mouse tumor-induced 32 kD protein (p32) mRNA, complete |
| HMOX1 | m33203 | 0.33 | 10.00 | 20.00 | 10.33 | cds |

Cytokine related

| | | | | | | |
|---|---|---|---|---|---|---|
| TGFBI | L19932 | 0.00 | 10.00 | 30.33 | 10.00 | Mouse (beta-ig-h3) mRNA, complete cds |
| | | | | | | Mus musculus TGF-beta-inducible protein (TSC-36) mRNA, |
| FSTL | M91380 | 0.00 | 10.00 | 20.00 | 10.00 | complete cds |
| | | | | | | Mus musculus NIH 3T3 chemokine rantes (Scya5) gene, |
| SCYA5 | u02298 | 0.00 | 10.00 | 22.33 | 10.00 | complete cds |
| SCYD1 | u92565 | −1.50 | 11.50 | 30.33 | 10.00 | Mus musculus fractalkine mRNA, complete cds. |

Parvalbumin

| | | | | | | |
|---|---|---|---|---|---|---|
| PVA | x67141 | −2.67 | 28.00 | 10.00 | 25.33 | M. musculus Pva mRNA for parvalbumin. |

Autoantigen

| | | | | | | |
|---|---|---|---|---|---|---|
| SNRPD1 | M58558 | 1.00 | 10.00 | 20.33 | 11.00 | Murine sm D small nuclear ribonucleoprotein sequence. |

Adhesion molecules

| | | | | | | |
|---|---|---|---|---|---|---|
| VCAM1 | x67783 | 0.00 | 10.00 | 52.00 | 10.00 | M. musculus VCAM-1 mRNA. |

Proto-cncogene

| | | | | | | |
|---|---|---|---|---|---|---|
| RAC2 | X53247 | 4.00 | 13.00 | 59.67 | 17.00 | M. musculus EN-7 mRNA. |
| RRAS | M21019 | 2.00 | 16.00 | 43.33 | 18.00 | Mouse R-ras mRNA, complete cds |

Retinoic response elements

| | | | | | | |
|---|---|---|---|---|---|---|
| LAPTM5 | u29539 | 1.08 | 10.25 | 27.33 | 11.33 | Mus musculus retinoic acid-inducibhle E3 protein mR |
| | | | | | | House mouse; Musculus domesticus mRNA for 14-3-3 eta, |
| YWHAH | d87661 | −0.50 | 10.50 | 22.00 | 10.00 | complete cds |

Retrovirus related

| | | | | | | |
|---|---|---|---|---|---|---|
| | | | | | | X52622 Mouse IN gene for the integrase of an endogenous |
| IN | x52622 | 4.75 | 10.25 | 20.33 | 15.00 | retrovirus |
| GLVR1 | M73696 | 0.67 | 10.00 | 20.67 | 10.67 | Murine Glvr-1 mRNA, complete cds |
| | | | | | | Mus musculus viral envelope like protein (G7e) gene, |
| D17NKI7 | U69488 | 0.00 | 10.00 | 22.33 | 10.00 | complete cds |
| | | | | | | Mouse lyn A protein tyrosine kinase (lynA) mRNA, complete |
| LYN | m57696 | 2.08 | 14.25 | 30.00 | 16.33 | cds |
| E_POL | aa087673 | 1.67 | 10.00 | 81.67 | 11.67 | AA087673 mm27b09.r1 Mus musculus cDNA, 5′ end |

G protein related

| | | | | | | |
|---|---|---|---|---|---|---|
| | | | | | | Mus musculus G protein signaling regulator RGS2 (rgs2) |
| U67187 | U67187 | 2.67 | 10.00 | 24.33 | 12.67 | mRNA, complete cds. |
| GNB1 | U29055 | 3.58 | 11.75 | 28.33 | 15.33 | Mus musculus G protein beta 36 subunit mRNA, compl |

Metallochaperones

| | | | | | | |
|---|---|---|---|---|---|---|
| | | | | | | Mus musculus copper transport protein Atox1 (ATOX1) |
| AF004591 | AF004591 | −0.58 | 44.25 | 90.00 | 43.67 | mRNA, complete cds. |

Clostridlum perfringens enterotoxin

| | | | | | | |
|---|---|---|---|---|---|---|
| | | | | | | Mus musculus mCPE-R mRNA for CPE-receptor, complete |
| AB000713 | AB000713 | 0.00 | 10.00 | 23.00 | 10.00 | cds. |
| | | | | | | Mus musculus mCPE-R mRNA for CPE-receptor, complete |
| AB000713 | AB000713 | 2.33 | 16.00 | 48.67 | 18.33 | cds. |

Need to bin

| | | | | | | |
|---|---|---|---|---|---|---|
| VIM | x51433 | 0.08 | 20.25 | 75.00 | 20.33 | Mouse mRNA for vimentin. |
| LCN2 | x81627 | 0.00 | 10.00 | 81.67 | 10.00 | M. musculus 24p3 gene. |
| M33863 | m33863 | −1.17 | 11.50 | 25.00 | 10.33 | Mouse 2′-5′ oligo A synthetase mRNA, complete cds. |
| | | | | | | Mouse CD14 mRNA for myelid cell-specific leucine-rich |
| CD14 | x13333 | 4.17 | 25.50 | 89.33 | 29.67 | glycoprotein. |
| AB009287 | ab009287 | 2.67 | 10.00 | 23.33 | 12.67 | Mus musculus gene for Macrosialin, complete cds. |
| CD80 | m55561 | 0.00 | 10.00 | 31.33 | 10.00 | Mouse phosphatidylinositol-linked antigen (pB7) mR |
| | | | | | | Mus musculus lymphocyte specific transcript (LST) mRNA, |
| LST1 | U72643 | 5.00 | 11.00 | 29.33 | 16.00 | partial cds. |
| TPM_I2 | m22479 | 1.33 | 20.00 | 60.00 | 21.33 | Mouse tropomyosin isoform 2 mRNA, complete cds |
| x15373-2 | x15373 | −1.92 | 42.25 | 20.00 | 40.33 | Mouse cerebellum mRNA for P400 protein. |
| MPS1 | I20315 | 0.00 | 10.00 | 30.00 | 10.00 | L20315 Mus musculus MPS1 gene and mRNA, 3′ end |
| | | | | | | Mouse mRNA for mastocytoma proteoglycan core protein, |
| SERGLYCIN | x16133 | 0.08 | 18.25 | 51.33 | 18.33 | serglycin. |
| | | | | | | Mus musculus GDP-dissociation inhibitor mRNA, |
| ARHGDIB | L07918 | 0.00 | 10.00 | 26.00 | 10.00 | preferentially expressed in hematopoietic cells, complete |
| | | | | | | cds |
| MKI67 | X82786 | 0.00 | 10.00 | 21.33 | 10.00 | M. musculus mRNA for Ki-67. |
| AF032466 | af032466 | 2.08 | 10.25 | 21.33 | 12.33 | Mus musculus arginase II mRNA, complete cds. |
| | | | | | | Mouse mRNA for secretory protein containing |
| ADAMTS1 | D67076 | 0.00 | 10.00 | 36.00 | 10.00 | thrombospondin motifs, complete cds. |
| | | | | | | Mus musculus ion channel homolog RIC mRNA, complete |
| U72680 | U72680 | 1.42 | 10.25 | 31.00 | 11.67 | cds. |
| HN1 | U90123 | 0.00 | 10.00 | 23.67 | 10.00 | Mus musculus HN1 (Hn1) mRNA, complete cds. |

Immunoglobulin

| | | | | | | |
|---|---|---|---|---|---|---|
| ET62984 | ET62984 | 2.00 | 10.00 | 66.00 | 12.00 | M. musculus mRNA (3C10) for IgA V-D-J-heavy chain. |
| ET62983 | ET62983 | 4.00 | 11.00 | 56.00 | 15.00 | M. musculus mRNA (2F7) for IgA V-D-J-heavy chain. |
| IGA_VDJ | x94418 | 3.92 | 11.75 | 60.00 | 15.67 | X94418 M. musculus mRNA (2F7) for IgA V-D-J-heavy chain |
| | | | | | | Mus musculus anti-DNA immunoglobulin heavy chain IgM |
| ET61802 | ET61802 | 2.25 | 10.75 | 22.00 | 13.00 | mRNA, antibody 373p.72, partial cds. |
| | | | | | | Mus musculus anti-DNA immunoglobulin heavy chain variable |
| ET61285 | ET61285 | 0.00 | 10.00 | 52.00 | 10.00 | region, clone 4B2, partial cds. |
| | | | | | | Mus musculus anti-DNA immunoglobulin heavy chain variable |
| ET61286 | ET61286 | 0.00 | 10.00 | 49.33 | 10.00 | region, clone 20F4, partial cds. |
| | | | | | | Mus musculus anti-DNA immunoglobulin heavy chain variable |
| ET61287 | ET61287 | 0.33 | 10.00 | 44.33 | 10.33 | region, clone 8D8, partial cds. |
| | | | | | | Mus musculus anti-DNA immunoglobulin heavy chain variable |
| E761288 | ET61288 | 0.67 | 10.00 | 23.00 | 10.67 | region, clone 22F8, partial cds. |
| | | | | | | Mus musculus anti-DNA immunoglobulin light chain variable |
| ET61296 | ET61296 | 0.00 | 10.00 | 33.33 | 10.00 | region, clone 22F8, partial cds. |
| | | | | | | Mus musculus anti-glycoprotein-B of human Cytomegalovirus |
| ET61420 | ET61420 | 0.00 | 10.00 | 65.67 | 10.00 | immunoglobulin Vh chain gene, partial cds. |
| | | | | | | Mus musculus immunoglobulin heavy chain mRNA, V, D, and |
| ET61464 | ET61464 | 0.00 | 10.00 | 23.00 | 10.00 | J segments, partial cds. |
| | | | | | | Mus musculus IgG rearranged heavy chain mRNA, variable |
| ET61520 | ET61520 | 0.00 | 10.00 | 45.00 | 10.00 | region partial cds. |
| | | | | | | Mus musculus monocional antibody against hepatitis B |
| | | | | | | surface antigen, IgG light chain variable region gene, partial |
| ET61599 | ET61599 | 1.00 | 10.00 | 42.00 | 11.00 | cds. |
| | | | | | | Mus musculus clone 1G2 IgG anti-nucleosome heavy chain |
| ET61660 | ET61660 | 0.00 | 10.00 | 53.33 | 10.00 | variable region mRNA, partial cds. |
| | | | | | | Mus musculus clone 4F7 IgG anti-nucleosome heavy chain |
| ET61662 | ET61662 | 0.00 | 10.00 | 21.33 | 10.00 | variable region mRNA, partial cds. |
| | | | | | | Mus musculus Ig 2G11.E2 heavy chain mRNA, specific for rat |
| ET61727 | ET61727 | 0.00 | 10.00 | 29.00 | 10.00 | (mouse) cytochrome c, partial cds. |
| | | | | | | Mus musculus Ig 2G3.H5 heavy chain mRNA, specific for rat |
| ET61730 | ET61730 | 0.67 | 10.00 | 37.67 | 10.67 | (mouse) cytochrome c, partical cds. |
| | | | | | | Mus musculus Ig 5C12.A4 heavy chain mRNA, specific for rat |
| ET61732 | ET61732 | 0.00 | 10.00 | 30.33 | 10.00 | (mouse) cytochrome c, partial cds. |
| | | | | | | Mus musculus Ig 7A12.A4 heavy chain mRNA, specific for rat |
| ET61733 | ET61733 | 0.00 | 10.00 | 32.67 | 10.00 | (mouse) cytochrome c, partial cds. |
| | | | | | | Mus musculus Ig 9G7.A10 heavy chain mRNA, specific for rat |
| ET61736 | ET61736 | 0.00 | 10.00 | 44.67 | 10.00 | (mouse) cytochrome c, partial cds. |
| | | | | | | Mus musculus Ig 3A6.A5 heavy chain mRNA, specific for rat |
| ET61737 | ET61737 | 0.00 | 10.00 | 30.33 | 10.00 | (mouse) cytochrome c, partial cds. |
| | | | | | | Mus musculus Ig 7D1.B8 heavy chain mRNA, specific for rat |
| ET61739 | ET61739 | 0.00 | 10.00 | 23.67 | 10.00 | (mouse) cytochrome c, partial cds. |
| | | | | | | Mus musculus Ig 2C9.B12 heavy chain mRNA, specific for rat |
| ET61741 | ET61741 | 0.00 | 10.00 | 31.33 | 10.00 | (mouse) cytochrome c, partial cds. |
| | | | | | | Mus musculus Ig 3F10.C9 heavy chain mRNA, specific for rat |
| ET61744 | ET61744 | 0.00 | 10.00 | 20.00 | 10.00 | (mouse) cytochrome c, partial cds. |
| | | | | | | Mus musculus Ig 4A6.A8 heavy chain mRNA, specific for rat |
| ET61746 | ET61746 | 0.00 | 10.00 | 43.00 | 10.00 | (mouse) cytochrome c, partial cds. |
| | | | | | | Mus musculus Ig 4C4.A10 heavy chain mRNA, specific for rat |
| ET61747 | ET61747 | 2.00 | 10.00 | 40.67 | 12.00 | (mouse) cytochrome c, partial cds. |
| | | | | | | Mus musculus Ig 4C5.A11 heavy chain mRNA, specific for rat |
| ET61748 | ET61748 | 0.33 | 10.00 | 35.67 | 10.33 | (mouse) cytochrome c, partial cds. |
| | | | | | | Mus musculus Ig 6C3.B8 heavy chain mRNA, specific for rat |
| ET61749 | ET61749 | 0.67 | 10.00 | 21.00 | 10.67 | (mouse) cytochrome c, partial cds. |
| | | | | | | Mus musculus Ig 10B7.A1 heavy chain mRNA, specific for rat |
| ET61753 | ET61753 | 0.00 | 10.00 | 25.67 | 10.00 | (mouse) cytochrome c, partial cds. |
| | | | | | | Mus musculus anti-DNA immunoglobulin heavy chain IgM |
| ET61783 | ET61783 | 0.67 | 10.00 | 63.33 | 10.67 | mRNA, antibody 363p.138, partial cds. |
| | | | | | | Mus musculus anti-DNA immunoglobulin heavy chain IgM |
| ET61785 | ET61785 | 1.00 | 10.00 | 85.33 | 11.00 | mRNA, antibody 363p.168, partial cds. |
| | | | | | | Mus musculus anti-DNA immunoglobulin heavy chain IgM |
| ET61788 | ET61788 | 0.00 | 10.00 | 58.67 | 10.00 | mRNA, antibody 363p.197, partial cds. |
| | | | | | | Mus musculus anti-DNA immunoglobulin heavy chain IgG |
| ET61791 | ET61791 | 0.00 | 10.00 | 21.00 | 10.00 | mRNA, antibody 363p.24, partial cds. |
| | | | | | | Mus musculus anti-DNA immunoglobulin heavy chain IgG |
| ET61792 | ET61792 | 0.00 | 10.00 | 33.00 | 10.00 | mRNA, antibody 363p.8, partial cds. |
| | | | | | | Mus musculus anti-DNA immunoglobulin heavy chain IgG |
| ET61798 | ET61798 | 0.00 | 10.00 | 49.33 | 10.00 | mRNA, antibody 363s.68, partial cds. |
| | | | | | | Mus musculus anti-DNA immunoglobulin heavy chain IgG |
| ET61800 | ET61800 | 0.00 | 10.00 | 35.33 | 10.00 | mRNA, antibody 363s.73, partial cds. |
| | | | | | | Mus musculus anti-DNA immunoglobulin heavy chain IgM |
| ET61801 | ET61801 | 0.00 | 10.00 | 36.00 | 10.00 | mRNA, antibody 373p.95, partial cds. |
| | | | | | | Mus musculus anti-DNA immunoglobulin heavy chain IgM |
| ET61809 | ET61809 | 0.00 | 10.00 | 33.67 | 10.00 | mRNA, antibody 373s.83, partial cds. |
| | | | | | | Mus musculus anti-DNA immunoglobulin heavy chain IgM |
| ET61810 | ET61810 | 0.33 | 10.00 | 39.33 | 10.33 | mRNA, antibody 373s.70, partial cds. |
| | | | | | | Mus musculus anti-DNA immunoglobulin heavy chain IgG |
| ET61814 | ET61814 | 0.00 | 10.00 | 41.67 | 10.00 | mRNA, antibody 373p.5, partial cds. |
| | | | | | | Mus musculus anti-DNA immunoglobulin heavy chain IgG |
| ET61815 | ET61815 | 2.00 | 10.00 | 81.00 | 12.00 | mRNA, antibody 373p.51, partial cds. |
| | | | | | | Mus musculus anti-DNA immunoglobulin heavy chain IgG |
| ET61821 | ET61821 | 0.00 | 10.00 | 32.33 | 10.00 | mRNA, antibody 373p.32, partial cds. |
| | | | | | | Mus musculus anti-DNA immunoglobulin heavy chain IgG |
| ET61832 | ET61832 | 0.00 | 10.00 | 22.67 | 10.00 | mRNA, antibody 384p.113, partial cds. |
| | | | | | | Mus musculus anti-DNA immunoglobulin heavy chain IgG |
| ET61833 | ET61833 | 4.67 | 10.00 | 96.67 | 14.67 | mRNA, antibody 384p.20, partial cds. |
| | | | | | | Mus musculus anti-DNA immunoglobulin heavy chain IgG |
| ET61837 | ET61837 | 0.00 | 10.00 | 27.33 | 10.00 | mRNA, antibody 384s.73, partial cds. |
| | | | | | | Mus musculus anti-DNA immunoglobulin heavy chain IgG |
| ET61838 | ET61838 | 0.00 | 10.00 | 20.33 | 10.00 | mRNA, antibody 384s.80, partial cds. |
| | | | | | | Mus musculus anti-DNA immunoglobulin heavy chain IgG |
| ET61839 | ET61839 | 2.33 | 10.00 | 68.33 | 12.33 | mRNA, antibody 384s.95, partial cds. |
| | | | | | | Mus musculus anti-DNA immunoglobulin heavy chain IgG |
| ET61841 | ET61841 | 1.33 | 10.00 | 28.33 | 11.33 | mRNA, antibody 384s.17, partial cds. |
| | | | | | | Mus musculus anti-DNA immunoglobulin heavy chain IgG |
| ET61845 | ET61845 | 0.00 | 10.00 | 43.00 | 10.00 | mRNA, antibody 384s.14, partial cds. |
| | | | | | | Mus musculus anti-DNA immunoglobulin heavy chain IgG |
| ET61846 | ET61846 | 0.00 | 10.00 | 28.33 | 10.00 | mRNA, antibody 384s.15, partial cds. |
| | | | | | | Mus musculus anti-DNA immunoglobulin heavy chain IgG |
| ET61851 | ET61851 | 0.00 | 10.00 | 27.33 | 10.00 | mRNA, antibody 423p.78, partial cds. |
| | | | | | | Mus musculus anti-DNA immunoglobulin heavy chain IgG |
| ET61853 | ET61853 | 0.67 | 10.00 | 48.00 | 10.67 | mRNA, antibody 423p.83, partial cds. |
| | | | | | | Mus musculus anti-DNA immunoglobulin heavy chain IgG |
| ET61854 | ET61854 | 0.00 | 10.00 | 37.00 | 10.00 | mRNA, antibody 423p.107, partial cds. |
| | | | | | | Mus musculus anti-DNA immunoglobulin heavy chain IgG |
| ET61855 | ET61855 | 0.67 | 10.00 | 46.67 | 10.67 | mRNA, antibody 423p.135, partial cds. |
| | | | | | | Mus musculus anti-DNA immunoglobulin heavy chain IgG |
| ET61857 | ET61857 | 0.00 | 10.00 | 57.33 | 10.00 | mRNA, antibody 423p.195, partial cds. |
| | | | | | | Mus musculus anti-DNA immunoglobulin heavy chain IgG |
| ET61859 | ET61859 | 0.00 | 10.00 | 33.67 | 10.00 | mRNA, antibody 423p.226, partial cds. |
| | | | | | | Mus musculus anti-DNA immunoglobulin heavy chain IgG |
| ET61863 | ET61863 | 0.00 | 10.00 | 26.67 | 10.00 | mRNA, antibody 423p.38, partial cds. |
| | | | | | | Mus musculus anti-DNA immunoglobulin heavy chain IgM |
| ET61870 | ET61870 | 0.00 | 10.00 | 39.33 | 10.00 | mRNA, antibody 452p.17, partial cds. |
| | | | | | | Mus musculus anti-DNA immunoglobulin heavy chain IgM |
| ET61871 | ET61871 | 0.00 | 10.00 | 20.67 | 10.00 | mRNA, antibody 452p.18, partial cds. |
| | | | | | | Mus musculus anti-DNA immunoglobulin heavy chain IgM |
| ET61873 | ET61873 | 1.00 | 10.00 | 31.00 | 11.00 | mRNA, antibody 452p.53, partial cds. |
| | | | | | | Mus musculus anti-DNA immunoglobulin heavy chain IgM |
| ET61874 | ET61874 | 0.00 | 10.00 | 21.67 | 10.00 | mRNA, antibody 452p.71m, partial cds. |
| | | | | | | Mus musculus anti-DNA immunoglobulin heavy chain IgM |
| ET61876 | ET61876 | 0.00 | 10.00 | 73.33 | 10.00 | mRNA, antibody 452p.70, partial cds. |
| | | | | | | Mus musculus anti-DNA immunoglobulin heavy chain IgG |
| ET61885 | ET61885 | 2.33 | 10.00 | 66.33 | 12.33 | mRNA, antibody 452p.33, partial cds. |
| | | | | | | Mus musculus anti-DNA immunoglobulin heavy chain IgG |
| ET61908 | ET61908 | 0.67 | 10.00 | 49.00 | 10.67 | mRNA, antibody 452s.5, partial cds. |
| | | | | | | Mus musculus anti-DNA immunoglobulin heavy chain IgG |
| ET61909 | ET61909 | 0.33 | 10.00 | 31.67 | 10.33 | mRNA, antibody 452s.43, partial cds. |
| | | | | | | Mus musculus anti-DNA immunoglobulin light chain IgM |
| ET61916 | ET61916 | 5.00 | 10.00 | 44.67 | 15.00 | mRNA, antibody 363p.193, partial cds. |
| | | | | | | Mus musculus anti-DNA immunoglobulin light chain IgM |
| ET61918 | ET61918 | 0.00 | 10.00 | 72.33 | 10.00 | mRNA, antibody 363s.202, partial cds. |
| | | | | | | Mus musculus anti-DNA immunoglobulin light chain IgM |
| ET61919 | ET61919 | 0.00 | 10.00 | 30.33 | 10.00 | mRNA, antibody 363s.57, partial cds. |
| | | | | | | Mus musculus anti-DNA immunoglobulin light chain IgG, |
| ET61921 | ET61921 | 1.67 | 10.00 | 33.67 | 11.67 | mRNA, antibody 363p.8, partial cds. |
| | | | | | | Mus musculus anti-DNA immunoglobulin light chain IgG, |
| ET61925 | ET61925 | 3.00 | 10.00 | 65.67 | 13.00 | mRNA, antibody 363s.71, partial cds. |
| | | | | | | Mus musculus anti-DNA immunoglobulin light chain IgM |
| ET61937 | ET61937 | 0.00 | 10.00 | 24.67 | 10.00 | mRNA, antibody 373s.70, partial cds. |
| | | | | | | Mus musculus anti-DNA immunoglobulin light chain IgG, |
| ET61942 | ET61942 | 4.33 | 10.00 | 77.67 | 14.33 | antibody 373s.51, partial cds. |
| | | | | | | Mus musculus anti-DNA immunoglobulin light chain IgG, |
| ET61947 | ET61947 | 0.00 | 10.00 | 25.33 | 10.00 | antibody 373s.20, partial cds. |
| | | | | | | Mus musculus anti-DNA immunoglobulin light chain IgG, |
| ET61955 | ET61955 | 0.00 | 10.00 | 22.33 | 10.00 | antibody 373s.116, partial cds. |
| | | | | | | Mus musculus anti-DNA immunoglobulin light chain IgG, |
| ET61957 | ET61957 | 4.25 | 10.75 | 73.00 | 15.00 | antibody 384p.41, partial cds. |
| | | | | | | Mus musculus anti-DNA immunoglobulin light chain IgG, |
| ET61965 | ET61965 | 0.00 | 10.00 | 20.67 | 10.00 | antibody 384s.80, partial cds. |
| | | | | | | Mus musculus anti-DNA immunoglobulin light chain IgG, |
| ET61970 | ET61970 | 3.00 | 10.00 | 33.67 | 13.00 | antibody 384s.63, partial cds. |
| | | | | | | Mus musculus anti-DNA immunoglobulin light chain IgG, |
| ET61976 | ET61976 | 1.00 | 10.00 | 29.67 | 11.00 | antibody 384s.89, partial cds. |
| | | | | | | Mus musculus anti-DNA immunoglobulin light chain IgG, |
| ET61984 | ET61984 | 1.00 | 10.00 | 33.33 | 11.00 | antibody 423p.195, partial cds. |
| | | | | | | Mus musculus anti-DNA immunoglobulin light chain IgG, |
| ET62015 | ET62015 | 0.00 | 10.00 | 20.00 | 10.00 | antibody 452p.151, partial cds. |
| | | | | | | Mus musculus anti-DNA immunoglobulin light chain IgG, |
| ET62023 | ET62023 | 0.00 | 10.00 | 20.00 | 10.00 | antibody 452s.36, partial cds. |
| | | | | | | Mus musculus anti-DNA immunoglobulin light chain IgG, |
| ET62026 | ET62026 | 0.00 | 10.00 | 25.00 | 10.00 | antibody 452s.88, partial cds. |
| | | | | | | Mus musculus anti-DNA immunoglobulin light chain IgG, |
| ET62039 | ET62039 | 3.00 | 10.00 | 46.33 | 13.00 | antibody 452s.61, partial cds. |
| | | | | | | Mus musculus immunoglobulin rearranged gamma-1 chain |
| ET62052 | ET62052 | 0.00 | 10.00 | 101.33 | 10.00 | mRNA, partial cds. |
| ET62112 | ET62112 | 0.00 | 10.00 | 22.33 | 10.00 | Mus musculus J558+ IgM heavy chain mRNA, partial cds. |
| | | | | | | Mus musculus anti-PAH immunoglobulin Fab 10C10 heavy |
| ET62172 | ET62172 | 0.00 | 10.00 | 61.00 | 10.00 | chain V and CH1 regions gene, partial cds. |
| | | | | | | Mus musculus Ig anti-DNA heavy chain VDJ (J558) mRNA, |
| ET62188 | ET62188 | 0.00 | 10.00 | 34.00 | 10.00 | partial cds. |
| | | | | | | Mus musculus Ig anti-DNA heavy chain VDJ (J558) mRNA, |
| ET62191 | ET62191 | 1.67 | 10.00 | 58.67 | 11.67 | partial cds. |
| | | | | | | Mus musculus Ig anti-DNA heavy chain VDJ (J558) mRNA, |
| ET62192 | ET62192 | 0.00 | 10.00 | 27.67 | 10.00 | partial cds. |
| | | | | | | Mus musculus Ig anti-DNA light chain (Vk4/5) mRNA, |
| ET62199 | ET62199 | 1.33 | 10.00 | 46.00 | 11.33 | partial cds. |
| | | | | | | Mus musculus anti-digoxin immunoglobulin heavy chain, |
| ET62206 | ET62206 | 2.00 | 10.00 | 27.67 | 12.00 | variable region precursor mRNA, partial cds. |
| | | | | | | Mus musculus immunoglobulin heavy chain variable region |
| ET62224 | ET62224 | 2.33 | 10.00 | 31.33 | 12.33 | mRNA, partial cds. |
| | | | | | | Mus musculus polyreactive autoantibody, immunoglobulin IgM |
| ET62233 | ET62233 | 0.00 | 10.00 | 32.00 | 10.00 | heavy chain mRNA, partial cds. |
| | | | | | | Mus musculus polyreactive autoantibody, immunoglobulin IgM |
| ET62234 | ET62234 | 0.00 | 10.00 | 26.67 | 10.00 | heavy chain mRNA, partial cds. |
| | | | | | | Mus musculus anti-PAH immunoglobulin Fab 4D5 heavy |
| ET62256 | ET62256 | 0.00 | 10.00 | 36.00 | 10.00 | chain V and CH1 regions mRNA, partial cds. |
| | | | | | | Mus musculus immunoglobulin light chain variable region |
| ET62260 | ET62260 | 3.33 | 10.00 | 37.67 | 13.33 | mRNA, partial cds. |
| | | | | | | Mus musculus type II collagen antibody heavy chain variable |
| ET62422 | ET62422 | 0.00 | 10.00 | 22.00 | 10.00 | region mRNA, partial cds. |
| ET62430 | ET62430 | 0.00 | 10.00 | 21.33 | 10.00 | Mus musculus Ig heavy chain Fv fragment mRNA, partial cds. |
| | | | | | | Mus musculus Ig light chain Fv fragment specific for human |
| ET62459 | ET62459 | 0.00 | 10.00 | 20.33 | 10.00 | apolipoprotein A-I, mRNA, partial cds. |
| | | | | | | Mus musculus anti-DNA antibody heavy chain variable region |
| ET62705 | ET62705 | 0.33 | 10.00 | 65.00 | 10.33 | mRNA, partial cds. |
| | | | | | | Mus musculus anti-DNA antibody heavy chain variable region |
| ET62707 | ET62707 | 0.67 | 10.00 | 25.67 | 10.67 | mRNA, partial cds. |
| | | | | | | Mus musculus anti-DNA antibody heavy chain variable region |
| ET62717 | ET62717 | 0.00 | 10.00 | 25.33 | 10.00 | mRNA, partial cds. |
| | | | | | | Mus musculus anti-DNA antibody heavy chain variable region |
| ET62725 | ET62725 | 2.00 | 10.00 | 81.33 | 12.00 | mRNA, partial cds. |
| | | | | | | Mus musculus IgM heavy chain variable region |
| ET62779 | ET62779 | 0.00 | 10.00 | 65.67 | 10.00 | mRNA, partial cds. |
| | | | | | | Mus musculus anti-CD8 immunoglobulin heavy chain V region |
| ET62868 | ET62868 | 0.00 | 10.00 | 33.67 | 10.00 | mRNA, partial cds. |
| ET62923 | ET62923 | 0.00 | 10.00 | 56.67 | 10.00 | M. musculus antibody heavy chain variable region (354bp). |
| ET62924 | ET62924 | 0.00 | 10.00 | 59.67 | 10.00 | M. musculus antibody heavy chain variable region (363bp). |
| ET62925 | ET62925 | 1.33 | 10.00 | 74.67 | 11.33 | M. musculus antibody heavy chain variable region (372bp). |
| ET62926 | ET62926 | 0.00 | 10.00 | 30.00 | 10.00 | M. musculus antibody heavy chain variable region (354bp). |
| ET62928 | ET62928 | 0.67 | 11.00 | 23.00 | 11.67 | M. musculus antibody heavy chain variable region (366bp). |
| ET62932 | ET62932 | 0.00 | 10.00 | 22.00 | 10.00 | M. musculus antibody heavy chain variable region (372bp). |
| ET62933 | ET62933 | 0.00 | 10.00 | 25.67 | 10.00 | M. musculus antibody heavy chain variable region (360bp). |
| ET62934 | ET62934 | 0.00 | 10.00 | 30.33 | 10.00 | M. musculus antibody heavy chain variable region (348bp). |
| ET62936 | ET62936 | 0.00 | 10.00 | 24.67 | 10.00 | M. musculus antibody heavy chain variable region (375bp). |
| ET62941 | ET62941 | 0.33 | 10.00 | 37.33 | 10.33 | M. musculus antibody light chain variable region (318bp). |
| ET62942 | ET62942 | 1.33 | 10.00 | 44.00 | 11.33 | M. musculus antibody light chain variable region (324bp). |
| ET62985 | ET62985 | 0.00 | 10.00 | 39.00 | 10.00 | M. musculus mRNA (1B5) for IgA V-D-J-heavy chain. |
| | | | | | | M. musculus mRNA for immunoglobulin variable region, heavy |
| ET63027 | ET63027 | 0.67 | 10.00 | 24.33 | 10.67 | chain. |
| ET63039 | ET63039 | 0.00 | 10.00 | 77.33 | 10.00 | M. musculus mRNA for variable heavy chain. |
| ET63041 | ET63041 | 0.00 | 10.00 | 55.00 | 10.00 | M. musculus mRNA for immunoglobulin heavy variable region. |
| ET63042 | ET63042 | 0.00 | 10.00 | 29.00 | 10.00 | M. musculus mRNA for immunoglobulin kappa variable region. |
| | | | | | | M. musculus mRNA for monoclonal antibody heavy chain |
| ET63085 | ET63085 | 0.00 | 10.00 | 49.33 | 10.00 | variable region. |
| | | | | | | M. musculus mRNA for immunoglobulin heavy chain variable |
| ET63093 | ET63093 | 1.00 | 10.00 | 34.00 | 11.00 | domain, subgroup IIb. |
| | | | | | | M. musculus mRNA for immunoglobulin heavy chain variable |
| ET63106 | ET63106 | 0.00 | 10.00 | 22.33 | 10.00 | region, isolate 205. |
| | | | | | | M. musculus mRNA for immunoglobulin kappa light chain |
| ET63107 | ET63107 | 0.00 | 10.00 | 32.67 | 10.00 | variable region. |
| | | | | | | M. musculus mRNA for anti folate binding protein, MOv19 |
| ET63126 | ET63126 | 2.00 | 10.00 | 30.00 | 12.00 | Vkappa. |
| | | | | | | M. domesticus IgG variable region.)PIR:PH1015 (Ig heavy |
| ET63271 | ET63271 | −1.00 | 11.00 | 23.67 | 10.00 | chain V region (clone 111.55) - mouse (fragment) |
| | | | | | | M. domesticus IgG variable region.)PIR:PH1001 (Ig heavy |
| ET63274 | ET63274 | 0.00 | 10.00 | 51.33 | 10.00 | chain V region (clone 111.68) - mouse (fragment) |
| | | | | | | M. domesticus IgM variable region.)PIR:S26746 (Ig heavy |
| | | | | | | chain J region JH3 - mouse)PIR:PH0985 (Ig heavy chain V |
| ET63276 | ET63276 | 3.00 | 10.00 | 85.67 | 13.00 | region (clone 163.100) - mouse (fragment) |
| | | | | | | M. domesticus IgG variable region.)PIR:PH1007 (Ig heavy |
| ET63278 | ET63278 | 0.00 | 10.00 | 38.33 | 10.00 | chain V region (clone 163-c1) - mouse (fragment) |
| | | | | | | M. domesticus IgM variable region.)PIR:PH0975 (Ig heavy |
| ET63288 | ET63288 | 0.00 | 10.00 | 40.67 | 10.00 | chain V region (clone 163.72) - mouse (fragment) |
| | | | | | | M. domesticus IgK variable region.)PIR:PH1066 (Ig light |
| ET63290 | ET63290 | 0.00 | 10.00 | 40.67 | 10.00 | chain V region (clone 165.14) - mouse (fragment) |
| | | | | | | M. domesticus IgM variable region.)PIR:S26747 (Ig heavy |
| ET63295 | ET63295 | 2.67 | 10.00 | 75.33 | 12.67 | chain J region JH4 - mouse |
| | | | | | | M. domesticus IgG variable region.)PIR:PH0983 (Ig heavy |
| ET63300 | ET63300 | 0.00 | 10.00 | 63.00 | 10.00 | chain V region (clone 165.49) - mouse (fragment) |
| | | | | | | M. domesticus IgM variable region.)PIR:S26747 (Ig heavy |
| | | | | | | chain J region JH4 - mouse)PIR:PH1012 (Ig heavy chain V |
| ET63314 | ET63314 | 1.33 | 10.00 | 45.67 | 11.33 | region (clone 17p.73) - mouse (fragment) |
| | | | | | | M. domesticus IgM variable region.)PIR:PH0972 (Ig heavy |
| ET63320 | ET63320 | 1.00 | 10.00 | 57.00 | 11.00 | chain V region (clone 17s.128) - mouse (fragment) |
| | | | | | | M. domesticus IgK variable region.)PIR:PH1073 (Ig light |
| ET63322 | ET63322 | 0.00 | 10.00 | 27.00 | 10.00 | chain V region (clone 17s.130) - mouse (fragment) |
| | | | | | | M. domesticus IgM variable region.)PIR:PH0980 (Ig heavy |
| ET63324 | ET63324 | 0.00 | 10.00 | 35.67 | 10.00 | chain V region (clone 17s.13) - mouse (fragment) |
| | | | | | | M. domesticus IgM variable region.)PIR:PH0978 (Ig heavy |
| ET63328 | ET63328 | 0.00 | 10.00 | 55.67 | 10.00 | chain V region (clone 17s.166) - mouse (fragment) |
| | | | | | | M. domesticus IgG variable region.)PIR:PH0988 (Ig heavy |
| ET63331 | ET63331 | 0.00 | 10.00 | 33.33 | 10.00 | chain V region (clone 17s-c3) - mouse (fragment) |
| ET63333 | ET63333 | 1.33 | 10.00 | 78.33 | 11.33 | M. domesticus IgG variable region. |
| | | | | | | M. domesticus IgG variable region.)PIR:PH1009 (Ig heavy |
| ET63337 | ET63337 | 0.00 | 10.00 | 22.33 | 10.00 | chain V region (clone 17s.5) - mouse (fragment) |
| | | | | | | M. domesticus IgM variable region.)PIR:PH0986 (Ig heavy |
| ET63339 | ET63339 | 0.00 | 10.00 | 42.33 | 10.00 | chain V region (clone 17s.-c6) - mouse (fragment) |
| | | | | | | M. domesticus IgG variable region.)PIR:PH0984 (Ig heavy |
| ET63341 | ET63341 | 0.00 | 10.00 | 54.33 | 10.00 | chain V region (clone 17s.83) - mouse (fragment) |
| | | | | | | M. domesticus IgG variable region.)PIR:S26747 (Ig heavy |
| | | | | | | chain J region JH4 - mouse)PIR:PH1000 (Ig heavy chain V |
| ET63348 | ET63348 | 0.00 | 10.00 | 46.33 | 10.00 | region (clone 202.105) - mouse (fragment) |
| | | | | | | M. domesticus IgM variable region.)PIR:PH1006 (Ig heavy |
| ET63351 | ET63351 | 0.00 | 10.00 | 34.00 | 10.00 | chain V region (clone 202.33) - mouse (fragment) |
| | | | | | | M. domesticus IgM variable region.)PIR:PH0995 (Ig heavy |
| ET63354 | ET63354 | 1.33 | 10.00 | 64.33 | 11.33 | chain V region (clone 202.61) - mouse (fragment) |
| | | | | | | M. domesticus IgK variable region.)PIR:PH1046 (Ig light |
| | | | | | | chain V region (clone 202.9) - mouse) (fragment))PIR:PH1048 (Ig |
| ET63358 | ET63358 | 0.00 | 10.00 | 42.00 | 10.00 | light chain V region (clone 165.49) - mouse |
| | | | | | | M. domesticus IgM variable region.)PIR:PH1011 (Ig heavy |
| ET63359 | ET63359 | 0.00 | 10.00 | 35.67 | 10.00 | chain V region (clone 202.38m) - mouse (fragment) |
| | | | | | | M. domesticus IgM variable region.)PIR:PH0976 (Ig heavy |
| ET63363 | ET63363 | 0.00 | 10.00 | 43.00 | 10.00 | chain V region (clone 25.12m) - mouse (fragment) |
| ET63365 | ET63365 | 1.67 | 10.00 | 64.33 | 11.67 | M. domesticus IgG variable region. |
| | | | | | | M. domesticus IgK variable region.)PIR:PH1076 (Ig light |
| ET63368 | ET63368 | 0.00 | 10.00 | 30.00 | 10.00 | chain V region (clone 74-c2) - mouse (fragment) |
| ET63369 | ET63369 | 0.00 | 10.00 | 24.33 | 10.00 | M. domesticus IgG variable region. |
| ET63387 | ET63387 | 0.00 | 10.00 | 48.67 | 10.00 | Artificial mRNA for single chain antibody scFv (scFvP25). |
| | | | | | | Mus musculus mRNA for IgGf/kappa antibody, scFv(glyc)- |
| | | | | | | CK.)PIR:PH1043 (Ig light chain V region (clone 111.68) - |
| ET63415 | ET63415 | 0.33 | 10.00 | 34.67 | 10.33 | mouse (fragment))PIR:PH1042 (Ig light chain V region (clone |
| | | | | | | Mouse VK gene for kappa light chain variable region and J4 |
| IGK_V20 | X16678 | 5.00 | 10.00 | 36.33 | 15.00 | sequence. |
| | | | | | | Mus musculus CB17 SCID immunoglobulin heavy chain V |
| U23089 | u23089 | 0.67 | 10.00 | 30.67 | 10.67 | region mRNA, clone 58 − 53, partial cds. |
| | | | | | | M12813 Mouse Ig germline H-chain gene H10 V-region (V), |
| IGH_VH10 | m12813 | 4.67 | 10.00 | 33.33 | 14.67 | exons 1 and 2 |
| IGH_6 | z22111 | 0.00 | 10.00 | 43.67 | 10.00 | Z22111 M. domesticus IgG variable region |
| | | | | | | Z70662 Artificial mRNA for single chain antibody scFv |
| IGH_4 | z70662 | 0.00 | 10.00 | 39.00 | 10.00 | (scFvP25) |
| | | | | | | Mouse germline IgH chain gene, DJC region: segment D- |
| IGH_6 | J00475 | 0.00 | 10.00 | 59.33 | 10.00 | FL16.1 |
| | | | | | | Mouse lysozyme-binding Ig kappa chain (HyHEL-10) V23-J2 |
| IGK_V23 | M35667 | 0.00 | 10.00 | 36.67 | 10.00 | region mRNA, partial cds. |
| IGH_4 | M60429 | 0.00 | 10.00 | 79.00 | 10.00 | Mouse Ig rearranged H-chain mRNA constant region. |
| M86751 | M86751 | 1.00 | 10.00 | 30.00 | 11.00 | Mouse Ig L-chain gene variable region, complete cds. |

Unknown

| | | | | | | |
|---|---|---|---|---|---|---|
| | | | | | | mt74d01.r1 Soars mouse lymph node NbMLN Mus |
| E_TC17629 | AA165775 | −3.58 | 30.25 | 14.67 | 26.67 | musculus cDNA clone 635617 5′ |
| | | | | | | vl56h09.r1 Stratagene mouse skin (#937313) Mus musculus |
| | | | | | | cDNA clone 976289 5′ similar to gb:X06753 Mouse pro- |
| E_COLA1 | aa562685 | 1.17 | 11.50 | 58.33 | 12.67 | alpha1 (MOUSE); |
| E_CTSS | aa089333 | 0.00 | 10.00 | 45.33 | 10.00 | AA08933 mo60e02.r1 Mus musculus cDNA, 5′ end |
| E_CTSS | aa146437 | 0.00 | 10.00 | 42.67 | 10.00 | AA146437 mr05a08.r1 Mus musculus cDNA, 5′ end |
| | | | | | | vo54d12.r1 Barstead mouse irradiated colon MPLRB7 Mus |
| AA638539 | aa638539 | 1.42 | 11.25 | 47.33 | 12.67 | musculus cDNA clone 1053719 5′, mRNA sequence. |
| | | | | | | ma89d07.r1 Soares mouse p3NMF19.5 Mus musculus cDNA |
| | | | | | | clone 317869 5′ similar to gb:X57352 INTERFERON- |
| E_TC22736 | w12941 | 1.33 | 31.00 | 121.33 | 32.33 | INDUCIBLE PROTEIN 1-8U (HUMAN);, mRNA sequence. |
| | | | | | | mx25h11.r1 Soares mouse NML Mus musculus cDNA clone |
| | | | | | | 6812855 5′ similar to gb:X02415_ma3 FIBRINOGEN GAMMA- |
| E_X02415 | aa244836 | 0.33 | 10.00 | 37.00 | 10.33 | A CHAIN PRECURSOR (HUMAN); |
| PTMB4 | w41883 | −4.75 | 83.75 | 272.00 | 79.00 | W41883 mc64g08.r1 Mus musculus cDNA, 5′ end |
| E_1 | w20873 | 0.00 | 10.00 | 32.00 | 10.00 | W20873 mb92c11.r1 Mus musculus cDNA, 5′ end |
| E_TUBB1 | w12548 | 1.08 | 16.25 | 52.00 | 17.33 | W12548 ma59d04.r1 Mus musculus cDNA, 5′ end |
| | | | | | | Mouse 3.5-dpc blastocyst cDNA Mus musculus cDNA clone |
| C80103 | c80103 | 0.33 | 10.00 | 31.67 | 10.33 | J0076E08 3′, mRNA sequence. |
| | | | | | | vj03d05.r1 Barstead mouse pooled organs MPLRB4 Mus |
| | | | | | | musculus cDNA clone 920649 5′ similar to TR:G881954 |
| E_TC31065 | aa538285 | 2.50 | 13.50 | 42.00 | 16.00 | G881954 RNPL.; |
| E_G1P3 | aa120109 | 2.17 | 26.50 | 79.00 | 28.67 | AA120109 mq09a11.r1 Mus musculus cDNA, 5′ end |
| | | | | | | mw28h11.r1 Soares mouse 3NME12 5′ Mus musculus cDNA |
| | | | | | | clone 672069 5′ similar to gb:X61399 Mouse F52 mRNA for a |
| E_X61399 | aa245242 | 3.08 | 11.25 | 31.00 | 14.33 | novel protein (MOUSE); |
| CTSC | aa144887 | 0.00 | 10.00 | 26.33 | 10.00 | AA144887 mr11d06.r1 Mus musculus cDNA, 5′ end |
| | | | | | | mt65a03.r1 Soares mouse lymph node NbMLN Mus |
| E_TC34530 | aa163096 | −1.58 | 17.25 | 45.00 | 15.67 | musculus cDNA clone 634732 5′ |
| | | | | | | Mouse chromosome 6 BAC-284H12 (Research Genetics |
| HCPH_geneP | AC002397 | 5.00 | 10.00 | 25.00 | 15.00 | mouse BAC library) complete sequence. |
| W98864 | w98864 | 1.33 | 12.00 | 29.33 | 13.33 | W98864 mg11h11.r1 Mus musculus cDNA, 5′ end |
| MDK | aa072643 | 1.83 | 15.50 | 37.67 | 17.33 | AA072643 mm75a09.r1 Mus musculus cDNA, 5′ end |
| | | | | | | mx94f04.r1 Soares mouse NML Mus musculus cDNA clone |
| CD8B | aa238483 | 3.67 | 13.00 | 31.33 | 16.67 | 694015 5′ similar to TR:G806566 G806566 SM PROTEIN G. |
| | | | | | | vu57b03.r1 Soares mouse mammary gland NbMMG Mus |
| AA690738 | aa690738 | −2.83 | 15.50 | 36.33 | 12.67 | musculus cDNA clone 1195469 5′, mRNA sequence. |
| | | | | | | Mouse 3.5-dpc blastocyst cDNA Mus musculus cDNA clone |
| | | | | | | J0101H11 3′ similar to Mouse ribonucleotide reductase M2 |
| RRM2 | C81593 | 0.00 | 10.00 | 23.00 | 10.00 | subunit mRNA, mRNA sequence. |
| | | | | | | vt83b09.r1 Soares mouse mammary gland NbMMG Mus |
| | | | | | | musculus cDNA clone 850361 5′ similar to WP:C14B1.3 |
| E_TC39517 | aa451220 | 5.00 | 10.00 | 22.00 | 15.00 | CE00900; |
| | | | | | | ma41d10.r1 Soares mouse p3NMF19.5 Mus musculus cDNA |
| CD39L1 | W10995 | 3.67 | 11.00 | 23.00 | 14.67 | clone 313267 5′, mRNA sequence. |
| ACTC1 | aa117701 | 0.92 | 10.75 | 22.33 | 11.67 | AA117701 mo64d03.r1 Mus musculus cDNA, 5′ end |
| | | | | | | vb41e05.r1 Soares mouse lymph node NbMLN Mus musculus |
| E_TC33572 | aa396029 | 0.67 | 10.00 | 20.67 | 10.67 | cDNA clone 751520 5′ |
| E_W50888 | w50888 | 3.67 | 12.00 | 24.67 | 15.67 | W50888 ma23e03.r1 Mus musculus cDNA, 5′ end |
| | | | | | | EST03133 Mouse 7.5 dpc embryo ectoplacental cone cDNA |
| E_TC32548 | aa408672 | −0.58 | 39.25 | 80.00 | 38.67 | library Mus musculus cDNA clone C0031D07 3′ |
| E_JUN | w09701 | −1.58 | 16.25 | 32.33 | 14.67 | W09701 ma56e02.r1 Mus musculus cDNA, 5′ end |
| | | | | | | mg45b10.r1 Soares mouse embryo NbME13.5 14.5 Mus |
| E_TC18790 | aa002761 | 0.33 | 10.00 | 22.67 | 10.33 | musculus cDNA clone 426715 5′. |
| | | | | | | mi15h02.r1 Soares mouse p3NMF19.5 Mus musculus cDNA |
| E_TC39388 | aa028770 | 0.00 | 10.00 | 20.00 | 10.00 | clone 463635 5′ |
| | | | | | | mi22g02.r1 Soares mouse embryo NbME13.5 14.5 Mus |
| E_TC23744 | AA030688 | −0.25 | 10.25 | 25.67 | 10.00 | musculus cDNA clone 464306 5′ |
| | | | | | | mj76a06.r1 Soares mouse p3NMF19.5 Mus musculus cDNA |
| E_TC27896 | aa059883 | 3.17 | 10.50 | 21.33 | 13.67 | clone 482002 5′ |
| | | | | | | mp09d07.r1 Life Tech mouse embryo 8 5dpc 10664019 Mus |
| SPI6 | aa108054 | 0.33 | 10.00 | 23.33 | 10.33 | musculus cDNA clone 568717 5′ |
| | | | | | | mp39a05.r1 Barstead MPLRB1 Mus musculus cDNA clone |
| E_TC28792 | aa108677 | 4.33 | 10.00 | 21.00 | 14.33 | 571568 5′ |
| | | | | | | mp71g11.r1 Soares 2NbMI Mus musculus cDNA clone |
| | | | | | | 574724 5′ similar to gb:D21261 SM22-ALPHA HOMOLOG |
| E_D21261 | aa120653 | 2.08 | 35.25 | 124.67 | 37.33 | (HUMAN); |
| | | | | | | mn33e03.r1 Beddington mouse embryonic region Mus |
| | | | | | | musculus cDNA clone 539740 5′ similar to TR:E236822 |
| E_TC15056 | aa122622 | −1.25 | 11.25 | 25.33 | 10.00 | E236822 HYPOTHETICAL 26.5 KD PROTEIN.; |
| | | | | | | mq98h01.r1 Soares mouse 3NbMS Mus musculus cDNA |
| E_TC17285 | aa137292 | 4.42 | 16.25 | 32.33 | 20.67 | clone 596017 5′ |
| | | | | | | mr31f05.r1 Soares mouse 3NbMS Mus musculus cDNA |
| E_TC37973 | aa172851 | 4.67 | 10.00 | 21.67 | 14.67 | clone 599073 5′ |
| | | | | | | ms77e07.r1 Soares mouse 3NbMS Mus musculus cDNA |
| E_TC27387 | aa174883 | 0.67 | 25.00 | 65.67 | 25.67 | clone 617604 5′ |
| | | | | | | mt22f04.r1 Soares mouse 3NbMS Mus musculus cDNA |
| E_TC21726 | aa184116 | 0.58 | 11.75 | 28.00 | 12.33 | clone 621823 5′ |
| | | | | | | mu72h03.r1 Soares mouse lymph node NbMLN Mus |
| E_TC27481 | aa210359 | 1.00 | 13.00 | 29.33 | 14.00 | musculus cDNA clone 644981 5′ |
| | | | | | | EST02956 Mouse 7.5 dpc embryo ectoplacental cone cDNA |
| | | | | | | library Mus musculus cDNA clone C0028E12 3′, mRNA |
| TSTAP198_7 | AA408475 | 3.00 | 11.00 | 24.33 | 14.00 | sequence. |
| | | | | | | vj53e12.r1 Knowles Solter mouse blastocyst B1 Mus |
| E_TC35691 | aa538477 | 0.00 | 11.00 | 22.67 | 11.00 | musculus cDNA clone 932782 5′ |
| | | | | | | vk43h10.r1 Soares mouse mammary gland NbMMG Mus |
| E_TC39260 | aa542220 | −0.83 | 14.50 | 42.67 | 13.67 | musculus cDNA clone 949411 5′ |
| | | | | | | vo16a05.r1 Barstead mouse myotubes MPLRB5 Mus |
| AA596794 | aa596794 | −1.00 | 33.00 | 92.67 | 32.00 | musculus cDNA clone 1050032 5′, mRNA sequence. |
| | | | | | | vm91d04.r1 Knowles Solter mouse blastocyst B1 Mus |
| | | | | | | musculus cDNA clone 1005607 5′ similar to TR:G497940 |
| AA606926 | aa606926 | −4.92 | 15.25 | 35.00 | 10.33 | G497940 MAJOR VAULT PROTEIN.;, mRNA sequence. |
| | | | | | | vo50d04.r1 Barstead mouse irradiated colon MPLRB7 Mus |
| AA616243 | AA616243 | 0.33 | 10.00 | 21.33 | 10.33 | musculus cDNA clone 1053319 5′, mRNA sequence. |
| | | | | | | vq87c07.r1 Knowles Solter mouse blastocyst B3 Mus |
| AA666918 | aa666918 | −1.08 | 11.75 | 25.33 | 10.67 | musculus cDNA clone 1109292 5′, mRNA sequence. |
| | | | | | | vq57g08.r1 Barstead mouse proximal colon MPLRB6 Mus |
| POU2F2 | aa674986 | −1.75 | 11.75 | 37.67 | 10.00 | musculus cDNA clone 1106462 5′, mRNA sequence. |
| | | | | | | vt42f07.r1 Barstead mouse proximal colon MPLRB6 Mus |
| AA710451 | aa710451 | 0.33 | 10.00 | 46.33 | 10.33 | musculus cDNA clone 1165765 5′, mRNA sequence. |
| | | | | | | Mouse 3.5-dpc blastocyst cDNA Mus musculus cDNA clone |
| C76523 | c76523 | −1.17 | 11.50 | 30.67 | 10.33 | J0012E07 3′, mRNA sequence. |
| | | | | | | Mouse 3.5-dpc blastocyst cDNA Mus musculus cDNA clone |
| C76523 | c76523 | 0.00 | 10.00 | 23.00 | 10.00 | J0012E07 3′, mRNA sequence. |
| | | | | | | Mouse 3.5-dpc blastocyst cDNA Mus musculus cDNA clone |
| | | | | | | J0020H05 3′ similar to Mus musculus ribosomal protein S26 |
| C76830 | C76830 | −0.42 | 11.75 | 27.33 | 11.33 | (RPS26) mRNA, mRNA sequence. |
| | | | | | | Mouse 3.5-dpc blastocyst cDNA Mus musculus cDNA clone |
| | | | | | | J0038G08 3′ similar to Rattus norvegicus major vault protein |
| C77881 | C77861 | 0.17 | 16.50 | 35.67 | 16.67 | mRNA, mRNA sequence. |
| | | | | | | Mouse 3.5-dpc blastocyst cDNA Mus musculus cDNA clone |
| C80574 | C80574 | −3.00 | 28.00 | 60.67 | 25.00 | J0084D04 3′ similar to Human clone 23665 mRNA sequence. |
| E_W48951 | w48951 | 0.00 | 10.00 | 20.00 | 10.00 | W48951 md24g11.r1 Mus musculus cDNA, 5′ end |
| RRAS | w41501 | −0.25 | 10.25 | 21.67 | 10.00 | W41501 mc43d11.r1 Mus musculus cDNA, 5′ end |
| W50898 | w50898 | 2.92 | 15.75 | 40.33 | 18.67 | W50898 ma23g03.r1 Mus musculus cDNA, 5′ end |
| W57485 | w57485 | 0.00 | 10.00 | 23.67 | 10.00 | W57485 ma34h02.r1 Mus musculus cDNA, 5′ end |
| E_LAP18 | aa117100 | 1.83 | 11.50 | 24.33 | 13.33 | AA117100 mo60a10.r1 Mus musculus cDNA, 5′ end |
| AA011784 | aa011784 | 3.17 | 17.50 | 67.67 | 20.67 | AA011784 mg92b08.r1 Mus musculus cDNA, 5′ end |
| E_HSPB1 | aa015026 | 4.42 | 12.25 | 38.67 | 16.67 | AA015026 mh26f03.r1 Mus musculus cDNA, 5′ end |
| E_HSPB1 | aa015458 | −0.50 | 10.50 | 24.67 | 10.00 | AA015458 mh22b09.r1 Mus musculus cDNA, 5′ end |
| E_ABP1 | aa023491 | 0.00 | 10.00 | 38.33 | 10.00 | AA023491 mh74e11.r1 Mus musculus cDNA, 5′ end |
| LCN2 | w13166 | 0.00 | 10.00 | 70.00 | 10.00 | W13166 ma93f11.r1 Mus musculus cDNA, 5′ end |
| TUBA2 | aa030759 | −0.67 | 14.00 | 40.33 | 13.33 | AA030759 mi32e11.r1 Mus musculus cDNA, 5′ end |
| E_HSPB1 | aa034638 | 0.00 | 10.00 | 20.00 | 10.00 | AA034638 mh17a07.r1 Mus musculus cDNA, 5′ end |
| E_PEA15 | aa108330 | 2.50 | 11.50 | 40.00 | 14.00 | AA108330 mp28b03.r1 Mus musculus cDNA, 5′ end |
| E_ABP1 | aa107847 | 0.00 | 10.00 | 34.67 | 10.00 | AA107847 mo49d08.r1 Mus musculus cDNA, 5′ end |
| E_ABP1 | aa104688 | 0.00 | 10.00 | 42.67 | 10.00 | AA104688 mo55c10.r1 Mus musculus cDNA, 5′ end |
| E_PRKM1 | aa104744 | 0.00 | 10.00 | 28.67 | 10.00 | AA104744 mo56d02.r1 Mus musculus cDNA, 5′ end |
| E_ABP1 | aa109909 | 0.00 | 10.00 | 28.67 | 10.00 | AA109909 mp10d09.r1 Mus musculus cDNA, 5′ end |
| E_HSPB1 | w08057 | 2.33 | 10.00 | 48.00 | 12.33 | W08057 mb37e05.r1 Mus musculus cDNA, 5′ end |
| E_LGALS3 | w10936 | 0.00 | 10.00 | 27.33 | 10.00 | W10936 ma03e09.r1 Mus musculus cDNA, 5′ end |
| E_FLNA | w29429 | 2.67 | 10.00 | 33.67 | 12.67 | W29429 mb99d03.r1 Mus musculus cDNA, 5′ end |
| E_CCL64 | w90837 | −0.75 | 10.75 | 33.00 | 10.00 | W90837 mf78g07.r1 Mus musculus cDNA, 5′ end |

TABLE 7
Genes Normalized by AntiB7

| | | | | | Untr | |
|---|---|---|---|---|---|---|
| | | | | | 42w/12w | |
| | | | | | (fold | delta aB7- |
| Accession# | Gene description | Avg.Untr12w | Avg.Untr42w | Avg.aB7.50w | change) | unt12w |
| AA014127 | DNA segment, Chr 15, Wayne State | 17.50 | 58.00 | 21.67 | 3.31 | 4.17 |
| | University 77, expressed | | | | | |
| AA014427 | ESTs, Moderately similar to KIAA1398 | 18.50 | 40.67 | 20.00 | 2.20 | 1.50 |
| | protein [H. sapiens] | | | | | |
| AA028499 | mi19b09.r1 Soares mouse p3NMF19.5 | 10.00 | 22.00 | 10.00 | 2.20 | 0.00 |
| | Mus musculus cDNA clone 463961 5′ | | | | | |
| AA030185 | mh88g01.r1 Soares mouse placenta | 11.75 | 24.67 | 10.33 | 2.10 | −1.42 |
| | 4NbMP13.5 14.5 Mus musculus cDNA | | | | | |
| | clone 458064 5′ | | | | | |
| AA030551 | mi26a09.r1 Soares mouse embryo | 12.75 | 30.00 | 15.00 | 2.35 | 2.25 |
| | NbME13.5 14.5 Mus musculus cDNA clone | | | | | |
| | 464632 5′ | | | | | |
| AA033074 | flotillin 1 | 24.75 | 54.00 | 25.67 | 2.18 | 0.92 |
| AA124813 | mp80d03.r1 Soares 2NbMT Mus musculus | 10.00 | 22.67 | 14.67 | 2.27 | 4.67 |
| | cDNA clone 575525 5′ | | | | | |
| AA146509 | mr06e04.r1 Soares mouse 3NbMS Mus | 10.75 | 23.00 | 15.67 | 2.14 | 4.92 |
| | musculus cDNA clone 596670 5′ | | | | | |
| AA170668 | ESTs, Weakly similar to lysophospholipase | 10.50 | 29.67 | 13.33 | 2.83 | 2.83 |
| | l [M. musculus] | | | | | |
| AA177556 | NS1-associated protein 1 | 11.75 | 28.00 | 10.33 | 2.38 | −1.42 |
| AA178134 | mt14c11.r1 Soares mouse 3NbMS Mus | 10.25 | 21.00 | 14.00 | 2.05 | 3.75 |
| | musculus cDNA clone 621044 5′ | | | | | |
| AA178671 | mt18g04.r1 Soares mouse 3NbMS Mus | 13.25 | 29.00 | 15.33 | 2.19 | 2.08 |
| | musculus cDNA clone 621462 5′ | | | | | |
| AA183094 | mt84a04.r1 Soares mouse lymph node | 13.00 | 33.33 | 12.33 | 2.56 | −0.67 |
| | NbMLN Mus musculus cDNA clone 636558 | | | | | |
| | 5′ | | | | | |
| AA189422 | ESTs, Weakly similar to scaffold | 10.75 | 22.00 | 14.00 | 2.05 | 3.25 |
| | attachment factor B [R. norvegicus] | | | | | |
| AA209083 | mw74f12.r1 Soares mouse NML Mus | 10.00 | 20.00 | 13.00 | 2.00 | 3.00 |
| | musculus cDNA clone 676463 5′ | | | | | |
| AA254293 | synaptotagmin 11 | 10.50 | 33.00 | 14.00 | 3.14 | 3.50 |
| AA267679 | ESTs, Weakly similar to contains | 10.00 | 24.33 | 10.33 | 2.43 | 0.33 |
| | transmembrane [M. musculus] | | | | | |
| AA267968 | ESTs, Moderately similar to unnamed | 11.00 | 22.67 | 14.00 | 2.06 | 3.00 |
| | protein product [H. sapiens] | | | | | |
| AA271910 | ESTs, Highly similar to HYPOTHETICAL | 12.50 | 34.33 | 15.67 | 2.75 | 3.17 |
| | 13.6 KD PROTEIN IN NUP170-ILS1 | | | | | |
| | INTERGENIC REGION [Saccharomyces | | | | | |
| | cerevisiae] | | | | | |
| AA407697 | 3-monooxgenase/tryptophan 5-monooxgenase | 43.00 | 108.67 | 42.00 | 2.53 | −1.00 |
| | activation protein, gamma | | | | | |
| | polypeptide | | | | | |
| AA408675 | ESTs, Highly similar to unnamed protein | 13.25 | 31.00 | 16.67 | 2.34 | 3.42 |
| | product [H. sapiens] | | | | | |
| AA409818 | DNA segment, Chr 2, Wayne State | 10.50 | 22.00 | 12.67 | 2.10 | 2.17 |
| | University 58, expressed | | | | | |
| AA414142 | DNA segment, Chr 19, Wayne State | 28.75 | 60.33 | 31.00 | 2.10 | 2.25 |
| | University 162, expressed | | | | | |
| AA415044 | early development regulator 2 (homolog of | 11.75 | 27.67 | 13.67 | 2.35 | 1.92 |
| | polyhomeotic 2) | | | | | |
| | Mus musculus Balb/c zinc finger protein | | | | | |
| AA415813 | PZF (Pzf) mRNA, complete cds | 10.25 | 23.00 | 14.33 | 2.24 | 4.08 |
| AA498750 | programmed cell death 4 | 10.00 | 23.67 | 14.33 | 2.37 | 4.33 |
| AA537405 | ESTs, Weakly similar to KIAA0308 | 11.25 | 24.00 | 14.67 | 2.13 | 3.42 |
| | [H. sapiens] | | | | | |
| AA543807 | DNA segment, Chr 11, ERATO Doi 9, | 10.50 | 21.00 | 11.33 | 2.00 | 0.83 |
| | expressed | | | | | |
| AA544203 | ESTs, Highly similar to UBP7_HUMAN | 17.50 | 39.33 | 21.00 | 2.25 | 3.50 |
| | UBIQUITIN CARBOXYL-TERMINAL | | | | | |
| | HYDROLASE 7 [H. sapiens] | | | | | |
| AA571242 | ESTs, Highly similar to HYPOTHETICAL | 10.25 | 23.00 | 13.67 | 2.24 | 3.42 |
| | 13.5 KD PROTEIN C45G9.7 IN | | | | | |
| | CHROMOSOME III [Caenorhabditis | | | | | |
| | elegans] | | | | | |
| AA589418 | vl46g07.s1 Stratagene mouse skin | 10.25 | 25.33 | 11.33 | 2.47 | 1.08 |
| | (#937313) Mus musculus cDNA clone | | | | | |
| | 975324 3′, mRNA sequence. | | | | | |
| AA591007 | ESTs, Highly similar to AHNK_HUMAN | 35.25 | 88.67 | 36.00 | 2.52 | 0.75 |
| | NEUROBLAST DIFFERENTIATION | | | | | |
| | ASSOCIATED PROTEIN AHNAK | | | | | |
| | [H. sapiens] | | | | | |
| | heterogeneous nuclear ribonucleoprotein | | | | | |
| AA616337 | A/B | 33.25 | 67.00 | 37.67 | 2.02 | 4.42 |
| AA624011 | ESTs, Highly similar to MYOSIN HEAVY | 26.00 | 91.00 | 23.67 | 3.50 | −2.33 |
| | CHAIN, NONMUSOLE [Gallus gallus] | | | | | |
| AA638759 | vn03c04.r1 Knowles Solter mouse | 10.00 | 21.33 | 10.00 | 2.13 | 0.00 |
| | blastocyst B1 Mus musculus cDNA clone | | | | | |
| | 1006662 5′, mRNA sequence. | | | | | |
| AA673970 | vo87b04.r1 Barstead mouse irradiated | 10.75 | 22.00 | 10.00 | 2.05 | −0.75 |
| | colon MPLRB7 Mus musculus cDNA clone | | | | | |
| | 1066063 5′, mRNA sequence. | | | | | |
| AA675026 | ethanol induced 1 | 10.75 | 25.67 | 13.33 | 2.39 | 2.58 |
| AA690887 | E26 avian leukemia oncogene 2, 3′ domain | 10.75 | 22.00 | 12.33 | 2.05 | 1.58 |
| AA726578 | DNA segment, Chr 7, ERATO Doi 257, | 19.00 | 41.67 | 14.67 | 2.19 | −4.33 |
| | expressed | | | | | |
| AB006787 | mitogen activated protein kinase kinase | 10.00 | 21.00 | 10.67 | 2.10 | 0.67 |
| | kinase 5 | | | | | |
| AF013490 | protein tyrosine phosphatase, non-receptor | 14.75 | 34.67 | 18.67 | 2.35 | 3.92 |
| | type 9 | | | | | |
| AF022992 | period homolog (Drosophila) | 18.25 | 52.00 | 22.00 | 2.85 | 3.75 |
| C77188 | Mouse 3,5-dpc blastocyst cDNA Mus | 10.00 | 21.33 | 11.00 | 2.13 | 1.00 |
| | musculus cDNA clone J0027B07 3′, mRNA | | | | | |
| | sequence. | | | | | |
| D10576 | ubiquitin-activating enzyme E1, Chr X | 51.75 | 103.67 | 56.33 | 2.00 | 4.58 |
| D16141 | lethal giant larvae homolog | 16.00 | 34.00 | 21.00 | 2.13 | 5.00 |
| J03535 | embigin | 11.50 | 23.00 | 16.00 | 2.00 | 4.50 |
| J04696 | glutathione S-transferase, mu 2 | 20.00 | 39.67 | 24.67 | 1.98 | 4.67 |
| L07264 | heparin binding epidermal growth factor-like | 10.00 | 23.00 | 14.33 | 2.30 | 4.33 |
| | growth factor | | | | | |
| M22326 | early growth response 1 | 10.00 | 28.00 | 10.67 | 2.80 | 0.67 |
| M26270 | stearoyl-Coenzyme A desaturase 2 | 12.00 | 28.67 | 13.00 | 2.39 | 1.00 |
| M33272 | defensin related cryptdin, related sequence | 10.00 | 20.33 | 13.33 | 2.03 | 3.33 |
| | 2 | | | | | |
| M64292 | B-cell translocation gene 2, anti-proliferative | 10.00 | 31.00 | 12.67 | 3.10 | 2.67 |
| W44201 | stearoyl-Coenzyme A desaturase 2 | 24.00 | 51.00 | 27.33 | 2.13 | 3.33 |
| M16362 | trinucleotide repeat containing 11 | 10.50 | 22.67 | 13.67 | 2.16 | 3.17 |
| | (THR-associated protein, 230 kDa subunit) | | | | | |
| W65634 | valyl-tRNA synthetase 2 | 10.00 | 25.00 | 10.00 | 2.50 | 0.00 |
| W71798 | silent mating type information regulation 2, | 12.75 | 26.00 | 13.67 | 2.04 | 0.92 |
| | (S. cerevisiae, homolog)-like 3 | | | | | |
| W75814 | defender against cell death 1 | 14.00 | 32.33 | 15.33 | 2.31 | 1.33 |
| D50050 | HGF-regulated tyrosine kinase substrate | 13.25 | 29.67 | 16.00 | 2.24 | 2.75 |
| AA030895 | aplysia ras-related homolog 9 (RhoC) | 12.25 | 28.00 | 13.67 | 2.29 | 1.42 |
| X84014 | laminin, alpha 3 | 14.00 | 29.00 | 13.00 | 2.07 | −1.00 |
| X51397 | myeloid differentiation primary response | 11.25 | 23.33 | 13.67 | 2.07 | 2.42 |
| | gene 88 | | | | | |
| W90864 | valyl-tRNA synthetase 2 | 10.50 | 24.67 | 10.00 | 2.35 | −0.50 |
| AA170104 | ATPase, Ca++ transporting, cardiac | 22.75 | 46.67 | 22.33 | 2.05 | −0.42 |
| | muscle, slow twitch 2 | | | | | |
| AA104459 | ESTs, Highly similar to EUKARYOTIC | 17.00 | 39.33 | 15.67 | 2.31 | −1.33 |
| | INITIATION FACTOR 4 GAMMA | | | | | |
| | [Oryctolagus cuniculus] | | | | | |
| M29325 | Mouse L1Md-9 repetitive sequence | 13.75 | 38.00 | 12.67 | 2.76 | −1.08 |
| | (EXTRACTED 3′UTR) | | | | | |
| W10739 | regulator of G-protein signaling 2 | 10.00 | 20.67 | 10.67 | 2.07 | 0.67 |
| S68108 | SWI/SNF related, matrix associated, actin | 12.50 | 29.00 | 16.00 | 2.32 | 3.50 |
| | dependent regulator of chromatin, | | | | | |
| | subfamily a, member 4 | | | | | |
| U10115 | dishevelled, dsh homolog (Drosophila) | 22.00 | 48.67 | 21.67 | 2.21 | −0.33 |
| U25096 | Kruppel-like factor 2 (lung) | 10.00 | 33.33 | 14.33 | 3.33 | 4.33 |
| U27455 | serine palmitoyltransferase, long chain | 2.00 | 62.00 | 22.00 | 2.82 | 0.00 |
| | base subunit 2 | | | | | |
| U29173 | lymphotoxin B receptor | 16.00 | 41.33 | 19.00 | 2.58 | 3.00 |
| U37501 | laminin, alpha 5 | 10.00 | 20.33 | 10.00 | 2.03 | 0.00 |
| U54638 | rhotekin | 10.00 | 26.67 | 14.67 | 2.67 | 4.67 |
| U66887 | RAD50 homolog (S. cerevisiae) | 10.00 | 20.33 | 13.33 | 2.03 | 3.33 |
| U84411 | protein tyrosine phosphatase 4a1 | 35.25 | 77.67 | 39.67 | 2.20 | 4.42 |
| W10325 | ESTs, Moderately similar to unnamed | 13.00 | 41.33 | 13.00 | 3.18 | 0.00 |
| | protein product [H. sapiens] | | | | | |
| W64108 | ESTs, Weakly similar to A57514 RNA | 10.75 | 29.33 | 13.67 | 2.73 | 2.92 |
| | helicase HEL117 - rat [R. norvegicus] | | | | | |
| W83347 | IQ motif containing GTPase activating | 15.75 | 38.67 | 13.67 | 2.46 | −2.08 |
| | protein 1 | | | | | |
| X16670 | carbon catabolite repression 4 homolog (S. | 42.00 | 86.67 | 45.00 | 2.06 | 3.00 |
| | cerevisiae) | | | | | |
| X60831 | transcription factor UBF | 12.00 | 24.00 | 14.00 | 2.00 | 2.00 |
| X61800 | CCAAT/enhancer binding protein (C/EBP), | 10.00 | 29.00 | 11.33 | 2.90 | 1.33 |
| | delta | | | | | |
| X64414 | low density lipoprotein receptor | 15.50 | 36.00 | 13.00 | 2.32 | −2.50 |
| X65635 | melanocortin 1 receptor | 16.50 | 36.67 | 21.00 | 2.22 | 4.50 |
| X97490 | phospholipase c neighboring | 10.50 | 24.67 | 10.00 | 2.35 | −0.50 |
| X99592 | paired box gene 8 | 29.50 | 65.67 | 31.00 | 2.23 | 1.50 |
| D87661 | tyrosirie 3-monooxygenase/tryptophan | 10.50 | 27.33 | 12.00 | 2.60 | 1.50 |
| | 5-monooxygenase activation protein, eta | | | | | |
| | polypeptide | | | | | |
| W50898 | ma23g03.r1 Mus musculus cDNA, 5′end | 15.75 | 31.67 | 17.67 | 2.01 | 1.92 |
| W20873 | ESTs, Highly similar to INTERFERON- | 10.00 | 34.67 | 13.33 | 3.47 | 3.33 |
| | INDUCIBLE PROTEIN [Rattus norvegicus] | | | | | |
| W18503 | Mus musculus cytoplasmic dynein heavy | 12.25 | 31.67 | 13.00 | 2.59 | 0.75 |
| | chain mRNA, complete cds | | | | | |
| W11954 | Mus musculus cytoplasmic dynein heavy | 12.75 | 34.67 | 13.33 | 2.72 | 0.58 |
| | chain mRNA, complete cds | | | | | |
| W08057 | mb37e05.r1 Mus musculus cDNA, 5′end | 10.00 | 59.00 | 11.00 | 5.90 | 1.00 |
| C76523 | Mouse 3,5-dpc blastocyst cDNA Mus | 11.50 | 40.33 | 13.67 | 3.51 | 2.17 |
| | musculus cDNA clone J0012E07 3′, mRNA | | | | | |
| | sequence. | | | | | |
| C75983 | Mouse 3,5-dpc blastocyst cDNA Mus | 14.50 | 73.33 | 16.33 | 5.06 | 1.83 |
| | musculus cDNA clone J0001E09 3′ similar | | | | | |
| | to Unannotatable data, mRNA sequence. | | | | | |
| AA606926 | ESTs, Moderately similar to 153908 major | 15.25 | 46.00 | 13.33 | 3.02 | −1.92 |
| | vault protein - rat [R. norvegicus] | | | | | |
| AA197973 | ESTs, Weakly similar to A34337 propionyl-CoA | 46.00 | 21.67 | 41.33 | 0.47 | −4.67 |
| | carboxylase [R. norvegicus] | | | | | |
| AA172851 | ESTs, Highly similar to LEUCINE-RICH | 10.00 | 58.33 | 10.67 | 5.83 | 0.67 |
| | ALPHA-2-GLYCOPROTEIN [Homo | | | | | |
| | sapiens] | | | | | |
| AA168865 | ms38c08.r1 Mus musculus cDNA, 5′ end | 11.25 | 37.33 | 11.67 | 3.32 | 0.42 |
| | mi14h12.r1 Soares mouse p3NMF19.5 | | | | | |
| AA028657 | Mus musculus cDNA clone 463559 5′ | 28.75 | 79.00 | 31.67 | 2.75 | 2.92 |
| AA004011 | ESTs, Weakly similar to CG9591 gene | 10.00 | 24.33 | 10.33 | 2.43 | 0.33 |
| | product [D. melanogaster] | | | | | |
| AA003358 | ESTs, Moderately similar to T00076 | 20.50 | 66.33 | 18.67 | 3.24 | −1.83 |
| | hypothetical protein KIAA0462 - human | | | | | |
| | [H. sapiens] | | | | | |
| AA002653 | ESTs, Highly similar to KIAA0169 protein | 12.25 | 40.00 | 14.33 | 3.27 | 2.08 |
| | [H. sapiens] | | | | | |
| AA396029 | signal transducer and activator of | 10.00 | 34.00 | 11.00 | 3.40 | 1.00 |
| | transcription 3 | | | | | |
| M73696 | solute carrier family 20, member 1 | 10.00 | 31.67 | 14.00 | 3.17 | 4.00 |
| W41501 | Harvey rat sarcoma oncogene, subgroup R | 10.25 | 25.67 | 10.00 | 2.50 | −0.25 |
| X67141 | parvalbumin | 28.00 | 11.00 | 24.33 | 0.39 | −3.67 |
| AA108330 | phosphoprotein enriched in astrocytes 15 | 11.50 | 51.33 | 13.00 | 4.46 | 1.50 |
| AA104744 | mitogen activated protein kinase 1 | 10.00 | 23.00 | 10.00 | 2.30 | 0.00 |
| | potassium inwardly rectifying channel, | | | | | |
| D50581 | subfamily J, member 11 | 10.50 | 31.00 | 15.00 | 2.95 | 4.50 |
| W09701 | Jun oncogene | 16.25 | 32.67 | 13.67 | 2.01 | −2.58 |
| AA034638 | heat shock protein, 25 kDa | 10.00 | 29.67 | 10.00 | 2.97 | 0.00 |
| AA038607 | heat shock protein, 25 kDa | 12.50 | 52.00 | 15.33 | 4.16 | 2.83 |
| U29055 | guanine nucleotide binding protein, beta 1 | 12.00 | 26.00 | 15.67 | 2.17 | 3.67 |
| W10995 | ectonucleoside triphosphate | 11.00 | 22.67 | 12.00 | 2.06 | 1.00 |
| | diphosphohydrolase 2 | | | | | |
| W98531 | eukaryotic translation elongation factor 2 | 11.50 | 37.33 | 13.33 | 3.25 | 1.83 |
| AA666918 | IQ motif containing GTPase activating | 11.75 | 31.33 | 13.00 | 2.67 | 1.25 |
| | protein 1 | | | | | |
| AA472016 | DNA segment, Chr 5, Wayne State | 39.75 | 18.67 | 36.67 | 0.47 | −3.08 |
| | University 31, expressed | | | | | |
| U49430 | ceruloplasmin | 20.50 | 157.67 | 22.33 | 7.69 | 1.83 |
| Z19543 | calponin 2 | 15.25 | 35.33 | 17.00 | 2.32 | 1.75 |
| AB000713 | claudin 4 | 16.00 | 107.33 | 21.00 | 6.71 | 5.00 |
| X62600 | CCAAT/enhancer binding protein (C/EBP), | 10.00 | 27.33 | 10.00 | 2.73 | 0.00 |
| | beta | | | | | |
| X13333 | CD14 antigen | 25.50 | 95.33 | 28.67 | 3.74 | 3.17 |
| X04120 | carbon catabolite repression 4 homolog (S. | 50.00 | 134.67 | 53.33 | 2.69 | 3.33 |
| | cerevisiae) | | | | | |
| X80638 | aplysia ras-related homolog 9 (RhoC) | 47.00 | 147.33 | 50.33 | 3.13 | 3.33 |
| W98864 | annexin A5 | 12.00 | 30.33 | 14.00 | 2.53 | 2.00 |
| D67076 | a disintegrin-like and metalloprotease | 10.00 | 46.33 | 10.33 | 4.63 | 0.33 |
| | (reprolysin type) with thrombospondin type | | | | | |
| | 1 motif, 1 | | | | | |
| AA444568 | apoptotic chromatin condensation inducer | 10.00 | 33.00 | 10.00 | 3.30 | 0.00 |
| | in the nucleus | | | | | |

TABLE 8
Genes Abnormally Expressed Prior to Onset of Nephritis

Accession Nos.
X52634
AA268913
aa277082
L31958
Msa.30568
aa597269
aa277082
U12473
aa683909
Msa.27790.0
c75983
D84391
Msa.43183.0
N28179
j00544
c76162
Msa.383.0
ET62448
ET63281
Msa.2529.0
ET62053
Msa.29071.0

What is claimed is:

1. A method of diagnosing a subject with systemic lupus erythematosus, the method comprising the step of comparing:
a) a level of expression of a marker in a sample from the subject, wherein the marker is selected from the group consisting of markers listed in Tables 1 and 3-8, and
b) a normal level of expression of the marker in a control sample,
wherein a substantial difference between the level of expression of the marker in the sample from the subject and the normal level is an indication that the subject is afflicted with systemic lupus erythematosus.

2. The method of claim 1, wherein the marker corresponds to a transcribed polynucleotide or a portion thereof.

3. The method of claim 2, wherein the sample is collected from kidney tissue.

4. The method of claim 1, wherein the control sample is from a non-diseased subject and the substantial difference is a factor of at least about 2.

5. The method of claim 1, wherein the control sample is from non-involved tissue of the subject and the substantial difference is a factor of at least about 2.

6. The method of claim 1, wherein the control sample is from non-involved tissue of the subject and the substantial difference is a factor of at least about 5.

7. The method of claim 1, wherein the level of expression of the marker in the sample is assessed by detecting the presence in the sample of a protein corresponding to the marker.

8. The method of claim 7, wherein the presence of the protein is detected using a reagent which specifically binds with the protein.

9. The method of claim 8, wherein the reagent comprises an antibody or fragments thereof.

10. The method of claim 1, wherein the marker is selected from the markers listed in Tables 3-4.

11. The method of claim 1, wherein the marker is selected from the markers which are normalized by rapamycin as listed in Table 5.

12. The method of claim 1, wherein the marker is selected from the markers listed in Table 7.

13. The method of claim 1, wherein the marker is selected from the markers listed in Table 8.

14. The method of claim 1, wherein the level of expression of the marker in the sample is assessed by detecting the presence in the sample of a transcribed polynucleotide or portion thereof, wherein the transcribed polynucleotide comprises the marker.

15. The method of claim 14, wherein the transcribed polynucleotide is a mRNA.

16. The method of claim 14, wherein the transcribed polynucleotide is a cDNA.

17. The method of claim 1, wherein the level of expression of the marker in the sample is assessed by detecting the presence in the sample of a transcribed polynucleotide or a portion thereof which hybridizes with a labeled probe under stringent conditions, wherein the transcribed polynucleotide comprises the marker.

18. A method of diagnosing a subject with systemic lupus erythematosus, the method comprising the step of comparing:
a) a level of expression in a sample of the subject of each of a panel of markers independently selected from the markers listed in Tables 1 and 3-8, and
b) a normal level of expression of the panel of markers obtained from a control sample,
wherein the level of expression of the panel of markers is substantially different, relative to the corresponding normal level of expression of the panel of markers, indicates that the subject is afflicted with systemic lupus erythematosus.

19. The method of claim 18, wherein the panel of markers comprises at least 5 markers.

20. The method of claim 18, wherein the control sample is from a non-diseased subject.

21. The method of claim 18, wherein the control sample is from non-involved tissue of the subject.

22. The method of claim 18, wherein the panel of markers comprises markers listed in Table 8.

23. A method for monitoring the progression of systemic lupus erythematosus in a subject, the method comprising the steps of:
a) detecting in a subject sample at a first point in time, a level of expression of at least one marker, wherein the at least one marker is selected from the group consisting of markers listed in Tables 1 and 3-8;
b) repeating step a) at a subsequent point in time with the at least one marker;
c) detecting a substantial difference between the levels of expression detected in steps a) and b);
wherein the substantial difference between the levels of expression indicates that the subject has progressed to a different stage of systemic lupus erythematosus.

24. The method of claim 23, wherein at least 5 markers are selected from the group of markers listed in Tables and 3-8.

25. The method of claim 23, wherein the at least one marker corresponds to a transcribed polynucleotide or portion thereof.

26. The method of claim 25, wherein the samples are collected from kidney tissue.

27. A method of assessing the efficacy of a test compound for inhibiting systemic lupus erythematosus in a subject, the method comprising the step of comparing:
a) expression of a marker in a first sample obtained from the subject and exposed to the test compound, wherein the marker is selected from the group consisting of markers listed in Tables 1 and 3-8, and
b) expression of the same marker in a second sample obtained from the subject, wherein the second sample is not exposed to the test compound,
wherein a substantially different level of expression of the marker in the first sample, relative to the second sample, is an indication that the test compound is efficacious for inhibiting systemic lupus erythematosus in the subject.

28. The method of claim 27, wherein the first and second samples are portions of a single sample obtained from the subject.

29. The method of claim 28, wherein the level of expression in the first sample approximates the level of expression in a control sample.

30. A method of assessing the efficacy of a therapy for inhibiting systemic lupus erythematosus in a subject, the method comprising the steps of comparing:
a) expression of a marker in a first sample obtained from the subject prior to providing at least a portion of the therapy to the subject, wherein the marker is selected from the group consisting of markers listed in Tables 1 and 3-8, and
b) expression of the marker in a second sample following provision of the portion of the therapy,
wherein a substantially different level of expression of the marker in the second sample, relative to the first sample, is an indication that the test compound is efficacious for inhibiting systemic lupus erythematosus in the subject.

31. The method of claim 30, wherein a substantially similar level of expression in the second sample, relative to the control sample, is an additional indication that the test compound is efficacious for inhibiting systemic lupus erythematosus.

32. The method of claim 31, further comparing expression of the marker in a control sample, wherein expression of the marker in the second sample is similar to expression of the marker in the control sample.

33. A method of screening test compounds for inhibitors of systemic lupus erythematosus, the method comprising the steps of:
a) obtaining a sample comprising cells from the subject;
b) separately maintaining aliquots of the sample in the presence of a plurality of test compounds;
c) comparing the expression levels of a marker in each of the aliquots, wherein the marker is selected from the group consisting of markers listed in Tables 1 and 3-8; and
d) selecting one of the test compounds which induces a substantially different level of expression of the marker in the aliquot containing that test compound, relative to other test compounds.

34. The method of claim 33, wherein the substantially different level of expression is a substantially lower level of expression.

35. The method of claim 34, wherein the substantially different level of expression is a substantially enhanced level of expression.

36. A kit for diagnosing a subject with systemic lupus erythematosus, the kit comprising reagents for assessing expression of a marker selected from the group consisting of markers listed in Tables 1 and 3-8.

37. A kit for diagnosing systemic lupus erythematosus in a subject, the kit comprising a nucleic acid probe wherein the probe specifically binds with a transcribed polynucleotide corresponding to a marker selected from the group consisting of markers listed in Tables 1 and 3-8.

38. A kit for assessing the suitability of each of a plurality of compounds for inhibiting progression of systemic lupus erythematosus in a subject, the kit comprising:
a) the plurality of compounds; and
b) a reagent for assessing expression of a marker selected from the group consisting of markers listed in Tables 1 and 3-8.

39. A kit for diagnosing systemic lupus erythematosus in a subject, the kit comprising an antibody, wherein the antibody specifically binds with a protein corresponding to a marker selected from the group consisting of markers listed in Tables 1 and 3-8.

40. A method of modulating a level of expression of a marker selected from the markers listed in Tables 1 and 3-8, the method comprising providing to diseased cells of a subject an antisense oligonucleotide complementary to a polynucleotide corresponding to the marker.

41. A method of modulating a level of expression of a marker selected from the markers listed in Tables 1 and 3-8, the method comprising providing to diseased cells of a subject a protein.

42. The method of claim 41, wherein the protein is provided to the cells by providing a vector comprising a polynucleotide encoding the protein to the cells.

43. A method of modulating a level of expression of a marker selected from the markers listed in Tables 1 and 3-8, the method comprising providing to diseased cells of a subject an antibody.

44. The method according to claim 43, wherein the method further comprises a therapeutic moiety conjugated to the antibody.

45. The method according to claim 43, wherein the method further comprises providing to the diseased cells an additional antibody.

46. A method of localizing a therapeutic moiety to diseased tissue comprising exposing the tissue to an antibody which is specific to a protein encoded from a marker listed in Tables 1 and 3-8.

47. A method of localizing a therapeutic moiety to diseased tissue comprising exposing the tissue to a plurality of antibodies which are each specific to a protein encoded from a marker listed in Tables 1 and 3-8.

48. A method of screening for a test compound capable of modulating the activity of a protein encoded from a marker listed in Tables 1 and 3-8, the method comprising combining the protein and the test compound, and determining the effect of the test compound on the therapeutic efficacy of the protein.

49. A method of screening for a bioactive agent capable of interfering with the binding of a protein encoded from a marker listed in Tables 1 and 3-8 and an antibody which binds to the protein, the method comprising:
a) combining the protein, a bioactive agent and an antibody which binds to the protein; and
b) determining the binding of the protein or fragment thereof and the antibody.

50. An antibody which specifically binds to a protein encoded from a marker listed in Tables 1 and 3-8.

51. The antibody of claim 48, wherein the antibody is a monoclonal antibody.

52. The antibody of claim 49, wherein the antibody is a humanized antibody.

53. A peptide encoded from markers listed in Tables 1 and 3-8.

54. A composition comprising the peptide of claim 51.

55. A composition capable of modulating an immune response in a subject, the composition comprising a protein encoded from a marker listed in Tables 1 and 3-8, and a pharmaceutically acceptable carrier.

56. A biochip comprising a panel of markers selected from the group of markers listed in Tables 1 and 3-8.

57. The biochip of claim 56, wherein the markers are selected for subjects suspected of having systemic lupus nephritis, wherein the subjects display signs nephritis.

58. The biochip of claim 56, wherein the markers are selected for subjects suspected of having systemic lupus nephritis, wherein the subjects display signs of facial lesions.
